SEQUENCE LISTING (69 sequences)

SEQ ID NO: 1 - DNA, 20 bp, Artificial Sequence
  qRT-ALB_Fw_P1
cttcctgggctcgtttttgt

SEQ ID NO: 2 - DNA, 20 bp, Artificial Sequence
  qRT-ALB_Rv_P1
acagcattcctccagtgtgg

SEQ ID NO: 3 - DNA, 20 bp, Artificial Sequence
  qRT-CSN1S1_Fw_P1
agtgttgagcagaagcacat

SEQ ID NO: 4 - DNA, 20 bp, Artificial Sequence
  qRT-CSN1S1_Rv_P1
gttgggcatggattccctct

SEQ ID NO: 5 - DNA, 20 bp, Artificial Sequence
  qRT-CSN1S2_Fw_P1
gctgttgcccttgcaaagaa

SEQ ID NO: 6 - DNA, 20 bp, Artificial Sequence
  qRT-CSN1S2_Rv_P1
tccttgcagaatgtggagca

SEQ ID NO: 7 - DNA, 23 bp, Artificial Sequence
  qRT-CSN2_Fw_P1
cagtgaggaatctattacacgca

SEQ ID NO: 8 - DNA, 20 bp, Artificial Sequence
  qRT-CSN2_Rv_P1
tgggcaaaggggtggatttt

SEQ ID NO: 9 - DNA, 20 bp, Artificial Sequence
  qRT-CSN3_Fw_P1
accattgctagtggtgagcc

SEQ ID NO: 10 - DNA, 20 bp, Artificial Sequence
  qRT-CSN3_Rv_P1
tgtgttgatctcaggtgggc

SEQ ID NO: 11 - DNA, 20 bp, Artificial Sequence
  qRT-LALBA_Fw_P1
tctctgctcctggtaggcat

SEQ ID NO: 12 - DNA, 20 bp, Artificial Sequence
  qRT-LALBA_Rv_P1
ggcaaactgacacctccgta

SEQ ID NO: 13 - DNA, 20 bp, Artificial Sequence
  qRT-LACB_Fw_P1
agatccctgcggtgttcaag

SEQ ID NO: 14 - DNA, 20 bp, Artificial Sequence
  qRT-LACB_Rv_P1
ggctcagcactgttctccat

SEQ ID NO: 15 - DNA, 40 bp, Artificial Sequence
  ALB Fw
gcgccgtctcgctcgaatgaagtgggtgacttttatttct

SEQ ID NO: 16 - DNA, 40 bp, Artificial Sequence
  ALB Rev
gcgccgtctcgctcaaagcttaggctaaggctgtttgagt

SEQ ID NO: 17 - DNA, 39 bp, Artificial Sequence
  CSN1S1Fw
gcgccgtctcgctcgaatgaaacttctcatccttacctg

SEQ ID NO: 18 - DNA, 39 bp, Artificial Sequence
  CSN1S1 Rev
gcgccgtctcgctcaaagctcaccacagtggcatagtag

SEQ ID NO: 19 - DNA, 40 bp, Artificial Sequence
  CSN1S2 Fw
gcgccgtctcgctcgaatgaagttcttcatctttacctgc

SEQ ID NO: 20 - DNA, 40 bp, Artificial Sequence
  CSN1S2 Rev
gcgccgtctcgctcaaagcttaaaggtacctcacataggg

SEQ ID NO: 21 - DNA, 36 bp, Artificial Sequence
  CSN2 Fw
gcgccgtctcgctcgaatgaaggtcctcatccttgc

SEQ ID NO: 22 - DNA, 42 bp, Artificial Sequence
  CSN2 Rev
gcgccgtctcgctcaaagcttagacaataatagggaagggtc

SEQ ID NO: 23 - DNA, 41 bp, Artificial Sequence
  CSN3Fw
gcgccgtctcgctcgaatgatgaagagttttttcctagttg

SEQ ID NO: 24 - DNA, 40 bp, Artificial Sequence
  CSN3 Rev
gcgccgtctcgctcaaagcttagaccgcagttgaagtaac

SEQ ID NO: 25 - DNA, 36 bp, Artificial Sequence
  LACB Fw
gcgccgtctcgctcgaatgaagtgcctcctgcttgc

SEQ ID NO: 26 - DNA, 39 bp, Artificial Sequence
  LACB Rev
gcgccgtctcgctcaaagcctagatgtggcactgctcct

SEQ ID NO: 27 - DNA, 37 bp, Artificial Sequence
  LALBA Fw
gcgccgtctcgctcgaatgatgtcctttgtctctctg

SEQ ID NO: 28 - DNA, 39 bp, Artificial Sequence
  LALBA Rev
gcgccgtctcgctcaaagctcacaacttctcacagagcc

SEQ ID NO: 29 - DNA, 1824 bp, Bos taurus
atgaagtgggtgacttttatttctcttctccttctcttcagctctgcttattccaggggtgtgtttcgtcgagatacacacaagagtgagattgctcatcggtttaaagatttgggagaagaacattttaaaggcctggtactgattgccttttctcagtatctccagcagtgtccatttgatgagcatgtaaaattagtgaacgaactaactgagtttgcaaaaacatgtgttgctgatgagtcccatgccggctgtgaaaagtcacttcacactctctttggagatgaattgtgtaaagttgcatcccttcgtgaaacctatggtgacatggctgactgctgtgcgaaacaagagcctgaaagaaatgaatgcttcctgagccacaaagatgatagcccagacctccctaaattgaaaccagaccccaatactttgtgtgatgagtttaaggcagatgaaaagaagttttggggaaaatacctatacgaaattgctagaagacatccctacttttatgcaccagaactcctttactatgctaataaatataatggagtttttcaagaatgctgccaagctgaagataaaggtgcctgcctgctaccaaagattgaaactatgagagaaaaagtactgacttcatctgccagacagagactcaggtgtgccagtattcaaaaatttggagaaagagctttaaaagcatggtcagtagctcgcctgagccagaaatttcccaaggctgagtttgtagaagttaccaagctagtgacagatctcacaaaagtccacaaggaatgctgccatggtgacctacttgaatgcgcagatgacagggcagatcttgccaagtacatatgtgataatcaagatacaatctccagtaaactgaaggaatgctgtgataagcctttgttggaaaaatcccactgcattgctgaggtggaaaaagatgccatacctgaaaacctgcccccattaactgctgactttgctgaagataaggatgtttgcaaaaactatcaggaagcaaaagatgccttcctgggctcgtttttgtatgaatattcaagaaggcatcctgaatatgctgtctcagtgctattgagacttgccaaggaatatgaagccacactggaggaatgctgtgccaaagatgatccacatgcatgctattccacagtgtttgacaaacttaagcatcttgtggatgagcctcagaatttaatcaaacaaaactgtgaccaattcgaaaaacttggagagtatggattccaaaatgagctcatagttcgttacaccaggaaagtaccccaagtgtcaactccaactctcgtggaggtttcaagaagcctaggaaaagtgggtactaggtgttgtacaaagccggaatcagaaagaatgccctgtgctgaagactatctgagcttgatcctgaaccggttgtgcgtgctgcatgagaagacaccagtgagtgaaaaagtcaccaagtgctgcacagagtcattggtgaacagacggccatgtttctctgctctgacacctgatgaaacatatgtacccaaagcctttgatgagaaattgttcaccttccatgcagatatatgcacacttcccgatactgagaaacaaatcaagaaacaaactgcacttgttgagctgttgaaacacaagcccaaggcaacagaggaacaactgaaaaccgtcatggagaattttgtggcttttgtaggcaagtgctgtgcagctgatgacaaagaggcctgctttgctgtggagggtccaaaacttgttgtttcaactcaaacagccttagcctaa

SEQ ID NO: 30 - DNA, 645 bp, Bos taurus
atgaaacttctcatccttacctgtcttgtggctgttgctcttgccaggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattctctgacatccctaatcccattggctctgagaacagtgaaaagactactatgccactgtggtga

SEQ ID NO: 31 - DNA, 645 bp, Bos taurus; identical to SEQ ID NO: 30

SEQ ID NO: 32 - DNA, 675 bp, Bos taurus
atgaaggtcctcatccttgcctgcctggtggctctggcccttgcaagagagctggaagaactcaatgtacctggtgagattgtggaaagcctttcaagcagtgaggaatctattacacgcatcaataagaaaattgagaagtttcagagtgaggaacagcagcaaacagaggatgaactccaggataaaatccacccctttgcccagacacagtctctagtctatcccttccctgggcccatccataacagcctcccacaaaacatccctcctcttactcaaacccctgtggtggtgccgcctttccttcagcctgaagtaatgggagtctccaaagtgaaggaggctatggctcctaagcacaaagaaatgcccttccctaaatatccagttgagccctttactgaaaggcagagcctgactctcactgatgttgaaaatctgcaccttcctctgcctctgctccagtcttggatgcaccagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccaggagcctgtactcggtcctgtccggggacccttccctattattgtctaa

SEQ ID NO: 33 - DNA, 573 bp, Bos taurus
atgatgaagagttttttcctagttgtgactatcctggcattaaccctgccatttttgggtgcccaggagcaaaaccaagaacaaccaatacgctgtgagaaagatgaaagattcttcagtgacaaaatagccaaatatatcccaattcagtatgtgctgagtaggtatcctagttatggactcaattactaccaacagaaaccagttgcactaattaataatcaatttctgccatacccatattatgcaaagccagctgcagttaggtcacctgcccaaattcttcaatggcaagttttgtcaaatactgtgcctgccaagtcctgccaagcccagccaactaccatggcacgtcacccacacccacatttatcatttatggccattccaccaaagaaaaatcaggataaaacagaaatccctaccatcaataccattgctagtggtgagcctacaagtacacctaccatcgaagcagtagagagcactgtagctactctagaagcttctccagaagttattgagagcccacctgagatcaacacagtccaagttacttcaactgcggtctaa

SEQ ID NO: 34 - DNA, 537 bp, Bos taurus
atgaagtgcctcctgcttgccctggccctcacttgtggcgcccaggccctcattgtcacccagaccatgaagggcctggatatccagaaggtggcggggacttggtactccttggccatggcggccagcgacatctccctgctggacgcccagagtgcccccctgagagtgtatgtggaggagctgaagcccacccctgagggcgacctggagatcctgctgcagaaatgggagaacggtgagtgtgctcagaagaagatcattgcagaaaaaaccaagatccctgcggtgttcaagatcgatgccttgaatgagaacaaagtccttgtgctggacaccgactacaaaaagtacctgctcttctgcatggagaacagtgctgagcccgagcaaagcctggcctgccagtgcctggtcaggaccccggaggtggacgacgaggccctggagaaattcgacaaagccctcaaggccctgcccatgcacatccggctgtccttcaacccaacccagctggaggagcagtgccacatctag

SEQ ID NO: 35 - DNA, 429 bp, Bos taurus
atgatgtcctttgtctctctgctcctggtaggcatcctattccatgccacccaggctgaacagttaacaaaatgtgaggtgttccgggagctgaaagacttgaagggctacggaggtgtcagtttgcctgaatgggtctgtaccacgtttcataccagtggttatgacacacaagccatagtacaaaacaatgacagcacagaatatggactcttccagataaataataaaatttggtgcaaagacgaccagaaccctcactcaagcaacatctgtaacatctcctgtgacaagttcctggatgatgatcttactgatgacattatgtgtgtcaagaagattctggataaagtaggaattaactactggttggcccataaagcactctgttctgagaagctggatcagtggctctgtgagaagttgtga

SEQ ID NO: 36 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCAKQEPERNECFLSHKDDSPDLPKLKPDPNTLCDEFKADEKKFWGKYLYEIARRHPYFYAPELLYYANKYNGVFQECCQAEDKGACLLPKIETMREKVLTSSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNELIVRYTRKVPQVSTPTLVEVSRSLGKVGTRCCTKPESERMPCAEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVGKCCAADDKEACFAVEGPKLVVSTQTALA

SEQ ID NO: 37 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 38 - protein, 222 aa, Bos taurus
MKFFIFTCLLAVALAKNTMEHVSSSEESIISQETYKQEKNMDINPSKENLCSTFCKEVVRNANEEEYSIGSSSEESAEVATEEVKITVDDKHYQKALNEINQFYRKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 39 - protein, 224 aa, Bos taurus
MKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 40 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 41 - protein, 178 aa, Bos taurus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 42 - protein, 142 aa, Bos taurus
MMSFVSLLLVGILFHATQAEQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 43 - DNA, 8192 bp, Artificial Sequence
  pDGB-alpha1 ALB (Serum Albumin)
cgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacgaattcgtctcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaagg

SEQ ID NO: 44 - DNA, 8192 bp, Artificial Sequence
  pDGB-alpha2 CSN1S1 (alpha-S1-Casein)
cgcttgagacgaagctttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacaagcttcgtctcagtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaa

SEQ ID NO: 45 - DNA, 8192 bp, Artificial Sequence; identical to SEQ ID NO: 43
  pDGB-alpha1 CSN1S2 (alpha-S2-Casein)

SEQ ID NO: 46 - DNA, 8192 bp, Artificial Sequence; identical to SEQ ID NO: 44
  pDGB-alpha2 CSN2 (beta-Casein)

SEQ ID NO: 47 - DNA, 8192 bp, Artificial Sequence; identical to SEQ ID NO: 43
  pDGB-alpha1 CSN3 (kappa-Casein)

SEQ ID NO: 48 - DNA, 8192 bp, Artificial Sequence; identical to SEQ ID NO: 44
  pDGB-alpha2 LALABA (alpha-lactalbumin; LALBA)

SEQ ID NO: 49 - DNA, 8192 bp, Artificial Sequence; identical to SEQ ID NO: 44
  pDGB-alpha2 LGB (beta-lactoglobulin; LACB)

SEQ ID NO: 50 - DNA, 37928 bp, Artificial Sequence
  pDGB-omega1  Seven Bovine Milk Genes
ttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatgaagtgggtgacttttatttctcttctccttctcttcagctctgcttattccaggggtgtgtttcgtcgagatacacacaagagtgagattgctcatcggtttaaagatttgggagaagaacattttaaaggcctggtactgattgccttttctcagtatctccagcagtgtccatttgatgagcatgtaaaattagtgaacgaactaactgagtttgcaaaaacatgtgttgctgatgagtcccatgccggctgtgaaaagtcacttcacactctctttggagatgaattgtgtaaagttgcatcccttcgtgaaacctatggtgacatggctgactgctgtgcgaaacaagagcctgaaagaaatgaatgcttcctgagccacaaagatgatagcccagacctccctaaattgaaaccagaccccaatactttgtgtgatgagtttaaggcagatgaaaagaagttttggggaaaatacctatacgaaattgctagaagacatccctacttttatgcaccagaactcctttactatgctaataaatataatggagtttttcaagaatgctgccaagctgaagataaaggtgcctgcctgctaccaaagattgaaactatgagagaaaaagtactgacttcatctgccagacagagactcaggtgtgccagtattcaaaaatttggagaaagagctttaaaagcatggtcagtagctcgcctgagccagaaatttcccaaggctgagtttgtagaagttaccaagctagtgacagatctcacaaaagtccacaaggaatgctgccatggtgacctacttgaatgcgcagatgacagggcagatcttgccaagtacatatgtgataatcaagatacaatctccagtaaactgaaggaatgctgtgataagcctttgttggaaaaatcccactgcattgctgaggtggaaaaagatgccatacctgaaaacctgcccccattaactgctgactttgctgaagataaggatgtttgcaaaaactatcaggaagcaaaagatgccttcctgggctcgtttttgtatgaatattcaagaaggcatcctgaatatgctgtctcagtgctattgagacttgccaaggaatatgaagccacactggaggaatgctgtgccaaagatgatccacatgcatgctattccacagtgtttgacaaacttaagcatcttgtggatgagcctcagaatttaatcaaacaaaactgtgaccaattcgaaaaacttggagagtatggattccaaaatgagctcatagttcgttacaccaggaaagtaccccaagtgtcaactccaactctcgtggaggtttcaagaagcctaggaaaagtgggtactaggtgttgtacaaagccggaatcagaaagaatgccctgtgctgaagactatctgagcttgatcctgaaccggttgtgcgtgctgcatgagaagacaccagtgagtgaaaaagtcaccaagtgctgcacagagtcattggtgaacagacggccatgtttctctgctctgacacctgatgaaacatatgtacccaaagcctttgatgagaaattgttcaccttccatgcagatatatgcacacttcccgatactgagaaacaaatcaagaaacaaactgcacttgttgagctgttgaaacacaagcccaaggcaacagaggaacaactgaaaaccgtcatggagaattttgtggcttttgtaggcaagtgctgtgcagctgatgacaaagaggcctgctttgctgtggagggtccaaaacttgttgtttcaactcaaacagccttagcctaagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatgaaacttctcatccttacctgtcttgtggctgttgctcttgccaggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattctctgacatccctaatcccattggctctgagaacagtgaaaagactactatgccactgtggtgagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatgaagttcttcatctttacctgccttttggctgttgcccttgcaaagaatacgatggaacatgtctcctccagtgaggaatctatcatctcccaggaaacatataagcaggaaaagaatatggacattaatcccagcaaggagaacctttgctccacattctgcaaggaagttgtaaggaacgcaaatgaagaggaatattctatcggctcatctagtgaggaatctgctgaagttgccacagaggaagttaagattactgtggacgataagcactaccagaaagcactgaatgaaatcaatcagttttatcggaagttcccccagtatctccagtatctgtatcaaggtccaattgttttgaacccatgggatcaggttaagagaaatgctgttcccattactcccactctgaacagagagcagctctccaccagtgaggaaaattcaaagaagaccgttgacatggaatcaacagaagtattcactaagaaaactaaactgactgaagaagaaaagaatcgcctaaattttctgaaaaaaatcagccagcgttaccagaaattcgccttgccccagtatctcaaaactgtttatcagcatcagaaagctatgaagccatggattcaacctaagacaaaggttattccctatgtgaggtacctttaagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatgaaggtcctcatccttgcctgcctggtggctctggcccttgcaagagagctggaagaactcaatgtacctggtgagattgtggaaagcctttcaagcagtgaggaatctattacacgcatcaataagaaaattgagaagtttcagagtgaggaacagcagcaaacagaggatgaactccaggataaaatccacccctttgcccagacacagtctctagtctatcccttccctgggcccatccataacagcctcccacaaaacatccctcctcttactcaaacccctgtggtggtgccgcctttccttcagcctgaagtaatgggagtctccaaagtgaaggaggctatggctcctaagcacaaagaaatgcccttccctaaatatccagttgagccctttactgaaaggcagagcctgactctcactgatgttgaaaatctgcaccttcctctgcctctgctccagtcttggatgcaccagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccaggagcctgtactcggtcctgtccggggacccttccctattattgtctaagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcatgagaccggatcctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcaccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggtagcagcgaagtcgaggcatttctgtcctggctggaacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacggatccggtctcaggagagcgatcagcttgcatgccggtcgatctagtaacatagtagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctaggggtcatcagatttcggtgacgggcaggaccggacggggcggcaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggatacgtacacggtcgactcggccgtccagtcgtaggcgttgcgtgccttccagggacccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataacgtgactcccttaattctcatgtatgatactccgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatgatgtcctttgtctctctgctcctggtaggcatcctattccatgccacccaggctgaacagttaacaaaatgtgaggtgttccgggagctgaaagacttgaagggctacggaggtgtcagtttgcctgaatgggtctgtaccacgtttcataccagtggttatgacacacaagccatagtacaaaacaatgacagcacagaatatggactcttccagataaataataaaatttggtgcaaagacgaccagaaccctcactcaagcaacatctgtaacatctcctgtgacaagttcctggatgatgatcttactgatgacattatgtgtgtcaagaagattctggataaagtaggaattaactactggttggcccataaagcactctgttctgagaagctggatcagtggctctgtgagaagttgtgagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatgatgaagagttttttcctagttgtgactatcctggcattaaccctgccatttttgggtgcccaggagcaaaaccaagaacaaccaatacgctgtgagaaagatgaaagattcttcagtgacaaaatagccaaatatatcccaattcagtatgtgctgagtaggtatcctagttatggactcaattactaccaacagaaaccagttgcactaattaataatcaatttctgccatacccatattatgcaaagccagctgcagttaggtcacctgcccaaattcttcaatggcaagttttgtcaaatactgtgcctgccaagtcctgccaagcccagccaactaccatggcacgtcacccacacccacatttatcatttatggccattccaccaaagaaaaatcaggataaaacagaaatccctaccatcaataccattgctagtggtgagcctacaagtacacctaccatcgaagcagtagagagcactgtagctactctagaagcttctccagaagttattgagagcccacctgagatcaacacagtccaagttacttcaactgcggtctaagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatgaagtgcctcctgcttgccctggccctcacttgtggcgcccaggccctcattgtcacccagaccatgaagggcctggatatccagaaggtggcggggacttggtactccttggccatggcggccagcgacatctccctgctggacgcccagagtgcccccctgagagtgtatgtggaggagctgaagcccacccctgagggcgacctggagatcctgctgcagaaatgggagaacggtgagtgtgctcagaagaagatcattgcagaaaaaaccaagatccctgcggtgttcaagatcgatgccttgaatgagaacaaagtccttgtgctggacaccgactacaaaaagtacctgctcttctgcatggagaacagtgctgagcccgagcaaagcctggcctgccagtgcctggtcaggaccccggaggtggacgacgaggccctggagaaattcgacaaagccctcaaggccctgcccatgcacatccggctgtccttcaacccaacccagctggaggagcagtgccacatctaggcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgt

SEQ ID NO: 51 - DNA, 1396 bp, Glycine max
aacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagcta

SEQ ID NO: 52 - DNA, 1398 bp, Glycine max
tacattttgagttgtttcaggttccattgccttattgctaaaactccaactaaaataacaaatagcacatgcaggtgcaaacaacacgttactctgatgaaggtgatgtgcctctagcagtctagcttatgaggctcgctgcttatcaacgattcatcattccccaagacgtgtacgcagattaaacaatggacaaaacttcaatcgattatagaataataattttaacagtgccgacttttttctgtaaacaaaaggccagaatcatatcgcacatcatcttgaatgcagtgtcgagtttggaccatttgagtacaaagccaatattgaatgatttttcgattttacatgtgtgaatcagacaaaagtgcatgcaatcacttgcaagtaaattaaggatactaatctattcctttcattttatatgctccacttttatataaaaaaatatacattattatatatgcattattaattattgcagtattatgctattggttttatggccctgctaaataacctaaatgagtctaactattgcatatgaatcaaatgaaggaagaatcatgatctaaacctgagtacccaatgcaataaaatgcgtcctattacctaaacttcaaacacacattgccatcggacgtataaattaatgcatatagattattttgagaaaagaaaacatcaaaagctctaaaacttcttttaactttgaaataagctgataaaaatacgctttaaatcaactgtgtgctgtatataagctgcaatttcacattttaccaaaccgaaacaagaatggtaacagtgaggcaaaaatttgaaaaatgtcctacttcacattcacatcaaattaattacaactaaataaataaacatcgtgattcaagcagtaatgaaagtcgaaatcagatagaatatacacgtttaacatcaattgaatttttttttaaatggatatatacaagtttactattttatatataatgaaaattcattttgtgttagcacaaaacttacagaaagagataaattttaaataaagagaattatatccaattttataatccaaaataatcaaattaaagaatattggctagatagaccggctttttcactgcccctgctggataatgaaaattcatatcaaaacaatacagaagttctagtttaataataaaaaagttggcaaactgtcattccctgttggtttttaagccaaatcacaattcaattacgtatcagaaattaatttaaaccaaatatatagctacgagggaacttcttcagtcattactagctagctcactaatcactatatatacgacatgctacaagtgaagtgaccatatcttaatttcaaatcataaaattcttccaccaagttatgggtttcct

SEQ ID NO: 53 - DNA, 1500 bp, Glycine max
attatttctgttagtacatagctaatactcaatcaacggaattagtatatggttcttcatataggagagtacttatttattctattgaattttaacatataagcataataaaatacttttggactctcgtataaagttcgattttaatctttttaataattcaatctaaatgtttaattccctcttaaatgcaaaattcagttttcgttcctttaatgtgacaccattaggtcacatgaaccggaaatgacgtggtgatcgaattatgacttgaatccattgaccacattagcatttcacctatggtcactagtatgaaggatgaaaacaagtctatttctcaaattataaatgaaaacgtttaactttaaacctgaggatccaaaaacgaattttactaaattttgaagaactaaaaaatatttaatctagtaaaacgcgtgtctatctaatataacatgcacgctcgtcatgtaatcaattaggcataaaaatagtgtttgattttttgacacattattaagtgttttatttttaagtttaaaagcattggtatcctttcataaaaggaggtaatcttatttaagtcaaggagaattattatgggaaataaaacctttttttttaaagtgtttaatataattatatactcaaaattcgatttatgattaaatctaagtgacatttaaaaaaaattagtgtgaaaataatttatatataattttgaaaaatttatcattaatttttttttataaataaatgttaatttattagtttttattataaatgtgaatagaatggattcgaagcagcaatttctctctttctccttttccatgccaaccttatatatggtgacgaactgcatatacagtaaaacagttcaaattgagaaagattttaaacatcatagtatttgatatatatcttttacagagacaattatgctgcaggagttagataagattattgtggatgtcattttcttttttaatatttaacgcattatataaaagatgatatagtatggttataaaaaaattatttaacagtttataaaacctttttttttatcttttacagtaatattatttattttatttcacatttttttcatatccttatctcatttataaaggaaattaattgtataaaaaaaatatgatgcactgaatagaatgctgatcttattgtataaggaggatagaatttgagacgcggagaatctgtagagggggaccattcagggtgcctgcaattttggtgttgttcatgtacggttgcagatataaacgaagcatagcttatgtatgaggtgtaacaaaattggaaacaatagccatgcaaggtgaagaatgtcaccaactcagaaacccttcttcattgacgtgtccctcactcactctcctctcttcactataaatcgccactcttcgtgttctccacttcaccaactccttcaaacttattaacactttccttagttcaatatggggaagc

SEQ ID NO: 54 - DNA, 1493 bp, Glycine max
aacttaatcgtatataaaaaattcaatatatgaataattctaagtgagtttttaagaaaaaataaaattagtaacgaagtaatttatatataattttgaaaaattatcactaaatttgtgatccactgttaacattaatttattcctcttgtattgaataaaatagttcagacatggtcccagtctttaatcaattattcatgcttctctgtctctcacttatataatcctgtaatccaaacattactcagatagctagatccaccgatcaatcgtatatatatacgcataaaatcgacgcctctgtattttttagactgtagcccaaattcactatccgaataaaataagggaggcacgtgtacgtaatttatatcatatgatagccatgcatatgcacacgtgcagaagagctgttaccctctatacgtgtactcaccttctcatcctctctgaatattttgagtgctcttcctagttatctagtaatgcatgaaattaaacttactaaatgtttcttcaatttaaagaaataattgtttatctgtttcaatttttttaagagaattttaaaaagataattgtttcggggagagagatataaaaaagaaaagggagaaatattaaaatgtactaaataatatgataagaaaagagagaaaaataaaagagaaaatttgtatatagttataattattcatgtaataaggattcatctctcaactgaaaatatacttaatgcagaagaaaaaatcattatttacaaacgttgagtcttgagtgggaaaagaggaggcgccgttactatacaatataagatcatagtactgacaaaatgcacagtaaaacagttcaaattgagaaggattcttaacacaccatagtatttaatatatatctttacagagacaattatgctggaggattcaggcaaagattatatattgtggatttgttttttaataattaacgcatcatatgaaagatcgatgatatatactaatggttataagaaaaatatttaacagtttctataacctttttcttttatcttttactgtaatattatttattttatttcacatttttaatcagcttatctcatttataaacgaaattgtataaaaatatacatgatgaactgaatagaacaatattgatctgatattctcatattgtataagaggatagactttgagacgcggagaatctgtaggaggggaccattcagagtgcctccaattttggtgttgttcattgtaccattgcaaatataaacgaagcatgcatgcttatgtatgaggtgtaacaaaattggaaacaatagccatgcaaggtgaagaatgtcacaaactcagcaacccttattcattgacgtgtccctcagtcactctcctctcatacctataaatcaccactcctcatgttctttccaattaccaactccttcaaacttaattattaacacttccttagttcaatatggggaagcc

SEQ ID NO: 55 - DNA, 1608 bp, Glycine max
ataattataaaattgtcactgcgttcaaaacgacaatggttttgggacaactatcattaatcgtgcattgtaaaaaggtgtgtttttagtagtggaccctcgataaattgactgtgatgattgttacatgttgttaagtctcacctataagaaaaaaactaaacatatatatagatcccaattttggggtcaggtgtatagatgaaaaaaagaaacaaatagacaaataaaaaaataaaagaaaaaaaattgatagatgtgagaaatgatgagaagagaagtgcaaataacacactctttctaacattattttactattgattaaaatttattgaaaattactatataatataaaaagtgaaactagttaaactatagtcaataattgagaatatttaaaaatttagaaaatacattacttatatttcttaaaataaaaaatataaataaaaatagaaaaaatggagtaaaatgagatagaagagaagttaggtttataaatacattagttccgcctacaatatatttaaattagctagattaatgcagtaaatttttggcatttacttgattttattttctttaaaagcattctttgtattcttcactgatggttttttttcttcatctgcattatgaattaaatcatttactttgtgtcacaattgcatttagcgaggtcatgcattggttagaccgacggtgtattatgtcatgacttaggtcttgaaggttgttggttacttattatggtccatgggtacacgcgttggttagattcgataggcaaattttgtgaacgatagaaatttatctttattaaataaaccacactatatatatatatatatatatatatatatatatatatatatatattaattcgtaatttcttttctgtctttcattttgattttcttttatggcttttatctttaaaaattttccccttctttaaaatttacaacactttataatcacaataaaataaaataatttaaaatattacataaataataacacaaatatttataaatctgaaatgacataaaataacattataatcacaaaaagtatttaataaaaataaaattacataaataaaatattgtgaaaactaagtaaaaggtatcatgcacgtaatcatatgaaaatagctttagaaaaaatatcaaggcaagtaccgcacgtacgataaatgaaaaaagattaaaaagaaatataataaataataatactaaattaatggtgaataaaatactaaaaaaataaatttataattaaataatatgtattacaaacacaaataagaaataatagtacataatattataataaatagtagtatataacatatcataaatatgtttaaaataatgataaaatattgagtttcttttagtggaactatttgtcaaaatgtgaacacctggatatgaaaaggcatcttaggtagatgatatgatgcgatagaacgtaaaagaaaaatgagaaatgttgatgagaggttaaaaatacccttcataacaagcacacatctataagtagtcttattcacccaacaacgttgcttattcacgcaactaaataagaaatgaagagtactat

SEQ ID NO: 56 - DNA, 1545 bp, Glycine max
tataaacaccactttaatttgactcggatacatgcatccataaagactacaaaaggcaaaaagagaaggaaatgagatacgaatatatgtcataagtatatataggtgacaagggcaaattaaataggttggtatttaaatgcaaaatcctatgtttgataaagaatggtatgaaaaacaggcaaagttaattgcaattcaaaggtgaacaaagcatttctttgtctacactaatggcatgtctaagtaaattattagtcttgtatctatatgtccacaagttattaattagtcttatactatcaaaaacaagttaagttgcaaatcaaacatgaacaaagcatttgtgttgtaacctacgaaaaaataccctaacatactgatacgaataatgtggcctaaattgatcgtttaccaaattacggtgctggaaaaaaaaattgctcctttaccaacaaaattaagaactgatacatcttgttttttgtcactgaagataaacacgtgatctttggcaaaacataaaggccaacaaaacaaacttgtctcatccctgaatgattcgaatgccatcgtatgcgtgtcacaaagtggaatacagcaatgaacaaatgctatcctcttgagaaaagtgaatgcagcagcagcagcagactagagtgctacaaatgcttatcctcttgagaaaagtgaatgcagcggcagcagacctgagtgctatatacaattagacacagggtctattaattgaaattgtcttattattaaatatttcgttttatattaattttttaaattttaattaaatttatatatattatatttaagacagatatatttatttgtgattataaatgtgtcactttttcttttagtccatgtattcttctattttttcaatttaactttttatttttatttttaagtcactcttgatcaagaaaacattgttgacataaaactattaacataaaattatgttaacatgtgataacatcatattttactaatataacgtcgcattttaacgtttttttaacaaatatcgactgtaagagtaaaaatgaaatgtttgaaaaggttaattgcatactaactattttttttcctataagtaatcttttttgggatcaattgtatatcattgagatacgatattaaatatgggtaccttttcacaaaacctaacccttgttagtcaaaccacacataagagaggatggatttaaaccagtcagcaccgtaagtatatagtgaagaaggctgataacacactctattattgttagtacgtacgtatttccttttttgtttagtttttgaatttaattaattaaaatatatatgctaacaacattaaattttaaatttacgtctaattatatattgtgatgtataataaattgtcaacctttaaaaattataaaagaaatattaattttgataaacaacttttgaaaagtacccaataatgctagtataaataggggcatgactccccatgcatcacagtgcaatttagctgaagcaaagcaatggctactt

SEQ ID NO: 57 - DNA, 20 bp, Artificial Sequence
  DNA encoding Guide 1 RNA for GY1, GY2, GY3
tatacggttatccggtttga

SEQ ID NO: 58 - DNA, 20 bp, Artificial Sequence
  DNA encoding Guide 2 RNA for GY1, GY2, GY3
agagggcaacaccggcacac

SEQ ID NO: 59 - DNA, 20 bp, Artificial Sequence
  DNA encoding Guide 1 RNA for GY4, GY5
ggcttccccatattgaacta

SEQ ID NO: 60 - DNA, 20 bp, Artificial Sequence
  DNA encoding Guide 2 RNA for GY4, GY5
caccgcgttgagtccgaagg

SEQ ID NO: 61 - DNA, 20 bp, Artificial Sequence
  DNA encoding Guide 1 RNA for alpha-conglycinin and alpha-prime conglycinin
cggttcccattactgttgct

SEQ ID NO: 62 - DNA, 20 bp, Artificial Sequence
  DNA encoding Guide 2 RNA for alpha-conglycinin and alpha-prime conglycinin
tcgttgcaacctccttaagg

SEQ ID NO: 63 - DNA, 20 bp, Artificial Sequence
  DNA encoding Guide 1 RNA for beta-conglycinin
ttagagcttctcaagtagaa

SEQ ID NO: 64 - DNA, 20 bp, Artificial Sequence
  DNA encoding Guide 2 RNA for beta-conglycinin
tgggggagaaggattgtgtt

SEQ ID NO: 65 - DNA, 20 bp, Artificial Sequence
  DNA encoding Guide 1 RNA for FAD2-1A and FAD2-1B
ttgagttggccaacagtgaa

SEQ ID NO: 66 - DNA, 20 bp, Artificial Sequence
  DNA encoding Guide 2 RNA for FAD2-1A and FAD2-1B
aatagattggccatgcaatg

SEQ ID NO: 67 - DNA, 20 bp, Artificial Sequence
  DNA encoding Guide 1 RNA for SACPD-C
agtgctagcggcgtaaggaa

SEQ ID NO: 68 - DNA, 20 bp, Artificial Sequence
  DNA encoding Guide 2 RNA for SACPD-C
gaagtttatgcgaatttatg

SEQ ID NO: 69 - DNA, 40838 bp, Artificial Sequence
  pDGB-alpha1-Seven Genes+CSY4/Cas9+gRNA
taacgaattcgtctcaggagaacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccatggccaagctaaatgaaggtcctcatccttgcctgcctggtggctctggcccttgcaagagagctggaagaactcaatgtacctggtgagattgtggaaagcctttcaagcagtgaggaatctattacacgcatcaataagaaaattgagaagtttcagagtgaggaacagcagcaaacagaggatgaactccaggataaaatccacccctttgcccagacacagtctctagtctatcccttccctgggcccatccataacagcctcccacaaaacatccctcctcttactcaaacccctgtggtggtgccgcctttccttcagcctgaagtaatgggagtctccaaagtgaaggaggctatggctcctaagcacaaagaaatgcccttccctaaatatccagttgagccctttactgaaaggcagagcctgactctcactgatgttgaaaatctgcaccttcctctgcctctgctccagtcttggatgcaccagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccaggagcctgtactcggtcctgtccggggacccttccctattattgtctaagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggagaacttaatcgtatataaaaaattcaatatatgaataattctaagtgagtttttaagaaaaaataaaattagtaacgaagtaatttatatataattttgaaaaattatcactaaatttgtgatccactgttaacattaatttattcctcttgtattgaataaaatagttcagacatggtcccagtctttaatcaattattcatgcttctctgtctctcacttatataatcctgtaatccaaacattactcagatagctagatccaccgatcaatcgtatatatatacgcataaaatcgacgcctctgtattttttagactgtagcccaaattcactatccgaataaaataagggaggcacgtgtacgtaatttatatcatatgatagccatgcatatgcacacgtgcagaagagctgttaccctctatacgtgtactcaccttctcatcctctctgaatattttgagtgctcttcctagttatctagtaatgcatgaaattaaacttactaaatgtttcttcaatttaaagaaataattgtttatctgtttcaatttttttaagagaattttaaaaagataattgtttcggggagagagatataaaaaagaaaagggagaaatattaaaatgtactaaataatatgataagaaaagagagaaaaataaaagagaaaatttgtatatagttataattattcatgtaataaggattcatctctcaactgaaaatatacttaatgcagaagaaaaaatcattatttacaaacgttgagtcttgagtgggaaaagaggaggcgccgttactatacaatataagatcatagtactgacaaaatgcacagtaaaacagttcaaattgagaaggattcttaacacaccatagtatttaatatatatctttacagagacaattatgctggaggattcaggcaaagattatatattgtggatttgttttttaataattaacgcatcatatgaaagatcgatgatatatactaatggttataagaaaaatatttaacagtttctataacctttttcttttatcttttactgtaatattatttattttatttcacatttttaatcagcttatctcatttataaacgaaattgtataaaaatatacatgatgaactgaatagaacaatattgatctgatattctcatattgtataagaggatagactttgaggcgcggagaatctgtaggaggggaccattcagagtgcctccaattttggtgttgttcattgtaccattgcaaatataaacgaagcatgcatgcttatgtatgaggtgtaacaaaattggaaacaatagccatgcaaggtgaagaatgtcacaaactcagcaacccttattcattgacgtgtccctcagtcactctcctctcatacctataaatcaccactcctcatgttctttccaattaccaactccttcaaacttaattattaacacttccttagttcaatatggggaagccaatgaaacttctcatccttacctgtcttgtggctgttgctcttgccaggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattctctgacatccctaatcccattggctctgagaacagtgaaaagactactatgccactgtggtgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggagtacattttgagttgtttcaggttccattgccttattgctaaaactccaactaaaataacaaatagcacatgcaggtgcaaacaacacgttactctgatgaaggtgatgtgcctctagcagtctagcttatgaggctcgctgcttatcaacgattcatcattccccaagacgtgtacgcagattaaacaatggacaaaacttcaatcgattatagaataataattttaacagtgccgacttttttctgtaaacaaaaggccagaatcatatcgcacatcatcttgaatgcagtgtcgagtttggaccatttgagtacaaagccaatattgaatgatttttcgattttacatgtgtgaatcagacaaaagtgcatgcaatcacttgcaagtaaattaaggatactaatctattcctttcattttatatgctccacttttatataaaaaaatatacattattatatatgcattattaattattgcagtattatgctattggttttatggccctgctaaataacctaaatgagtctaactattgcatatgaatcaaatgaaggaagaatcatgatctaaacctgagtacccaatgcaataaaatgcgtcctattacctaaacttcaaacacacattgccatcggacgtataaattaatgcatatagattattttgagaaaagaaaacatcaaaagctctaaaacttcttttaactttgaaataagctgataaaaatacgctttaaatcaactgtgtgctgtatataagctgcaatttcacattttaccaaaccgaaacaagaatggtaacagtgaggcaaaaatttgaaaaatgtcctacttcacattcacatcaaattaattacaactaaataaataaacatcgtgattcaagcagtaatgaaagtcgaaatcagatagaatatacacgtttaacatcaattgaatttttttttaaatggatatatacaagtttactattttatatataatgaaaattcattttgtgttagcacaaaacttacagaaagagataaattttaaataaagagaattatatccaattttataatccaaaataatcaaattaaagaatattggctagatagaccggctttttcactgcccctgctggataatgaaaattcatatcaaaacaatacagaagttctagtttaataataaaaaagttggcaaactgtcattccctgttggtttttaagccaaatcacaattcaattacgtatcagaaattaatttaaaccaaatatatagctacgagggaacttcttcagtcattactagctagctcactaatcactatatatacgacatgctacaagtgaagtgaccatatcttaatttcaaatcataaaattcttccaccaagttatgggtttcctaatgatgaagagttttttcctagttgtgactatcctggcattaaccctgccatttttgggtgcccaggagcaaaaccaagaacaaccaatacgctgtgagaaagatgaaagattcttcagtgacaaaatagccaaatatatcccaattcagtatgtgctgagtaggtatcctagttatggactcaattactaccaacagaaaccagttgcactaattaataatcaatttctgccatacccatattatgcaaagccagctgcagttaggtcacctgcccaaattcttcaatggcaagttttgtcaaatactgtgcctgccaagtcctgccaagcccagccaactaccatggcacgtcacccacacccacatttatcatttatggccattccaccaaagaaaaatcaggataaaacagaaatccctaccatcaataccattgctagtggtgagcctacaagtacacctaccatcgaagcagtagagagcactgtagctactctagaagcttctccagaagttattgagagcccacctgagatcaacacagtccaagttacttcaactgcggtctaagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgctgtcaggagattatttctgttagtacatagctaatactcaatcaacggaattagtatatggttcttcatataggagagtacttatttattctattgaattttaacatataagcataataaaatacttttggactctcgtataaagttcgattttaatctttttaataattcaatctaaatgtttaattccctcttaaatgcaaaattcagttttcgttcctttaatgtgacaccattaggtcacatgaaccggaaatgacgtggtgatcgaattatgacttgaatccattgaccacattagcatttcacctatggtcactagtatgaaggatgaaaacaagtctatttctcaaattataaatgaaaacgtttaactttaaacctgaggatccaaaaacgaattttactaaattttgaagaactaaaaaatatttaatctagtaaaacgcgtgtctatctaatataacatgcacgctcgtcatgtaatcaattaggcataaaaatagtgtttgattttttgacacattattaagtgttttatttttaagtttaaaagcattggtatcctttcataaaaggaggtaatcttatttaagtcaaggagaattattatgggaaataaaacctttttttttaaagtgtttaatataattatatactcaaaattcgatttatgattaaatctaagtgacatttaaaaaaaattagtgtgaaaataatttatatataattttgaaaaatttatcattaatttttttttataaataaatgttaatttattagtttttattataaatgtgaatagaatggattcgaagcagcaatttctctctttctccttttccatgccaaccttatatatggtgacgaactgcatatacagtaaaacagttcaaattgagaaagattttaaacatcatagtatttgatatatatcttttacagagacaattatgctgcaggagttagataagattattgtggatgtcattttcttttttaatatttaacgcattatataaaagatgatatagtatggttataaaaaaattatttaacagtttataaaacctttttttttatcttttacagtaatattatttattttatttcacatttttttcatatccttatctcatttataaaggaaattaattgtataaaaaaaatatgatgcactgaatagaatgctgatcttattgtataaggaggatagaatttgagacacggagaatctgtagagggggaccattcagggtgcctgcaattttggtgttgttcatgtacggttgcagatataaacgaagcatagcttatgtatgaggtgtaacaaaattggaaacaatagccatgcaaggtgaagaatgtcaccaactcagaaacccttcttcattgacgtgtccctcactcactctcctctcttcactataaatcgccactcttcgtgttctccacttcaccaactccttcaaacttattaacactttccttagttcaatatggggaagcaatgaagttcttcatctttacctgccttttggctgttgcccttgcaaagaatacgatggaacatgtctcctccagtgaggaatctatcatctcccaggaaacatataagcaggaaaagaatatggacattaatcccagcaaggagaacctttgctccacattctgcaaggaagttgtaaggaacgcaaatgaagaggaatattctatcggctcatctagtgaggaatctgctgaagttgccacagaggaagttaagattactgtggacgataagcactaccagaaagcactgaatgaaatcaatcagttttatcggaagttcccccagtatctccagtatctgtatcaaggtccaattgttttgaacccatgggatcaggttaagagaaatgctgttcccattactcccactctgaacagagagcagctctccaccagtgaggaaaattcaaagaagaccgttgacatggaatcaacagaagtattcactaagaaaactaaactgactgaagaagaaaagaatcgcctaaattttctgaaaaaaatcagccagcgttaccagaaattcgccttgccccagtatctcaaaactgtttatcagcatcagaaagctatgaagccatggattcaacctaagacaaaggttattccctatgtgaggtacctttaagcttaagctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaacttgaatgttcactactactcattgaccataatgtttaacatacatagctcaatagtatttttgtgaatatggcaacacaaacagtccaaaacaattgtctcttactataccaaaccaagggcgccgcttgtttgccactctttgtgtgcaatagtgtgattaccacacgctgtcaggagtacattttgagttgtttcaggttccattgccttattgctaaaactccaactaaaataacaaatagcacatgcaggtgcaaacaacacgttactctgatgaaggtgatgtgcctctagcagtctagcttatgaggctcgctgcttatcaacgattcatcattccccaagacgtgtacgcagattaaacaatggacaaaacttcaatcgattatagaataataattttaacagtgccgacttttttctgtaaacaaaaggccagaatcatatcgcacatcatcttgaatgcagtgtcgagtttggaccatttgagtacaaagccaatattgaatgatttttcgattttacatgtgtgaatcagacaaaagtgcatgcaatcacttgcaagtaaattaaggatactaatctattcctttcattttatatgctccacttttatataaaaaaatatacattattatatatgcattattaattattgcagtattatgctattggttttatggccctgctaaataacctaaatgagtctaactattgcatatgaatcaaatgaaggaagaatcatgatctaaacctgagtacccaatgcaataaaatgcgtcctattacctaaacttcaaacacacattgccatcggacgtataaattaatgcatatagattattttgagaaaagaaaacatcaaaagctctaaaacttcttttaactttgaaataagctgataaaaatacgctttaaatcaactgtgtgctgtatataagctgcaatttcacattttaccaaaccgaaacaagaatggtaacagtgaggcaaaaatttgaaaaatgtcctacttcacattcacatcaaattaattacaactaaataaataaacatcgtgattcaagcagtaatgaaagtcgaaatcagatagaatatacacgtttaacatcaattgaatttttttttaaatggatatatacaagtttactattttatatataatgaaaattcattttgtgttagcacaaaacttacagaaagagataaattttaaataaagagaattatatccaattttataatccaaaataatcaaattaaagaatattggctagatagaccggctttttcactgcccctgctggataatgaaaattcatatcaaaacaatacagaagttctagtttaataataaaaaagttggcaaactgtcattccctgttggtttttaagccaaatcacaattcaattacgtatcagaaattaatttaaaccaaatatatagctacgagggaacttcttcagtcattactagctagctcactaatcactatatatacgacatgctacaagtgaagtgaccatatcttaatttcaaatcataaaattcttccaccaagttatgggtttcctaatgaagtgcctcctgcttgccctggccctcacttgtggcgcccaggccctcattgtcacccagaccatgaagggcctggatatccagaaggtggcggggacttggtactccttggccatggcggccagcgacatctccctgctggacgcccagagtgcccccctgagagtgtatgtggaggagctgaagcccacccctgagggcgacctggagatcctgctgcagaaatgggagaacggtgagtgtgctcagaagaagatcattgcagaaaaaaccaagatccctgcggtgttcaagatcgatgccttgaatgagaacaaagtccttgtgctggacaccgactacaaaaagtacctgctcttctgcatggagaacagtgctgagcccgagcaaagcctggcctgccagtgcctggtcaggaccccggaggtggacgacgaggccctggagaaattcgacaaagccctcaaggccctgcccatgcacatccggctgtccttcaacccaacccagctggaggagcagtgccacatctaggcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgctgtcaggagtataaacaccactttaatttgactcggatacatgcatccataaagactacaaaaggcaaaaagagaaggaaatgagatacgaatatatgtcataagtatatataggtgacaagggcaaattaaataggttggtatttaaatgcaaaatcctatgtttgataaagaatggtatgaaaaacaggcaaagttaattgcaattcaaaggtgaacaaagcatttctttgtctacactaatggcatgtctaagtaaattattagtcttgtatctatatgtccacaagttattaattagtcttatactatcaaaaacaagttaagttgcaaatcaaacatgaacaaagcatttgtgttgtaacctacgaaaaaataccctaacatactgatacgaataatgtggcctaaattgatcgtttaccaaattacggtgctggaaaaaaaaattgctcctttaccaacaaaattaagaactgatacatcttgttttttgtcactgaagataaacacgtgatctttggcaaaacataaaggccaacaaaacaaacttgtctcatccctgaatgattcgaatgccatcgtatgcgtgtcacaaagtggaatacagcaatgaacaaatgctatcctcttgagaaaagtgaatgcagcagcagcagcagactagagtgctacaaatgcttatcctcttgagaaaagtgaatgcagcggcagcagacctgagtgctatatacaattagacacagggtctattaattgaaattgtcttattattaaatatttcgttttatattaattttttaaattttaattaaatttatatatattatatttaagacagatatatttatttgtgattataaatgtgtcactttttcttttagtccatgtattcttctattttttcaatttaactttttatttttatttttaagtcactcttgatcaagaaaacattgttgacataaaactattaacataaaattatgttaacatgtgataacatcatattttactaatataacgtcgcattttaacgtttttttaacaaatatcgactgtaagagtaaaaatgaaatgtttgaaaaggttaattgcatactaactattttttttcctataagtaatcttttttgggatcaattgtatatcattgagatacgatattaaatatgggtaccttttcacaaaacctaacccttgttagtcaaaccacacataagagaggatggatttaaaccagtcagcaccgtaagtatatagtgaagaaggctgataacacactctattattgttagtacgtacgtatttccttttttgtttagtttttgaatttaattaattaaaatatatatgctaacaacattaaattttaaatttacgtctaattatatattgtgatgtataataaattgtcaacctttaaaaattataaaagaaatattaattttgataaacaacttttgaaaagtacccaataatgctagtataaataggggcatgactccccatgcatcacagtgcaatttagctgaagcaaagcaatggctacttaatgatgtcctttgtctctctgctcctggtaggcatcctattccatgccacccaggctgaacagttaacaaaatgtgaggtgttccgggagctgaaagacttgaagggctacggaggtgtcagtttgcctgaatgggtctgtaccacgtttcataccagtggttatgacacacaagccatagtacaaaacaatgacagcacagaatatggactcttccagataaataataaaatttggtgcaaagacgaccagaaccctcactcaagcaacatctgtaacatctcctgtgacaagttcctggatgatgatcttactgatgacattatgtgtgtcaagaagattctggataaagtaggaattaactactggttggcccataaagcactctgttctgagaagctggatcagtggctctgtgagaagttgtgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgctgtcaggagagcgatcagcttgcatgccggtcgatctagtaacatagtagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttgactctaggggtcatcagatttcggtgacgggcaggaccggacggggcggcaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggatacgtacacggtcgactcggccgtccagtcgtaggcgttgcgtgccttccagggacccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcccctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataacgtgactcccttaattctcatgtatgatactccgtcaggagataattataaaattgtcactgcgttcaaaacgacaatggttttgggacaactatcattaatcgtgcattgtaaaaaggtgtgtttttagtagtggaccctcgataaattgactgtgatgattgttacatgttgttaagtctcacctataagaaaaaaactaaacatatatatagatcccaattttggggtcaggtgtatagatgaaaaaaagaaacaaatagacaaataaaaaaataaaagaaaaaaaattgatagatgtgagaaatgatgagaagagaagtgcaaataacacactctttctaacattattttactattgattaaaatttattgaaaattactatataatataaaaagtgaaactagttaaactatagtcaataattgagaatatttaaaaatttagaaaatacattacttatatttcttaaaataaaaaatataaataaaaatagaaaaaatggagtaaaatgagatagaagagaagttaggtttataaatacattagttccgcctacaatatatttaaattagctagattaatgcagtaaatttttggcatttacttgattttattttctttaaaagcattctttgtattcttcactgatggttttttttcttcatctgcattatgaattaaatcatttactttgtgtcacaattgcatttagcgaggtcatgcattggttagaccgacggtgtattatgtcatgacttaggtcttgaaggttgttggttacttattatggtccatgggtacacgcgttggttagattcgataggcaaattttgtgaacgatagaaatttatctttattaaataaaccacactatatatatatatatatatatatatatatatatatatatatatattaattcgtaatttcttttctgtctttcattttgattttcttttatggcttttatctttaaaaattttccccttctttaaaatttacaacactttataatcacaataaaataaaataatttaaaatattacataaataataacacaaatatttataaatctgaaatgacataaaataacattataatcacaaaaagtatttaataaaaataaaattacataaataaaatattgtgaaaactaagtaaaaggtatcatgcacgtaatcatatgaaaatagctttagaaaaaatatcaaggcaagtaccgcacgtacgataaatgaaaaaagattaaaaagaaatataataaataataatactaaattaatggtgaataaaatactaaaaaaataaatttataattaaataatatgtattacaaacacaaataagaaataatagtacataatattataataaatagtagtatataacatatcataaatatgtttaaaataatgataaaatattgagtttcttttagtggaactatttgtcaaaatgtgaacacctggatatgaaaaggcatcttaggtagatgatatgatgcgatagaacgtaaaagaaaaatgagaaatgttgatgagaggttaaaaatacccttcataacaagcacacatctataagtagtcttattcacccaacaacgttgcttattcacgcaactaaataagaaatgaagagtactataatgaagtgggtgacttttatttctcttctccttctcttcagctctgcttattccaggggtgtgtttcgtcgagatacacacaagagtgagattgctcatcggtttaaagatttgggagaagaacattttaaaggcctggtactgattgccttttctcagtatctccagcagtgtccatttgatgagcatgtaaaattagtgaacgaactaactgagtttgcaaaaacatgtgttgctgatgagtcccatgccggctgtgaaaagtcacttcacactctctttggagatgaattgtgtaaagttgcatcccttcgtgaaacctatggtgacatggctgactgctgtgcgaaacaagagcctgaaagaaatgaatgcttcctgagccacaaagatgatagcccagacctccctaaattgaaaccagaccccaatactttgtgtgatgagtttaaggcagatgaaaagaagttttggggaaaatacctatacgaaattgctagaagacatccctacttttatgcaccagaactcctttactatgctaataaatataatggagtttttcaagaatgctgccaagctgaagataaaggtgcctgcctgctaccaaagattgaaactatgagagaaaaagtactgacttcatctgccagacagagactcaggtgtgccagtattcaaaaatttggagaaagagctttaaaagcatggtcagtagctcgcctgagccagaaatttcccaaggctgagtttgtagaagttaccaagctagtgacagatctcacaaaagtccacaaggaatgctgccatggtgacctacttgaatgcgcagatgacagggcagatcttgccaagtacatatgtgataatcaagatacaatctccagtaaactgaaggaatgctgtgataagcctttgttggaaaaatcccactgcattgctgaggtggaaaaagatgccatacctgaaaacctgcccccattaactgctgactttgctgaagataaggatgtttgcaaaaactatcaggaagcaaaagatgccttcctgggctcgtttttgtatgaatattcaagaaggcatcctgaatatgctgtctcagtgctattgagacttgccaaggaatatgaagccacactggaggaatgctgtgccaaagatgatccacatgcatgctattccacagtgtttgacaaacttaagcatcttgtggatgagcctcagaatttaatcaaacaaaactgtgaccaattcgaaaaacttggagagtatggattccaaaatgagctcatagttcgttacaccaggaaagtaccccaagtgtcaactccaactctcgtggaggtttcaagaagcctaggaaaagtgggtactaggtgttgtacaaagccggaatcagaaagaatgccctgtgctgaagactatctgagcttgatcctgaaccggttgtgcgtgctgcatgagaagacaccagtgagtgaaaaagtcaccaagtgctgcacagagtcattggtgaacagacggccatgtttctctgctctgacacctgatgaaacatatgtacccaaagcctttgatgagaaattgttcaccttccatgcagatatatgcacacttcccgatactgagaaacaaatcaagaaacaaactgcacttgttgagctgttgaaacacaagcccaaggcaacagaggaacaactgaaaaccgtcatggagaattttgtggcttttgtaggcaagtgctgtgcagctgatgacaaagaggcctgctttgctgtggagggtccaaaacttgttgtttcaactcaaacagccttagcctaagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggagagcgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcattgcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgattcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtatctccgtcaggaggtcaactaccccaatttaaattttatttgattaagatatttttatggacctactttataattaaaaatattttctatttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcagaatggatcattatcttgatattagacttagacctgatccagaatttccaccagctcaacttatgtctgttctttttggaaaacttcatcaagctcttgttgctcaaggaggagatagaattggagtttcttttcctgatcttgatgaatcaagatcaagacttggagaaagacttagaattcatgcttctgctgatgatcttagagctttgcttgctagaccttggcttgaaggacttagagatcatcttcaatttggagaaccagctgttgttccacatccaactccttatagacaagtttcaagagttcaagctaaatctaatccagaaagacttagaagaagacttatgagaagacatgatctttctgaagaagaagctagaaaaagaattcctgatactgttgctagagctttggatttgccttttgttacacttagatcacaatctactggacaacattttagactttttattagacatggaccacttcaagttactgctgaagaaggaggatttacttgttatggactttctaagggaggttttgttccttggtttggatctggagctactaatttttctcttcttaagcaagctggagatgttgaagaaaatcctggacccatgatggatccccgggatcatctacttctgaagactcagactcagactaagcaggtgacgaacgtcaccaatcccaattcgatctacatcgataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgagcttgttgtggttgtctggttgcgtctgttgcccgttgtctgttgcccattgtggtggttgtgtttgtatgatggtcgttaaggatcatcaatgtgttttcgctttttgttccattctgtttctcatttgtgaataataatggtatctttatgaatatgcagtttgtggtttcttttctgattgcagttctgagcattttgtttttgcttccgtttactataccacttacagtttgcactaatttagttgatatgcgagccatctgatgtttgatgattcaaatggcgtttatgtaactcgtacccgagtggatggagaagagctccattgccggtttgtttcatgggtggcggagggcaactcctgggaaggaacaaaagaaaaaccgtgatacgagttcatgggtgagagctccagcttgatcccttctctgtcgatcaaatttgaatttttggatcacggcaggctcacaagataatccaaagtaaaacataatgaatagtacttctcaatgatcacttatttttagcaaatcagcaattgtgcatgtcaaatgatttcggtgtaagagaaagagttgatgaatcaaaatatctgtagctggatcaagaatctgaggcagttgtatgtatcaatgatctttccgctacaatgatgttagctatccgagtcaaattgttgtagaattgcatacttcggcatcacattctggatgacataataaataggaagtcttcagatccctaaaaaattgagagctaataacattagtcctagatgtaactgggtgacaaccaagaaagagacatgcaaatactacttttgtttgaaggagcatccctggtttgacatattttttctgaatatcaaactttgaaactctacctagtctaatgtctaacgacagatcttactggtttaactgcagtgatatctactatcttttggaatgttttctccttcagttatacatcaagttccaagatgcaggtgtgcttgattgatgtacatggctgtgagaagtgcatcctgatgttcagatgatggttcattctaatgtcttttccttcaatcagttttctcagtctgacttagcttgtttcatctgcatgtttgaatgttcgtttactcatagtaattgcatttttgtagcagaacatatcattggtcatggtttcaactgtgcgcgagtcttatgcttattcaaactaggaaagcctccgtctagagggtacacgagttgttgctctgtgtgcgtcagtccatagtattaatcttgctagttgtagtatattgtttatgtggactcggaattcatcatatgctccttctttgcatcaagtaaggcaaggtaatgtatagaagctttttaactctttcatggaagctggcctttgccagcataccatccagaagatatcaaccctgcatcttggctgccgcgctgtcaggagtctcaatggtaactttactctttatttaaccatacatttttttttatttttttcactttgttcttcatccactattgttctttgttcatcttgaacaaaagctccctccttctttgttcttcatccaccattgttcttcatcaatcatttcgctgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgaaatggatctgactagtcctgcaggttcactgccgtataggcagtatacggttatccggtttgagttttagagctagaaatagcaagttaaaataaggctagtccgttatcaacttgaaaaagtggcaccgagtcggtgcgttcactgccgtataggcagcgacaagagtagcaagcaaagttttagagctagaaatagcaagttaaaataaggctagtccgttatcaacttgaaaaagtggcaccgagtcggtgcgttcactgccgtataggcagcggttcccattactgttgctgttttagagctagaaatagcaagttaaaataaggctagtccgttatcaacttgaaaaagtggcaccgagtcggtgcgttcactgccgtataggcagttagagcttctcaagtagaagttttagagctagaaatagcaagttaaaataaggctagtccgttatcaacttgaaaaagtggcaccgagtcggtgcgttcactgccgtataggcagttgagttggccaacagtgaagttttagagctagaaatagcaagttaaaataaggctagtccgttatcaacttgaaaaagtggcaccgagtcggtgcgttcactgccgtataggcagagtgctagcggcgtaaggaagttttagagctagaaatagcaagttaaaataaggctagtccgttatcaacttgaaaaagtggcaccgagtcggtgcgttcactgccgtataggcagagagggcaacaccggcacacgttttagagctagaaatagcaagttaaaataaggctagtccgttatcaacttgaaaaagtggcaccgagtcggtgcgttcactgcttcgtataggcagcaccgcgttgagtccgaagggttttagagctagaaatagcaagttaaaataaggctagtccgttatcaacttgaaaaagtggcaccgagtcggtgcgttcactgccgtataggcagtcgttgcaacctccttaagggttttagagctagaaatagcaagttaaaataaggctagtccgttatcaacttgaaaaagtggcaccgagtcggtgcgttcactgccgtataggcaggtgggggagaaggattgtgttgttttagagctagaaatagcaagttaaaataaggctagtccgttatcaacttgaaaaagtggcaccgagtcggtgcgttcactgccgtataggcagaatagattggccatgcaatggttttagagctagaaatagcaagttaaaataaggctagtccgttatcaacttgaaaaagtggcaccgagtcggtgcgttcactgccgtataggcaggaagtttatgcgaatttatggttttagagctagaaatagcaagttaaaataaggctagtccgttatcaacttgaaaaagtggcaccgagtcggtgcgttcactgccgtataggcagtcgatcgacaagggtacctaggcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgctgtcatgagacgaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaaca

(19) United States
(12) Patent Application Publication
(10) Pub. No.: US 2023/0034320 A1
(43) Pub. Date: Feb. 2, 2023

(54) PLANT EXPRESSING ANIMAL MILK PROTEINS

(71) Applicant: YEDA RESEARCH AND DEVELOPMENT CO. LTD., Rehovot (IL)

(72) Inventors: Asaph AHARONI, Tel-Aviv (IL); Aviel EVEN, Rehovot (IL); Dan EVEN, Rehovot (IL)

(73) Assignee: YEDA RESEARCH AND DEVELOPMENT CO. LTD., Rehovot (IL)

(21) Appl. No.: 17/489,824

(22) Filed: Sep. 30, 2021

Related U.S. Application Data
(63) Continuation-in-part of application No. PCT/IL2020/050400, filed on Apr. 2, 2020.

(30) Foreign Application Priority Data
Apr. 4, 2019 (IL) 265841

(51) Int. Cl. C12N 15/82

(52) U.S. Cl. CPC C12N 15/8257; C12N 15/8234; C12N 15/8251

(57) ABSTRACT

Disclosed herein are genetically modified plants expressing multiple animal milk proteins. Further disclosed are vectors for expressing in a plant and parts thereof, multiple mammalian milk proteins; genetically modified and or gene-edited plants having at least one cell expressing and producing at least two recombinant mammalian milk proteins wherein expression may be in a seed, bean, grain, fruit, nut, legume, leaf, stem, root, or portion thereof, the recombinant protein being produced by the plant cell; and a method of producing a food, medicament, cosmetic or blocking composition from the genetically modified or gene-edited plant. Reduction or elimination of seed storage proteins in a cell or cells wherein the milk proteins are expressed, or reduction of plant enzymes that can increase the content of oleic and/or stearic fatty acids and/or reduce the content of saturated fats in the plants or plant products are also disclosed.

### CROSS-REFERENCE TO RELATED APPLICATIONS

This application is a Continuation in Part Application of PCT International Application No. PCT/IL2020/050400, International Filing Date Apr. 2, 2020, claiming the benefit of Israel Application No. 265841, filed Apr. 4, 2019, which are hereby incorporated by reference.

The instant application contains a Sequence Listing which has been submitted electronically in ASCII format and is hereby incorporated by reference in its entirety. Said ASCII copy, created on Mar. 31, 2020, is named P-583829-US-SQL-31MAR20_ST25 and is 216 Kilo bytes in size.

### FIELD OF THE INVENTION

The present invention relates to key genes in the biosynthesis of animal milk proteins and to genetically modified or gene edited plants with altered content of animal milk proteins, particularly to plants with increased content of animal milk proteins and any of their derivatives. The present invention also relates to plant-based food, medicament, cosmetic, or blocking compositions comprising animal milk proteins and methods of making the same. Additionally, the present invention relates to genetically modified or gene edited plants with de novo content of animal milk proteins and any of their derivatives and with reduced plant proteins, including plant proteins implicated in human allergies to said plants and/or plant proteins. The present invention also relates to the reduction of plant enzymes that can increase the content of oleic and/or stearic fatty acids and/or reduce the content of saturated fats in said plants or plant products.

### BACKGROUND OF THE INVENTION

There is a global challenge to feed the fast-growing world population. With an estimated number of 793 million people undernourished as of 2015 (FAO Statistical, FAO Statistical Pocketbook 2015, p. 14 (Rome 2015) [“FAO Statistical 2015”]), it is clear why the United Nation assembly proclaimed the decade of action on nutrition on its 1 Apr. 2016 resolution, which aims to trigger intensified action to end hunger worldwide (United Nations, Decade of Action on Nutrition at the UN General Assembly (71^{st} Session) (2016) [“UN 2016”]). To help meet humanity's need for food, biotechnology's immense power could be harvested. Genetic engineering can improve both the yield and nutritional values of food crops (Borlaug (2000) Plant Physiol. 124(2): 487-490 [“Borlaug 2000”]; Kishore et al. (May 1999) Proc. Natl. Acad. Sci. 96(11): 5968-5972 [“Kishore 1999”]), as in the case of Golden Rice (Ye et al. (2000) Science (80-) 287(5451): 303-305 [“Ye 2000”]). For example, by genetically modifying rice endosperm to express the biosynthetic pathway of provitamin-A (Ye 2000), the Golden Rice can impact the lives of more than 250 million children suffering from Vitamin-A deficiency, which can lead to blindness and even death (World Health Organization, “Global prevalence of vitamin A deficiency in populations at risk 1995-2005: WHO global database on vitamin A deficiency,” WHO Iris, p. 55 (2009) [“WHO 2009”]). The use of genetically modified crops in general, and of Golden Rice in particular, has recently received the support of 107 Nobel laureates, who advocated these crops to be as safe as those derived from traditional breeding methods (Achenbach (2016) “107 Nobel laureates just signed a letter slamming Greenpeace over GMOs,” Washington Post [available: https://www.sciencealert.com/107-nobel-laureates-just-signed-a-letter-slamming-greenpeace-about-gmos; accessed: 29 Nov. 2018] [“Achenbach 2016”]). While biotechnology becomes a promising player in the effort to solve world hunger, animal-based agriculture plays a pivotal role in aggravating it (Shepon et al. (March 2018) Proc. Natl. Acad. Sci., p. 201713820 [“Shepon 2018”]). According to the United Nations Environment Program the calories lost by feeding farm animals with cereals and other plant crops, could alternatively nourish 3.5 billion people (FAO Statistical 2015). Despite that the world's diet is shifting towards an increased consumption of animal-based products such as milk, meat and eggs (FAO Statistical 2015).

With an estimated annual production of 800 million liters and $328 billion market value, the global milk industry is rapidly expanding (FAO (2015) Food Outlook Biannual Report on Global Food Markets [“FAO Food Outlook 2015”]; FAO Statistical 2015). Historically, “milk” is “the normal mammary secretion of milking animals” (FAO, Codex Alimentarius, “Milk” (Codex Stan 206-1999) [http://www.fao.org/fao-who-codexalimentarius/en/] [“FAO Codex 1999”]). While domestic cows are the source of most commercial milk production, other farm animal sources include buffalo, goat, sheep, camel, donkey, horse, reindeer, yak, moose, bison, bison/cow hybrid, and pig.

Global milk production and consumption is growing steadily and is projected to be doubled by 2050 (FAO (2012) World agriculture towards 2030/2050: the 2012 revision, p. 75 “FAO World Agriculture 2012”]). Milk is nutritionally beneficial to humans, since it contains essential vitamins, minerals, fats and proteins as well as high caloric values (FAO World Agriculture 2012; Muehlhoff et al. (May 2013) Milk and dairy products in human nutrition, FAO UN 67(2): 303-304 [“Muehlhoff 2013”; see also Haug et al. (September 2007) Lipids Health Dis. 6(1): 25 et seq. [“Haug 2007”]). Casein, the most abundant protein in milk, considered to be a quality protein source with a high digestibility index according to the World Health Organization. Furthermore, whey proteins and Caseins facilitate the absorption of essential minerals, such as calcium, phosphate, iron and zinc, by binding and maintaining them as an easily ingestible suspension (Vegarud et al. (2000) Br. J. Nutr. 84(S1): S91-S98 [“Vegarud 2000”]). On the contrary some ingredients of milk, such as cholesterol, saturated fat lactose and antibiotics residues have been associated with negative effects on human health (Goodland, The Westernization of diets: the assessment of impacts in developing countries—with special reference to China, www.worldbank.org (2001) [“Goodland 2001”]) Furthermore, during milking, a variety of pathogenic bacteria are inoculated into the milk originated from abundant infections in the cows' udder. These include multi-drug resistant bacteria, which could in turn infect people consuming dairy products [Goodland 2001; Spoor et al. (August 2013) MBio 4(4): 1-6 [“Spoor 2013”]; Cabello (1 Jul. 2006) Environ. Microbiol. 8(7): 1137-1144 [“Cabello 2006”]; see also Witte (November 2000) Int. J. Antimicrob. Agents 16(Supp. 1; no. 0924-8579): S19-S24 [“Witte 2000”]). While milk is a valuable food source for humanity, its production comes with great costs. In addition to reducing cereal availability for consumption by weak populations in developing countries (Cassidy et al. (2013) Environ. Res. Lett. 8(3): 1-8 (034015) [“Cassidy 2013”]), milk production contributes significantly to environmental pollution and emission of greenhouse gases (Cassidy 2013; FAO (2006) Livestock's long shadow—environmental issues and options, FAO, pp. 112-114 [“FAO Livestock 2006”]; see also FAO Assessment (2010) Greenhouse gas emissions from the dairy sector, Africa(Lond.), p. 98 [“FAO 2010”]), and raises moral and ethical dilemmas regarding the housing of farm animals in the dairy industry (Beggs et al. (August 2015) J. Dairy Sci. 98(8): 5330-5338 [“Beggs 2015”]).

From the above arises a need to find alternatives for the current ways of milk production, which will allow to feed the fast-growing world population in a more sustainable and healthy manner. One such possibility is to produce milk alternatives in animal-free systems. Only a few attempts have been engaged to deal with this important task; since 2014 the “Perfect Day Foods” enterprise has been working on composing a milk-like drink by combining cow's milk proteins extracted from transgenic yeast, fatty acids derived from plants and minerals and sugar from other sources (U.S. Pat. No. 9,924,728). This milk alternative is based on mixing ingredients from several sources, which requires advanced laboratory equipment and a well-trained staff, putting in doubt the possibility of going on a global large-scale production of their product, especially in developing countries.

The major components of milk are fatty acids, lactose and proteins, the last of which are similar in their relative content both in cow's milk and in commercial soy-based drinks (“Soy milk”) (Hajirostamloo (2009) Proc. World Acad. Sci. Eng. Technol. 57(9): 436-438 [“Hajirostamloo 2009”]). Fatty acids are essential for human health, yet the high composition of saturated fatty acids in milk can lead to a rise in blood cholesterol levels (Mensink et al. (May 2003) Am. J. Clin. Nutri. 77(5): 1146-1155; [“Mensink 2003”]), cardiovascular diseases and obesity [Mensink 2003; Schaefer (2002) Am. J. Clin. Nutr. 75: 191-212 [“Schaefer 2002”]; Farvid et al. (October 2014) Circulation 130(18): 1568-1578 [“Farvid 2014”]). In comparison to 70% saturated fat in milk (Bodkowski et al. (2016) J. Dairy Sci. 99(1): 57-67 [“Bodkowski 2016”]), soybean extract contains only 15% (Haun et al. (2014) Plant Biotechnol. J. 12(7): 934-940 [“Haun 2014”]). Moreover, soy drinks are a high-quality source for vitamins, including vitamin B, C, E and K, together with beneficial minerals such as calcium, magnesium, iron, phosphorus and zinc (Hajirostamloo 2009). In addition, soybeans are a source for all essential amino acids that are of utmost importance for human health (Kuiken et al. (1949) J. Biol. Chem. 177: 29-36 [“Kuiken 1949”]; Wu (2009) Amino Acids 37: 1-17 [“Wu 2009”]). Finally, soy drink does not contain cholesterol, mammalian growth hormones, antibiotic residues, human opportunistic pathogenic bacteria, or lactose. It is noteworthy that about 30% of ethnically Western Europeans and 70% of decedents from Africa, Eastern Asia and Oceania have difficulties digesting lactose (Muehlhoff 2013).

The increasing global population and the ensuing demand for the nutrients found in milk, together with concerns about environmentally sustainable farming and dietary difficulties in some populations, have contributed to the demand for an animal-free, plant-based milk alternative having a nutrient content comparable to that of milk. There is also a demand for milk alternatives in situations in which the mother is unable to nurse her young.

In addition, there is a demand for a method of producing an animal-free, plant-based milk alternative in such a manner to enable all ingredients to be simply isolated, exuded, secreted, or extracted from a single organism.

There is also a demand for an animal-free, plant-based milk alternative having a reduced content of potential plant allergens, thereby reducing the potential for allergic reactions during human consumption of the plant-based milk alternative.

Moreover, due to modern dietary concerns about the health risks associated with saturated fat intake, there is also a demand for a milk alternative with decreased levels of saturated fat.

Thus, there is a demand for, and it would be highly advantageous to have, a high-quality animal-free milk alternative having a nutrient content comparable to that of milk, as well as means and method for obtaining an animal-free milk alternative from a readily available single organism, such as crop plant, and with a reduction of potential allergens and/or saturated fats.

### SUMMARY OF DISCLOSURE

Disclosed herein in one aspect is a genetically modified plant comprising at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source.

In a related aspect, the at least one cell further comprises; (a) reduced expression of at least one globulin protein as compared to the expression thereof in a corresponding unmodified plant and wherein said at least one cell comprises a reduced content of the at least one globulin protein as compared to the content thereof in a corresponding unmodified plant; or (b) reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant; or (c) reduced expression of at least one seed storage protein as compared to the expression thereof in a corresponding unmodified plan and wherein said at least one cell comprises a reduced content of the at least one seed storage protein as compared to the content thereof in a corresponding unmodified plant; or (d) a combination thereof.

In a related aspect, the relative protein content of each of said at least two milk proteins is at least 70% of the relative protein content of the corresponding mammalian milk protein in the mammal's milk.

In another related aspect, the at least one cell comprises a seed, or a bean, grain, fruit, nut, legume, leaf, stem or root cell.

In another related aspect, the at least two milk proteins are from a non-human mammal. In a further related aspect, the non-human mammal is Bos taurus or Bubalus bubalis. In yet a further related aspect, the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29; the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30; the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31; the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32; the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33; the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In another related aspect, the at least one cell comprises reduced protein content of at least one globulin or derivative thereof, or of at least one desaturase or derivative thereof, or of at least one seed storage protein, or a combination thereof, compared to the protein content thereof in a corresponding unmodified plant.

In another related aspect, the at least one plant cell comprises an increased content of at least one oleic acid or derivative thereof, or at least one stearic acid or derivative thereof, or a reduced content of at least one saturated fat, or any combination thereof, compared to the content thereof in a corresponding unmodified plant.

In another related aspect, the at least one globulin gene is selected from the group consisting of a gene encoding glycinin 1 (GY1), a gene encoding glycinin 2 (GY2), a gene encoding glycinin 3 (GY3), a gene encoding glycinin 4 (GLY4), a gene encoding glycinin 5 (GY5), a gene encoding alpha-conglycinin, a gene encoding alpha-prime-conglycinin, and a gene encoding beta-conglycinin; or the at least one desaturase gene is selected from the group consisting of a gene encoding fatty acid desaturase 1A (FAD2-1A), a gene encoding fatty acid desaturase 1B (FAD2-1B), and a gene encoding delta-9-stearoyl-acyl-carrier protein desaturase (SACPD); or a combination thereof.

In another related aspect, plant comprises a Solanaceae family plant, a Fabaceae family plant, a Poaceae family plant, a Amaranthaceae family plant, a Lamiaceae family plant, a Pedaliaceae family plant, a Cucurbitaceae family plant, a Asteraceae family plant, a Linaceae family plant, a Cannabaceae family plant, a Juglandaceae family plant, a Rosaceae family plant, a Anacardiaceae family plant, a Betalaceae family plant, or a Aracaceae family plant; an algal plant selected from the group consisting of a chlorophyte, a rhodophyte, and a phaeo-phyte; or an algal plant wherein said alga is a C. reinhardtii. In a further related aspect, the plant is selected from the Cannabaceae family and is a Cannabis sativa, Cannabis indica, or Cannabis ruderalis plant; the Solanaceae family and is a Nicotiana benthamiana plant; the Fabacea family and is a soybean plant (Glycine max); the Poaceae family and is an Asian rice (Orya sativa) or an African rice (Oryza glaberrima) plant; or the Aracaceae family, Lemnoidea subfamily, and is duckweed.

In another related aspect, the expression of each of said at least two milk proteins is independently under control of a seed promoter selected from a Seed 1, Seed2, Seed3, Seed4, Seed5, or a Seed6 promoter. In another related aspect, the expression of each of said at least two milk proteins is independently under control of a seed promoter, wherein: expression of beta-casein is under the control of Seed 1 promoter having a nucleotide sequence set forth in SEQ ID NO: 51; expression of kappa-casein is under the control of Seed 2 promoter having a nucleotide sequence set forth in SEQ ID NO: 52; expression of beta-lactoglobulin is under the control of Seed 2 promoter having a nucleotide sequence set forth in SEQ ID NO: 52; expression of alpha-S2-casein is under the control of Seed 3 promoter having a nucleotide sequence set forth in SEQ ID NO: 53; expression of alpha-S1-casein is under the control of Seed 4 promoter having a nucleotide sequence set forth in SEQ ID NO: 54; expression of serum albumin is under the control of Seed 5 promoter having a nucleotide sequence set forth in SEQ ID NO: 55; and expression of alpha-lactalbumin is under the control of Seed 6 promoter having a nucleotide sequence set forth in SEQ ID NO: 56).

In another related aspect, the at least one cell further comprises at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-4B) or a portion thereof, and a gene encoding delta-9-stearoyl-acyl-carrier protein desaturase (SACPD) or a portion thereof; or at least one third series silencer targeted to a polynucleotide encoding at least one seed storage protein or a portion thereof; or a combination thereof.

In one aspect, disclosed herein is a food, medicament, cosmetic or blocking composition comprising a genetically modified plant or a portion, product, isolate, exudate, secretion, or extract thereof, said genetically modified plant or portion, product, isolate, exudate, secretion, or extract thereof comprising at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source.

In a related aspect, the at least one cell further comprises: (a) reduced expression of at least one globulin protein as compared to the expression thereof in a corresponding unmodified plant and wherein said at least one cell comprises a reduced content of the at least one globulin protein as compared to the content thereof in a corresponding unmodified plant; or (b) reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant; or (c) reduced expression of at least one seed storage protein as compared to the expression thereof in a corresponding unmodified plan and wherein said at least one cell comprises a reduced content of the at least one seed storage protein as compared to the content thereof in a corresponding unmodified plant; or (d) a combination thereof.

In another related aspect, the relative protein content of each of said at least two milk proteins is at least 70% of the relative protein content of the corresponding mammalian milk protein in the mammal's milk.

In another related aspect, the at least one cell comprises a seed, or a bean, grain, fruit, nut, legume, leaf, stem or root cell.

In another related aspect, the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29; the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30; the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31; the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32, the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33; the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In another related aspect, the at least one cell further comprises at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof; or at least one third series silencer targeted to a polynucleotide encoding at least one seed storage protein or a portion thereof; or a combination thereof.

In another further related aspect, the milk from a mammal is expressed and has a final concentration of between 1%-60% milk from a mammal or further comprising an unmodified milk alternative from a plant.

In one aspect, disclosed herein is a DNA binary vector or viral vector expressing at least two milk proteins from a mammal, the vector comprising: a selectable marker; polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least two proteins is at least 90% identical to the amino acid sequence of a corresponding mammalian milk protein from the same mammalian source.

In a related aspect, the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29; the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30; the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31; the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32; the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33; the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In another related aspect, the expression of each of said at least two milk proteins is independently under control of a seed promoter, wherein the promoter is selected from any of a Seed1-Seed6 promoter. In a further related aspect, the expression of beta-casein is under the control of Seed 1 promoter having a nucleotide sequence set forth in SEQ ID NO: 51; expression of kappa-casein is under the control of Seed 2 promoter having a nucleotide sequence set forth in SEQ ID NO: 52; expression of beta-lactoglobulin is under the control of Seed 2 promoter having a nucleotide sequence set forth in SEQ ID NO: 52; expression of alpha-S2-casein is under the control of Seed 3 promoter having a nucleotide sequence set forth in SEQ ID NO: 53; expression of alpha-S1-casein is under the control of Seed 4 promoter having a nucleotide sequence set forth in SEQ ID NO: 54; expression of serum albumin is under the control of Seed 5 promoter having a nucleotide sequence set forth in SEQ ID NO: 55; and expression of alpha-lactalbumin is under the control of Seed 6 promoter having a nucleotide sequence set forth in SEQ ID NO: 56).

In a further related aspect, a DNA binary vector or viral vector disclosed herein, further comprises a polynucleotide sequence comprising a silencing element under the control of a promotor targeted to at least one globulin gene; or at least one desaturase gene; or at least one seed storage protein; or a combination thereof. In another related aspect, the silencing element comprises at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof; or at least one third series silencer targeted to a polynucleotide encoding at least one seed storage protein or a portion thereof; or a combination thereof.

In another related aspect, the selectable marker is a BASTA resistance marker.

In another related aspect, the vector comprises a sequence at least 90% identical to the sequence set forth in SEQ ID NO: 50 or at least 90% identical to the sequence set forth in SEQ ID NO: 69.

In one aspect, disclosed herein is a genetically modified plant cell comprising any vector described herein.

In one aspect, disclosed herein is a method of producing a food, medicament, cosmetic or blocking composition comprising a genetically modified plant or portion, product, isolate, exudate, secretion, or extract thereof, the method comprising: providing a DNA binary vector or viral vector for differentially expressing in a plant, proteins from the milk of a mammal, the vector comprising: a selectable marker; polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under control of a promoter, wherein: wherein the amino acid sequence of each of said at least two proteins is at least 90% identical to the amino acid sequence of a corresponding mammalian milk protein from the same mammalian source; and wherein expression of each of said at least two milk proteins is independently under the control of a seed promoter for obtaining a relative protein content of each of said at least two milk proteins of at least 70% of the relative protein content of the corresponding mammalian milk protein in the mammal's milk; transfecting at least one cell of said plant with the DNA binary vector or viral vector; differentially expressing the at least two milk proteins in said at least one plant cell; and optionally adding milk of a mammal to the food, medicament, cosmetic or blocking composition of step (c). In a further related aspect, the endogenous protein is encoded by a globulin gene; an at least one desaturase gene; or an at least one seed storage protein; or a combination thereof.

In another related aspect, the vector comprises a sequence at least 90% identical to the sequence set forth in SEQ ID NO: 50 or at least 90% identical to the sequence set forth in SEQ ID NO: 69.

In a further related aspect of the method of producing a food medicament, cosmetic or blocking composition comprising a genetically modified plant or portion, product, isolate, exudate, secretion, or extract thereof, the DNA binary vector or viral vector further comprises a polynucleotide sequence comprising a silencing element under the control of a promotor targeted to at least one globulin gene; or at least one desaturase gene; or at least one seed storage protein; or a combination thereof.

### BRIEF DESCRIPTION OF THE FIGURES

FIGS. 1A-1G present maps of T-DNA pDGBα binary vector constructs coding for seven cow's milk proteins, each under the control of Solanum lycopersicum ubiquitin promoter 10 (SlPrUbiq10). (FIG. 1A) ALB (serum albumin) (Uniprot id: ALB-P02769); (FIG. 1B) CSN1S1 (α-S1-casein; alpha-S1-casein) (Uniprot id: CSN1S1-P02662); (FIG. 1C) CSN1S2 (α-S2-casein; alpha-S2-casein) (Uniprot id: CSN1S2-P02663); (FIG. 1D) CSN2 (β casein; beta-casein) (Uniprot id: CSN2-P02666); (FIG. 1E) CSN3 (κ casein; kappa-casein) (Uniprot id: CSN3-P02668); (FIG. 1F) LALBA (α-lactalbumin; alpha-lactalbumin) (Uniprot id: LALBA-P00711); and (FIG. 1G) LGB (β-lactoglobulin; beta-lactoglobulin; LACB; progestagen-associated endometrial protein [PAEP]) (Uniprot id: LGB-P02754).

FIG. 2 depicts a histogram showing the relative gene expression of the seven cow's milk genes in transformed Nicotiana benthamiana leaves as a function of mRNA expression as protein. Relative gene expression is presented as fold change compared with non-transformed leaves and normalized to the housekeeping gene F-BOX: ALB (serum albumin), CSN1S1 (α-S1-casein; alpha-S1-casein), CSN1S2 (α-S2-casein; alpha-S2-casein), CSN2 (β casein; beta casein), CSN3 (u casein; kappa casein), LGB (β-lactoglobulin; beta-lactoglobulin), and LALBA (α-lactalbumin; alpha-lactalbumin).

FIGS. 3A-3E show LC-MS/MS proteomic analysis of transiently transformed N. benthamiana leaves. Leaf samples of transiently transformed N. benthamiana were collected five days post-transformation and total protein content was extracted and analyzed using LC-MS/MS. Proteins measured were: (FIG. 3A) CSN1S1 (α-S1-casein; alpha-S1-casein), (FIG. 3B) ALB (serum albumin), (FIG. 3C) CSN2 (β casein; beta casein), (FIG. 3D) LALBA (α-lactalbumin; alpha-lactalbumin), and (FIG. 3E) LGB (LACB) (0-lactoglobulin; beta-lactoglobulin).

FIG. 4 shows a map of pDGB-Ω1 (pDGB-omega1)-seven bovine milk genes, a T-DNA binary plasmid coding for seven major cow's milk proteins and the BASTA resistance gene. The seven major cow's milk proteins are expressed under the control of SlPrUbiq10 (presented as TeUbiq in the figure itself). The seven major cow's milk proteins in the T-DNA plasmid shown are: ALB (serum albumin), CSN1S1 (α-S1-casein; alpha-S1-casein), CSN1S2 (α-S2-casein; alpha-S2-casein), CSN2 (R casein; beta casein), LALBA (α-lactalbumin; alpha-lactalbumin), CSN3 (κ casein; kappa casein), and LGB (β-lactoglobulin; beta-lactoglobulin).

FIG. 5 shows a map of pDGB-α1-SevenGenes+CSY4/Cas9+gRNA (pDGB-alpha1-SevenGenes+CSY4/Cas9+gRNA), a T-DNA plasmid coding for seven major cow's milk proteins, CSY4/CRISPR-Cas9/CRISPR, guide RNA multiplex array, and the BASTA resistance gene. The seven major cow's milk proteins are expressed under control of soybean seed-specific promoters. CSY4/CRISPR and Cas9/CRISPR are expressed under control of one SlPrUbiq10; guide-RNA multiarray complex is expressed under the control of CaMV-35S-promoter (p35S). The seven major cow's milk proteins, each independently expressed under the promotors shown in TABLE 3, are: CSN2 (β casein; beta casein), CSN1S1 (α-S1-casein; alpha-S1-casein), CSN3 (κ casein; kappa casein), CSN1S2 (α-S2-casein; alpha-S2-casein), LGB (0-lactoglobulin; beta-lactoglobulin), LALBA (α-lactalbumin; alpha-lactalbumin), and ALB (serum albumin).

FIGS. 6A-6D show LC-MS/MS proteomic analysis of samples of stably transformed soybean Glycine max plant leaves. Leaf samples were collected, and total protein was extracted and analyzed using nano-UPLC coupled to a quadrupole orbitrap mass spectrometer. Each line is an independent transgenic soybean plant. Proteins produced in each line were: (FIG. 6A) line #54 showing production of CSN2 (β casein) and LALBA (α-lactalbumin), (FIG. 6B) line #55 showing production of CSN2 (β casein) and LALBA (α-lactalbumin), (FIG. 6C) line #61 showing production of CSN2 (β casein) and LALBA (α-lactalbumin), and (FIG. 6D) line #9 showing production of LGB (β-lactoglobulin) and LALBA (α-lactalbumin).

FIG. 7 shows the results of analyzed soybean seed samples of representative transgenic plant lines that produce at least two mammalian milk proteins. Each line is an independent transgenic soybean plant. Mammalian milk proteins produced included alpha-S1-casein, alpha-S2-casein, beta-casein, kappa-casein, and alpha-lactalbumin, wherein the seeds of transgenic line #9 produced all five milk proteins.

### DETAILED DESCRIPTION

It is desirable to provide a nutritional appropriate replacement for humanity's need for milk in an animal-free system that relies on traditional plant agriculture. In addition to the use of milk and other dairy products for drinking and for food, other uses include, but are not limited to, as a medicament (e.g., nutritional supplement or treatment for sunburn, insect bites, rashes, and the like); in a cosmetic anti-aging product or method (e.g., milk baths or rinses for skin or hair); as a medicament or cosmetic treatment for acne, wrinkles, or other blemishes; as a cleaning product; and as a blocking agent for laboratory screening methods (e.g., protein assays).

The present invention utilizes a plant as a tool for harvesting the necessary nutrients for composing a milk-like liquid (milk alternative) or in other words animal-free milk.

To produce animal-free milk in plants, soybean endosperm is genetically modified to produce up to 90% of the cow's milk protein content, up to 95% of the cow's milk protein content, or up to 99% of the cow's milk protein content, with a healthier fatty acid profile which is enriched with non-saturated fats and naturally abundant sugars, minerals and vitamins (see von Schacky (15 Jan. 2007) Cardiovascular Res. 73(2): 310-315 [“von Schacky 2007”]). Although cow's milk contains hundreds of proteins, only seven proteins compose up to 99% of its content: α-s1 casein, α-s2 casein, β-casein, κ-casein, β-lactoglobulin, α-lactalbumin and serum albumin (Reinhardt et al. (April 2013) J. Proteomics 82: 141-154 [“Reinhardt 2013”]). Therefore, introducing these seven genes into the soybean would suffice to imitate the cow's milk protein content. Furthermore, this approach enriches the fatty acid profile of the soybeans, with non-saturated fats, and naturally abundant sugars, minerals and vitamins.

In some embodiments, a genetically modified plant comprises at least one cell expressing at least 1-7 milk proteins. In some embodiments, a genetically modified plant comprises at least one cell expressing at least 2-7 milk proteins. In some embodiments, a genetically modified plant comprises at least one cell expressing at least 3-7 milk proteins. In some embodiments, a genetically modified plant comprises at least one cell expressing at least 4-7 milk proteins. In some embodiments, a genetically modified plant comprises at least one cell expressing at least 5-7 milk proteins. In some embodiments, a genetically modified plant comprises at least one cell expressing at least 6-7 milk proteins. In some embodiments, a genetically modified plant comprises at least one cell expressing 7 milk proteins.

In some embodiments, a genetically modified plant comprises at least one cell expressing at least 1-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 3-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 6-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing at least 1-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a bean cell and said milk proteins are selected from α-s1 casein, α-s2 casein, β-casein, κ-casein, 6-lactoglobulin, α-lactalbumin and serum albumin. In some embodiments, a genetically modified plant comprises at least one cell expressing at least 3-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a bean cell and said milk proteins are selected from α-s1 casein, α-s2 casein, β-casein, κ-casein, 3-lactoglobulin, α-lactalbumin and serum albumin. In some embodiments, a genetically modified plant comprises at least one cell expressing at least 6-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing at least 1-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 3-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 6-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing at least 1-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 3-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 6-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing at least 1-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 3-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a nut cell and said milk proteins are selected from α-s1 casein, α-s2 casein, β-casein, κ-casein, 8-lactoglobulin, α-lactalbumin and serum albumin. In some embodiments, a genetically modified plant comprises at least one cell expressing at least 6-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing at least 1-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 3-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 6-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing at least 1-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 3-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 6-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing at least 1-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 3-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 6-7 milk proteins, wherein the genetically modified plant cell comprises a stem cell and said milk proteins are selected from α-s1 casein, α-s2 casein, β-casein, κ-casein, 6-lactoglobulin, α-lactalbumin and serum albumin. In some embodiments, a genetically modified plant comprises at least one cell expressing 7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing at least 1-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 2-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 3-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 4-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 5-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing at least 6-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing 1-7 milk proteins. In some embodiments, a genetically modified plant comprises at least one cell expressing 2-7 milk proteins. In some embodiments, a genetically modified plant comprises at least one cell expressing 3-7 milk proteins. In some embodiments, a genetically modified plant comprises at least one cell expressing 4-7 milk proteins. In some embodiments, a genetically modified plant comprises at least one cell expressing 5-7 milk proteins. In some embodiments, a genetically modified plant comprises at least one cell expressing 6-7 milk proteins. In some embodiments, a genetically modified plant comprises at least one cell expressing 1, 2, 3, 4, 5, 6, or 7 milk proteins.

In some embodiments, a genetically modified plant comprises at least one cell expressing 1-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a seed cell and said milk proteins are selected from α-s1 casein, α-s2 casein, β-casein, κ-casein, 3-lactoglobulin, α-lactalbumin and serum albumin. In some embodiments, a genetically modified plant comprises at least one cell expressing 3-7 milk proteins, wherein the genetically modified plant cell comprises a seed cell and said milk proteins are selected from α-s1 casein, α-s2 casein, β-casein, κ-casein, 6-lactoglobulin, α-lactalbumin and serum albumin. In some embodiments, a genetically modified plant comprises at least one cell expressing 4-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a seed cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing 1-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 3-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 4-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a bean cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing 1-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 3-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 4-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a grain cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing 1-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 3-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 4-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a fruit cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing 1-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 3-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 4-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a nut cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing 1-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 3-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 4-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a legume cell and said milk proteins are selected from α-s1 casein, α-s2 casein, β-casein, κ-casein, 3-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a legume cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing 1-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a leaf cell and said milk proteins are selected from α-s1 casein, α-s2 casein, β-casein, κ-casein, 3-lactoglobulin, α-lactalbumin and serum albumin. In some embodiments, a genetically modified plant comprises at least one cell expressing 3-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 4-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a leaf cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a leaf cell and said milk proteins are selected from α-s1 casein, α-s2 casein, 3-casein, κ-casein, β-lactoglobulin, α-lactalbumin and serum albumin.

In some embodiments, a genetically modified plant comprises at least one cell expressing 1-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 3-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 4-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a stem cell and said milk proteins are selected from α-s1 casein, α-s2 casein, β-casein, κ-casein, β-lactoglobulin, α-lactalbumin and serum albumin.

In some embodiments, a genetically modified plant comprises at least one cell expressing 1-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 2-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 3-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 4-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 5-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 6-7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin. In some embodiments, a genetically modified plant comprises at least one cell expressing 1, 2, 3, 4, 5, 6, or 7 milk proteins, wherein the genetically modified plant cell comprises a root cell and said milk proteins are selected from α-s1 casein, α-s2 casein, β-casein, κ-casein, β-lactoglobulin, α-lactalbumin and serum albumin.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

In some embodiments, the milk proteins expressed in a plant cell are targeted to a specific location in the seed. In some embodiments, targeting comprises the use of a native plant promotor or targeting element of the plant. In some embodiments, targeting comprises the use of targeting elements of native soybean seed storage proteins. In some embodiments, targeting comprises the use of targeting elements of native soybean seed storage proteins for example but not limited to globulins. In some embodiments, targeting comprises the use of targeting elements of native soybean seed storage proteins and the plant comprises a soybean plant. In some embodiments, targeting comprises the use of targeting elements of native soybean seed storage proteins and the plant comprises a plant other than a soybean plant.

Furthermore, extraction of this animal-free-milk from the modified soybeans of the present invention can rely on industrial techniques based on existing production lines for soy-drinks. Alternatively, the modified soybeans can be manually ground and filtered without the use of special equipment nor electricity. Other methods for obtaining the milk include, but are not limited to, exudation (e.g., from a plant root) or secretion, as well as ingestion, with or without grinding or filtering, of the plant, or of a seed, bean, grain, fruit, nut, legume, leaf, stem, root, portion, or product thereof. Since the production of soy requires significantly less water and energy resources, compared to traditional milk production, our animal-free-milk alternative will serve as a sustainable food source. Furthermore, this plant-based food source will be able to provide children and weak populations in developing countries, a nutritional replacement of milk that could be autonomously grown in rural areas by local population, relying on conventional agriculture techniques. The ‘green milk’ producing soybeans could potentially help feeding children in locations where milk-producing farm animals are not available and liberate villagers from dependency on animal farming.

Alternatively, non-soy plants (e.g., nicotine, rice, peanuts, pea) are used. In some embodiments, the plant is a tobacco plant. In some embodiments, the plant is a rice plant. In some embodiments, the plant is a peanut plant. In some embodiments, the plant is a pea plant. Methods for obtaining the milk include, but are not limited to, isolation, extraction, exudation (e.g., from a plant root), or secretion, as well as ingestion, with or without grinding or filtering, of the plant, or of a seed, bean, grain, fruit, nut, legume, leaf, stem, root, portion, or product thereof.

In some embodiments, the expressed milk proteins are targeted to a specific location in the cell. In some embodiments, the expressed milk proteins are targeted to a protein storage vacuole PSV) in the cell. In some embodiments, the expressed milk proteins are targeted to the endoplasmic reticulum. Methods of targeting proteins to specific locations in a cell is well known in the art.

Additionally, purified proteins from the plant could be incorporated into a capsule, tablet, or other orally taken format as a nutritional supplement. In some embodiments, the purified protein(s) is introduced into a wet or dry food product.

In some embodiments, disclosed herein is a genetically modified plant comprising at least one cell expressing at least two milk proteins from a mammal, where the at least two milk proteins are selected from the group consisting of serum albumin, alpha-S1-casein, alpha-S2-casein, beta-casein, kappa-casein, beta-lactoglobulin, and alpha-lactalbumin, where the amino acid sequence of each of the at least two proteins is at least 90% identical to the amino acid sequence of a corresponding mammalian milk protein from the same mammalian source, and where the at least one cell further comprises: (a) decreased expression of at least one globulin gene as compared to the expression thereof in a corresponding unmodified plant; (b) decreased expression of at least one desaturase gene as compared to the expression thereof in a corresponding unmodified plant; or (c) a combination thereof.

In some embodiments, disclosed herein is a genetically modified plant comprising at least one cell expressing at least three milk proteins from a mammal, where the at least three milk proteins are selected from the group consisting of serum albumin, alpha-S1-casein, alpha-S2-casein, beta-casein, kappa-casein, beta-lactoglobulin, and alpha-lactalbumin, where the amino acid sequence of each of the at least three proteins is at least 90% identical to the amino acid sequence of a corresponding mammalian milk protein from the same mammalian source, and where the at least one cell further comprises: (a) decreased expression of at least one globulin gene as compared to the expression thereof in a corresponding unmodified plant; (b) decreased expression of at least one desaturase gene as compared to the expression thereof in a corresponding unmodified plant; or (c) a combination thereof.

In some embodiments the genetically modified plant comprises at least one cell expressing at least two milk proteins, at least three milk proteins, at least four milk proteins, at least five milk proteins, at least six milk proteins, or at least seven milk proteins from a mammal. In some embodiments the genetically modified plant comprises at least one cell expressing all the milk proteins of serum albumin, alpha-S1-casein, alpha-S2-casein, beta-casein, kappa-casein, beta-lactoglobulin, and alpha-lactalbumin.

In some embodiments, the relative protein content of each of the at least two milk proteins is at least 70% of the relative protein content of the corresponding mammalian milk protein in the mammal's milk. In some embodiments, the relative protein content of each of the at least three milk proteins is at least 70% of the relative protein content of the corresponding mammalian milk protein in the mammal's milk. In some embodiments, the relative protein content of each of the at least four milk proteins is at least 70% of the relative protein content of the corresponding mammalian milk protein in the mammal's milk. In some embodiments, the relative protein content of each of the at least five milk proteins is at least 70% of the relative protein content of the corresponding mammalian milk protein in the mammal's milk. In some embodiments, the relative protein content of each of the at least six milk proteins is at least 70% of the relative protein content of the corresponding mammalian milk protein in the mammal's milk. In some embodiments, the relative protein content of each of the at least seven milk proteins is at least 70% of the relative protein content of the corresponding mammalian milk protein in the mammal's milk.

A skilled artisan would appreciate that the term “relative protein content” of a protein may encompass a proportion (or percentage) of that specific protein within the total protein measured. In some embodiments, the protein content comprises the protein content of a mammal's milk, such as cow's milk. In some embodiments, the protein content comprises the protein content in a plant or portion of a plant, such as a cell, leaf, stem, root, fruit etc. In some embodiments, the protein content comprises the protein content of a genetically modified plant. In some embodiments, the protein content comprises the protein content of an unmodified plant.

It will be appreciated that the “relative protein content of a mammalian milk protein” is the relative measurable amount of a specific milk protein in the mammal's milk, for example, the percent of serum albumin within the total protein in cow's milk. A skilled artisan would be familiar with the relative protein content of each milk protein, for example, caseins represent about 80% of total bovine milk proteins, and within the caseins each of the five different types of caseins, namely alpha-S1-casein, alpha-S2-casein, beta-casein, kappa-casein, and gamma-casein, would have their own average proportion in cow's milk, for example, 38, 10, 35, and 12%, respectively. Accordingly, a skilled artisan would appreciate that the term “70% of the relative protein content of the corresponding mammalian milk protein in the mammal's milk” would mean 70% of the proportion of that protein naturally found in cow's milk. For example, for alpha-S1-casein having an average protein content of 38% in cow's milk, a relative protein content of 70% would mean that alpha-S1-casein has a 26% relative protein content in the genetically modified plant or plant cell.

In some embodiments, the relative protein content of each of the at least two milk proteins is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the relative protein content of the corresponding mammalian milk protein in the mammal's milk. In some embodiments, the relative protein content of each of the at least three milk proteins is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the relative protein content of the corresponding mammalian milk protein in the mammal's milk. In some embodiments, the relative protein content of each of the at least 2, 3, 4, 5, 6, or 7 milk proteins is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the relative protein content of the corresponding mammalian milk protein in the mammal's milk.

In some embodiments, the relative protein content of each of the at least two milk proteins is 100%, or up to 150% of the relative protein content of the corresponding mammalian milk protein in the mammal's milk. In some embodiments, the relative protein content of each of the at least three milk proteins is 100%, or up to 150% of the relative protein content of the corresponding mammalian milk protein in the mammal's milk. In some embodiments, the relative protein content of each of the at least 2, 3, 4, 5, 6, or 7 milk proteins is 100%, or up to 150% of the relative protein content of the corresponding mammalian milk protein in the mammal's milk.

In some embodiments, the genetically modified plant cell comprises a seed, or a bean, grain, fruit, nut, legume, leaf, stem or root cell.

In some embodiments, the milk proteins are from a non-human mammal. In some embodiments the non-human mammal is Bos taurus. In some embodiments the non-human mammal is Bubalus bubalis

In some embodiments, the genetically modified plant comprises at least one cell expressing at least two milk proteins from a mammal selected from the group consisting of serum albumin, alpha-S1-casein, alpha-S2-casein, beta-casein, kappa-casein, beta-lactoglobulin, and alpha-lactalbumin, wherein
a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34;
g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the genetically modified plant comprises at least one cell expressing at least three milk proteins from a mammal selected from the group consisting of serum albumin, alpha-S1-casein, alpha-S2-casein, beta-casein, kappa-casein, beta-lactoglobulin, and alpha-lactalbumin, wherein
a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34;
g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the genetically modified plant comprises at least one cell expressing at least 2, 3, 4, 5, 6, or 7 milk proteins from a mammal selected from the group consisting of serum albumin, alpha-S1-casein, alpha-S2-casein, beta-casein, kappa-casein, beta-lactoglobulin, and alpha-lactalbumin, wherein
a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34;
g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the at least one cell of the genetically modified plant expressing at least three milk proteins further comprises reduced content of a natural cell product. For example, but not limited to in some embodiments in a seed, at least three milk proteins are expressed and there is reduce expression of a natural seed storage protein.

In some embodiments, the seed storage protein comprises a globulin. Removal of globulins, which are seed storage proteins, are not only for removal of allergens. Reduction or removal of a natural seed storage protein may in some embodiments, also allow the cell to produce high amounts of the milk proteins if other naturally seed produced proteins are reduced.

In some embodiments, the at least one cell of the genetically modified plant expressing at least three milk proteins further comprises reduced content of a natural cell product compared to a corresponding unmodified plant, wherein the cell comprises a cell of a plant organ other than a seed.

In some embodiments, the genetically modified plant comprises at least one cell expressing at least three milk proteins and comprises reduced protein content of at least a seed storage protein, compared to the protein content thereof in a corresponding unmodified plant. In some embodiments, the seed storage protein comprises a globulin. In some embodiments, the seed storage protein comprises a globulin and the plant is a soybean plant.

In some embodiments, the at least one cell expressing milk proteins comprises reduced protein content of a native, endogenous protein. In some embodiments, the at least one cell expressing milk proteins comprises reduced protein content of a natural seed storage protein. In some embodiments, the at least one cell expressing milk proteins comprises reduced protein content of at least one globulin or derivative thereof, or of at least one desaturase or derivative thereof, or reduction of a seed storage protein, or a combination thereof, compared to the protein content thereof in a corresponding unmodified plant.

In some embodiments, the genetically modified plant comprises at least one cell comprising an increased content of at least one oleic acid or derivative thereof, or at least one stearic acid or derivative thereof, or a reduced content of at least one saturated fat, or any combination thereof, compared to the content thereof in a corresponding unmodified plant.

In some embodiments, the globulin gene is selected from the group consisting of a gene encoding glycinin 1 (GY1), a gene encoding glycinin 2 (GY2), a gene encoding glycinin 3 (GY3), a gene encoding glycinin 4 (GLY4), a gene encoding glycinin 5 (GY5), a gene encoding alpha-conglycinin, a gene encoding alpha-prime-conglycinin, and a gene encoding beta-conglycinin.

In some embodiments, the desaturase gene is selected from the group consisting of a gene encoding fatty acid desaturase 1A (FAD2-1A), a gene encoding fatty acid desaturase 1B (FAD2-1B), and a gene encoding delta-9-stearoyl-acyl-carrier protein desaturase (SACPD).

In some embodiments, the genetically modified plant comprises:
a) a Solanaceae family plant, a Fabaceae family plant, a Poaceae family plant, a Amaranthaceae family plant, a Lamiaceae family plant, a Pedaliaceae family plant, a Cucurbitaceae family plant, an Asteraceae family plant, a Linaceae family plant, a Cannabaceae family plant, a Juglandaceae family plant, a Rosaceae family plant, a Anacardiaceae family plant, a Betalaceae family plant, or a Aracaceae family plant;
b) an algal plant selected from the group consisting of a chlorophyte, a rhodophyte, and a phaeo-phyte; or
c) an algal plant wherein said alga is a C. reinhardtii.

In some embodiments, the genetically modified plant comprises a plant from the Solanaceae family and is a Nicotiana benthamiana plant. In some embodiments, the genetically modified plant comprises a plant from the Fabacea family and is a soybean plant (Glycine max). In some embodiments, the genetically modified plant comprises a plant from the Poaceae family and is an Asian rice (Oryza sativa). In some embodiments, the genetically modified plant comprises a plant from the Poaceae family and is an African rice (Oryza glaberrima) plant.

In some embodiments, the genetically modified plant comprises at least one cell expressing at least two milk proteins from a mammal selected from the group consisting of serum albumin, alpha-S1-casein, alpha-S2-casein, beta-casein, kappa-casein, beta-lactoglobulin, and alpha-lactalbumin, where the expression is under the control of a plant seed promoter. In some embodiments, the
a) expression of beta-casein is under the control of Seed 1 promoter having a nucleotide sequence set forth in SEQ ID NO: 51;
b) expression of kappa-casein is under the control of Seed 2 promoter having a nucleotide sequence set forth in SEQ ID NO: 52;
c) expression of beta-lactoglobulin is under the control of Seed 2 promoter having a nucleotide sequence set forth in SEQ ID NO: 52;
d) expression of alpha-S2-casein is under the control of Seed 3 promoter having a nucleotide sequence set forth in SEQ ID NO: 53;
e) expression of alpha-S1-casein is under the control of Seed 4 promoter having a nucleotide sequence set forth in SEQ ID NO: 54;
f) expression of serum albumin is under the control of Seed 5 promoter having a nucleotide sequence set forth in SEQ ID NO: 55; and
g) expression of alpha-lactalbumin is under the control of Seed 6 promoter having a nucleotide sequence set forth in SEQ ID NO: 56).

In some embodiments, the genetically modified plant comprises at least one cell expressing at least three milk proteins from a mammal selected from the group consisting of serum albumin, alpha-S1-casein, alpha-S2-casein, beta-casein, kappa-casein, beta-lactoglobulin, and alpha-lactalbumin, where the expression is under the control of a plant seed promoter. In some embodiments, the
a) expression of beta-casein is under the control of Seed 1 promoter having a nucleotide sequence set forth in SEQ ID NO: 51;
b) expression of kappa-casein is under the control of Seed 2 promoter having a nucleotide sequence set forth in SEQ ID NO: 52;
c) expression of beta-lactoglobulin is under the control of Seed 2 promoter having a nucleotide sequence set forth in SEQ ID NO: 52;
d) expression of alpha-S2-casein is under the control of Seed 3 promoter having a nucleotide sequence set forth in SEQ ID NO: 53;
e) expression of alpha-S1-casein is under the control of Seed 4 promoter having a nucleotide sequence set forth in SEQ ID NO: 54;
f) expression of serum albumin is under the control of Seed 5 promoter having a nucleotide sequence set forth in SEQ ID NO: 55; and
g) expression of alpha-lactalbumin is under the control of Seed 6 promoter having a nucleotide sequence set forth in SEQ ID NO: 56).

In some embodiments, the genetically modified plant comprises at least one cell expressing at least 2, 3, 4, 5, 6, or 7 milk proteins from a mammal selected from the group consisting of serum albumin, alpha-S1-casein, alpha-S2-casein, beta-casein, kappa-casein, beta-lactoglobulin, and alpha-lactalbumin, where the expression is under the control of a plant seed promoter. In some embodiments, the
a) expression of beta-casein is under the control of Seed 1 promoter having a nucleotide sequence set forth in SEQ ID NO: 51;
b) expression of kappa-casein is under the control of Seed 2 promoter having a nucleotide sequence set forth in SEQ ID NO: 52;
c) expression of beta-lactoglobulin is under the control of Seed 2 promoter having a nucleotide sequence set forth in SEQ ID NO: 52;
d) expression of alpha-S2-casein is under the control of Seed 3 promoter having a nucleotide sequence set forth in SEQ ID NO: 53;
e) expression of alpha-S1-casein is under the control of Seed 4 promoter having a nucleotide sequence set forth in SEQ ID NO: 54;
f) expression of serum albumin is under the control of Seed 5 promoter having a nucleotide sequence set forth in SEQ ID NO: 55; and
g) expression of alpha-lactalbumin is under the control of Seed 6 promoter having a nucleotide sequence set forth in SEQ ID NO: 56).

While certain embodiments reflect control of milk proteins under the control of a seed promoter, one skilled in the art would appreciate that other promoters could be utilized here, including but not limited to inducible promoter, constitutive promoters, specific plant part promoters, specific plant developmental promoters, or other endogenous promoters present in the plant cell.

In some embodiments, the genetically modified plant comprises at least one cell comprising at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof.

In some embodiments, the genetically modified plant comprises at least one cell comprising at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, the genetically modified plant comprises at least one cell comprising at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof, and at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, disclosed herein is a food, medicament, cosmetic or blocking composition comprising a genetically modified plant or a portion, product, isolate, exudate, secretion, or extract thereof, the genetically modified plant or portion, product, isolate, exudate, secretion, or extract thereof comprising at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source, and wherein the at least one cell further comprises a decreased expression of at least one globulin gene as compared to the expression thereof in a corresponding unmodified plant, decreased expression of at least one desaturase gene as compared to the expression thereof in a corresponding unmodified plant, or a combination thereof.

In some embodiments, disclosed herein is a food, medicament, cosmetic or blocking composition comprising a genetically modified plant or a portion, product, isolate, exudate, secretion, or extract thereof, the genetically modified plant or portion, product, isolate, exudate, secretion, or extract thereof comprising at least one cell expressing at least three milk proteins from a mammal, the at least three milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least three proteins is at least 90% identical to the amino acid sequence of a corresponding mammalian milk protein from the same mammalian source, and wherein the at least one cell further comprises a decreased expression of at least one globulin gene as compared to the expression thereof in a corresponding unmodified plant, decreased expression of at least one desaturase gene as compared to the expression thereof in a corresponding unmodified plant, or a combination thereof.

In some embodiments, disclosed herein is a food, medicament, cosmetic or blocking composition comprising a genetically modified plant or a portion, product, isolate, exudate, secretion, or extract thereof, the genetically modified plant or portion, product, isolate, exudate, secretion, or extract thereof comprising at least one cell expressing at least 2, 3, 4, 5, 6, or 7 milk proteins from a mammal, the at least 2, 3, 4, 5, 6, or 7 milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least 2, 3, 4, 5, 6, or 7 proteins is at least 90% identical to the amino acid sequence of a corresponding mammalian milk protein from the same mammalian source, and wherein the at least one cell further comprises a decreased expression of at least one globulin gene as compared to the expression thereof in a corresponding unmodified plant, decreased expression of at least one desaturase gene as compared to the expression thereof in a corresponding unmodified plant, or a combination thereof.

In some embodiments, the food, medicament, cosmetic or blocking composition comprises a genetically modified plant cell comprising at least two milk proteins, at least three milk proteins, at least four milk proteins, at least five milk proteins, at least six milk proteins, or at least seven milk proteins from a mammal. In some embodiments the food, medicament, cosmetic or blocking composition comprises a genetically modified plant cell comprising the milk proteins of serum albumin, alpha-S1-casein, alpha-S2-casein, beta-casein, kappa-casein, beta-lactoglobulin, and alpha-lactalbumin.

In some embodiments, the relative protein content of each of the at least two milk proteins is at least 70% of the relative protein content of the corresponding mammalian milk protein in the mammal's milk. In some embodiments, the relative protein content of each of the at least three milk proteins is at least 70% of the relative protein content of the corresponding mammalian milk protein in the mammal's milk. In some embodiments, the relative protein content of each of the at least 2, 3, 4, 5, 6, or 7 milk proteins is at least 70% of the relative protein content of the corresponding mammalian milk protein in the mammal's milk.

In some embodiments, the food, medicament, cosmetic or blocking composition comprises a genetically modified plant cell comprising a seed, or a bean, grain, fruit, nut, legume, leaf, stem or root cell.

In some embodiments, the food, medicament, cosmetic or blocking composition comprises a genetically modified plant comprising at least one cell expressing at least two milk proteins from a mammal selected from the group consisting of serum albumin, alpha-S1-casein, alpha-S2-casein, beta-casein, kappa-casein, beta-lactoglobulin, and alpha-lactalbumin, wherein
(a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
(d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
(e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the food, medicament, cosmetic or blocking composition comprises a genetically modified plant comprising at least one cell expressing at least three milk proteins from a mammal selected from the group consisting of serum albumin, alpha-S1-casein, alpha-S2-casein, beta-casein, kappa-casein, beta-lactoglobulin, and alpha-lactalbumin, wherein
(a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
(d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
(e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the food, medicament, cosmetic or blocking composition comprises a genetically modified plant comprising at least one cell expressing at least 2, 3, 4, 5, 6, or 7 milk proteins from a mammal selected from the group consisting of serum albumin, alpha-S1-casein, alpha-S2-casein, beta-casein, kappa-casein, beta-lactoglobulin, and alpha-lactalbumin, wherein
(a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
(d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
(e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the food, medicament, cosmetic or blocking composition comprises a genetically modified plant comprising at least one cell comprising at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof.

In some embodiments, the food, medicament, cosmetic or blocking composition comprises a genetically modified plant comprising at least one cell comprising at least one second series silencer targeted to a polynucleotide encoding at least one desaturase protein or a portion thereof selected from the group consisting of fatty acid desaturase 1A (FAD2-4A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, the food, medicament, cosmetic or blocking composition comprises a genetically modified plant comprising at least one cell comprising at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof, and at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, the food, medicament, cosmetic or blocking composition comprises milk from a mammal for a final concentration of between 1%-60% milk from a mammal or further comprising an unmodified milk alternative from a plant.

In some embodiments, disclosed herein is a DNA binary vector or viral vector expressing at least two milk proteins from a mammal, the vector comprising a selectable marker, polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least two proteins is at least 90% identical to the amino acid sequence of a corresponding mammalian milk protein from the same mammalian source, and a polynucleotide sequence comprising a silencing element under the control of a promotor targeted to at least one globulin gene; at least one desaturase gene; or a combination thereof. In some embodiments, disclosed herein is a DNA binary vector or viral vector expressing at least three milk proteins from a mammal, the vector comprising a selectable marker, polynucleotide sequences encoding at least three milk proteins from a mammal, wherein said at least three milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least three proteins is at least 90% identical to the amino acid sequence of a corresponding mammalian milk protein from the same mammalian source, and a polynucleotide sequence comprising a silencing element under the control of a promotor targeted to at least one globulin gene; at least one desaturase gene; or a combination thereof. In some embodiments, disclosed herein is a DNA binary vector or viral vector expressing at least 2, 3, 4, 5, 6, or 7 milk proteins from a mammal, the vector comprising a selectable marker, polynucleotide sequences encoding at least 2, 3, 4, 5, 6, or 7 milk proteins from a mammal, wherein said at least 2, 3, 4, 5, 6, or 7 milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least 2, 3, 4, 5, 6, or 7 proteins is at least 90% identical to the amino acid sequence of a corresponding mammalian milk protein from the same mammalian source, and a polynucleotide sequence comprising a silencing element under the control of a promotor targeted to at least one globulin gene; at least one desaturase gene; or a combination thereof.

In some embodiments, the DNA binary vector or viral vector expresses at least two milk proteins, at least three milk proteins, at least four milk proteins, at least five milk proteins, at least six milk proteins, or at least seven milk proteins from a mammal. In some embodiments the DNA binary vector or viral vector expresses the milk proteins of serum albumin, alpha-S1-casein, alpha-S2-casein, beta-casein, kappa-casein, beta-lactoglobulin, and alpha-lactalbumin. In some embodiments, the DNA binary vector or viral vector expresses at least three milk proteins, at least three milk proteins, at least four milk proteins, at least five milk proteins, at least six milk proteins, or at least seven milk proteins from a mammal. In some embodiments the DNA binary vector or viral vector expresses the milk proteins of serum albumin, alpha-S1-casein, alpha-S2-casein, beta-casein, kappa-casein, beta-lactoglobulin, and alpha-lactalbumin. In some embodiments, the DNA binary vector or viral vector expresses at least 2, 3, 4, 5, 6, or 7 milk proteins, at least three milk proteins, at least four milk proteins, at least five milk proteins, at least six milk proteins, or at least seven milk proteins from a mammal. In some embodiments the DNA binary vector or viral vector expresses the milk proteins of serum albumin, alpha-S1-casein, alpha-S2-casein, beta-casein, kappa-casein, beta-lactoglobulin, and alpha-lactalbumin.

In some embodiments, the DNA binary vector or viral vector expresses at least two milk proteins selected from the group comprising serum albumin, alpha-S1-casein, alpha-S2-casein, beta-casein, kappa-casein, beta-lactoglobulin, and alpha-lactalbumin, wherein
(a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
(d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
(e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the DNA binary vector or viral vector expresses at least three milk proteins selected from the group comprising serum albumin, alpha-S1-casein, alpha-S2-casein, beta-casein, kappa-casein, beta-lactoglobulin, and alpha-lactalbumin, wherein
(a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
(d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
(e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the DNA binary vector or viral vector expresses at least 2, 3, 4, 5, 6, or 7 milk proteins selected from the group comprising serum albumin, alpha-S1-casein, alpha-S2-casein, beta-casein, kappa-casein, beta-lactoglobulin, and alpha-lactalbumin, wherein
(a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
(d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
(e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

In some embodiments, the DNA binary vector or viral vector expresses milk proteins from a mammal selected from the group consisting of serum albumin, alpha-S1-casein, alpha-S2-casein, beta-casein, kappa-casein, beta-lactoglobulin, and alpha-lactalbumin, where the expression is independently under control of an endogenous promoter. In some embodiments, the DNA binary vector or viral vector expresses at least two milk proteins from a mammal selected from the group consisting of serum albumin, alpha-S1-casein, alpha-S2-casein, beta-casein, kappa-casein, beta-lactoglobulin, and alpha-lactalbumin, where the expression is independently under control of a seed promoter. In some embodiments, the DNA binary vector or viral vector expresses at least three milk proteins from a mammal selected from the group consisting of serum albumin, alpha-S1-casein, alpha-S2-casein, beta-casein, kappa-casein, beta-lactoglobulin, and alpha-lactalbumin, where the expression is independently under control of a seed promoter. In some embodiments, the DNA binary vector or viral vector expresses at least 2, 3, 4, 5, 6, or 7 milk proteins from a mammal selected from the group consisting of serum albumin, alpha-S1-casein, alpha-S2-casein, beta-casein, kappa-casein, beta-lactoglobulin, and alpha-lactalbumin, where the expression is independently under control of a seed promoter.

In some embodiments, the
(a) expression of beta-casein is under the control of Seed 1 promoter having a nucleotide sequence set forth in SEQ ID NO: 51;
(b) expression of kappa-casein is under the control of Seed 2 promoter having a nucleotide sequence set forth in SEQ ID NO: 52;
(c) expression of beta-lactoglobulin is under the control of Seed 2 promoter having a nucleotide sequence set forth in SEQ ID NO: 52;
(d) expression of alpha-S2-casein is under the control of Seed 3 promoter having a nucleotide sequence set forth in SEQ ID NO: 53;
(e) expression of alpha-S1-casein is under the control of Seed 4 promoter having a nucleotide sequence set forth in SEQ ID NO: 54;
(f) expression of serum albumin is under the control of Seed 5 promoter having a nucleotide sequence set forth in SEQ ID NO: 55; and
(g) expression of alpha-lactalbumin is under the control of Seed 6 promoter having a nucleotide sequence set forth in SEQ ID NO: 56).

In some embodiments, the DNA binary vector or viral vector comprises a silencing element. In some embodiments, the silencing element comprises at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof.

In some embodiments, the silencing element comprises at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, a silencing element described herein comprises at least one third series silencer targeted to a polynucleotide encoding at least a seed storage protein. Design and use of silencing elements are well known in the art.

In some embodiments, the silencing element comprises at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof, and at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, the DNA binary vector or viral vector comprises a selectable marker. In some embodiments, the selectable marker comprises a BASTA resistance marker.

In some embodiments, the DNA binary vector or viral vector comprises a sequence at least 90% identical to S sequence set forth in EQ ID NO: 50.

In some embodiments, the DNA binary vector or viral vector comprises a sequence at least 90% identical to sequence set forth in SEQ ID NO: 69.

In some embodiments, disclosed herein is a genetically modified plant cell comprising the DNA binary vector or viral vector described herein in detail.

In some embodiments, disclosed herein is a method of producing a food, medicament, cosmetic or blocking composition comprising a genetically modified plant or portion, product, isolate, exudate, secretion, or extract thereof, the method comprising:
(a) providing a DNA binary vector or viral vector for differentially expressing in a plant, proteins from the milk of a mammal, the vector comprising:
   (i) a selectable marker;
   (ii) polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under control of a promoter, wherein:
      (1) wherein the amino acid sequence of each of said at least two proteins is at least 90% identical to the amino acid sequence of a corresponding mammalian milk protein from the same mammalian source; and
      (2) wherein expression of each of said at least two milk proteins is independently under the control of a seed promoter for obtaining a relative protein content of each of said at least two milk proteins of at least 70% of the relative protein content of the corresponding mammalian milk protein in the mammal's milk;
(b) transfecting at least one cell of said plant with the DNA binary vector or viral vector;
(c) differentially expressing the at least two milk proteins in said at least one plant cell; and
(d) optionally adding milk of a mammal to the food, medicament, cosmetic or blocking composition of step (c).

In some embodiments of the methods of producing a food, medicaments, cosmetic, or blocking composition, the polynucleotide sequences of (ii) further comprise a polynucleotide sequence comprising a silencing element under the control of a promotor targeted to at least one globulin gene; at least one desaturase gene; or at least one seed storage protein; or a combination thereof.

In some embodiments, disclosed herein is a method of producing a food, medicament, cosmetic or blocking composition comprising a genetically modified plant or portion, product, isolate, exudate, secretion, or extract thereof, the method comprising
(a) providing a DNA binary vector or viral vector for differentially expressing in a plant, proteins from the milk of a mammal, the vector comprising:
   (i) a selectable marker;
   (ii) polynucleotide sequences encoding at least 2, 3, 4, 5, 6, or 7, milk proteins from a mammal, wherein the at least two milk proteins are selected from the group consisting of serum albumin, alpha-S1-casein, alpha-S2-casein, beta-casein, kappa-casein, beta-lactoglobulin, and alpha-lactalbumin, each independently under control of a promoter, wherein:
      (1) wherein the amino acid sequence of each of the at least two proteins is at least 90% identical to the amino acid sequence of a corresponding mammalian milk protein from the same mammalian source; and
      (2) wherein expression of each of said at least two milk proteins is independently under the control of a seed promoter for obtaining a relative protein content of each of said at least two milk proteins of at least 70% of the relative protein content of the corresponding mammalian milk protein in the mammal's milk;
      and
   (iii) a polynucleotide sequence comprising a silencing element under the control of a promotor targeted to at least one globulin gene; at least one desaturase gene; at least one seed storage protein, or a combination thereof;
(b) transfecting at least one cell of said plant with the DNA binary vector or viral vector; and
(c) differentially expressing the at least 2, 3, 4, 5, 6, or 7 milk proteins in said at least one plant cell.

One skilled in the art would appreciate that expression of milk proteins described herein comprises expression of more than a single milk protein in a cell. In some embodiments, 2 milk proteins are expressed in an at least one plant cell. In some embodiments, 3 milk proteins are expressed in an at least one plant cell. In some embodiments, 4 milk proteins are expressed in an at least one plant cell. In some embodiments, 5 milk proteins are expressed in an at least one plant cell. In some embodiments, 6 milk proteins are expressed in an at least one plant cell. In some embodiments, 7 milk proteins are expressed in an at least one plant cell. In some embodiments, 2-7 milk proteins are expressed in an at least one plant cell. In some embodiments, 3-7 milk proteins are expressed in an at least one plant cell. In some embodiments, 4-7 milk proteins are expressed in an at least one plant cell. In some embodiments, 5-7 milk proteins are expressed in an at least one plant cell. In some embodiments, 6-7 milk proteins are expressed in an at least one plant cell. In some embodiments, 2, 3, 4, 5, 6, or 7 milk proteins are expressed in an at least one plant cell.

In some embodiments, a method of producing a food, medicament, cosmetic or blocking composition further comprises the step of adding milk of a mammal to the food, medicament, cosmetic or blocking composition.

In some embodiments of a method of producing a food, medicament, cosmetic or blocking composition, the DNA binary vector or viral vector comprises a sequence at least 90% identical to S sequence set forth in EQ ID NO. 50. In some embodiments, the DNA binary vector or viral vector comprises a sequence at least 90% identical to sequence set forth in SEQ ID NO: 69.

According to one aspect, the present invention provides a genetically modified plant comprising at least one cell expressing at least one protein from the milk of a mammal, the at least one protein being selected from the group consisting of serum albumin, alpha-S1-casein, alpha-S2-casein, beta-casein, kappa-casein, beta-lactoglobulin, and alpha-lactalbumin and expressed in the genetically modified plant or a seed, bean, grain, fruit, nut, legume, leaf, stem, root, or portion, thereof, wherein each of said at least one protein is a recombinant protein at least 90% identical to the corresponding mammalian protein amino acid sequence, said recombinant protein being produced by the plant cell.

In one embodiment, the plant does not produce or comprise any other milk proteins aside from serum albumin, alpha-S1-casein, alpha-S2-casein, beta-casein, kappa-casein, beta-lactoglobulin, or alpha-lactalbumin.

In one embodiment, the mammal is selected from the Bos genus and
(a) the amino acid sequence of the serum albumin is at least 90% identical to the amino acid sequence set forth in SEQ ID NO: 36, or the polynucleotide encoding the serum albumin encodes a serum albumin that is at least 90% identical to the serum albumin encoded by the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide encoding the alpha-S1-casein encodes an alpha-S1-casein that is at least 90% identical to the alpha-S1-casein encoded by the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide encoding the alpha-S2-casein encodes an alpha-S2-casein that is at least 90% identical to the alpha-S2-casein encoded by the polynucleotide sequence set forth in SEQ ID NO: 31;
(d) the amino acid sequence of the beta-casein is at least 90% identical to the amino acid sequence set forth in SEQ ID NO: 39 or the polynucleotide encoding the beta-casein encodes a beta-casein that is at least 90% identical to the beta-casein encoded by the polynucleotide sequence set forth in SEQ ID NO: 32;
(e) the amino acid sequence of the kappa-casein is at least 90% identical to the amino acid sequence set forth in SEQ ID NO: 40 or the polynucleotide encoding the kappa-casein encodes a kappa-casein that is at least 90% identical to the kappa-casein encoded by the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide encoding the beta-lactoglobulin encodes a beta-lactoglobulin that is at least 90% identical to the beta-lactoglobulin encoded by the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide encoding the alpha-lactalbumin encodes an alpha-lactalbumin that is at least 90% identical to the alpha-lactalbumin encoded by the polynucleotide sequence set forth in SEQ ID NO: 35.

In one embodiment, the at least one protein from the milk of a mammal is from a human mammal. Alternatively, the at least one protein from the milk of a mammal is from a non-human mammal. In one embodiment, the non-human mammal is from the Bovidae family. In one embodiment, the non-human mammal is from a genus of the Bovidae family selected from the group consisting of the Bos genus, the Capra genus, the Bubalus genus, the Syncerus genus, the Ovis genus, and the Bison genus. In one embodiment, the at least one protein from the milk of a mammal is from a mammal selected from the Bovidae family, the Bos genus, or Bos taurus. In one embodiment, the at least one protein from the milk of a mammal is selected from the Bubalus genus or Bubalus bubalis (water buffalo).

In one embodiment, the at least one cell further comprises: decreased expression of at least one globulin gene protein; or decreased expression of at least one desaturase gene, wherein expression of the at least one globulin gene protein or expression of the at least one desaturase gene protein is reduced in the modified plant compared to its expression in a corresponding unmodified plant, thereby the modified plant comprises reduced content of at least one globulin or derivative thereof, or of at least one desaturase or derivative thereof, or comprises an increased content of at least one oleic acid or derivative thereof or at least one stearic acid or derivative thereof or a reduced content of at least one saturated fat, compared to the corresponding unmodified plant.

In one embodiment, the plant is from the Solanaceae family, the Nicotiana genus, or Nicotiana benthamiana. In another embodiment, the plant is from the Fabaceae family, the Glycine genus, or Glycine max (soy/soybean). Alternatively, the plant is from the Fabaceae family, but is selected from the group consisting of the Cicer genus (e.g., Cicer arietinum [chickpea, garbanzo bean]), the Phaseolus genus (e.g., Phaseolus vilgaris [string bean, common bean, French bean]), the Pisum genus (e.g., Pisum sativum [pea]), the Arachis genus (e.g., Arachis hypogaea [peanut]), and the Lupinus genus (e.g., Lupinus albus [lupin/lupine]). In yet another embodiment, the plant is from the Poaceae family, the Oryza genus (e.g., rice), or is selected from the group consisting of Oryza sativa and Oryza glaberrima. Alternatively, the plant is from the Poaceae family, but is selected from the group consisting of the Hordeum genus (e.g., Hordeum vulgare [barley]), the Avena genus (e.g., Avena sativa [oat]), and the Triticum genus (e.g., Triticum spelta [spelt]). In still another embodiment, the plant is from the Amaranthaceae family, the Chenopodium genus, or Chenopodium quinoa (quinoa). In still another embodiment, the plant is from the Lamiaceae family, the Salvia genus, or Salvia hispanica (chia). In still another embodiment, the plant is from the Pedaliaceae family, the Sesamum genus, or Sesamum indicum (sesame, benne). In still another embodiment, the plant is from the Cucurbitaceae family or the Cucurbita genus (e.g., squash/pumpkin, including, but not limited to, Cucurbita pepo, Cucurbita maxima, Cucurbita argyrosperma, or Cucurbita moschata). In still another embodiment, the plant is from the Asteraceae family, the Helianthus genus, or is selected from the group consisting of Helianthus anmus (sunflower), Helianthus verticallatus (whorled sunflower) and Helianthus tuberosus (Jerusalem artichoke). In still another embodiment, the plant is from the Linaceae family, the Linum genus, or Linum usitatissimum (flax, linseed). In still another embodiment, the plant is from the Cannabaceae family (e.g., hemp, including Cannabis sativa, or (Cannabis indica, or Cannabis ruderalis). In still another embodiment, the plant is from the Betalaceae family or the Corylus genus (e.g., hazel/hazelnut/cobnut/filbert nut, including, but not limited to, Corylus avellana). In still another embodiment, the plant is from the Juglandaceae family, the Juglans genus, or is selected from the group consisting of Juglans regia (Persian or English walnut), Juglans nigra (black walnut), and Juglans cinera (butternut). In still another embodiment, the plant is from the Rosaceae family, the Prunus genus, or is Prunus dulcis (almond) or Prunus amygdalus. In still another embodiment, the plant is from the Anacardiaceae family, or is selected from the group consisting of the Anacardium genus (e.g., Anacardium occidentale [cashew]) and the Pistacia genus (e.g., Pistacia vera [pistachio]). In still another embodiment, the plant is from the Aracaceae family (e.g., from the Lemnoidea subfamily [duckweed], or the Cocus genus, or the plant is Cocus nucifera (e.g., coconut). In one embodiment, the plant is any one of a variety of algae, including, but not limited to, chlorophytes (green algae), rhodophytes (red algae), or phaeo-phytes (brown algae). In one embodiment, the green algae is C. reinhardtii.

According to another aspect, the present invention provides a genetically modified plant comprising at least one cell expressing at least one protein from the milk of a mammal, the at least one protein being selected from the group consisting of serum albumin, alpha-S1-casein, alpha-S2-casein, beta-casein, kappa-casein, beta-lactoglobulin, and alpha-lactalbumin and differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical mammalian species, wherein each of said at least one protein is a recombinant protein at least 90% identical to the corresponding mammalian protein amino acid sequence, said recombinant protein being produced by the plant cell.

In one embodiment, the plant does not produce or comprise any other milk proteins aside from serum albumin, alpha-S1-casein, alpha-S2-casein, beta-casein, kappa-casein, beta-lactoglobulin, or alpha-lactalbumin.

In one embodiment, the at least one protein from the milk of a mammal is from a mammal selected from the Bovidae family, the Bos genus, or Bas taurus.

In one embodiment, the plant is from the Fabaceae family, the Glycine genus, or Glycine max.

In one embodiment, the mammal is selected from the Bos genus and:
(a) the amino acid sequence of the serum albumin is at least 90% identical to the amino acid sequence set forth in SEQ ID NO: 36, or the polynucleotide encoding the serum albumin encodes a serum albumin that is at least 90% identical to the serum albumin encoded by the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide encoding the alpha-S1-casein encodes an alpha-S1-casein that is at least 90% identical to the alpha-S1-casein encoded by the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide encoding the alpha-S2-casein encodes an alpha-S2-casein that is at least 90% identical to the alpha-S2-casein encoded by the polynucleotide sequence set forth in SEQ ID NO: 31;
(d) the amino acid sequence of the beta-casein is at least 90% identical to the amino acid sequence set forth in SEQ ID NO: 39 or the polynucleotide encoding the beta-casein encodes a beta-casein that is at least 90% identical to the beta-casein encoded by the polynucleotide sequence set forth in SEQ ID NO: 32;
(e) the amino acid sequence of the kappa-casein is at least 90% identical to the amino acid sequence set forth in SEQ ID NO: 40 or the polynucleotide encoding the kappa-casein encodes a kappa-casein that is at least 90% identical to the kappa-casein encoded by the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide encoding the beta-lactoglobulin encodes a beta-lactoglobulin that is at least 90% identical to the beta-lactoglobulin encoded by the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide encoding the alpha-lactalbumin encodes an alpha-lactalbumin that is at least 90% identical to the alpha-lactalbumin encoded by the polynucleotide sequence set forth in SEQ ID NO: 35.

In one embodiment, the plant is selected from the genus Glycine and expression of each of the at least one protein from the milk of a mammal is independently under control of a seed promoter. Alternatively, the plant is selected from a non-Glycine genus and expression of each of the at least one protein from the milk of a mammal is independently under control of a seed promoter. In one embodiment, the seed promoter is selected independently from the group consisting of Seed 1, Seed 2, Seed 3, Seed 4, Seed 5, and Seed 6.

One skilled in the art would appreciate that though particular milk proteins have been exemplified below, wherein their expression is under the control of a specific promoter, any of the promoters Seed 1-Seed 6 may in certain embodiments be pair with any of the 7 milk proteins being expressed. For example, but not limited to, in some embodiments, serum albumin is expressed under the control of any of the promoters Seed 1-Seed6. In some embodiments, alpha-S1-casein is expressed under the control of any of the promoters Seed 1-Seed6. In some embodiments, alpha-S2-casein is expressed under the control of any of the promoters Seed 1-Seed6. In some embodiments, beta-casein is expressed under the control of any of the promoters Seed 1-Seed6. In some embodiments, kappa-casein is expressed under the control of any of the promoters Seed 1-Seed6. In some embodiments, beta-lactoglobulin is expressed under the control of any of the promoters Seed 1-Seed6. In some embodiments, alpha-lactalbumin is expressed under the control of any of the promoters Seed 1-Seed6.

In one embodiment, the plant is selected from the genus Glycine, and the at least one cell further comprises:
(a) decreased expression of at least one globulin gene protein selected from the group consisting of a gene encoding glycinin 1 (GY1), a gene encoding glycinin 2 (GY2), a gene encoding glycinin 3 (GY3), a gene encoding glycinin 4 (GLY4), a gene encoding glycinin 5 (GY5), a gene encoding alpha-conglycinin, a gene encoding alpha-prime-conglycinin, and a gene encoding beta-conglycinin; or
(b) decreased expression of at least one desaturase gene selected from the group consisting of a gene encoding fatty acid desaturase 1A (FAD2-1A), a gene encoding fatty acid desaturase 1B (FAD2-1B), and a gene encoding delta-9-stearoyl-acyl-carrier protein desaturase (SACPD) compared to its expression in a corresponding unmodified plant,
wherein expression of the at least one globulin gene protein or expression of the at least one desaturase gene protein is reduced in the modified plant compared to its expression in a corresponding unmodified plant, thereby the modified plant comprises reduced content of at least one globulin or derivative thereof, or of at least one desaturase or derivative thereof, or comprises an increased content of at least one oleic acid or derivative thereof or at least one stearic acid or derivative thereof or a reduced content of at least one saturated fat, compared to the corresponding unmodified plant.

In one embodiment, the expression of the at least one gene or any combination thereof is decreased, the decrease comprising mutagenizing the at least one gene, wherein the mutagenesis comprises introduction of one or more point mutations, or genome editing, or use of a bacterial CRISPR/CAS system, or a combination thereof.

In one embodiment, the genetically modified plant is a transgenic plant comprising at least one cell comprising at least one first series silencer targeted to a polynucleotide encoding at least one globulin protein or fragment thereof, selected from the group consisting of a fragment of a gene encoding glycinin 1 (GY1) or a complementary sequence thereof, a fragment of a gene encoding glycinin 2 (GY2) or a complementary sequence thereof, a fragment of a gene encoding glycinin 3 (GY3) or a complementary sequence thereof, a fragment of a gene encoding glycinin 4 (GLY4) or a complementary sequence thereof, a fragment of a gene encoding glycinin 5 (GY5) or a complementary sequence thereof, a fragment of a gene encoding alpha-conglycinin or a complementary sequence thereof, a fragment of a gene encoding alpha-prime-conglycinin or a complementary sequence thereof, and a fragment of a gene encoding beta-conglycinin or a complementary sequence thereof, or wherein the transgenic plant comprises a polynucleotide encoding at least one protein selected from the group consisting of glycinin 1 (GY1), glycinin 2 (GY2), glycinin 3 (GY3), glycinin 4 (GLY4), glycinin 5 (GY5), alpha-conglycinin, alpha-prime-conglycinin, and beta-conglycinin, wherein expression of the polynucleotide is selectively silenced, repressed, or reduced.

In one embodiment, the polynucleotide has been selectively edited by deletion, insertion, or modification to silence, repress, or reduce expression thereof, or the genetically modified plant is a progeny of the transgenic plant. In some embodiments, a nucleotide expressing an endogenous plant protein is edited such that the endogenous protein has reduced expression compared with a non-modified plant. In some embodiments, a nucleotide expressing an endogenous plant protein is edited such that the endogenous protein is not expressed at all compared with a non-modified plant. In some embodiments, a nucleotide expressing an endogenous seed storage plant protein is edited such that the seed storage protein has reduced expression compared with a non-modified plant. In some embodiments, a nucleotide expressing an endogenous seed storage plant protein is edited such that the seed storage protein is not expressed at all compared with a non-modified plant. In some embodiments, a nucleotide expressing an endogenous globulin protein is edited such that the seed storage protein has reduced expression compared with a non-modified plant. In some embodiments, a nucleotide expressing an endogenous globulin plant protein is edited such that the seed storage protein is not expressed at all compared with a non-modified plant. In some embodiments, a nucleotide expressing an endogenous desaturase protein is edited such that the desaturase protein has reduced expression compared with a non-modified plant. In some embodiments, a nucleotide expressing an endogenous desaturase plant protein is edited such that the desaturase protein is not expressed at all compared with a non-modified plant.

In some embodiments, a gene expressing an endogenous plant protein is edited such that the endogenous protein has reduced expression compared with a non-modified plant. In some embodiments, a gene expressing an endogenous plant protein is edited such that the endogenous protein is not expressed at all compared with a non-modified plant. In some embodiments, a gene expressing an endogenous seed storage plant protein is edited such that the seed storage protein has reduced expression compared with a non-modified plant. In some embodiments, a gene expressing an endogenous seed storage plant protein is edited such that the seed storage protein is not expressed at all compared with a non-modified plant. In some embodiments, a gene expressing an endogenous globulin protein is edited such that the seed storage protein has reduced expression compared with a non-modified plant. In some embodiments, a gene expressing an endogenous globulin plant protein is edited such that the seed storage protein is not expressed at all compared with a non-modified plant. In some embodiments, a gene expressing an endogenous desaturase protein is edited such that the desaturase protein has reduced expression compared with a non-modified plant. In some embodiments, a gene expressing an endogenous desaturase plant protein is edited such that the desaturase protein is not expressed at all compared with a non-modified plant.

In one embodiment, the at least one first series silencer comprises at least one guide-RNA pair targeted to a 5′-translated region of a polynucleotide encoding at least one globulin protein or a portion thereof selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof.

In one embodiment, the at least one guide-RNA pair is selected from the group consisting of (a) the guide-RNA pair encoded by SEQ ID NO: 57 and SEQ ID NO: 58, (b) the guide-RNA pair encoded by SEQ ID NO: 59 and SEQ ID NO: 60, (c) the guide-RNA pair encoded by SEQ ID NO: 61 and SEQ ID NO: 62, and (d) the guide-RNA pair encoded by SEQ ID NO: 63 and SEQ ID NO: 64.

In one embodiment, the genetically modified plant is a transgenic plant or gene edited plant comprising at least one cell comprising at least one second series silencer targeted to a polynucleotide encoding at least one desaturase protein or a portion thereof, selected from the group consisting of a fragment of a gene encoding fatty acid desaturase 1A (FAD2-1A) or a complementary sequence thereof, a fragment of a gene encoding fatty acid desaturase 1B (FAD2-1B) or a complementary sequence thereof, and a fragment of a gene encoding delta-9-stearoyl-acyl-carrier protein desaturase (SACPD) or a complementary sequence thereof, or the transgenic plant comprises a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and delta-9-stearoyl-acyl-carrier protein desaturase (SACPD) or a portion thereof, wherein expression of the polynucleotide is selectively silenced, repressed, or reduced.

In one embodiment, the polynucleotide has been selectively edited by deletion, insertion, or modification to silence, repress, or reduce expression thereof, or the genetically modified plant is a progeny of the transgenic plant.

In one embodiment, the at least one second series silencer comprises at least one guide-RNA pair targeted to a 5′-translated region of a polynucleotide encoding at least one desaturase protein or a portion thereof, selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In one embodiment, the at least one guide-RNA pair is selected from the group consisting of (a) the guide-RNA pair encoded by SEQ ID NO: 65 and SEQ ID NO: 66, and (b) the guide-RNA pair encoded by SEQ ID NO: 67 and SEQ ID NO: 68.

In one embodiment, the genetically modified plant further comprises at least one cell expressing at least three proteins from the milk of a mammal of the Bos genus, wherein the plant is selected from the genus Glycine and wherein:
(a) the at least three proteins are selected from the group consisting of serum albumin, alpha-S1-casein, alpha-S2-casein, beta-casein, kappa-casein, beta-lactoglobulin, and alpha-lactalbumin, wherein:
   (i) the amino acid sequence of the serum albumin is at least 90% identical to the amino acid sequence set forth in SEQ ID NO: 36, or the polynucleotide encoding the serum albumin encodes a serum albumin that is at least 90% identical to the serum albumin encoded by the polynucleotide sequence set forth in SEQ ID NO: 29;
   (ii) the amino acid sequence of the alpha-S1-casein is at least 90% identical to the amino acid sequence set forth in SEQ ID NO: 37, or the polynucleotide encoding the alpha-S1-casein encodes an alpha-S1-casein that is at least 90% identical to the alpha-S1-casein encoded by the polynucleotide sequence set forth in SEQ ID NO: 30;
   (iii) the amino acid sequence of the alpha-S2-casein is at least 90% identical to the amino acid sequence set forth in SEQ ID NO: 38 or the polynucleotide encoding the alpha-S2-casein encodes an alpha-S2-casein that is at least 90% identical to the alpha-S2-casein encoded by the polynucleotide sequence set forth in SEQ ID NO: 31;
   (iv) the amino acid sequence of the beta-casein is at least 90% identical to the amino acid sequence set forth in SEQ ID NO: 39 or the polynucleotide encoding the beta-casein encodes a beta-casein that is at least 90% identical to the beta-casein encoded by the polynucleotide sequence set forth in SEQ ID NO: 32;
   (v) the amino acid sequence of the kappa-casein is at least 90% identical to the amino acid sequence set forth in SEQ ID NO: 40 or the polynucleotide encoding the kappa-casein encodes a kappa-casein that is at least 90% identical to the kappa-casein encoded by the polynucleotide sequence set forth in SEQ ID NO: 33;
   (vi) the amino acid sequence of the beta-lactoglobulin is at least 90% identical to the amino acid sequence set forth in SEQ ID NO: 41 or the polynucleotide encoding the beta-lactoglobulin encodes a beta-lactoglobulin that is at least 90% identical to the beta-lactoglobulin encoded by the polynucleotide sequence set forth in SEQ ID NO: 34; and
   (vii) the amino acid sequence of the alpha-lactalbumin is at least 90% identical to the amino acid sequence set forth in SEQ ID NO: 42 or the polynucleotide encoding the alpha-lactalbumin encodes an alpha-lactalbumin that is at least 90% identical to the alpha-lactalbumin encoded by the polynucleotide sequence set forth in SEQ ID NO: 35,
      wherein each of said at least three proteins is a recombinant protein produced by the plant cell and wherein expression of each said recombinant protein is independently under control of a promoter selected from the group consisting of seed promoters of the genus Glycine, each said recombinant protein being expressed in the cell at a relative abundance of at least 75% when compared to the relative abundance of protein in the milk of the mammal of the Bos genus; and
(b) the at least one cell further comprises:
   (i) decreased expression of at least one globulin gene selected from the group consisting of a gene encoding glycinin 1 (GY1), a gene encoding glycinin 2 (GY2), a gene encoding glycinin 3 (GY3), a gene encoding glycinin 4 (GLY4), a gene encoding glycinin 5 (GY5), a gene encoding alpha-conglycinin, a gene encoding alpha-prime-conglycinin, and a gene encoding beta-conglycinin compared to its expression in a corresponding unmodified plant, wherein the at least one cell further comprises at least one first series silencer; and
   (ii) decreased expression of at least one desaturase gene selected from the group consisting of a gene encoding fatty acid desaturase 1A (FAD2-1A), a gene encoding fatty acid desaturase 1B (FAD2-1B), and a gene encoding delta-9-stearoyl-acyl-carrier protein desaturase (SACPD) compared to its expression in a corresponding unmodified plant, wherein the at least one cell further comprises at least one second series silencer,
      wherein expression of the at least one globulin gene or expression of the at least one desaturase gene is reduced in the modified plant compared to its expression in a corresponding unmodified plant, the modified plant comprising reduced content of at least one globulin or derivative thereof, or of at least one desaturase or derivative thereof, or comprises an increased content of at least one oleic acid or derivative thereof or stearic acid or derivative thereof or a reduced content of at least one saturated fat, compared to the corresponding unmodified plant, compared to the corresponding unmodified plant.

In one embodiment, the genetically modified plant further comprises at least one cell expressing at least five proteins from the milk of a mammal of the Bos genus, wherein:
(a) the at least five proteins are selected from the group consisting of serum albumin, alpha-S1-casein, alpha-S2-casein, beta-casein, kappa-casein, beta-lactoglobulin, and alpha-lactalbumin;
(b) each of the at least five proteins is differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical Bos species.

In one embodiment, the genetically modified plant, further comprises at least one cell expressing proteins from the milk of a mammal of the Bos genus, wherein:
(a) the proteins from the milk of a mammal consist of serum albumin, alpha-S1-casein, alpha-S2-casein, beta-casein, kappa-casein, beta-lactoglobulin, and alpha-lactalbumin; and
(b) each of the proteins is differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical Bos species.

In one embodiment, expression of each protein from the milk of a mammal is independently under control of a seed promoter, wherein:
(a) expression of beta-casein is controlled by Seed 1 (SEQ ID NO: 51);
(b) expression of kappa-casein and beta-lactoglobulin are controlled by Seed 2 (SEQ ID NO: 52);
(c) expression of alpha-S2-casein is controlled by Seed 3 (SEQ ID NO: 53);
(d) expression of alpha-S1-casein is controlled by Seed 4 (SEQ ID NO: 54);
(e) expression of serum albumin is controlled by Seed 5 (SEQ ID NO: 55); and
(f) expression of alpha-lactalbumin is controlled by Seed 6 (SEQ ID NO: 56).

In one embodiment, each of the proteins is differentially expressed to produce a content profile in the genetically modified plant or a seed, bean, grain, fruit, nut, legume, leaf, stem, root, portion, product, isolate, exudate, secretion, or extract thereof of at least 75% of a content profile in milk of the identical Bos species.

In one embodiment, each of the proteins is differentially expressed to produce a content profile in the genetically modified plant or a seed, bean, grain, fruit, nut, legume, leaf, stem, root, portion, product, isolate, exudate, secretion, or extract thereof having no greater than 150% of a content profile in milk of the identical Bos species.

In one embodiment:
(a) the at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; and
(b) the at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In one embodiment:
(a) the at least one first series silencer comprises at least one guide-RNA pair selected from the group consisting of (i) the guide-RNA pair encoded by SEQ ID NO: 57 and SEQ ID NO: 58, (ii) the guide-RNA pair encoded by SEQ ID NO: 59 and SEQ ID NO: 60, (iii) the guide-RNA pair encoded by SEQ ID NO: 61 and SEQ ID NO: 62, and (iv) the guide-RNA pair encoded by SEQ ID NO: 63 and SEQ ID NO: 64; and
(b) the at least one second series silencer comprises at least one guide-RNA pair selected from the group consisting of (i) the guide-RNA pair encoded by SEQ ID NO: 65 and SEQ ID NO: 66, and (ii) the guide-RNA pair encoded by SEQ ID NO: 67 and SEQ ID NO: 68.

In one embodiment:
(a) the first series silencer comprises: (i) a guide-RNA pair encoded by SEQ ID NO: 57 and SEQ ID NO: 58, (ii) a pair encoded by SEQ ID NO: 59 and SEQ ID NO: 60, (iii) a guide-RNA pair encoded by SEQ ID NO: 61 and SEQ ID NO: 62, and (iv) a guide-RNA pair encoded by SEQ ID NO: 63 and SEQ ID NO: 64, and
(b) the second series silencer comprises: (i) a guide-RNA pair encoded by SEQ ID NO: 65 and SEQ ID NO: 66, and (ii) a guide-RNA pair encoded by SEQ ID NO: 67 and SEQ ID NO: 68.

According to yet another aspect, the present invention comprises a food, medicament, cosmetic or blocking composition comprising the genetically modified plant as described or a seed, bean, grain, fruit, nut, legume, leaf, stem, root, portion, product, isolate, exudate, secretion, or extract thereof, the food, medicament, cosmetic or blocking composition comprising at least one protein from the milk of a mammal of the Bovidae family.

In one embodiment, the food, medicament, cosmetic or blocking composition comprises mammalian proteins of a Bos species consisting of serum albumin, alpha-S1-casein, alpha-S2-casein, beta-casein, kappa-casein, beta-lactoglobulin, and alpha-lactalbumin, wherein each of the proteins is differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical Bos species.

In one embodiment, each of the proteins is differentially expressed to produce a content profile in the genetically modified plant or a seed, bean, grain, fruit, nut, legume, leaf, stem, root, portion, product, isolate, exudate, secretion, or extract thereof of at least 75% of a content profile in milk of the identical Bos species.

In one embodiment, each of the proteins is differentially expressed to produce a content profile in the genetically modified plant or a seed, bean, grain, fruit, nut, legume, leaf, stem, root, portion, product, isolate, exudate, secretion, or extract thereof of no greater than 150% of a content profile in milk of the identical Bos species.

In one embodiment:
(a) the level of each of glycinin 1 (GY1), glycinin 2 (GY2), glycinin 3 (GY3), glycinin 4 (GLY4 glycinin 5 (GY5), alpha-conglycinin, alpha-prime-conglycinin, and beta-conglycinin is reduced as compared with the respective level of each in a non-genetically modified plant of the same species;
(b) the level of each of fatty acid desaturase 1A (FAD2-1A), fatty acid desaturase 1B (FAD2-1B), and delta-9-stearoyl-acyl-carrier protein desaturase (SACPD) is reduced as compared with the respective level of each in a non-genetically modified plant of the same species; and
(c) the food, medicament, cosmetic or blocking composition does not comprise any other milk proteins aside from serum albumin, alpha-S1-casein, alpha-S2-casein, beta-casein, kappa-casein, beta-lactoglobulin, or alpha-lactalbumin.

According to yet another aspect, the present invention provides a DNA binary vector or viral vector for expressing in a plant, proteins from the milk of a mammal, the vector comprising:
(a) a selectable marker;
(b) polynucleotide sequences encoding at least three proteins from the milk of a mammal, wherein the at least three proteins are selected from the group consisting of serum albumin, alpha-S1-casein, alpha-S2-casein, beta-casein, kappa-casein, beta-lactoglobulin, and alpha-lactalbumin, each independently under control of a promoter, wherein: each of said recombinant proteins is at least 90% identical to the corresponding mammalian protein amino acid sequence.

In one embodiment, the vector has a sequence at least 90% identical to SEQ ID NO: 50 or at least 90% identical to SEQ ID NO: 69.

According to still another aspect, the present invention provides a DNA binary vector or viral vector for expressing in a plant, proteins from the milk of a mammal, the vector comprising:
(a) a selectable marker; and
(b) a polynucleotide sequence encoding at least one recombinant protein from the milk of a mammal, wherein the proteins are selected from the group consisting of serum albumin, alpha-S1-casein, alpha-S2-casein, beta-casein, kappa-casein, beta-lactoglobulin, and alpha-lactalbumin, each independently under control of a promoter, wherein:
   (i) each of said recombinant proteins is at least 90% identical to the corresponding mammalian protein amino acid sequence; and
   (ii) each of the recombinant proteins is differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical mammalian species.

According to yet another aspect, the present invention provides a DNA binary vector or viral vector for differentially expressing in a plant, proteins from the milk of a mammal, the vector comprising:
(a) a selectable marker;
(b) polynucleotide sequences encoding at least three proteins from the milk of a mammal, wherein the at least three proteins are selected from the group consisting of serum albumin, alpha-S1-casein, alpha-S2-casein, beta-casein, kappa-casein, beta-lactoglobulin, and alpha-lactalbumin, each independently under control of a promoter, wherein:
   (i) each of said recombinant proteins is at least 90% identical to the corresponding mammalian protein amino acid sequence; and
   (ii) wherein each of the promoters for each of the polynucleotide sequences encoding proteins from the milk of a mammal differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical mammalian species.

In one embodiment, the DNA binary vector or viral vector further comprises polynucleotide sequences encoding at least five proteins from the milk of a mammal, wherein the at least five proteins are selected from the group consisting of serum albumin, alpha-S1-casein, alpha-S2-casein, beta-casein, kappa-casein, beta-lactoglobulin, and alpha-lactalbumin, each independently under control of a promoter.

In one embodiment, the DNA binary vector or viral vector further comprises polynucleotide sequences encoding seven proteins from the milk of a mammal, wherein the proteins from the milk of a mammal consist of serum albumin, alpha-S1-casein, alpha-S2-casein, beta-casein, kappa-casein, beta-lactoglobulin, and alpha-lactalbumin.

In one embodiment, the mammal is selected from the Bos genus and wherein:
(a) the amino acid sequence of the serum albumin is at least 90% identical to the amino acid sequence set forth in SEQ ID NO: 36, or the polynucleotide encoding the serum albumin encodes a serum albumin that is at least 90% identical to the serum albumin encoded by the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide encoding the alpha-S2-casein encodes an alpha-S1-casein that is at least 90% identical to the alpha-S1-casein encoded by the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide encoding the alpha-S2-casein encodes an alpha-S2-casein that is at least 90% identical to the alpha-S2-casein encoded by the polynucleotide sequence set forth in SEQ ID NO: 31;
(d) the amino acid sequence of the beta-casein is at least 90% identical to the amino acid sequence set forth in SEQ ID NO: 39 or the polynucleotide encoding the beta-casein encodes a beta-casein that is at least 90% identical to the beta-casein encoded by the polynucleotide sequence set forth in SEQ ID NO: 32;
(e) the amino acid sequence of the kappa-casein is at least 90% identical to the amino acid sequence set forth in SEQ ID NO: 40 or the polynucleotide encoding the kappa-casein encodes a kappa-casein that is at least 90% identical to the kappa-casein encoded by the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide encoding the beta-lactoglobulin encodes a beta-lactoglobulin that is at least 90% identical to the beta-lactoglobulin encoded by the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide encoding the alpha-lactalbumin encodes an alpha-lactalbumin that is at least 90% identical to the alpha-lactalbumin encoded by the polynucleotide sequence set forth in SEQ ID NO: 35.

In one embodiment, the plant is selected from the genus Glycine and wherein expression of each protein from the milk of a mammal is independently under control of a seed promoter. Alternatively, the plant is selected from a non-Glycine genus and wherein expression of each protein from the milk of a mammal is independently under control of a seed promoter.

In one embodiment:
(a) expression of beta-casein is controlled by Seed 1 (SEQ ID NO: 51);
(b) expression of kappa-casein and beta-lactoglobulin are controlled by Seed 2 (SEQ ID NO: 52);
(c) expression of alpha-S2-casein is controlled by Seed 3 (SEQ ID NO: 53);
(d) expression of alpha-S1-casein is controlled by Seed 4 (SEQ ID NO: 54);
(e) expression of serum albumin is controlled by Seed 5 (SEQ ID NO: 55); and
(f) expression of alpha-lactalbumin is controlled by Seed 6 (SEQ ID NO: 56).

In one embodiment, the DNA binary vector or viral vector further comprises:
(a) an expression sequence encoding CRISPR/CSY4;
(b) an expression sequence encoding CRISPR/Cas9;
(c) a guide-RNA expression multiarray complex under the control of an independent guide-RNA expression multiarray complex promotor, the guide-RNA expression multiarray complex encoding one or more guide-RNA pairs in an array cleavable by a CRISPR/CSY4 RNA endonuclease, wherein:
   (i) the at least one first series silencer guide-RNA pair is targeted to a polynucleotide encoding at least one globulin gene protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; or
   (ii) the at least one second series silencer guide-RNA pair is targeted to a polynucleotide encoding at least one desaturase gene protein or a portion thereof, selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In one embodiment, the guide-RNA expression multiarray complex encoding a first series silencer targeted to a 5′-translated region of a polynucleotide encoding a globulin protein or a portion thereof or a second series silencer target to a 5′-translated region of a polynucleotide encoding a desaturase protein or a portion thereof.

In one embodiment, the guide-RNA expression multiarray complex encoding a first series silencer and a second series silencer, wherein:
(a) the first series silencer comprises one or more guide-RNA pairs consisting of (α) the guide-RNA pair encoded by SEQ ID NO: 57 and SEQ ID NO: 58, (b) the guide-RNA pair encoded by SEQ ID NO: 59 and SEQ ID NO: 60, (c) the guide-RNA pair encoded by SEQ ID NO: 61 and SEQ ID NO: 62, and (d) the guide-RNA pair encoded by SEQ ID NO: 63 and SEQ ID NO: 64: and
(b) the second series silencer comprises one or more guide-RNA pairs consisting of (α) the guide-RNA pair encoded by SEQ ID NO: 65 and SEQ ID NO: 66, and (b) the guide-RNA pair encoded by SEQ ID NO: 67 and SEQ ID NO: 68.

In one embodiment, the guide-RNA expression multiarray complex encoding a first series silencer and a second series silencer, wherein:
(a) the first series silencer comprises: (a) a guide-RNA pair encoded by SEQ ID NO: 57 and SEQ ID NO: 58, (b) a pair encoded by SEQ ID NO: 59 and SEQ ID NO: 60, (c) a guide-RNA pair encoded by SEQ ID NO: 61 and SEQ ID NO: 62, and (d) a guide-RNA pair encoded by SEQ ID NO: 63 and SEQ ID NO: 64, and
(b) the second series silencer comprises: (a) a guide-RNA pair encoded by SEQ ID NO: 65 and SEQ ID NO: 66, and (b) a guide-RNA pair encoded by SEQ ID NO: 67 and SEQ ID NO: 68.

In one embodiment, the independent guide-RNA expression multiarray complex promotor is a CaMV-35S-promoter (p35s).

In one embodiment, the selectable marker is a BASTA resistance marker.

In one embodiment, the vector has a sequence at least 90% identical to SEQ ID NO: 69.

According to yet another aspect, the present invention provides a genetically modified plant cell comprising any one of the vectors.

According to still another aspect, the present invention provides a method of producing a food, medicament, cosmetic or blocking composition comprising a genetically modified plant or a seed, bean, grain, fruit, nut, legume, leaf, stem, root, portion, product, isolate, exudate, secretion, or extract thereof having at least 70% of a content profile in milk of a mammal, the method comprising:
(a) providing a DNA binary vector or viral vector for differentially expressing in a plant, proteins from the milk of a mammal, the vector comprising:
   (i) a selectable marker; and
   (ii) polynucleotide sequences encoding at least three recombinant proteins from the milk of a mammal, wherein the proteins are selected from the group consisting of serum albumin, alpha-S1-casein, alpha-S2-casein, beta-casein, kappa-casein, beta-lactoglobulin, and alpha-lactalbumin, each independently under control of a promoter, wherein:
      (1) each of said recombinant proteins is at least 90% identical to the corresponding mammalian protein amino acid sequence; and
      (2) wherein each of the promoters for each of the polynucleotide sequences encoding recombinant proteins from the milk of a mammal differentially activates expression of its corresponding polynucleotide sequence to produce a content profile in the genetically modified plant or a portion, seed, bean, grain, fruit, nut, legume, leaf, stem, root, product, isolate, exudate, secretion, or extract thereof having at least 70% of a content profile in milk from a mammal of the identical mammalian species;
   (b) transfecting at least one plant cell with the DNA binary vector or viral vector; and
   (c) differentially expressing the at least three recombinant proteins to produce a food, medicament, cosmetic or blocking composition comprising the genetically modified plant or a portion, seed, bean, grain, fruit, nut, legume, leaf, stem, root, product, isolate, exudate, secretion, or extract thereof having a content profile of at least 70% of a content profile in milk from a mammal of the identical mammalian species; and
   (d) optionally, adding milk of a mammal to the food, medicament, cosmetic or blocking composition of step c.

In one embodiment, the vector further comprises;
(a) an expression sequence encoding CRISPR/CSY4;
(b) an expression sequence encoding CRISPR/Cas9;
(c) a guide-RNA expression multiarray complex under the control of an independent guide-RNA expression multiarray complex promotor, the guide-RNA expression multiarray complex encoding one or more guide-RNA pairs in an array cleavable by a CRISPR/CSY4 RNA endonuclease, wherein:
   (i) the at least one first series silencer guide-RNA pair is targeted to a polynucleotide encoding at least one globulin gene protein selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; or
   (ii) the at least one second series silencer guide-RNA pair is targeted to a polynucleotide encoding at least one desaturase gene protein selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof,
      wherein expression of the at least one globulin gene protein or expression of the at least one desaturase gene protein is reduced in the modified plant compared to its expression in a corresponding unmodified plant, thereby the modified plant comprises reduced content of at least one globulin or derivative thereof, or of at least one desaturase or derivative thereof, or comprises an increased content of at least one oleic acid or derivative thereof or stearic acid or derivative thereof or a reduced content of at least one saturated fat, compared to the corresponding unmodified plant.

In one embodiment, the plant does not produce or comprise any other milk proteins aside from serum albumin, alpha-S1-casein, alpha-S2-casein, beta-casein, kappa-casein, beta-lactoglobulin, or alpha-lactalbumin.

Expression of the at least one gene encoding at least one protein from the milk of a mammal can be obtained by any method as is known to a person skilled in the art. According to certain embodiments, the present invention provides a genetically modified organism comprising at least one cell comprising at least one transcribable polynucleotide encoding at least one protein from the milk of a mammal, wherein the transgenic plant comprises elevated content of at least one protein selected from the group consisting of serum albumin or a portion or derivative thereof, α-S1-casein or a portion or derivative thereof, α-S2-casein or a portion or derivative thereof, β-casein or a portion or derivative thereof, κ-casein or a portion or derivative thereof, β-lactoglobulin or a portion or derivative thereof, and/or α-lactalbumin or a portion or derivative thereof compared to a corresponding non-transgenic plant.

According to some embodiments, the polynucleotides of the present invention are incorporated in a DNA construct enabling their expression in the plant cell. DNA constructs suitable for use in plants are known to a person skilled in the art. According to one embodiment, the DNA construct comprises at least one expression regulating element selected from the group consisting of a promoter, an enhancer, an origin of replication, a transcription termination sequence, a polyadenylation signal and the like.

The DNA constructs of the present invention are designed according to the results to be achieved. To yield a milk-like food, medicament, cosmetic or blocking composition in plants, it is desirable that the milk proteins (e.g., serum albumin, α-S1-casein [alpha-S1-casein], α-S2-casein [alpha-S2-casein], β-casein [beta-casein], κ-casein [kappa-casein], β-lactoglobulin [beta-lactoglobulin], and/or α-lactalbumin [alpha-lactalbumin] and/or portions and/or derivatives of any of these) in the plant be differentially expressed to provide a nutritional food, medicament, cosmetic or blocking composition having a relative abundance of the recombinant proteins from the plant of at least 70%, 75%, 80%, 85%, 90%, 95%, 100%, or up to 150% when compared to the relative abundance of the corresponding proteins in milk of the same mammalian species. Where multiple milk proteins are expressed, it is desirable that each milk protein in the plant be differentially expressed to provide a nutritional food, medicament, cosmetic or blocking composition having a relative abundance of each of the recombinant proteins from the plant of at least 70%, 75%, 80%, 85%, 90%, 95%, 100%, or up to 150% when compared to the relative abundance of the corresponding proteins in milk of the same mammalian species to mirror the nutritional content of milk with respect to these proteins.

On the other hand, some humans and other mammals are susceptible to plant allergies, including allergies to crop plants. Therefore, it is desirable to reduce allergenic proteins, such as globulins (e.g., 11S and/or 7S globulins). Examples of 11S globulins include, e.g., glycinin 1 (GY1), glycinin 2 (GY2), glycinin 3 (GY3), glycinin 4 (GY4), and glycinin 5 (GY5). Examples of 7S globulins include, e.g., α-conglycinin (alpha-conglycinin), α-prime-conglycinin (alpha-prime-conglycinin), and β-conglycinin (beta-conglycinin).

Moreover, increased content of oleic and/or stearic fatty acids is considered favorable and beneficial for human health. For example, deletions of fatty acid desaturases (e.g., FAD2-1A and/or FAD2-1B) increase oleic acid production in some plants (e.g., soybean). Likewise, deletion of stearoyl-acyl-carrier protein desaturase (e.g., Δ-9-stearoyl-acyl-carrier protein desaturase; delta-9-stearoyl-acyl-carrier protein desaturase [SACPD-C]) increases production of stearic acid in some plants (e.g., soybean).

According to certain embodiments, the DNA construct comprises a promoter. The promoter can be constitutive, induced or tissue specific as is known in the art. In some embodiments, the promoter comprises a constitutive promoter. In some embodiments, the promoter comprises an inducible promoter. In some embodiments, the promoter comprises a tissue specific promoter. In some embodiments, the promoter comprises a developmental specific promoter. Optionally, the DNA construct further comprises a selectable marker, enabling the convenient selection of the transformed cell/tissue. Additionally, or alternatively, a reporter gene can be incorporated into the construct, so as to enable selection of transformed cells or tissue expressing the reporter gene.

Suspensions of genetically modified or gene edited cells and tissue cultures derived from the genetically modified or gene edited cells are also encompassed within the scope of the present invention. The cell suspension and tissue cultures can be used for the production of desired steroidal glycoalkaloids and, which are then extracted from the cells or the growth medium. Alternatively, the genetically modified or gene edited cells and/or tissue culture are used for regenerating a transgenic plant having modified or gene edited expression of milk proteins from a mammal, therefore expressing milk proteins in a plant, and/or having modified or gene edited expression of globulin proteins, therefore having an altered risk of hyperallergenic response, and/or desaturases, therefore having modified content of oleic and/or stearic acids.

The present invention further encompasses seeds of the genetically modified or gene edited plant, wherein plants grown from said seeds and expressing milk proteins compared to plants grown from corresponding unmodified or unedited seeds, thereby containing at least one milk protein. Similarly, the present invention further encompasses seeds of the genetically modified or gene edited plant, wherein plants grown from said seeds and having reduced globulin proteins compared to plants grown from corresponding unmodified or unedited seeds, thereby reducing potential for allergic reaction. Likewise, the present invention further encompasses seeds of the genetically modified or gene edited plant, wherein plants grown from said seeds and having reduced desaturases compared to plants grown from corresponding unmodified or unedited seeds, thereby increasing oleic and/or stearic acids.

Viral vectors are useful for transformation of more transformation-resistant plants (e.g., soybean or common bean). In some embodiments, viral vectors, such as bean pod mottle virus (BPMV; genus Comovirus) vectors, are used for foreign gene expression and virus-induced gene silencing (VIGS) (Zhang et al. (May 2010) Plant Physiol. 153: 52-65 [“Zhang 2010”])). Cells are transformed, e.g., via biolistics or via direct DNA-rubbing inoculation (Zhang 2010).

In one embodiment, a gene gun or a biolistic particle delivery system (biolistics) is used for plant transformation to deliver exogenous DNA (transgenes) to cells (Rech et al. (2008) Nature Protocols 3(3): 410-418 [“Rech 2008”]). In some embodiments, the plasmid is designed and apical meristems of plants (e.g., soybean, bean, cotton) are bombarded with microparticle-coated DNA, followed by in vitro culture and selection of transgenic plants (Rech 2008). In other embodiments, a callus of undifferentiated plant cells or a group of immature embryos growing on gel medium in vitro. In some embodiments, the cells are then treated with a series of plant hormones, such as auxins or gibberellins to obtain plants.

“Transient expression” of the proteins may be achieved by various means known in the art. In one embodiment, transient expression of the proteins is achieved by the use of genetically modified viruses. In some embodiments, agroinfiltration is used to induce transient expression of genes in a plant or an isolated leaf or another portion of a plant. A suspension of Agrobacterium (e.g., Agrobacterium tumefaciens) is introduced into the plant by, e.g., direct injection or vacuum filtration, or is brought into association with plant cells immobilized on a porous support (plant cell packs). The bacteria transfer the desired gene into the plant cells via transfer of Ti plasmid-derived T-DNA.

In one embodiment, “grafting” methods are used to produce the animal milk in nut trees (e.g., almond, hazelnut/cobnut/filbert, walnut, butternut, pistachio, or cashew), in a coconut tree, or other types of trees. In one embodiment, a grafting method is used to produce the animal milk in a peanut plant.

### Genetically Modified Plants & Gene Edited Plants

Disclosed herein are genetically modified plants and gene edited plants, wherein expression of key genes encoding proteins found in mammal milk (or portions or derivatives thereof) has been added. Adding the expression of these genes results in concomitant addition of milk proteins in the plants and in products therefrom.

Also disclosed herein are genetically modified plants and gene edited plants, wherein expression of key genes expressing certain globulins have been altered. Altering the expression of these gene results in concomitant alteration in the globulin content of the plants and their products, decreasing the risk of hyperallergenic reaction to the plants and their products.

Also disclosed herein are genetically modified plants and gene edited plants, wherein expression of key genes (encoding desaturases) in the oleic acid and stearic acid metabolic pathways (biosynthesis pathway of oleic acids and derivatives thereof and stearic acids and derivatives thereof) have been altered. Altering the expression of these genes results in concomitant alteration in the oleic acid and/or stearic acid profile, namely in the decrease of desaturase levels and in the concomitant increase in oleic acids and/or stearic acids.

Changing the production level of steroidal alkaloid can result in improved plants comprising milk proteins (e.g., serum albumin, α-S1-casein, α-S2-casein, β-casein, κ-casein, β-lactoglobulin, α-lactoglobulin), whereby the plants or products of the plants (e.g., food, medicament, cosmetic or blocking compositions) contain milk proteins yielding an animal-free, milk-like, plant-based product, which, when further combined with a reduction in globulin proteins (e.g., glycinin (11S) globulin proteins [e.g., GY1, GY2, GY3, GY4, GY5] and/or β-conglycinin (7S) globulin proteins [e.g., α-conglycinin, α′-conglycinin, β-glycinin]), provides a milk alternative eliminating a risk of lactose intolerance on the one hand and plant allergies on the other. When still further combined with a decrease in desaturases (e.g., FAD2-1A, FAD2-1B, SACPD), the plants and plant products (e.g., food, medicament, cosmetic or blocking compositions) have increased levels of oleic and/or stearic acids, thereby improving nutritional value.

In certain embodiments, disclosed herein is a genetically modified plant comprising at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source. In some embodiments, the genetically modified plant comprises at least one cell expressing at least 3, 4, 5, 6, or 7 milk proteins from a mammal, the at least 3, 4, 5, 6, or 7 milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least 3, 4, 5, 6, or 7 proteins is at least 90% identical to the amino acid sequence of a corresponding mammalian milk protein from the same mammalian source.

In other embodiments, the at least one cell of a genetically modified plant disclosed herein further comprises reduced expression of at least one globulin protein as compared to the expression thereof in a corresponding unmodified plant and wherein said at least one cell comprises a reduced content of the at least one globulin protein as compared to the content thereof in a corresponding unmodified plant. In some embodiments, the genetically modified plant comprises at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source, wherein the at least one cell further comprises reduced expression of at least one globulin protein as compared to the expression thereof in a corresponding unmodified plant and wherein said at least one cell comprises a reduced content of the at least one globulin protein as compared to the content thereof in a corresponding unmodified plant. In some embodiments, the genetically modified plant comprises at least one cell expressing at least 3, 4, 5, 6, or 7 milk proteins from a mammal, the at least 3, 4, 5, 6, or 7 milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least 3, 4, 5, 6, or 7 proteins is at least 90% identical to the amino acid sequence of a corresponding mammalian milk protein from the same mammalian source, wherein the at least one cell further comprises reduced expression of at least one globulin protein as compared to the expression thereof in a corresponding unmodified plant and wherein said at least one cell comprises a reduced content of the at least one globulin protein as compared to the content thereof in a corresponding unmodified plant.

In other embodiments, the at least one cell of a genetically modified plant disclosed herein further comprises reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant. An at least one cell of a genetically modified plant that provides both (1) at least 2 milk proteins and (2) increased content of at least one oleic acid or derivative thereof or at least one stearic acid or derivative thereof, or reduced content of at least one saturated fat, or a combination thereof may in some embodiments be beneficial for any food, medicament, cosmetic or blocking composition proteins by providing both milk proteins, and oleic and or stearic acid, or reduced saturated fats. In some embodiments, the genetically modified plant comprises at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source, wherein the at least one cell further comprises reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant. In some embodiments, the genetically modified plant comprises at least one cell expressing at least 3, 4, 5, 6, or 7 milk proteins from a mammal, the at least 2, 3, 4, 5, 6, or 7 milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least 3, 4, 5, 6, or 7 proteins is at least 90% identical to the amino acid sequence of a corresponding mammalian milk protein from the same mammalian source, wherein the at least one cell further comprises reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant.

In other embodiments, the at least one cell of a genetically modified plant disclosed herein further comprises reduced expression of at least one seed storage protein as compared to the expression thereof in a corresponding unmodified plan and wherein said at least one cell comprises a reduced content of the at least one seed storage protein as compared to the content thereof in a corresponding unmodified plant. An at least one cell of a genetically modified plant that provides both (1) at least 2 milk proteins and (2) reduced expression of at least one seed storage protein as compared to the expression thereof in a corresponding unmodified plan and wherein said at least one cell comprises a reduced content of the at least one seed storage protein as compared to the content thereof in a corresponding unmodified plant, may in some embodiments, beneficially enhance the content of the at least 2 milk proteins. In some embodiments, the genetically modified plant comprises at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source, wherein the at least one cell further comprises reduced expression of at least one seed storage protein as compared to the expression thereof in a corresponding unmodified plan and wherein said at least one cell comprises a reduced content of the at least one seed storage protein as compared to the content thereof in a corresponding unmodified plant. In some embodiments, the genetically modified plant comprises at least one cell expressing at least 3, 4, 5, 6, or 7 milk proteins from a mammal, the at least 2, 3, 4, 5, 6, or 7 milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least 3, 4, 5, 6, or 7 proteins is at least 90% identical to the amino acid sequence of a corresponding mammalian milk protein from the same mammalian source, wherein the at least one cell further comprises reduced expression of at least one seed storage protein as compared to the expression thereof in a corresponding unmodified plan and wherein said at least one cell comprises a reduced content of the at least one seed storage protein as compared to the content thereof in a corresponding unmodified plant.

In other embodiments, the at least one cell of a genetically modified plant disclosed herein further comprises reduced expression of at least one seed storage protein as compared to the expression thereof in a corresponding unmodified plan and wherein said at least one cell comprises a reduced content of the at least one seed storage protein as compared to the content thereof in a corresponding unmodified plant, or comprises reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant; or comprises reduced expression of at least one seed storage protein as compared to the expression thereof in a corresponding unmodified plan and wherein said at least one cell comprises a reduced content of the at least one seed storage protein as compared to the content thereof in a corresponding unmodified plant; or comprises a combination thereof.

In particular, disclosed herein are the means and methods for producing crop plants of the Solanaceae family (including Nicotiana benthamiana and the Nicotiana genus), the Fabaceae family (including Glycine max and the Glycine genus), and the Poaceae family (including the Oryza genus, e.g., Oryza sativa and Oryza glaberrima) in which various milk proteins from mammals (including the Bovidae family, the Bos genus, and Bos taurus) are expressed. Also disclosed herein are the means and methods for producing crop plants of the Fabaceae family (including Glycine max and the Glycine genus) in which expression of globulin proteins (e.g., glycinin (11S) globulin proteins [e.g., GY1, GY2, GY3, GY4, GY5] and/or β-conglycinin (7S) globulin proteins [e.g., α-conglycinin, α′-conglycinin, β-glycinin]) is silenced or reduced. Also disclosed herein are the means and methods for producing crop plants of the Fabaceae family (including Glycine max and the Glycine genus) in which expression of desaturases (e.g., FAD2-1A, FAD2-1B, SACPD) is silenced or reduced. The plants, food, medicament, cosmetic or blocking compositions, vectors, cells, and methods disclosed herein are thus of significant nutritional and/or commercial value.

In certain embodiments, disclosed herein is a food, medicament, cosmetic or blocking composition comprising a genetically modified plant or a portion, product, isolate, exudate, secretion, or extract thereof, said genetically modified plant or portion, product, isolate, exudate, secretion, or extract thereof comprising at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source. In some embodiments, disclosed herein is a food, medicament, cosmetic or blocking composition comprising a genetically modified plant or a portion, product, isolate, exudate, secretion, or extract thereof, said genetically modified plant or portion, product, isolate, exudate, secretion, or extract thereof comprising at least one cell expressing at least 3, 4, 5, 6, or 7 milk proteins from a mammal, the at least 3, 4, 5, 6, or 7 milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least 3, 4, 5, 6, or 7 proteins is at least 90% identical to the amino acid sequence of a corresponding mammalian milk protein from the same mammalian source.

In other embodiments, the food, medicament, cosmetic or blocking composition comprising a genetically modified plant or a portion, product, isolate, exudate, secretion, or extract thereof, comprises at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source, and further comprises (a) reduced expression of at least one globulin protein as compared to the expression thereof in a corresponding unmodified plant and wherein said at least one cell comprises a reduced content of the at least one globulin protein as compared to the content thereof in a corresponding unmodified plant; or (b) reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant; or (c) reduced expression of at least one seed storage protein as compared to the expression thereof in a corresponding unmodified plan and wherein said at least one cell comprises a reduced content of the at least one seed storage protein as compared to the content thereof in a corresponding unmodified plant; or (d) a combination thereof.

In other embodiments, the food, medicament, cosmetic or blocking composition comprising a genetically modified plant or a portion, product, isolate, exudate, secretion, or extract thereof, said genetically modified plant or portion, product, isolate, exudate, secretion, or extract thereof comprises at least one cell expressing at least 3, 4, 5, 6, or 7 milk proteins from a mammal, the at least 3, 4, 5, 6, or 7 milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least 3, 4, 5, 6, or 7 proteins is at least 90% identical to the amino acid sequence of a corresponding mammalian milk protein from the same mammalian source, and further comprises (a) reduced expression of at least one globulin protein as compared to the expression thereof in a corresponding unmodified plant and wherein said at least one cell comprises a reduced content of the at least one globulin protein as compared to the content thereof in a corresponding unmodified plant; or (b) reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant; or (c) reduced expression of at least one seed storage protein as compared to the expression thereof in a corresponding unmodified plan and wherein said at least one cell comprises a reduced content of the at least one seed storage protein as compared to the content thereof in a corresponding unmodified plant; or (d) a combination thereof.

Disclosed herein is a DNA binary vector comprising a series of promotors (including the Seed promotors [e.g., Seed1, Seed2, Seed3, Seed4, Seed5, Seed6]) for differential expression of milk proteins in a plant, each milk protein independently under control of a promoter independently selected so as to result in a food, medicament, cosmetic or blocking composition in which the relative abundance of each plant-expressed milk protein is at least 70% and no more than 150% that of the corresponding protein in milk of the mammalian species from which the plant-based expression originates, in order to reflect the nutritional content of mammalian milk. Further, in some embodiments a DNA binary vector or viral vector disclosed herein may further comprise a polynucleotide sequence comprising a silencing element under the control of a promotor targeted to at least one globulin gene; or at least one desaturase gene; or at least one seed storage protein; or a combination thereof.

In certain embodiments, disclosed herein is a DNA binary vector or viral vector expressing at least two milk proteins from a mammal, the vector comprising: a selectable marker; polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least two proteins is at least 90% identical to the amino acid sequence of a corresponding mammalian milk protein from the same mammalian source. In some embodiments, disclosed herein is a DNA binary vector or viral vector expressing at least 3, 4, 5, 6, or 7 milk proteins from a mammal, the vector comprising: a selectable marker; polynucleotide sequences encoding at least 3, 4, 5, 6, or 7 milk proteins from a mammal, wherein said at least 3, 4, 5, 6, or 7, milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least 3, 4, 5, 6, or 7 proteins is at least 90% identical to the amino acid sequence of a corresponding mammalian milk protein from the same mammalian source.

In other embodiments, a DNA binary vector or viral vector disclosed herein further comprises a polynucleotide sequence comprising a silencing element under the control of a promotor targeted to at least one globulin gene; or at least one desaturase gene; or at least one seed storage protein; or a combination thereof. In certain embodiments, disclosed herein is a DNA binary vector or viral vector expressing at least two milk proteins from a mammal, the vector comprising: a selectable marker; polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least two proteins is at least 90% identical to the amino acid sequence of a corresponding mammalian milk protein from the same mammalian source; and a polynucleotide sequence comprising a silencing element under the control of a promotor targeted to at least one globulin gene; or at least one desaturase gene; or at least one seed storage protein; or a combination thereof. In some embodiments, disclosed herein is a DNA binary vector or viral vector expressing at least 3, 4, 5, 6, or 7 milk proteins from a mammal, the vector comprising: a selectable marker; polynucleotide sequences encoding at least 3, 4, 5, 6, or 7 milk proteins from a mammal, wherein said at least 3, 4, 5, 6, or 7 milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least 3, 4, 5, 6, or 7 proteins is at least 90% identical to the amino acid sequence of a corresponding mammalian milk protein from the same mammalian source; and a polynucleotide sequence comprising a silencing element under the control of a promotor targeted to at least one globulin gene; or at least one desaturase gene; or at least one seed storage protein; or a combination thereof.

In certain embodiments, disclosed herein is a method of producing a food, medicament, cosmetic or blocking composition comprising a genetically modified plant or portion, product, isolate, exudate, secretion, or extract thereof, the method comprising: providing a DNA binary vector or viral vector for differentially expressing in a plant, proteins from the milk of a mammal, the vector comprising: a selectable marker; polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under control of a promoter, wherein: the amino acid sequence of each of said at least two proteins is at least 90% identical to the amino acid sequence of a corresponding mammalian milk protein from the same mammalian source, and wherein expression of each of said at least two milk proteins is independently under the control of a seed promoter for obtaining a relative protein content of each of said at least two milk proteins of at least 70% of the relative protein content of the corresponding mammalian milk protein in the mammal's milk; transfecting at least one cell of said plant with the DNA binary vector or viral vector; differentially expressing the at least two milk proteins in said at least one plant cell; and optionally adding milk of a mammal to the food, medicament, cosmetic or blocking composition of step (c). In some embodiments, disclosed herein is a method of producing a food, medicament, cosmetic or blocking composition comprising a genetically modified plant or portion, product, isolate, exudate, secretion, or extract thereof, the method comprising: providing a DNA binary vector or viral vector for differentially expressing in a plant, proteins from the milk of a mammal, the vector comprising: a selectable marker; polynucleotide sequences encoding at least 3, 4, 5, 6, or 7 milk proteins from a mammal, wherein said at least 3, 4, 5, 6, or 7 milk proteins are selected from the group consisting of serum albumin, alpha-S1-casein, alpha-S2-casein, beta-casein, kappa-casein, beta-lactoglobulin, and alpha-lactalbumin, each independently under control of a promoter, wherein: the amino acid sequence of each of said at least 3, 4, 5, 6, or 7 proteins is at least 90% identical to the amino acid sequence of a corresponding mammalian milk protein from the same mammalian source; and wherein expression of each of said at least 3, 4, 5, 6, or 7 milk proteins is independently under the control of a seed promoter for obtaining a relative protein content of each of said at least 3, 4, 5, 6, or 7 milk proteins of at least 70% of the relative protein content of the corresponding mammalian milk protein in the mammal's milk; transfecting at least one cell of said plant with the DNA binary vector or viral vector; differentially expressing the at least 3, 4, 5, 6, or 7 milk proteins in said at least one plant cell, and optionally adding milk of a mammal to the food, medicament, cosmetic or blocking composition of step (c).

In other embodiments, the method of producing a food, medicament, cosmetic, or blocking agent further comprises polynucleotide sequences further comprising a polynucleotide sequence comprising a silencing element under the control of a promotor targeted to at least one globulin gene; at least one desaturase gene; or at least one seed storage protein; or a combination thereof. In certain embodiments, disclosed herein is a method of producing a food, medicament, cosmetic or blocking composition comprising a genetically modified plant or portion, product, isolate, exudate, secretion, or extract thereof, the method comprising: providing a DNA binary vector or viral vector for differentially expressing in a plant, proteins from the milk of a mammal, the vector comprising: a selectable marker; polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under control of a promoter, wherein: the amino acid sequence of each of said at least two proteins is at least 90% identical to the amino acid sequence of a corresponding mammalian milk protein from the same mammalian source; wherein expression of each of said at least two milk proteins is independently under the control of a seed promoter for obtaining a relative protein content of each of said at least two milk proteins of at least 70% of the relative protein content of the corresponding mammalian milk protein in the mammal's milk, and a polynucleotide sequence comprising a silencing element under the control of a promotor targeted to at least one globulin gene; at least one desaturase gene; or at least one seed storage protein; or a combination thereof; transfecting at least one cell of said plant with the DNA binary vector or viral vector; differentially expressing the at least two milk proteins in said at least one plant cell; and optionally adding milk of a mammal to the food, medicament, cosmetic or blocking composition of step (c). In some embodiments, disclosed herein is a method of producing a food, medicament, cosmetic or blocking composition comprising a genetically modified plant or portion, product, isolate, exudate, secretion, or extract thereof, the method comprising: providing a DNA binary vector or viral vector for differentially expressing in a plant, proteins from the milk of a mammal, the vector comprising: a selectable marker; polynucleotide sequences encoding at least 3, 4, 5, 6, or 7 milk proteins from a mammal, wherein said at least 3, 4, 5, 6, or 7 milk proteins are selected from the group consisting of serum albumin, alpha-S1-casein, alpha-S2-casein, beta-casein, kappa-casein, beta-lactoglobulin, and alpha-lactalbumin, each independently under control of a promoter, wherein: the amino acid sequence of each of said at least 3, 4, 5, 6, or 7 proteins is at least 90% identical to the amino acid sequence of a corresponding mammalian milk protein from the same mammalian source; and wherein expression of each of said at least 3, 4, 5, 6, or 7 milk proteins is independently under the control of a seed promoter for obtaining a relative protein content of each of said at least 3, 4, 5, 6, or 7 milk proteins of at least 70% of the relative protein content of the corresponding mammalian milk protein in the mammal's milk, and a polynucleotide sequence comprising a silencing element under the control of a promotor targeted to at least one globulin gene; at least one desaturase gene; or at least one seed storage protein; or a combination thereof; transfecting at least one cell of said plant with the DNA binary vector or viral vector; differentially expressing the at least 3, 4, 5, 6, or 7 milk proteins in said at least one plant cell; and optionally adding milk of a mammal to the food, medicament, cosmetic or blocking composition of step (c).

Disclosed herein is a guide-RNA expression multiarray under the control of an independent guide-RNA expression multiarray complex promoter, the guide-RNA expression multiarray complex encoding one or more guide-RNA pairs in an array cleavable by a CRISPR/CSY4 RNA endonuclease, including a first series silencer(s) targeted to globulin protein polynucleotides and/or a second series silencer(s) targeted to desaturase polynucleotides.

The plants and food, medicament, cosmetic or blocking compositions of the present invention are thus of significant nutritional and commercial value.

### Definitions

“Mammals” (class “Mammalia”) are endothermic vertebrates usually characterized by the presence of hair, three middle-ear bones, a neocortex, and in female mammals, mammary glands that secrete milk during lactation. With a few exceptions, mammals are viviparous. Mammals include, but are not limited to, humans, cows, buffalo, goats, sheep, camels, dromedaries, donkeys, horses, reindeer, yaks, moose, bison, bison/cow hybrids, pigs, dogs, cats, lions, tigers, panda bears, leopards, giraffes, whales, and dolphins. The term “milk protein component” refers to proteins or protein equivalents and variants found in milk such as casein, whey or the combination of casein and whey, including their subunits, which are derived from various sources and as further defined herein. Most commercially produced milk in Europe and North America is from the Bovidae biological family of cloven-hoofed, ruminant mammals, which includes, but is not limited to, cattle (e.g., domestic cows, Bos taurus), buffalo (e.g., water buffalo [e.g., Bubalus bubalis] and African/Cape buffalo [e.g., Syncerus caffer]), goats (e.g., domestic goats, Capra aegagrus), sheep (e.g., domestic sheep, Ovis aries), bison (e.g., Bison genus, American bison, European bison), yak (e.g., Bos grunniens), and bison/cow hybrids. Common non-Bovidae sources of commercial milk include, but are not limited to, members of the Camelidae (camels, dromedaries), Equidae (donkeys, horses), Cervidae (reindeer), and Suidae (pigs) families. Other sources of milk protein of particular interest include, but are not limited to humans, dogs, and cats.

As used herein, the term “milk” is the normal mammary secretion of lactating female mammals, including, but not limited to, “the normal mammary secretion of milking animals” (FAO, Codex Alimentarius, “Milk” (Codex Stan 206-1999) [http://www.fao.org/fao-who-codexalimentarius/en/] [“FAO Codex 1999”]). “Milk proteins” include proteins found in milk.

The term “milk protein” means a protein that is found in a mammal-produced milk or a protein having a sequence that is at least 80% identical (e.g., at least 85%, at least 90%, at least 95%, at least 96%, at least 97%, at least 98%, or at least 99% identical) to the sequence of a protein that is found in a mammal-produced milk. Examples of milk proteins include, but are not limited to, β-casein, κ-casein, α-S1-casein, α-S2-casein, α-lactalbumin, β-lactoglobulin, lactoferrin, transferrin, and serum albumin. Additional milk proteins are known in the art.

The term “casein protein” is art-known and represents a family of proteins that is present in mammal-produced milk and is capable of self-assembling with other proteins in the family to form micelles and/or precipitate out of an aqueous solution at an acidic pH. Examples of casein proteins include, but are not limited to, β-casein, κ-casein, α-S1-casein, α-S2-casein. Non-limiting examples of sequences for casein protein are provided herein. Additional sequences for other mammalian caseins are known in the art.

The term “mammal-produced milk” is art known and means a milk produced by a mammal.

The term “processed mammal-produced milk” means a mammal-produced milk that is processed using one or more steps known in the dairy industry (e.g., homogenization, pasteurization, irradiation, or supplementation).

The term “mammal-derived component” means a molecule or compound (e.g., a protein, a lipid, or a nucleic acid) obtained from the body of a mammal or a molecule obtained from a fluid or solid produced by a mammal.

The term “component of milk” or “milk component” is a molecule, compound, element, or an ion present in a mammal-produced milk.

The term “non-mammalian glycosylation pattern” means one of a difference in one or more location(s) of glycosylation in a protein, and/or a difference in the amount of and/or type of glycosylation at one or more location(s) in a protein produced and post-translational modified in a non-mammalian cell (e.g., a yeast cell, an insect cell, a bacterial cell, or a plant cell) as compared to a reference protein (e.g., the same protein produced and post-translationally modified in a mammalian cell, e.g., a CHO cell, a MEK cell, or a mammalian udder or breast cell).

The term “lipids” means one or more molecules (e.g., biomolecules) that include a fatty acyl group (e.g., saturated or unsaturated acyl chains). For example, the term lipids includes oils, phospholipids, free fatty acids, phospholipids, monoglycerides, diglycerides, and triglycerides. Additional examples of lipids are known in the art.

The term “plant-derived lipid” means a lipid obtained from and/or produced by a plant (e.g., monocot or dicot).

The term “milk substitute” and “milk alternative” refers to a composition that resembles, is similar to, is to equivalent to, or is nearly identical to a dairy milk. A “milk substitute” or “milk alternative” may be preferred or necessary in situations, e.g., in which an individual is unable to consume milk due to lactose intolerance or an allergy, where milk/breastmilk is unavailable for an individual for whom milk/breastmilk is necessary or preferable, or as a preferred nutritional component for a human or non-human animal.

In the present invention, milk from a mammal may be added to the food, medicament, cosmetic or blocking composition derived from the genetically modified plant or product thereof to provide, e.g., stability, consistency, flavor, or other qualities associated with milk from a mammal. Milk from a mammal may be added to the food, medicament, cosmetic or blocking composition for a final concentration of 1%, 2%, 3%, 5%, 10%, 15%, 20%, 25%, 30%, 35%, 40%, 45%, 50%, 55%, 60%, 65%, 70%, 75%, 80%, 85%, 90%, 95%, 97%, 98%, 99% milk from a mammal. An unmodified milk alternative from a plant may be added to the food, medicament, cosmetic or blocking composition for a final concentration of 1%, 2%, 3%, 5%, 10%, 15%, 20%, 25%, 30%, 35%, 40%, 45%, 50%, 55%, 60%, 65%, 70%, 75%, 80%, 85%, 90%, 95%, 97%, 98%, 99% milk alternative from a plant.

The term “flavor” refers to the taste and/or the aroma of a food or drink.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” optionally also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

One of ordinary skill in the art would appreciate that the term “gene” may encompass a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The skilled artisan would appreciate that the term “gene” optionally also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

In one embodiment, a gene comprises DNA sequence comprising upstream and downstream regions, as well as the coding region, which comprises exons and any intervening introns of the gene. In some embodiments, upstream and downstream regions comprise non-coding regulatory regions. In some embodiments, upstream and downstream regions comprise regulatory sequences, for example but not limited to promoters, enhancers, and silencers. Non-limiting examples of regulatory sequences include, but are not limited to, AGGA box, TATA box, Inr, DPE, ZmUbi1, PvUbi1, PvUbi2, CaMV, 35S, OsAct1, zE19, E8, TA29, A9, pDJ3S, B33, PAT1, alcA, G-box, ABRE, DRE, and PCNA. Regulatory regions, may in some embodiments, increase or decrease the expression of specific genes within a plant described herein.

In another embodiment, a gene comprises the coding regions of the gene, which comprises exons and any intervening introns of the gene. In another embodiment, a gene comprises its regulatory sequences. In another embodiment, a gene comprises the gene promoter. In another embodiment, a gene comprises its enhancer regions. In another embodiment, a gene comprises 5′ non-coding sequences. In another embodiment, a gene comprises 3′ non-coding sequences.

In one embodiment, the skilled artisan would appreciate that DNA comprises a gene, which may include upstream and downstream sequences, as well as the coding region of the gene. In another embodiment, DNA comprises a cDNA (complementary DNA). One of ordinary skill in the art would appreciate that cDNA may encompass synthetic DNA reverse transcribed from RNA through the action of a reverse transcriptase. The cDNA may be single stranded or double stranded and can include strands that have either or both of a sequence that is substantially identical to a part of the RNA sequence or a complement to a part of the RNA sequence. Further, cDNA may include upstream and downstream regulatory sequences. In still another embodiment, DNA comprises CDS (complete coding sequence). One of ordinary skill in the art would appreciate that CDS may encompass a DNA sequence, which encodes a full-length protein or polypeptide. A CDS typically begins with a start codon (“ATG”) and ends at (or one before) the first in-frame stop codon (“TAA”, “TAG”, or “TGA”). The skilled artisan would recognize that a cDNA, in one embodiment, comprises a CDS.

The terms “polynucleotide”, “polynucleotide sequence”, “nucleic acid sequence”, and “isolated polynucleotide” are used interchangeably herein. These terms encompass nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

The term “RNA interference” or “RNAi” refers to the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

Typically, the term RNAi molecule refers to single- or double-stranded RNA molecules comprising both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The term “construct” as used herein refers to an artificially assembled or isolated nucleic acid molecule which includes the polynucleotide of interest. In general, a construct may include the polynucleotide or polynucleotides of interest, a marker gene which in some cases can also be a gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

The terms “promoter element,” “promoter,” or “promoter sequence” as used herein, refer to a DNA sequence that is located at the 5′ end (i.e., precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Examples of promoters include, but are not limited to: Solamum lycopersicum ubiquitin promoter 10 (SlPrUbiq10); the cauliflower mosaic virus Pol-III promoter CaMV-35S-promoter (p35S); soybean seed-specific promoters SEED1, SEED2, SEED3, SEED4, SEED5, SEED6.

As used herein, the term an “enhancer” refers to a DNA sequence which can stimulate promoter activity and may be an innate element of the promoter or a heterologous element inserted to enhance the level or tissue-specificity of a promoter.

The term “expression”, as used herein, refers to the production of a functional end-product e.g., an mRNA and or a protein. One skilled in the art would appreciate that a genetically modified plant and cell thereof expressing at least two milk proteins encompasses plants and cells that express at least two genes encoding milk proteins, transcribing at least two mRNAs from the DNA of the genes encoding the at least two milk proteins, and translating the mRNAs into the functional end-product comprising at least two milk proteins.

The term “gene edited plant” refers to a plant comprising at least one cell comprising at least one gene edited by man. The gene editing includes deletion, insertion, silencing, or repression, such as of the “native genome” of the cell or of the “native genome” of the chloroplast of the cell. Methods for creating a gene edited plant include techniques such as zinc-finger nucleases (ZFN), transcription activator-like effector nucleases (TALEN), and clustered regularly interspersed short palindromic repeats (CRISPR)/Cas systems.

The term “genetically modified plant” refers to a plant comprising at least one cell genetically modified by man. The genetic modification includes modification of an endogenous gene(s) or an endogenous chloroplast gene(s) (Day et al. (2011) Plant Biotechnol. J. 9:540-553 [“Day 2011”]), for example by introducing mutation(s) deletions, insertions, transposable element(s) and the like into an endogenous polynucleotide or gene of interest. Additionally, or alternatively, the genetic modification includes transforming the plant cell with heterologous polynucleotide. A “genetically modified plant” and a “corresponding unmodified plant” as used herein refer to a plant comprising at least one genetically modified cell and to a plant of the same type lacking said modification, respectively.

One of ordinary skill in the art would appreciate that a genetically modified plant may encompass a plant comprising at least one cell genetically modified by man. In some embodiments, the genetic modification includes modification of an endogenous gene(s), for example by introducing mutation(s) deletions, insertions, transposable element(s) and the like into an endogenous polynucleotide or gene of interest. Additionally, or alternatively, in some embodiments, the genetic modification includes transforming at least one plant cell with a heterologous polynucleotide or multiple heterologous polynucleotides. The skilled artisan would appreciate that a genetically modified plant comprising transforming at least one plant cell with a heterologous polynucleotide or multiple heterologous polynucleotides may in certain embodiments be termed a “transgenic plant”.

A skilled artisan would appreciate that a comparison of a “genetically modified plant” to a “corresponding unmodified plant” as used herein encompasses comparing a plant comprising at least one genetically modified cell and to a plant of the same type lacking the modification.

The skilled artisan would appreciate that the term “transgenic” when used in reference to a plant as disclosed herein encompasses a plant that contains at least one heterologous transcribable polynucleotide in one or more of its cells. The term “transgenic material” encompasses broadly a plant or a part thereof, including at least one cell, multiple cells or tissues that contain at least one heterologous polynucleotide in at least one of cell. Thus, comparison of a “transgenic plant” and a “corresponding non transgenic plant”, or of a “genetically modified plant comprising at least one cell having altered expression, wherein said plant comprising at least one cell comprising a heterologous transcribable polynucleotide” and a “corresponding unmodified plant” encompasses comparison of the “transgenic plant” or “genetically modified plant” to a plant of the same type lacking said heterologous transcribable polynucleotide. A skilled artisan would appreciate that, in some embodiments, a “transcribable polynucleotide” comprises a polynucleotide that can be transcribed into an RNA molecule by an RNA polymerase.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. In contrast, the term “stable transformation” or “stably transformed” refers to the introduction and integration of one or more exogenous polynucleotides into the genome of a cell. The term “stable transformant” refers to a cell which has stably integrated one or more exogenous polynucleotides into the genomic or organellar DNA. It is to be understood that an organism or its cell transformed with the nucleic acids, constructs and/or vectors of the present invention can be transiently as well as stably transformed.

The skilled artisan would appreciate that the term “construct” may encompass an artificially assembled or isolated nucleic acid molecule which includes the polynucleotide of interest. In general, a construct may include the polynucleotide or polynucleotides of interest, a marker gene which in some cases can also be a gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The skilled artisan would appreciate that the term “expression” may encompass the production of a functional end-product e.g., an mRNA or a protein.

As used herein, the term “predominantly” or variations thereof will be understood to mean, for instance, a) in the context of fats the amount of a particular fatty acid composition relative to the total amount of fatty acid composition; b) in the context of protein the amount of a particular protein composition (e.g., β-casein) relative to the total amount of protein composition (e.g., α-, β-, and κ-casein).

The term “about,” “approximately,” or “similar to” means within an acceptable error range for the particular value as determined by one of ordinary skill in the art, which can depend in part on how the value is measured or determined, or on the limitations of the measurement system. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

The phrase “essentially free of” is used to indicate the indicated component, if present, is present in an amount that does not contribute, or contributes only in a de minimus fashion, to the properties of the composition. In various embodiments, where a composition is essentially free of a particular component, the component is present in less than a functional amount. In various embodiments, the component may be present in trace amounts. Particular limits will vary depending on the nature of the component, but may be, for example, selected from less than 10% by weight, less than 9% by weight, less than 8% by weight, less than 7% by weight, less than 6% by weight, less than 5% by weight, less than 4% by weight, less than 3% by weight, less than 2% by weight, less than 1% by weight, or less than 0.5% by weight.

As used herein, the term “consisting essentially of” means that consisting largely, but not necessarily entirely, of a recited element.

As used herein, the term “essentially free of” a particular carbohydrate, such as lactose is used to indicate that the food, medicament, cosmetic or blocking composition is substantially devoid of carbohydrate residues. Expressed in terms of purity, essentially free means that the amount of carbohydrate residues do not exceed 10%, and preferably is below 5%, more preferably below 1%, most preferably below 0.5%, wherein the percentages are by weight or by mole percent. Thus, substantially all of the carbohydrate residues in a food, medicament, cosmetic or blocking composition according to the present invention are free of, for example, lactose.

Unless indicated otherwise, percentage (%) of ingredients refer to total % by weight.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

As used in the specification and claims, the singular form “a”, “an” and “the” include plural references unless the context clearly dictates otherwise. For example, the term “a molecule” also includes a plurality of molecules.

The present invention now shows that mammalian milk proteins can be expressed in a plant.

According to certain exemplary embodiments, the genetically modified or gene edited plant or transgenic plant comprises at least one cell expressing one or more proteins from the milk of a mammal, wherein the one or more proteins is/are selected from the group consisting of serum albumin, α-S1-casein (alpha-S1-casein), α-S2-casein (alpha-S2-casein), β-casein (beta-casein), κ-casein (kappa-casein), β-lactoglobulin (beta-lactoglobulin), and/or α-lactalbumin (alpha-lactalbumin). According to other exemplary embodiments, the genetically modified or gene edited plant or transgenic plant does not produce or comprise any other milk proteins aside from serum albumin, α-S1-casein (alpha-S1-casein), α-S2-casein (alpha-S2-casein), β-casein (beta-casein), κ-casein (kappa-casein), β-lactoglobulin (beta-lactoglobulin), and/or α-lactalbumin (alpha-lactalbumin). Each possibility represents a separate embodiment of the present invention.

According to other exemplary embodiments, the genetically modified or gene edited plant or transgenic plant differentially expresses serum albumin, α-S1-casein (alpha-S1-casein), α-S2-casein (alpha-S2-casein), β-casein (beta-casein), κ-casein (kappa-casein), β-lactoglobulin (beta-lactoglobulin), and/or α-lactalbumin (alpha-lactalbumin) to be or to produce a food, medicament, cosmetic or blocking composition having a relative abundance of each of serum albumin, α-S1-casein (alpha-S1-casein), α-S2-casein (alpha-S2-casein), β-casein (beta-casein), κ-casein (kappa-casein), β-lactoglobulin (beta-lactoglobulin), and/or α-lactalbumin (alpha-lactalbumin) of at least 70% and no greater than 150% of the respective content of each of serum albumin, α-S1-casein (alpha-S1-casein), α-S2-casein (alpha-S2-casein), β-casein (beta-casein), κ-casein (kappa-casein), β-lactoglobulin (beta-lactoglobulin), and/or α-lactalbumin (alpha-lactalbumin) in the milk of a mammal.

According to certain exemplary embodiments, the genetically modified or gene edited plant or transgenic plant comprises at least on cell comprising at least one first series silencer targeted to at least one globulin gene, such as at least one 11S or 7S globulin gene selected from the group consisting of a gene encoding glycinin 1 (GY1), a gene encoding glycinin 2 (GY2), a gene encoding glycinin 3 (GY3), a gene encoding glycinin 4 (GY4), a gene encoding glycinin 5 (GY5), a gene encoding α-conglycinin (alpha-conglycinin), a gene encoding α′-conglycinin (alpha-prime-conglycinin), and β-conglycinin (beta-conglycinin). Each possibility represents a separate embodiment of the present invention.

According to certain exemplary embodiments, the genetically modified or gene edited plant or transgenic plant comprises at least one cell comprising at least one second series silencer targeted to at least one desaturase gene, such as a gene encoding fatty acid desaturase 1A (FAD2-1A), a gene encoding fatty acid desaturase 1B (FAD2-1B), and a gene encoding Δ-9-stearoyl-acyl-carrier protein desaturase (delta-9-stearoyl-acyl-carrier protein desaturase) (SACPD). Each possibility represents a separate embodiment of the present invention.

Down-regulation or inhibition of the gene expression can be effected on the genomic and/or the transcript level using a variety of molecules that interfere with transcription and/or translation (e.g., antisense, siRNA, Ribozyme, or DNAzyme), or on the protein level using, e.g., antagonists, enzymes that cleave the polypeptide, and the like.

The silencing molecule (silencer) targeted to at least one globulin gene (first series silencer) or to at least one desaturase gene (second series silencer) can be designed as is known to a person skilled in the art. According to certain embodiments, the silencer comprises a polynucleotide having a nucleic acid sequence substantially complementary to a region of a polynucleotide encoding the globulin or the desaturase targeted. According to certain embodiments, the silencer comprises a guide-RNA pair. According to certain embodiments, the guide-RNA pair is targeted to a 5′-translated region of a polynucleotide encoding the globulin or the desaturase. According to certain embodiments, multiple guide-RNA pairs target multiple globulins and/or multiple desaturases. According to certain embodiments, multiple guide-RNA (gRNA) pairs are encoded by a guide-RNA expression multiarray complex under the control of an independent guide-RNA expression multiarray complex promoter and in an array cleavable by a CRISPR/CSY4 RNA endonuclease. According to certain embodiments, a CRISPR/Case system for multiple gene targeting is used to construct the multiplex guide-RNA array of multiple guide-RNA pairs targeting the genes of interest.

### Antisense Molecules

Antisense technology is the process in which an antisense RNA or DNA molecule interacts with a target sense DNA or RNA strand. A sense strand is a 5′ to 3′ mRNA molecule or DNA molecule. The complementary strand, or mirror strand, to the sense is called an antisense. When an antisense strand interacts with a sense mRNA strand, the double helix is recognized as foreign to the cell and will be degraded, resulting in reduced or absent protein production. Although DNA is already a double stranded molecule, antisense technology can be applied to it, building a triplex formation.

One skilled in the art would appreciate that the terms “complementary” or “complement thereof” are used herein to encompass the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

RNA antisense strands can be either catalytic or non-catalytic. The catalytic antisense strands, also called ribozymes, cleave the RNA molecule at specific sequences. A non-catalytic RNA antisense strand blocks further RNA processing.

Antisense modulation of cells and/or tissue levels of the globulin genes of interest and/or desaturase genes of interest or any combination thereof may be effected by transforming the organism cells or tissues with at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog.

Antisense modulation of cells and/or tissue levels of the globulin genes of interest and/or desaturase genes of interest or any combination thereof may be effected by transforming the organism cells or tissues with at least one antisense compound, including antisense DNA, antisense RNA, a ribozyme, DNAzyme, a locked nucleic acid (LNA), and an aptamer. In some embodiments, the molecules are chemically modified. In other embodiments, the antisense molecule is antisense DNA or an antisense DNA analog.

### RNA Interference (RNAi) Molecules

RNAi refers to the introduction of homologous double stranded RNA (dsRNA) to target a specific gene product, resulting in post transcriptional silencing of that gene. This phenomenon was first reported in Caenorhabditis elegans by Guo and Kemphues (1995, Cell, 81(4):611-620) and subsequently Fire et al. (1998, Nature 391:806-811) discovered that it is the presence of dsRNA, formed from the annealing of sense and antisense strands present in the in vitro RNA preps, that is responsible for producing the interfering activity

In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available from commercial sources.

The dsRNA can be transcribed from the vectors as two separate strands. In other embodiments, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

One skilled in the art would appreciate that the terms “promoter element,” “promoter,” or “promoter sequence” may encompass a DNA sequence that is located at the 5′ end (i.e., precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full length of the gene or more.

The term “RNA interference” or “RNAi” refers to the silencing or decreasing of gene expression mediated by small double stranded RNAs. It is the process of sequence-specific, post-transcriptional gene silencing in animals and plants, initiated by inhibitory RNA (iRNA) that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

One of ordinary skill in the art would appreciate that the term RNAi molecule refers to single- or double-stranded RNA molecules comprising both a sense and antisense sequence. For example, the RNA interference molecule can be a double-stranded polynucleotide molecule comprising self-complementary sense and antisense regions, wherein the antisense region comprises complementarity to a target nucleic acid molecule. Alternatively the RNAi molecule can be a single-stranded hairpin polynucleotide having self-complementary sense and antisense regions, wherein the antisense region comprises complementarity to a target nucleic acid molecule or it can be a circular single-stranded polynucleotide having two or more loop structures and a stem comprising self-complementary sense and antisense regions, wherein the antisense region comprises complementarity to a target nucleic acid molecule, and wherein the circular polynucleotide can be processed either in vivo or in vitro to generate an active molecule capable of mediating RNAi.

In both plants and animals, RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs.

The dsRNA used to initiate RNAi, may be isolated from native source or produced by known means, e.g., transcribed from DNA. Plasmids and vectors for generating RNAi molecules against target sequence are now readily available as exemplified herein below.

The dsRNA can be transcribed from the vectors as two separate strands. In other embodiments, the two strands of DNA used to form the dsRNA may belong to the same or two different duplexes in which they each form with a DNA strand of at least partially complementary sequence. When the dsRNA is thus-produced, the DNA sequence to be transcribed is flanked by two promoters, one controlling the transcription of one of the strands, and the other that of the complementary strand. These two promoters may be identical or different. Alternatively, a single promoter can derive the transcription of single-stranded hairpin polynucleotide having self-complementary sense and antisense regions that anneal to produce the dsRNA.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases. There is no upper limit on the length of the dsRNA that can be used. For example, the dsRNA can range from about 21 base pairs (bp) of the gene to the full length of the gene or more.

### Co-Suppression Molecules

Another agent capable of downregulating the expression of a given gene, or a combination thereof is a Co-Suppression molecule. Co-suppression is a post-transcriptional mechanism where both the transgene and the endogenous gene are silenced.

### DNAzyme Molecules

Another agent capable of downregulating the expression of a given gene is a DNAzyme molecule, which is capable of specifically cleaving an mRNA transcript or a DNA sequence of said gene. DNAzymes are single-stranded polynucleotides that are capable of cleaving both single- and double-stranded target sequences. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (for review of DNAzymes, see: Khachigian, L. M. (2002) Curr Opin Mol Ther 4, 119-121).

Examples of construction and amplification of synthetic, engineered DNAzymes recognizing single- and double-stranded target cleavage sites are disclosed in U.S. Pat. No. 6,326,174.

### Enzymatic Oligonucleotide

The terms “enzymatic nucleic acid molecule” or “enzymatic oligonucleotide” refers to a nucleic acid molecule which has complementarity in a substrate binding region to a specified gene target and also has an enzymatic activity which is active to specifically cleave target RNA of a given gene, thereby silencing each of the genes. The complementary regions allow sufficient hybridization of the enzymatic nucleic acid molecule to the target RNA and subsequent cleavage. The term enzymatic nucleic acid is used interchangeably with for example, ribozymes, catalytic RNA, enzymatic RNA, catalytic DNA, aptazyme or aptamer-binding ribozyme, catalytic oligonucleotide, nucleozyme, DNAzyme, RNAenzyme. The specific enzymatic nucleic acid molecules described in the instant application are not limiting and an enzymatic nucleic acid molecule of this invention requires a specific substrate binding site which is complementary to one or more of the target nucleic acid regions, and that it have nucleotide sequences within or surrounding that substrate binding site which impart a nucleic acid cleaving and/or ligation activity to the molecule. U.S. Pat. No. 4,987,071 discloses examples of such molecules.

### Mutagenesis

Altering the expression of genes can be also achieved by the introduction of one or more point mutations into a nucleic acid molecule encoding the corresponding proteins. Mutations can be introduced using, for example, site-directed mutagenesis (see, e.g., Wu Ed., 1993 Meth. In Enzymol. Vol. 217, San Diego: Academic Press; Higuchi, “Recombinant PCR” in Innis et al. Eds., 1990 PCR Protocols, San Diego: Academic Press, Inc). Such mutagenesis can be used to introduce a specific, desired amino acid insertion, deletion or substitution. Several technologies for targeted mutagenesis are based on the targeted induction of double-strand breaks (DSBs) in the genome followed by error-prone DNA repair. Mostly commonly used for genome editing by these methods are custom designed nucleases, including zinc finger nucleases and Xanthomonas-derived transcription activator-like effector nuclease (TALEN) enzymes.

In some embodiments, when the expression of the at least one gene or combination thereof is altered, said altering comprises mutagenizing the at least one gene, said mutation present within a coding region of said at least one gene, or a regulatory sequence of said at least one gene, or a combination thereof.

Various types of mutagenesis can be used to modify genes and their encoded polypeptides in order to produce conservative or non-conservative variants. Any available mutagenesis procedure can be used. In some embodiments, the mutagenesis procedure comprises site-directed point mutagenesis. In some embodiments, the mutagenesis procedure comprises random point mutagenesis. In some embodiments, the mutagenesis procedure comprises in vitro or in vivo homologous recombination (DNA shuffling). In some embodiments, the mutagenesis procedure comprises mutagenesis using uracil-containing templates. In some embodiments, the mutagenesis procedure comprises oligonucleotide-directed mutagenesis. In some embodiments, the mutagenesis procedure comprises phosphorothioate-modified DNA mutagenesis. In some embodiments, the mutagenesis procedure comprises mutagenesis using gapped duplex DNA. In some embodiments, the mutagenesis procedure comprises point mismatch repair. In some embodiments, the mutagenesis procedure comprises mutagenesis using repair-deficient host strains. In some embodiments, the mutagenesis procedure comprises restriction-selection and restriction-purification. In some embodiments, the mutagenesis procedure comprises deletion mutagenesis. In some embodiments, the mutagenesis procedure comprises mutagenesis by total gene synthesis. In some embodiments, the mutagenesis procedure comprises double-strand break repair. In some embodiments, the mutagenesis procedure comprises mutagenesis by chimeric constructs. In some embodiments, the mutagenesis procedure comprises mutagenesis by CRISPR/Cas. In some embodiments, the mutagenesis procedure comprises mutagenesis by zinc-finger nucleases (ZFN). In some embodiments, the mutagenesis procedure comprises mutagenesis by transcription activator-like effector nucleases (TALEN). In some embodiments, the mutagenesis procedure comprises any other mutagenesis procedure known to a person skilled in the art.

In some embodiments, mutagenesis can be guided by known information about the naturally occurring molecule and/or the mutated molecule. By way of example, this known information may include sequence, sequence comparisons, physical properties, crystal structure and the like. In some embodiments, the mutagenesis is essentially random. In some embodiments the mutagenesis procedure is DNA shuffling.

In some embodiments, the genetic modification includes modification of an endogenous chloroplast gene(s), for example by introducing mutation(s) deletions, insertions, transposable element(s) and the like into an endogenous polynucleotide or gene of interest, such as using plastid transformation (Day et al. (2011) Plant Biotechnol. J. 9:540-553 [“Day 2011”]). For example, a selected marker is placed under the control of plastid expression signals, and homologous recombination through the flanking targeting arm directs integration into the recipient plastid genome (plastome) (e.g., using aadA-based plastid transformation and spectinomycin or spectinomycin streptomycin resistance) (Day 2011). Initially, only one copy of the polyploid plastome is heteroplasmic, but repeated rounds of cloning and selection can be used to obtain a homoplasmic clone (e.g., microalgae or cyanobacterium). In multicellular plants, each cell contains multiple plastids. Repeated rounds of propagation and selection are used to lead to a cell having a homoplasmic plastid, then to a cell having only homoplasmic plastids (but within a chimeric tissue overall), and finally to a non-chimeric homoplasmic plant, which can then provide homoplasmic cells for recover homoplasmic plants (Day 2011). In some embodiments, marker genes are excised or rotated (Day 2011). Alternatively, co-transformation (e.g., of two or more resistance markers) and segregation of marker-free plastid genomes (e.g., via switching selection) can be used to generate plants having a single resistance marker (Day 2011). Marker-free plants may also be generated using transient co-integration of the marker gene (e.g., aphA6 marker gene with kanamycin) (Day 2011). In one embodiment, stable integration of a marker gene into plastid DNA entails targeting the arms to enable a double crossover event in the homologous regions flanking the marker gene, creating an unstable co-integrate containing large direct repeats of the left and right targeting arms, and recombination between the repeated arms in the co-integrate results in excision of the marker genes (Day 2011).

In some embodiments, transient integration or co-integration

A skilled artisan would appreciate that clustered regularly interspaced short palindromic repeats (CRISPR)/CRISPR associated protein (Cas) system comprises genome engineering tools based on the bacterial CRISPR/Cas prokaryotic adaptive immune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belhaj K. et al., 2013. Plant Methods 2013, 9:39). In some embodiments, a CRISPR/Cas system comprises a CRISPR/Cas9 system.

In some embodiments, a CRISPR/Cas system comprises a single-guide RNA (sgRNA) and/or a Cas protein known in the art. In some embodiments, a CRISPR/Cas system comprises a single-guide RNA (sgRNA) and/or a Cas protein newly created to cleave at a preselected site. The skilled artisan would appreciate that the terms “single-guide RNA”, “sgRNA”, and “gRNA” are interchangeable having all the same qualities and meanings, wherein an sgRNA may encompass a chimeric RNA molecule which is composed of a CRISPR RNA (crRNA) and trans-encoded CRISPR RNA (tracrRNA). In some embodiments, a crRNA is complementary to a preselected region of a DNA of interest, wherein the crRNA “targets” the CRISPR associated polypeptide (Cas) nuclease protein to the preselected target site.

In some embodiments, the length of crRNA sequence complementary is 19-22 nucleotides long e.g., 19-22 consecutive nucleotides complementary to the target site. In another embodiment, the length of crRNA sequence complementary to the region of DNA is about 15-30 nucleotides long. In another embodiment, the length of crRNA sequence complementary to the region of DNA is about 15, 16, 17, 18, 19, 20, 21, 22, 23, 24, 25, 26, 27, 28, 29, or 30 nucleotides long. In another embodiment, the length of crRNA sequence complementary to the region of DNA is 20 nucleotides long. In some embodiments, the crRNA is located at the 5′ end of the sgRNA molecule. In another embodiment, the crRNA comprises 100% complementation within the preselected target sequence. In another embodiment, the crRNA comprises at least 80% complementation within the preselected target sequence. In another embodiment, the crRNA comprises at least 85% complementation within the preselected target sequence. In another embodiment, the crRNA comprises at least 90% complementation within the preselected target sequence. In another embodiment, the crRNA comprises at least 95% complementation within the preselected target sequence. In another embodiment, the crRNA comprises at least 97% complementation within the preselected target sequence. In another embodiment, the crRNA comprises at least 99% complementation within the preselected target sequence. In another embodiment, a tracrRNA is 100-300 nucleotides long and provides a binding site for the Cas nuclease, e.g., a Cas9 protein forming the CRISPR/Cas9 complex.

In one embodiment, a mutagenesis system comprises a CRISPR/Cas system. In another embodiment, a CRISPR/Cas system comprises a Cas nuclease and a gRNA molecule, wherein said gRNA molecule binds within said preselected endogenous target site thereby guiding said Cas nuclease to cleave the DNA within said preselected endogenous target site.

In some embodiments, a CRISPR/Cas system comprise an enzyme system including a guide RNA sequence (“gRNA” or “sgRNA”) that contains a nucleotide sequence complementary or substantially complementary to a region of a target polynucleotide, for example a preselected endogenous target site, and a protein with nuclease activity.

In another embodiment, a CRISPR/Cas system comprises a Type I CRISPR-Cas system, or a Type II CRISPR-Cas system, or a Type III CRISPR-Cas system, or derivatives thereof. In another embodiment, a CRISPR-Cas system comprises an engineered and/or programmed nuclease system derived from naturally accruing CRISPR-Cas systems. In another embodiment, a CRISPR-Cas system comprises engineered and/or mutated Cas proteins. In another embodiment, a CRISPR-Cas system comprises engineered and/or programmed guide RNA.

A skilled artisan would appreciate that a guide RNA may contain nucleotide sequences other than the region complementary or substantially complementary to a region of a target DNA sequence, for example a preselected endogenous target site. In another embodiment, a guide RNA comprises a crRNA or a derivative thereof. In another embodiment, a guide RNA comprises a crRNA: tracrRNA chimera.

In another embodiment, a gRNA molecule comprises a domain that is complementary to and binds to a preselected endogenous target site on at least one homologous chromosome. In another embodiment, a gRNA molecule comprises a domain that is complementary to and binds to a polymorphic allele on at least one homologous chromosome. In another embodiment, a gRNA molecule comprises a domain that is complementary to and binds to a preselected endogenous target site on both homologous chromosomes. In another embodiment, a gRNA molecule comprises a domain that is complementary to and binds to polymorphic alleles on both homologous chromosomes.

Cas enzymes comprise RNA-guided DNA endonuclease able to make double-stranded breaks (DSB) in DNA. The term “Cas enzyme” may be used interchangeably with the terms “CRISPR-associated endonucleases” or “CRISPR-associated polypeptides” having all the same qualities and meanings. In one embodiment, a Cas enzyme is selected from the group comprising Cas1, Cas1B, Cas2, Cas3, Cas4, Cas5, Cas6, Cas7, Cas8, Cas9, Cas10, C2cl, CasX, NgAgo, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, or homologs thereof, or modified versions thereof. In another embodiment, a Cas enzyme comprises Cas9. In another embodiment, a Cas enzyme comprises Cas1. In another embodiment, a Cas enzyme comprises Cas1B. In another embodiment, a Cas enzyme comprises Cas2. In another embodiment, a Cas enzyme comprises Cas3. In another embodiment, a Cas enzyme comprises Cas4. In another embodiment, a Cas enzyme comprises Cas5. In another embodiment, a Cas enzyme comprises Cas6/CSY4. In another embodiment, a Cas enzyme comprises Cas7. In another embodiment, a Cas enzyme comprises Cas8. In another embodiment, a Cas enzyme comprises Cas9. In another embodiment, a Cas enzyme comprises Cas10. In another embodiment, a Cas enzyme comprises Cpf1. In another embodiment, a Cas enzyme comprises Csy1. In another embodiment, a Cas enzyme comprises Csy2. In another embodiment, a Cas enzyme comprises Csy3. In another embodiment, a Cas enzyme comprises Cse1. In another embodiment, a Cas enzyme comprises Cse2. In another embodiment, a Cas enzyme comprises Csc1. In another embodiment, a Cas enzyme comprises Csc2. In another embodiment, a Cas enzyme comprises Csa5. In another embodiment, a Cas enzyme comprises Csn2. In another embodiment, a Cas enzyme comprises Csm2. In another embodiment, a Cas enzyme comprises Csm3. In another embodiment, a Cas enzyme comprises Csm4. In another embodiment, a Cas enzyme comprises Csm5. In another embodiment, a Cas enzyme comprises Csm6. In another embodiment, a Cas enzyme comprises Cmr1. In another embodiment, a Cas enzyme comprises Cmr3. In another embodiment, a Cas enzyme comprises Cmr4. In another embodiment, a Cas enzyme comprises Cmr5. In another embodiment, a Cas enzyme comprises Cmr6. In another embodiment, a Cas enzyme comprises Csb1. In another embodiment, a Cas enzyme comprises Csb2. In another embodiment, a Cas enzyme comprises Csb3. In another embodiment, a Cas enzyme comprises Csx17. In another embodiment, a Cas enzyme comprises Csx14. In another embodiment, a Cas enzyme comprises Csx10. In another embodiment, a Cas enzyme comprises Csx16, CsaX. In another embodiment, a Cas enzyme comprises Csx3. In another embodiment, a Cas enzyme comprises Csx1, Csx15, Csf1. In another embodiment, a Cas enzyme comprises Csf2. In another embodiment, a Cas enzyme comprises Csf3. In another embodiment, a Cas enzyme comprises Csf4. In another embodiment, a Cas enzyme comprises Cpf1. In another embodiment, a Cas enzyme comprises C2cl. In another embodiment, a Cas enzyme comprises CasX. In another embodiment, a Cas enzyme comprises NgAgo. In another embodiment, a Cas enzyme is Cas homologue. In another embodiment, a Cas enzyme is a Cas orthologue. In another embodiment, a Cas enzyme is a modified Cas enzyme. In another embodiment, a Cas enzyme is any CRISPR-associated endonucleases known in the art.

A skilled artisan would appreciate that the terms “zinc finger nuclease” or “ZFN” are interchangeable having all the same meanings and qualities, wherein a ZFN encompasses a chimeric protein molecule comprising at least one zinc finger DNA binding domain operatively linked to at least one nuclease capable of double strand cleaving of DNA. In some embodiments, a ZFN system comprises a ZFN known in the art. In some embodiments, a ZFN system comprises a ZFN newly created to cleave a preselected site.

In some embodiments, a ZFN creates a double-stranded break at a preselected endogenous target site. In some embodiments, a ZFN comprises a DNA-binding domain and a DNA-cleavage domain, wherein the DNA binding domain is comprised of at least one zinc finger and is operatively linked to a DNA-cleavage domain. In another embodiment, a zinc finger DNA-binding domain is at the N-terminus of the chimeric protein molecule and the DNA-cleavage domain is located at the C-terminus of the molecule. In another embodiment, a zinc finger DNA-binding domain is at the C-terminus of the chimeric protein molecule and the DNA-cleavage domain is located at the N-terminus of the molecule. In another embodiment, a zinc finger binding domain encompasses the region in a zinc finger nuclease that is capable of binding to a target locus, for example a preselected endogenous target site as disclosed herein. In another embodiment, a zinc finger DNA-binding domain comprises a protein domain that binds to a preselected endogenous target site on at least one homologous chromosome. In another embodiment, a zinc finger DNA-binding domain comprises a protein domain that binds to a polymorphic allele on at least one homologous chromosome. In another embodiment, a zinc finger DNA-binding domain comprises a protein domain that binds to a preselected endogenous target site on both homologous chromosomes. In another embodiment, a zinc finger DNA-binding domain comprises a protein domain that binds to polymorphic alleles on both homologous chromosomes.

The skilled artisan would appreciate that the term “chimeric protein” is used to describe a protein that has been expressed from a DNA molecule that has been created by operatively joining two or more DNA fragments. The DNA fragments may be from the same species, or they may be from a different species. The DNA fragments may be from the same or a different gene. The skilled artisan would appreciate that the term “DNA cleavage domain” of a ZFN encompasses the region in the zinc finger nuclease that is capable of breaking down the chemical bonds between nucleic acids in a nucleotide chain. Examples of proteins containing cleavage domains include restriction enzymes, topoisomerases, recombinases, integrases and DNAses.

In some embodiments, a TALEN system comprises a TAL effector DNA binding domain and a DNA cleavage domain, wherein said TAL effector DNA binding domain binds within said preselected endogenous target site, thereby targeting the DNA cleavage domain to cleave the DNA within said preselected endogenous target site.

A skilled artisan would appreciate that the terms “transcription activator-like effector nuclease”, “TALEN”, and “TAL effector nuclease” may be used interchangeably having all the same meanings and qualities, wherein a TALEN encompasses a nuclease capable of recognizing and cleaving its target site, for example a preselected endogenous target site as disclosed herein. In another embodiment, a TALEN comprises a fusion protein comprising a TALE domain and a nucleotide cleavage domain. In another embodiment, a TALE domain comprises a protein domain that binds to a nucleotide in a sequence-specific manner through one or more TALE-repeat modules. A skilled artisan would recognize that TALE-repeat modules comprise a variable number of about 34 amino acid repeats that recognize plant DNA sequences. Further, repeat modules can be rearranged according to a simple cipher to target new DNA sequences. In another embodiment, a TALE domain comprises a protein domain that binds to a preselected endogenous target site on at least one homologous chromosome. In another embodiment, a TALE domain comprises a protein domain that binds to a polymorphic allele on at least one homologous chromosome. In another embodiment, a TALE domain comprises a protein domain that binds to a preselected endogenous target site on both homologous chromosomes. In another embodiment, a TALE domain comprises a protein domain that binds to polymorphic alleles on both homologous chromosomes.

In one embodiment, a TALE domain comprises at least one of the TALE-repeat modules. In another embodiment, a TALE domain comprises from one to thirty TALE-repeat modules. In another embodiment, a TALE domain comprises more than thirty repeat modules. In another embodiment, a TALEN fusion protein comprises an N-terminal domain, one or more of TALE-repeat modules followed by a half-repeat module, a linker, and a nucleotide cleavage domain.

Chemical mutagenesis using an agent such as Ethyl Methyl Sulfonate (EMS) can be employed to obtain a population of point mutations and screen for mutants of the gene(s) of interest that may become silent or downregulated. In plants, methods relaying on introgression of genes from natural populations can be used. Cultured and wild type species are crossed repetitively such that a plant comprising a given segment of the wild genome is isolated. Certain plant species, for example, maize (corn) and snapdragon, have natural transposons. These transposons are either autonomous, i.e., the transposase is located within the transposon sequence or non-autonomous, without a transposase. A skilled person can cause transposons to “jump” and create mutations. Alternatively, a nucleic acid sequence can be synthesized having random nucleotides at one or more predetermined positions to generate random amino acid substituting.

In some embodiments, the expression of genes can be altered by the introduction of one or more point mutations into their regulatory sequences. In some embodiments, the expression of genes can be altered by the introduction of one or more point mutations into their regulatory sequences. A skilled artisan would appreciate that “regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. In some embodiments, regulatory sequences comprise promoters. In some embodiments, regulatory sequences comprise translation leader sequences. In some embodiments, regulatory sequences comprise introns. In some embodiments, regulatory sequences comprise polyadenylation recognition sequences. In some embodiments, regulatory sequences comprise RNA processing sites. In some embodiments, regulatory sequences comprise effector binding sites. In some embodiments, regulatory sequences comprise stem-loop structures.

A skilled artisan would appreciate that “promoter” refers to a DNA sequence capable of controlling the expression of a coding sequence or functional RNA. In some embodiments, a coding sequence is located 3′ to a promoter sequence. It is understood by those skilled in the art that different promoters may direct the expression of a gene in different tissues or cell types, or at different stages of development, or in response to different environmental or physiological conditions. In some embodiments, the promoter comprises a constitutive promoter, i.e., a promoter that causes a gene to be expressed in most cell types at most times. In some embodiments, the promoter comprises a regulated promoter, i.e., a promoter that causes a gene to be expressed in response to sporadic specific stimuli. It is further recognized that in many cases the exact boundaries of regulatory sequences have not been completely defined yet.

Examples of promoters include, but are not limited to, the Solanum lycopersicum ubiquitin promoter 10 (SlPrUbiq10), the cauliflower mosaic virus Pol-III promoter CaMV-35S-promoter (p35s), and the soybean seed-specific promoters (e.g., SEED1, SEED2, SEED3, SEED4, SEED5, and SEED 6).

A skilled artisan would appreciate that the term “3′ non-coding sequences” or “transcription terminator” refers to DNA sequences located downstream of a coding sequence. In some embodiments, 3′ non-coding sequences comprise polyadenylation recognition sequences. In some embodiments, 3′ non-coding sequences comprise sequences encoding regulatory signals capable of affecting mRNA processing. In some embodiments, 3′ non-coding sequences comprise sequences encoding regulatory signals capable of affecting gene expression. The polyadenylation signal is usually characterized by affecting the addition of polyadenylic acid tracts to the 3′ end of the mRNA precursor. In some embodiments, mutations in the 3′ non-coding sequences affect gene transcription. In some embodiments, mutations in the 3′ non-coding sequences affect RNA processing. In some embodiments, mutations in the 3′ non-coding sequences affect gene stability. In some embodiments, mutations in the 3′ non-coding sequences affect translation of the associated coding sequence.

### Biological Activity

In some embodiments, the biological activity of globulin gene proteins (e.g., GY1, GY2, GY3, GY4, GY5, alpha-conglycinin, alpha-prime-conglycinin, beta-conglycinin) is altered compared with a control globulin gene protein.

In some embodiments, the biological activity of desaturase proteins (e.g., fatty acid desaturase 1A [FAD2-1A], fatty acid desaturase 1B [FAD2-1B], delta-9-stearoyl-acyl-carrier protein desaturase [SACPD]) is altered compared with a control desaturase.

A skilled artisan would recognize that the term “biological activity” refers to any activity associated with a protein that can be measured by an assay. In some embodiments, the biological activity of a globulin affects the allergic response to the plant or a portion thereof. In some embodiments, the biological activity of a desaturase affects the levels of fatty acids in at least a part of a plant. In some embodiments, an altered biological activity comprises increased enzyme activity. In some embodiments, an altered biological activity comprises decreased enzyme activity. In some embodiments, an altered biological activity comprises increased stability of the polypeptide. In some embodiments, an altered biological activity comprises decreased stability of the polypeptide.

In some embodiments, the altered biological activity comprises
increased enzyme activity of a globulin or desaturase; or
increased stability of a globulin or desaturase; or
decreased enzyme activity of a globulin or desaturase; or
decreased stability of a globulin or desaturase;
compared to the biological activity in an unmodified or unedited plant.

In some embodiments, the biological activity of a globulin or desaturase is increased compared with a control globulin or desaturase. In some embodiments, the biological activity of a globulin or desaturase is decreased compared with a control globulin or desaturase. In some embodiments, a globulin or desaturase has increased stability compared with a control globulin or desaturase. In some embodiments, a globulin or desaturase has decreased stability compared with a control globulin or desaturase.

### Overexpression

According to yet additional embodiments the present invention provides a genetically modified or gene edited plant comprising at least one cell expressing at least one protein from the milk of a mammal, the at least one protein being selected from the group consisting of serum albumin, alpha-S1-casein, alpha-S2-casein, beta-casein, kappa-casein, beta-lactoglobulin, and alpha-lactalbumin and expressed in the genetically modified plant or a seed, bean, grain, fruit, nut, legume, leaf, stem, root, portion, product, isolate, exudate, secretion, or extract thereof.

Expression or over-expression of these proteins, or any combination thereof, can increase the content of milk proteins in plants.

### Transgenic Plants

Cloning of a polynucleotide encoding a protein of the present invention selected from the group consisting of serum albumin, alpha-S1-casein, alpha-S2-casein, beta-casein, kappa-casein, beta-lactoglobulin, and alpha-lactalbumin; guide-DNA pairs of the present invention or another molecule that silences a gene encoding a globulin or a desaturase can be performed by any method as is known to a person skilled in the art. Cloning of a polynucleotide encoding a milk protein polynucleotide of the present invention or a molecule that silences a gene encoding a globulin or desaturase can be performed by any method as is known to a person skilled in the art. Various DNA constructs may be used to express the desired gene or silencing molecule targeted to the gene in a desired organism.

According to certain embodiments, the gene or a silencing molecule targeted thereto form part of an expression vector comprising all necessary elements for expression of the gene or its silencing molecule. According to certain embodiments, the expression is controlled by a constitutive promoter. According to certain embodiments, the constitutive promoter is specific to a plant tissue. According to these embodiments, the tissue specific promoter is selected from the group consisting of root, tuber, leaves and fruit specific promoter. Root specific promoters are described, e.g., in Martinez, E. et al. 2003. Curr. Biol. 13:1435-1441. Fruit specific promoters are described among others in Estornell L. H. et al. 2009. Plant Biotechnol. J. 7:298-309 and Fernandez A. I. Et al. 2009 Plant Physiol. 151:1729-1740. Tuber specific promoters are described, e.g., in Rocha-Sosa M, et al., 1989. EMBO J. 8:23-29; McKibbin R. S. et al., 2006. Plant Biotechnol J. 4(4):409-18. Leaf specific promoters are described, e.g., in Yutao Yang, Guodong Yang, Shijuan Liu, Xingqi Guo and Chengchao Zheng. Science in China Series C: Life Sciences. 46: 651-660.

According to certain embodiments, the expression vector further comprises regulatory elements at the 3′ non-coding sequence. As used herein, the “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht I L et al. (1989. Plant Cell 1:671-680).

According to certain embodiments, a guide-RNA multiarray complex in a vector with CRISPR/Cas9 and CRISPR/CSY4 is controlled by a Pol-Ill promoter, Ca MV-35S-promoter (p35s), that allows expression of log RNA molecules, which will be processed into single guide-RNAs by a CRISPR/CSY4 RNA endonuclease.

Those skilled in the art will appreciate that the various components of the nucleic acid sequences and the transformation vectors described in the present invention are operatively linked, so as to result in expression of said nucleic acid or nucleic acid fragment. Techniques for operatively linking the components of the constructs and vectors of the present invention are well known to those skilled in the art. Such techniques include the use of linkers, such as synthetic linkers, for example including one or more restriction enzyme sites.

One skilled in the art would appreciate that the term “operably linked” may encompass the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

Methods for transforming a plant according to the teachings of the present invention are known to those skilled in the art. As used herein the term “transformation” or “transforming” describes a process by which a foreign DNA, such as a DNA construct, including expression vector, enters and changes a recipient cell into a transformed, genetically altered or transgenic cell. Transformation may be stable, wherein the nucleic acid sequence is integrated into the organism genome and as such represents a stable and inherited trait, or transient, wherein the nucleic acid sequence is expressed by the cell transformed but is not integrated into the genome, and as such represents a transient trait. According to preferred embodiments the nucleic acid sequence of the present invention is stably transformed into the plant cell.

The genetically altered plants having altered content of the desired milk proteins according to the teachings of the present invention are typically first selected based on the expression of the gene or protein. Plants having enhanced or aberrant expression of the gene or protein, are then analyzed for the content of milk proteins and optionally of silencers.

Detection is performed employing standard methods of molecular genetics, known to a person of ordinary skill in the art.

For measuring the gene's/genes' expression, cDNA or mRNA should be obtained from an organ in which the nucleic acid is expressed. The sample may be further processed before the detecting step. For example, the polynucleotides in the cell or tissue sample may be separated from other components of the sample, may be amplified, etc. All samples obtained from an organism, including those subjected to any sort of further processing are considered to be obtained from the organism.

Detection of the gene(s) or the silencing molecule(s) typically requires amplification of the polynucleotides taken from the candidate altered organism. Methods for DNA amplification are known to a person skilled in the art. Most commonly used method for DNA amplification is PCR (polymerase chain reaction; see, for example, PCR Basics: from background to Bench, Springer Verlag, 2000; Eckert et al., 1991. PCR Methods and Applications 1:17). Additional suitable amplification methods include the ligase chain reaction (LCR), transcription amplification and self-sustained sequence replication, and nucleic acid-based sequence amplification (NASBA).

According to certain embodiments, the nucleic acid sequence comprising the gene of interest further comprises a nucleic acid sequence encoding a selectable marker. According to certain embodiments, the selectable marker confers resistance to antibiotic or to an herbicide; in these embodiments the transgenic plants are selected according to their resistance to the antibiotic or herbicide.

### Breeding

In some embodiments, transformation techniques including breeding through transgene editing, use of transgenes, use of transient expression of a gene or genes, or use of molecular markers, or any combination thereof, may be used in the breeding of a plant having an altered expression. If transformation techniques require use of tissue culture, transformed cells may be regenerated into plants in accordance with techniques well known to those of skill in the art. Additionally, grafting may be used to facilitate expression of proteins in trees, including nuts in nut trees. The regenerated plants may then be grown and crossed with the same or different plant varieties using traditional breeding techniques to produce seeds, beans, grains, fruits, vegetables, nuts, or legumes, which are then selected under the appropriate conditions.

The content of milk proteins is measured as exemplified hereinbelow and as is known to a person skilled in the art.

In one embodiment, the plant is from a family selected from the group consisting of the Solanaceae family, the Fabaceae family, the Poaceae family, the Amaranthaceae family, the Lamiaceae family, the Pedaliaceae family, the Cucurbitaceae family, the Asteraceae family, the Linaceae family, the Cannabaceae family, the Juglandaceae family, the Rosaceae family, and the Anacardiaceae family, the Betalaceae family, and the Aracaceae family.

In one embodiment, the plant is any one of a variety of algae, including, but not limited to, chlorophytes (green algae), rhodophytes (red algae), or phaeo-phytes (brown algae). In one embodiment, the green algae is C. reinhardtii.

In one embodiment, the plant is from the Solanaceae family, the Nicotiana genus, or Nicotiana benthamiana. In another embodiment, the plant is from the Fabaceae family, the Glycine genus, or Glycine max (soy/soybean). Alternatively, the plant is from the Fabaceae family, but is selected from the group consisting of the Cicer genus (e.g., Cicer arietinum [chickpea, garbanzo bean]), the Pisum genus (e.g., Pisum sativum [pea]), the Arachis genus (e.g., Arachis hypogaea [peanut]), and the Lupinus genus (e.g., Lupinus albus [lupin/lupine]). In yet another embodiment, the plant is from the Poaceae family, the Oryza genus (e.g., rice), or is selected from the group consisting of Oryza sativa and Oryza glaberrima. Alternatively, the plant is from the Poaceae family, but is selected from the group consisting of the Hordeum genus (e.g., Hordeum vulgare [barley]), the Avena genus (e.g., Avena sativa [oat]), and the Triticum genus (e.g., Triticum spelta [spelt]). In still another embodiment, the plant is from the Amaranthaceae family, the Chenopodium genus, or Chenopodium quinoa (quinoa). In still another embodiment, the plant is from the Lamiaceae family, the Salvia genus, or Salvia hispanica (chia). In still another embodiment, the plant is from the Pedaliaceae family, the Sesamum genus, or Sesamum indicum (sesame, benne). In still another embodiment, the plant is from the Cucurbitaceae family or the Cucurbita genus (e.g., squash/pumpkin, including, but not limited to, Cucurbita pepo, Cucurbita maxima, Cucurbita argyrosperma, or Cucurbita moschata). In still another embodiment, the plant is from the Asteraceae family, the Helianthus genus, or is selected from the group consisting of Helianthus anmus (sunflower), Helianthus verticallatus (whorled sunflower) and Helianthus tuberosus (Jerusalem artichoke). In still another embodiment, the plant is from the Linaceae family, the Linum genus, or Linum usitatissimum (flax, linseed). In still another embodiment, the plant is from the Cannabaceae family (e.g., hemp, including Cannabis sativa). In still another embodiment, the plant is from the Betalaceae family or the Corylus genus (e.g., hazel/hazelnut/cobnut/filbert nut, including, but not limited to, Corylus avellana). In still another embodiment, the plant is from the Juglandaceae family, the Juglans genus, or is selected from the group consisting of Juglans regia (Persian or English walnut), Juglans nigra (black walnut), and Juglans cinera (butternut). In still another embodiment, the plant is from the Rosaceae family, the Prunus genus, or is Prunus dulcis (almond) or Prunus amygdalus. In still another embodiment, the plant is from the Anacardiaceae family, or is selected from the group consisting of the Anacardium genus (e.g., Anacardium occidentale [cashew]) and the Pistacia genus (e.g., Pistacia vera [pistachio]).

A skilled artisan would appreciate that plant breeding can be accomplished through many different techniques ranging from simply selecting plants with desirable characteristics for propagation, to methods that make use of knowledge of genetics and chromosomes, to more complex molecular techniques.

A skilled artisan would appreciate that the term “hybrid plant” may encompass a plant generated by crossing two plants of interest, propagating by seed or tissue and then growing the plants. When plants are crossed sexually, the step of pollination may include cross pollination or self-pollination or back crossing with an untransformed plant or another transformed plant. Hybrid plants include first generation and later generation plants. Disclosed herein is a method to manipulate and improve a plant trait, for a non-limiting example—increasing plant resistance, decreasing anti-nutritional properties in a plant, or decreasing toxins in a plant, or any combination thereof.

### Biomarkers

A skilled artisan would appreciate that the term “biomarker” comprises any measurable substance in an organism whose presence is indicative of a biological state or a condition of interest. In some embodiments, the presence of a biomarker is indicative of the presence of a compound or a group of compounds of interest. In some embodiments, the concentration of a biomarker is indicative of the concentration of a compound or a group of compounds of interest. In some embodiments, the concentration of a biomarker is indicative of an organism phenotype.

Further, one skilled in the art would appreciate that the term “comprising” used throughout is intended to mean that the genetically modified or gene edited plants disclosed herein, and methods of altering expression of genes, and altering production of SA and/or SGA within these genetically modified or gene edited plants includes the recited elements, but not excluding others which may be optional. “Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited.

Disclosed herein are genetically modified plants, product comprising such plants or plant parts, methods of making the genetically modified plants or products, and the vectors thereof. In some embodiments, disclosed herein is a genetically modified plant comprising at least one cell expressing at least one protein from the milk of a mammal, the at least one protein being selected from the group consisting of serum albumin, alpha-S1-casein, alpha-S2-casein, beta-casein, kappa-casein, beta-lactoglobulin, and alpha-lactalbumin and expressed in the genetically modified plant or a seed, bean, grain, fruit, nut, legume, leaf, stem, root, or portion thereof, wherein each of said at least one protein is a recombinant protein at least 90% identical to the corresponding mammalian protein amino acid sequence, said recombinant protein being produced by the plant cell.

In some embodiments, disclosed herein is a genetically modified plant comprising at least one cell expressing at least one protein from the milk of a mammal, the at least one protein being selected from the group consisting of serum albumin, alpha-S1-casein, alpha-S2-casein, beta-casein, kappa-casein, beta-lactoglobulin, and alpha-lactalbumin and differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical mammalian species, wherein each of said at least one protein is a recombinant protein at least 90% identical to the corresponding mammalian protein amino acid sequence, said recombinant protein being produced by the plant cell.

In some embodiments, as disclosed herein the plant does not produce or comprise any other milk proteins aside from serum albumin, alpha-S1-casein, alpha-S2-casein, beta-casein, kappa-casein, beta-lactoglobulin, or alpha-lactalbumin.

In some embodiments, as disclosed herein the at least one protein from the milk of a mammal is from a human or non-human mammal.

In some embodiments, as disclosed herein the at least one protein from the milk of a mammal is from a mammal selected from the Bovidae family.

In some embodiments, as disclosed herein the at least one protein from the milk of a mammal is from a mammal of a genus of the Bovidae family selected from the group consisting of the Bos genus, the Capra genus, the Bubalus genus, the Syncerus genus, the Ovis genus, and the Bison genus.

In some embodiments, as disclosed herein the at least one protein from the milk of a mammal is from a mammal that is Bos taurus or Bubalus bubalis.

In some embodiments, as disclosed herein the mammal is selected from the Bos genus and wherein: the amino acid sequence of the serum albumin is at least 90% identical to the amino acid sequence set forth in SEQ ID NO: 36, or the polynucleotide encoding the serum albumin encodes a serum albumin that is at least 90% identical to the serum albumin encoded by the polynucleotide sequence set forth in SEQ ID NO: 29, the amino acid sequence of the alpha-S1-casein is at least 90% identical to the amino acid sequence set forth in SEQ ID NO: 37, or the polynucleotide encoding the alpha-S1-casein encodes an alpha-S1-casein that is at least 90% identical to the alpha-S1-casein encoded by the polynucleotide sequence set forth in SEQ ID NO: 30; the amino acid sequence of the alpha-S2-casein is at least 90% identical to the amino acid sequence set forth in SEQ ID NO: 38 or the polynucleotide encoding the alpha-S2-casein encodes an alpha-S2-casein that is at least 90% identical to the alpha-S2-casein encoded by the polynucleotide sequence set forth in SEQ ID NO: 31; the amino acid sequence of the beta-casein is at least 90% identical to the amino acid sequence set forth in SEQ ID NO: 39 or the polynucleotide encoding the beta-casein encodes a beta-casein that is at least 90% identical to the beta-casein encoded by the polynucleotide sequence set forth in SEQ ID NO: 32; the amino acid sequence of the kappa-casein is at least 90% identical to the amino acid sequence set forth in SEQ ID NO: 40 or the polynucleotide encoding the kappa-casein encodes a kappa-casein that is at least 90% identical to the kappa-casein encoded by the polynucleotide sequence set forth in SEQ ID NO: 33; the amino acid sequence of the beta-lactoglobulin is at least 90% identical to the amino acid sequence set forth in SEQ ID NO: 41 or the polynucleotide encoding the beta-lactoglobulin encodes a beta-lactoglobulin that is at least 90% identical to the beta-lactoglobulin encoded by the polynucleotide sequence set forth in SEQ ID NO: 34; and the amino acid sequence of the alpha-lactalbumin is at least 90% identical to the amino acid sequence set forth in SEQ ID NO: 42 or the polynucleotide encoding the alpha-lactalbumin encodes an alpha-lactalbumin that is at least 90% identical to the alpha-lactalbumin encoded by the polynucleotide sequence set forth in SEQ ID NO: 35.

In some embodiments, as disclosed herein the at least one cell further comprises: decreased expression of at least one globulin gene protein; or decreased expression of at least one desaturase gene, wherein expression of the at least one globulin gene protein or expression of the at least one desaturase gene protein is reduced in the modified plant compared to its expression in a corresponding unmodified plant, thereby the modified plant comprises reduced content of at least one globulin or derivative thereof, or of at least one desaturase or derivative thereof, or comprises an increased content of at least one oleic acid or derivative thereof or at least one stearic acid or derivative thereof or a reduced content of at least one saturated fat, compared to the corresponding unmodified plant.

In some embodiments, as disclosed herein the plant is from a family selected from the group consisting of the Solanaceae family, the Fabaceae family, the Poaceae family, the Amaranthaceae family, the Lamiaceae family, the Pedaliaceae family, the Cucurbitaceae family, the Asteraceae family, the Linaceae family, the Cannabaceae family, the Juglandaceae family, the Rosaceae family, the Anacardiaceae family, the Betalaceae family, and the Aracaceae family;

the plant is an alga selected from the group consisting of a chlorophyte, a rhodophyte, and a phaeo-phyte; or the plant is C. reinhardtii.

In some embodiments, as disclosed herein the plant is from a genus of the Fabaceae family selected from the group consisting of Glycine, Cicer, Phaseolus, Pisum, Arachis, and Lupinus.

In some embodiments, as disclosed herein the plant is Glycine max.

In some embodiments, as disclosed herein the plant is from the Oryza genus of the Poaceae family.

In some embodiments, as disclosed herein the plant is selected from the group consisting of Oryza sativa or Oryza glaberrima.

In some embodiments, as disclosed herein the plant is Nicotiana benthamiana of the Solanaceae family.

In some embodiments, as disclosed herein expression of each of the at least one protein from the milk of a mammal is independently under control of a seed promoter.

In some embodiments, as disclosed herein the plant is selected from the genus Glycine and wherein the seed promoter is selected independently from the group consisting of Seed 1, Seed 2, Seed 3, Seed 4, Seed 5, and Seed 6.

In some embodiments, as disclosed herein the plant is selected from the genus Glycine, and wherein the at least one cell further comprises: decreased expression of at least one globulin gene protein selected from the group consisting of a gene encoding glycinin 1 (GY1), a gene encoding glycinin 2 (GY2), a gene encoding glycinin 3 (GY3), a gene encoding glycinin 4 (GLY4), a gene encoding glycinin 5 (GY5), a gene encoding alpha-conglycinin, a gene encoding alpha-prime-conglycinin, and a gene encoding beta-conglycinin; or decreased expression of at least one desaturase gene selected from the group consisting of a gene encoding fatty acid desaturase 1A (FAD2-1A), a gene encoding fatty acid desaturase 1B (FAD2-1B), and a gene encoding delta-9-stearoyl-acyl-carrier protein desaturase (SACPD) compared to its expression in a corresponding unmodified plant, wherein expression of the at least one globulin gene protein or expression of the at least one desaturase gene protein is reduced in the modified plant compared to its expression in a corresponding unmodified plant, thereby the modified plant comprises reduced content of at least one globulin or derivative thereof, or of at least one desaturase or derivative thereof, or comprises an increased content of at least one oleic acid or derivative thereof or at least one stearic acid or derivative thereof or a reduced content of at least one saturated fat, compared to the corresponding unmodified plant.

In some embodiments, as disclosed herein the expression of the at least one gene or any combination thereof is decreased, the decrease comprising mutagenizing the at least one gene, wherein the mutagenesis comprises introduction of one or more point mutations, or genome editing, or use of a bacterial CRISPR/CAS system, or a combination thereof.

In some embodiments, as disclosed herein the genetically modified plant is a transgenic or gene-edited plant comprising at least one cell comprising: at least one first series silencer targeted to a polynucleotide encoding at least one globulin protein or fragment thereof, selected from the group consisting of a fragment of a gene encoding glycinin 1 (GY1) or a complementary sequence thereof, a fragment of a gene encoding glycinin 2 (GY2) or a complementary sequence thereof, a fragment of a gene encoding glycinin 3 (GY3) or a complementary sequence thereof, a fragment of a gene encoding glycinin 4 (GLY4) or a complementary sequence thereof, a fragment of a gene encoding glycinin 5 (GY5) or a complementary sequence thereof, a fragment of a gene encoding alpha-conglycinin or a complementary sequence thereof, a fragment of a gene encoding alpha-prime-conglycinin or a complementary sequence thereof, and a fragment of a gene encoding beta-conglycinin or a complementary sequence thereof, or wherein the transgenic or gene edited plant comprises a polynucleotide encoding at least one protein selected from the group consisting of glycinin 1 (GY1), glycinin 2 (GY2), glycinin 3 (GY3), glycinin 4 (GLY4), glycinin 5 (GY5), alpha-conglycinin, alpha-prime-conglycinin, and beta-conglycinin, wherein expression of the polynucleotide is selectively silenced, repressed, or reduced; or at least one second series silencer targeted to a polynucleotide encoding at least one desaturase protein or a portion thereof, selected from the group consisting of a fragment of a gene encoding fatty acid desaturase 1A (FAD2-1A) or a complementary sequence thereof, a fragment of a gene encoding fatty acid desaturase 1B (FAD2-1B) or a complementary sequence thereof, and a fragment of a gene encoding delta-9-stearoyl-acyl-carrier protein desaturase (SACPD) or a complementary sequence thereof, or wherein the transgenic or gene-edited plant comprises a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and delta-9-stearoyl-acyl-carrier protein desaturase (SACPD) or a portion thereof, wherein expression of the polynucleotide is selectively silenced, repressed, or reduced.

In some embodiments, as disclosed herein the polynucleotide has been selectively edited by deletion, insertion, or modification to silence, repress, or reduce expression thereof, or wherein the genetically modified plant is a progeny of the transgenic or gene-edited plant.

In some embodiments, as disclosed herein the at least one first series silencer comprises at least one guide-RNA pair targeted to a 5′-translated region of a polynucleotide encoding at least one globulin protein or a portion thereof selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; or the at least one second series silencer comprises at least one guide-RNA pair targeted to a 5′-translated region of a polynucleotide encoding at least one desaturase protein or a portion thereof, selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, as disclosed herein the at least one guide-RNA pair is selected from the group consisting of (i) the guide-RNA pair encoded by SEQ ID NO: 57 and SEQ ID NO: 58, (ii) the guide-RNA pair encoded by SEQ ID NO: 59 and SEQ ID NO: 60, (iii) the guide-RNA pair encoded by SEQ ID NO: 61 and SEQ ID NO: 62, and (iv) the guide-RNA pair encoded by SEQ ID NO: 63 and SEQ ID NO: 64; or the at least one guide-RNA pair is selected from the group consisting of (i) the guide-RNA pair encoded by SEQ ID NO: 65 and SEQ ID NO: 66, and (ii) the guide-RNA pair encoded by SEQ ID NO: 67 and SEQ ID NO: 68.

In some embodiments, as disclosed herein the genetically modified plant is further comprising at least one cell expressing at least three proteins from the milk of a mammal of the Bos genus, wherein the plant is selected from the genus Glycine and wherein:

the at least three proteins are selected from the group consisting of serum albumin, alpha-S1-casein, alpha-S2-casein, beta-casein, kappa-casein, beta-lactoglobulin, and alpha-lactalbumin, wherein: the amino acid sequence of the serum albumin is at least 90% identical to the amino acid sequence set forth in SEQ ID NO: 36, or the polynucleotide encoding the serum albumin encodes a serum albumin that is at least 90% identical to the serum albumin encoded by the polynucleotide sequence set forth in SEQ ID NO: 29; the amino acid sequence of the alpha-S1-casein is at least 90% identical to the amino acid sequence set forth in SEQ ID NO: 37, or the polynucleotide encoding the alpha-S1-casein encodes an alpha-S1-casein that is at least 90% identical to the alpha-S1-casein encoded by the polynucleotide sequence set forth in SEQ ID NO: 30; the amino acid sequence of the alpha-S2-casein is at least 90% identical to the amino acid sequence set forth in SEQ ID NO: 38 or the polynucleotide encoding the alpha-S2-casein encodes an alpha-S2-casein that is at least 90% identical to the alpha-S2-casein encoded by the polynucleotide sequence set forth in SEQ ID NO: 31; the amino acid sequence of the beta-casein is at least 90% identical to the amino acid sequence set forth in SEQ ID NO: 39 or the polynucleotide encoding the beta-casein encodes a beta-casein that is at least 90% identical to the beta-casein encoded by the polynucleotide sequence set forth in SEQ ID NO: 32; the amino acid sequence of the kappa-casein is at least 90% identical to the amino acid sequence set forth in SEQ ID NO: 40 or the polynucleotide encoding the kappa-casein encodes a kappa-casein that is at least 90% identical to the kappa-casein encoded by the polynucleotide sequence set forth in SEQ ID NO: 33; the amino acid sequence of the beta-lactoglobulin is at least 90% identical to the amino acid sequence set forth in SEQ ID NO: 41 or the polynucleotide encoding the beta-lactoglobulin encodes a beta-lactoglobulin that is at least 90% identical to the beta-lactoglobulin encoded by the polynucleotide sequence set forth in SEQ ID NO: 34; and the amino acid sequence of the alpha-lactalbumin is at least 90% identical to the amino acid sequence set forth in SEQ ID NO: 42 or the polynucleotide encoding the alpha-lactalbumin encodes an alpha-lactalbumin that is at least 90% identical to the alpha-lactalbumin encoded by the polynucleotide sequence set forth in SEQ ID NO: 35, wherein each of said at least three proteins is a recombinant protein produced by the plant cell and wherein expression of each said recombinant protein is independently under control of a promoter selected from the group consisting of seed promoters of the genus Glycine, each said recombinant protein being expressed in the cell at a relative abundance of at least 75% when compared to the relative abundance of protein in the milk of the mammal of the Bos genus; and the at least one cell further comprises: decreased expression of at least one globulin gene selected from the group consisting of a gene encoding glycinin 1 (GY1), a gene encoding glycinin 2 (GY2), a gene encoding glycinin 3 (GY3), a gene encoding glycinin 4 (GLY4), a gene encoding glycinin 5 (GY5), a gene encoding alpha-conglycinin, a gene encoding alpha-prime-conglycinin, and a gene encoding beta-conglycinin compared to its expression in a corresponding unmodified plant, wherein the at least one cell further comprises at least one first series silencer; and decreased expression of at least one desaturase gene selected from the group consisting of a gene encoding fatty acid desaturase 1A (FAD2-1A), a gene encoding fatty acid desaturase 1B (FAD2-1B), and a gene encoding delta-9-stearoyl-acyl-carrier protein desaturase (SACPD) compared to its expression in a corresponding unmodified plant, wherein the at least one cell further comprises at least one second series silencer, wherein expression of the at least one globulin gene or expression of the at least one desaturase gene is reduced in the modified plant compared to its expression in a corresponding unmodified plant, the modified plant comprising reduced content of at least one globulin or derivative thereof, or of at least one desaturase or derivative thereof, or comprises an increased content of at least one oleic acid or derivative thereof or stearic acid or derivative thereof or a reduced content of at least one saturated fat, compared to the corresponding unmodified plant, compared to the corresponding unmodified plant.

In some embodiments, as disclosed herein wherein the genetically modified plant is further comprising at least one cell expressing proteins from the milk of a mammal of the Bos genus, wherein: the proteins from the milk of a mammal consist of serum albumin, alpha-S1-casein, alpha-S2-casein, beta-casein, kappa-casein, beta-lactoglobulin, and alpha-lactalbumin; and each of the proteins is differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical Bos species.

In some embodiments, as disclosed herein the expression of each protein from the milk of a mammal is independently under control of a seed promoter, wherein: expression of beta-casein is controlled by Seed 1 (SEQ ID NO: 51); expression of kappa-casein and beta-lactoglobulin are controlled by Seed 2 (SEQ ID NO: 52); expression of alpha-S2-casein is controlled by Seed 3 (SEQ ID NO: 53); expression of alpha-S1-casein is controlled by Seed 4 (SEQ ID NO: 54); expression of serum albumin is controlled by Seed 5 (SEQ ID NO: 55); and expression of alpha-lactalbumin is controlled by Seed 6 (SEQ ID NO: 56).

In some embodiments, as disclosed herein wherein each of the proteins is differentially expressed to produce a content profile in the genetically modified plant or a seed, bean, grain, fruit, nut, legume, leaf, stem, root, portion, product, isolate, exudate, secretion, or extract thereof of at least 75% and no greater than 150% of a content profile in milk of the identical Bos species.

In some embodiments, as disclosed herein wherein: the at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; and the at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, as disclosed herein wherein: the at least one first series silencer comprises at least one guide-RNA pair selected from the group consisting of (a) the guide-RNA pair encoded by SEQ ID NO: 57 and SEQ ID NO: 58, (b) the guide-RNA pair encoded by SEQ ID NO: 59 and SEQ ID NO: 60, (c) the guide-RNA pair encoded by SEQ ID NO. 61 and SEQ ID NO: 62, and (d) the guide-RNA pair encoded by SEQ ID NO: 63 and SEQ ID NO: 64; and the at least one second series silencer comprises at least one guide-RNA pair selected from the group consisting of (a) the guide-RNA pair encoded by SEQ ID NO: 65 and SEQ ID NO: 66, and (b) the guide-RNA pair encoded by SEQ ID NO: 67 and SEQ ID NO: 68.

In some embodiments, as disclosed herein wherein: the first series silencer comprises: (a) a guide-RNA pair encoded by SEQ ID NO: 57 and SEQ ID NO: 58, (b) a pair encoded by SEQ ID NO: 59 and SEQ ID NO: 60, (c) a guide-RNA pair encoded by SEQ ID NO: 61 and SEQ ID NO: 62, and (d) a guide-RNA pair encoded by SEQ ID NO: 63 and SEQ ID NO: 64; and the second series silencer comprises: (a) a guide-RNA pair encoded by SEQ ID NO: 65 and SEQ ID NO: 66, and (b) a guide-RNA pair encoded by SEQ ID NO: 67 and SEQ ID NO: 68.

In some embodiments, as disclosed herein is a food, medicament, cosmetic or blocking composition comprising: a genetically modified plant comprising at least one cell expressing at least one protein from the milk of a mammal, the at least one protein being selected from the group consisting of serum albumin, alpha-S1-casein, alpha-S2-casein, beta-casein, kappa-casein, beta-lactoglobulin, and alpha-lactalbumin and expressed in the genetically modified plant or a seed, bean, grain, fruit, nut, legume, leaf, stem, root, or portion thereof, wherein each of said at least one protein is a recombinant protein at least 90% identical to the corresponding mammalian protein amino acid sequence, said recombinant protein being produced by the plant cell.

In some embodiments, as disclosed herein a cell comprises a seed, bean, grain, fruit, nut, legume, leaf, stem, root, portion, product, isolate, exudate, secretion, or extract thereof, the food, medicament, cosmetic or blocking composition comprising at least one protein from the milk of a mammal.

In some embodiments, as disclosed herein the food, medicament, cosmetic or blocking composition comprising mammalian proteins from the milk of a mammal of the Bovidae family consisting of serum albumin, alpha-S1-casein, alpha-S2-casein, beta-casein, kappa-casein, beta-lactoglobulin, and alpha-lactalbumin, wherein each of the proteins is differentially expressed to produce a content profile in the genetically modified plant or a seed, bean, grain, fruit, nut, legume, leaf, stem, root, portion, product, isolate, exudate, secretion, or extract thereof of at least 70% and no greater than 150% of a content profile in milk of a mammal of the identical Bos species.

In some embodiments, as disclosed herein wherein: the level of each of glycinin 1 (GY1), glycinin 2 (GY2), glycinin 3 (GY3), glycinin 4 (GLY4 glycinin 5 (GY5), alpha-conglycinin, alpha-prime-conglycinin, and beta-conglycinin is reduced as compared with the respective level of each in a non-genetically modified plant of the same species; the level of each of fatty acid desaturase 1A (FAD2-1A), fatty acid desaturase 1B (FAD2-1B), and delta-9-stearoyl-acyl-carrier protein desaturase (SACPD) is reduced as compared with the respective level of each in a non-genetically modified plant of the same species; and the food, medicament, cosmetic or blocking composition does not comprise any other milk proteins aside from serum albumin, alpha-S1-casein, alpha-S2-casein, beta-casein, kappa-casein, beta-lactoglobulin, or alpha-lactalbumin.

In some embodiments, as disclosed herein said food product, medicament, cosmetic or blocking composition further comprises the addition of milk from a mammal for a final concentration of between 1%-60% milk from a mammal or further comprising the addition of an unmodified milk alternative from a plant.

In some embodiments, as disclosed herein is DNA binary vector or viral vector for expressing in a plant, proteins from the milk of a mammal, the vector comprising: a selectable marker; polynucleotide sequences encoding at least three proteins from the milk of a mammal, wherein the at least three proteins are selected from the group consisting of serum albumin, alpha-S1-casein, alpha-S2-casein, beta-casein, kappa-casein, beta-lactoglobulin, and alpha-lactalbumin, each independently under control of a promoter, wherein: each of said recombinant proteins is at least 90% identical to the corresponding mammalian protein amino acid sequence.

In some embodiments, as disclosed herein wherein each of the recombinant proteins is differentially expressed to produce a content profile in the genetically modified plant or a seed, bean, grain, fruit, nut, legume, leaf, stem, root, portion, product, isolate, exudate, secretion, or extract thereof of at least 70% of a content profile in milk of a mammal of the identical mammalian species.

In some embodiments, as disclosed herein the DNA binary vector or viral vector further comprising polynucleotide sequences encoding seven proteins from the milk of a mammal, wherein the proteins from the milk of a mammal consist of serum albumin, alpha-S1-casein, alpha-S2-casein, beta-casein, kappa-casein, beta-lactoglobulin, and alpha-lactalbumin.

In some embodiments, as disclosed herein wherein the mammal is selected from the Bos genus and wherein: the amino acid sequence of the serum albumin is at least 90% identical to the amino acid sequence set forth in SEQ ID NO: 36, or the polynucleotide encoding the serum albumin encodes a serum albumin that is at least 90% identical to the serum albumin encoded by the polynucleotide sequence set forth in SEQ ID NO: 29; the amino acid sequence of the alpha-S1-casein is at least 90% identical to the amino acid sequence set forth in SEQ ID NO: 37, or the polynucleotide encoding the alpha-S1-casein encodes an alpha-S1-casein that is at least 90% identical to the alpha-S1-casein encoded by the polynucleotide sequence set forth in SEQ ID NO: 30; the amino acid sequence of the alpha-S2-casein is at least 90% identical to the amino acid sequence set forth in SEQ ID NO: 38 or the polynucleotide encoding the alpha-S2-casein encodes an alpha-S2-casein that is at least 90% identical to the alpha-S2-casein encoded by the polynucleotide sequence set forth in SEQ ID NO: 31, the amino acid sequence of the beta-casein is at least 90% identical to the amino acid sequence set forth in SEQ ID NO: 39 or the polynucleotide encoding the beta-casein encodes a beta-casein that is at least 90% identical to the beta-casein encoded by the polynucleotide sequence set forth in SEQ ID NO: 32; the amino acid sequence of the kappa-casein is at least 90% identical to the amino acid sequence set forth in SEQ ID NO: 40 or the polynucleotide encoding the kappa-casein encodes a kappa-casein that is at least 90% identical to the kappa-casein encoded by the polynucleotide sequence set forth in SEQ ID NO: 33; the amino acid sequence of the beta-lactoglobulin is at least 90% identical to the amino acid sequence set forth in SEQ ID NO: 41 or the polynucleotide encoding the beta-lactoglobulin encodes a beta-lactoglobulin that is at least 90% identical to the beta-lactoglobulin encoded by the polynucleotide sequence set forth in SEQ ID NO: 34; and the amino acid sequence of the alpha-lactalbumin is at least 90% identical to the amino acid sequence set forth in SEQ ID NO: 42 or the polynucleotide encoding the alpha-lactalbumin encodes an alpha-lactalbumin that is at least 90% identical to the alpha-lactalbumin encoded by the polynucleotide sequence set forth in SEQ ID NO: 35.

In some embodiments, as disclosed herein the plant is selected from the genus Glycine and wherein expression of each protein from the milk of a mammal is independently under control of a seed promoter.

In some embodiments, as disclosed herein wherein: expression of beta-casein is controlled by Seed 1 (SEQ ID NO: 51); expression of kappa-casein and beta-lactoglobulin are controlled by Seed 2 (SEQ ID NO: 52); expression of alpha-S2-casein is controlled by Seed 3 (SEQ ID NO: 53); expression of alpha-S1-casein is controlled by Seed 4 (SEQ ID NO: 54); expression of serum albumin is controlled by Seed 5 (SEQ ID NO: 55); and expression of alpha-lactalbumin is controlled by Seed 6 (SEQ ID NO: 56).

In some embodiments, as disclosed herein the DNA binary vector or viral vector further comprises an expression sequence encoding CRISPR/CSY4; an expression sequence encoding CRISPR/Cas9; a guide-RNA expression multiarray complex under the control of an independent guide-RNA expression multiarray complex promotor, the guide-RNA expression multiarray complex encoding one or more guide-RNA pairs in an array cleavable by a CRISPR/CSY4 RNA endonuclease, wherein: the at least one first series silencer guide-RNA pair is targeted to a polynucleotide encoding at least one globulin gene protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; or the at least one second series silencer guide-RNA pair is targeted to a polynucleotide encoding at least one desaturase gene protein or a portion thereof, selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof.

In some embodiments, as disclosed herein the guide-RNA expression multiarray complex encoding a first series silencer targeted to a 5′-translated region of a polynucleotide encoding a globulin protein or a portion thereof or a second series silencer target to a 5′-translated region of a polynucleotide encoding a desaturase protein or a portion thereof.

In some embodiments, as disclosed herein the guide-RNA expression multiarray complex encoding a first series silencer and a second series silencer, wherein: the first series silencer comprises one or more guide-RNA pairs selected from the group consisting of (i) the guide-RNA pair encoded by SEQ ID NO: 57 and SEQ ID NO: 58, (ii) the guide-RNA pair encoded by SEQ ID NO: 59 and SEQ ID NO: 60, (iii) the guide-RNA pair encoded by SEQ ID NO: 61 and SEQ ID NO: 62, and (iv) the guide-RNA pair encoded by SEQ ID NO: 63 and SEQ ID NO: 64; and the second series silencer comprises one or more guide-RNA pairs selected from the group consisting of (i) the guide-RNA pair encoded by SEQ ID NO: 65 and SEQ ID NO: 66, and (ii) the guide-RNA pair encoded by SEQ ID NO: 67 and SEQ ID NO: 68.

In some embodiments, as disclosed herein the independent guide-RNA expression multiarray complex promotor is a CaMV-35S-promoter (p35s).

In some embodiments, as disclosed herein the selectable marker is a BASTA resistance marker.

In some embodiments, as disclosed herein the vector having a sequence at least 90% identical to SEQ ID NO: 50 or at least 90% identical to SEQ ID NO: 69.

In some embodiments, as disclosed herein is a genetically modified plant cell comprising the vector a described herein.

In some embodiments, as disclosed herein a method of producing a food, medicament, cosmetic or blocking composition comprising a genetically modified plant or a seed, bean, grain, fruit, nut, legume, leaf, stem, root, portion, product, isolate, exudate, secretion, or extract thereof having at least 70% of a content profile in milk of a mammal, the method comprising: providing a DNA binary vector or viral vector for differentially expressing in a plant, proteins from the milk of a mammal, the vector comprising: a selectable marker; and polynucleotide sequences encoding at least three recombinant proteins from the milk of a mammal, wherein the proteins are selected from the group consisting of serum albumin, alpha-S1-casein, alpha-S2-casein, beta-casein, kappa-casein, beta-lactoglobulin, and alpha-lactalbumin, each independently under control of a promoter, wherein: each of said recombinant proteins is at least 90% identical to the corresponding mammalian protein amino acid sequence: and wherein each of the promoters for each of the polynucleotide sequences encoding recombinant proteins from the milk of a mammal differentially activates expression of its corresponding polynucleotide sequence to produce a content profile in the genetically modified plant or a portion, seed, bean, grain, fruit, nut, legume, leaf, stem, root, product, isolate, exudate, secretion, or extract thereof having at least 70% of a content profile in milk from a mammal of the identical mammalian species; transfecting at least one plant cell with the DNA binary vector or viral vector; differentially expressing the at least three recombinant proteins to produce a food, medicament, cosmetic or blocking composition comprising the genetically modified plant or a portion, seed, bean, grain, fruit, nut, legume, leaf, stem, root, product, isolate, exudate, secretion, or extract thereof having a content profile of at least 70% of a content profile in milk from a mammal of the identical mammalian species; and optionally adding milk of a mammal to the food, medicament, cosmetic or blocking composition step.

In some embodiments, as disclosed herein the vector further comprises an expression sequence encoding CRISPR/CSY4; an expression sequence encoding CRISPR/Cas9; a guide-RNA expression multiarray complex under the control of an independent guide-RNA expression multiarray complex promotor, the guide-RNA expression multiarray complex encoding one or more guide-RNA pairs in an array cleavable by a CRISPR/CSY4 RNA endonuclease, wherein: the at least one first series silencer guide-RNA pair is targeted to a polynucleotide encoding at least one globulin gene protein selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof; or the at least one second series silencer guide-RNA pair is targeted to a polynucleotide encoding at least one desaturase gene protein selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof, wherein expression of the at least one globulin gene protein or expression of the at least one desaturase gene protein is reduced in the modified plant compared to its expression in a corresponding unmodified plant, thereby the modified plant comprises reduced content of at least one globulin or derivative thereof, or of at least one desaturase or derivative thereof, or comprises an increased content of at least one oleic acid or derivative thereof or stearic acid or derivative thereof or a reduced content of at least one saturated fat, compared to the corresponding unmodified plant.

In some embodiments, as disclosed herein the vector having a sequence at least 90% identical to SEQ ID NO: 50 or at least 90% identical to SEQ ID NO: 69.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials & Methods

### Plant Growth and Material

N. benthamiana plants were grown in a growth room maintained at 23±2° C. at required light intensity with 16-h day/8-h night.

### Quantitative Real-Time PCR

Gene expression analysis was performed with three biological replicates (n=3) for each genotype. RNA isolation was performed by the TRIZOL® method (SIGMA-ALDRICH®). DNaseI (SIGMA-ALDRICH®). Treated RNA was reverse transcribed using a high-capacity cDNA reverse transcription kit (APPLIED BIOSYSTEMS®). Gene-specific oligonucleotides were designed with Primer-BLAST™ (https://www.ncbi.nlm.nih.gov/tools/primer-blast/). The F-Box gene was used as an endogenous control for N. benthamiana samples. Oligonucleotides used are listed in TABLE 1.

TABLE 1
List of primers used for qRT-PCR analysis.

| Name | Sequence |
|---|---|
| qRT-ALB_Fw_P1 | CTTCCTGGGCTCGTTTTTGT |
| | (SEQ ID NO: 1) |
| qRT-ALB_Rv_P1 | ACAGCATTCCTCCAGTGTGG |
| | (SEQ ID NO: 2) |
| qRT-CSN1S1_Fw_P1 | AGTGITGAGCAGAAGCACAT |
| | (SEQ ID NO: 3) |
| qRT-CSN1S1_Rv_P1 | GTTGGGCATGGATTCCCTCT |
| | (SEQ ID NO: 4) |
| qRT-CSN1S2_Fw_P1 | GCTGTTGCCCTTGCAAAGAA |
| | (SEQ ID NO: 5) |
| qRT-CSN1S2_Rv_P1 | TCCTTGCAGAATGTGGAGCA |
| | (SEQ ID NO: 6) |
| qRT-CSN2_Fw_P1 | CAGTGAGGAATCTATTACACGCA |
| | (SEQ ID NO: 7) |
| qRT-CSN2_Rv_P1 | TGGGCAAAGGGGTGGATTTT |
| | (SEQ ID NO: 8) |
| qRT-CSN3_Fw_P1 | ACCATTGCTAGTGGTGAGCC |
| | (SEQ ID NO: 9) |
| qRT-CSN3_Rv_P1 | TGTGTTGATCTCAGGTGGGC |
| | (SEQ ID NO: 10) |
| qRT-LALBA_Fw_P1 | TCTCTCTCTCCTGGTAGGCAT |
| | (SEQ ID NO: 11) |
| qRT-LALBA_Rv_P1 | GGCAAACTGACACCTCCGTA |
| | (SEQ ID NO: 12) |
| qRT-LACB_Fw_P1 | AGATCCCTGCGGTGTTCAAG |
| | (SEQ ID NO: 13) |
| qRT-LACB_Rv_P1 | GGCTCAGCACTGTTCTCCAT |
| | (SEQ :ID NO: 14) |

Transient Expression in N. benthamiana

Transient gene expression assays in N. benthamiana with the following vectors: (α) pDGB-al ALB, (b) pDGB-α2 CSN1S1, (c) pDGB-α1 CSN1S2, (d) pDGB-α2 CSN2, (e) pDGB-α1 CSN3, (f) pDGB-α2 LALABA (LALBA) and (g) pDGB-α1 LGB (LACB), were based on a previously described agroinfiltration method by Sparkes 2006 (Sparkes et al. (2006) Nat. Protoc. 1(4): 2019-2025 [“Sparkes 2006”]). All constructs were transformed into the A. tumefaciens GV3101 strain. In all cases, agrobacteria were grown overnight in LB media and brought to a final OD₆₀₀ of 0.2 in infiltration buffer. Tissues used for subsequent liquid chromatography-mass spectrometry/mass spectrometry (LC-MS/MS) proteomics and quantitative reverse transcription-polymerase chain reaction (qRT-PCR) analysis were sampled from leaves 5 days post infiltration.

### Generation of DNA Constructs

Cow's milk genes were purchased as cDNA gene fragments based on a bacterial expression vector pUC18 from DHARMACON™. All vectors carrying the seven milk proteins were constructed using Goldenbraid cloning (Sarrion-Perdigones et al. (July 2013) PLANT Physiol. 162(3): 1618-1631 [“Sarrion-Perdigones 2013”]; see also https://gbcloning.upv.es/). ALB, CSN1S1, CSN1S2, CSN2, CSN3, LALBA (LALABA), and LGB (LACB) were initially amplified using PCR and gene specific primers (TABLE 2) and cloned into a pUPD2 vector. The pDGB-seven milk genes vector is a 301 (3-omega-1) vector. All vectors are based on a pCAMBIA backbone.

TABLE 2
List of primers used for amplification and cloning
of the cow's milk genes.
(Fw = forward: Rev = reverse)

| Name | Sequence |
|---|---|
| ALB Fw | GCGCCGTCTCGCTCGAATGAAGTEGGGFGACTTTTATTCT |
| | (SEQ ID NO: 15) |
| ALB Rev | GCGCCGTCTCGCTCAAAGCTTAGGCTAAGGCTGTTTGAGT |
| | (SEQ ED NO: 16) |
| CSN1S1Fw | GCGCCGTCTCGCTCGAATGAAACTTCTCATCCTTACCTG |
| | (SEQ ID NO: 17) |
| CSN1S1 | GCGCCGTCTCGCTCAAAGCTCACCACAGTGGCATAGTAG |
| Rev | (SEQ ID NO: 18) |
| CSN1S2 | GCGCCGTCTCGCTCGAATGAAGTTCTTCATCTTTACCTGC |
| Fw | (SEQ ID NO: 19) |
| CSN1S2 | GCGCCGTCTCGCTCAAAGCTTAAAGGTACCTCACATAGGG |
| Rev | (SEQ ID NO: 20) |
| CSN2 Fw | GCGCCGTCTCGCTCGAATGAAGGTCCTCATCCTTGC |
| | (SEQ ID NO: 21) |
| CSN2 Rev | GCGCCGTCTCGCTCAAAGCTTAGACAATAATAGGGAAGGGTC |
| | (SEQ ID NO: 22) |
| CSN3Fw | GCGCCGTCTCGCTCGAATGATGAAGAGTTTTTTCCTAGTTG |
| | (SEQ ID NO: 23) |
| CSN3 Rev | GCGCCGTCTCGCTCAAAGCTTAGACCGCAGTTGAAGTAAC |
| | (SEQ ID NO: 24) |
| LACB Fw | GCGCCGTCTCGCTCGAATGAAGTGCCTCCTGCTTGC |
| | (SEQ ID NO: 25) |
| LACE Rev | GCGCCTTCTCGGTCAAACTCCTAGATGTGGCACTCTCTCCT |
| | (SEQ ID NO: 26) |
| LALBA Fw | GCGCCGTCTCGCTCGAATGATGTCCTFTGTCTCTCTG |
| | (SEQ ID NO: 27) |
| LALBA Rev | GCGCCGTCTCGCTCAAAGCTCACAACTTCTCACAGAGCC |
| | (SEQ ID NO: 28) |

### CRISPR Design

CRISPR/Cas system for multiple gene targeting was used as previously described in Agustin and collaborators (Zsögön et al. (2017) Plant Sci. 256: 120-130 [“Zsogon 2017”]). CRISPR CSY4 and CRISPR Cas9 were cloned in the same reading frame with a separating linker into GB vector. A multiplex gRNA array of 6 pairs targeting the 8 genes of the 11S and 7S complexes and the 3 fatty desaturases genes, were synthesized by GENESCRIPT® (http://genscript.com) and were inserted to a GB cloning vector. CRISPR Cas9 guide RNAs were designed using CRISPER RGEN TOOLS™ (http://www.rgenome.net/cas-offinder/) with more than 2 mismatches to any other Glycine max genomic sequence.

### LC-MM/MS Proteomic Analysis

All chemicals were purchased from SIGMA-ALDRICH® unless stated otherwise. Samples were homogenized and loaded onto the commercial S-TRAP™ columns (PROTIFI™, USA) for washing the detergents, reduction with 5 mM dithiothreitol, 10 mM iodoacetamide and overnight digestion with trypsin (PROMEGA®) at 50:1 protein: trypsin ratio. Eluted peptides were dried using a vacuum centrifuge and stored in −80° C. Liquid chromatography-mass spectrometry (LC/MS) grade solvents were used for all chromatographic steps. Each sample was loaded using split-less nano-Ultra Performance Liquid Chromatography (10 kpsi NANOACQUITY™; WATERS®, Milford, Mass., USA). The mobile phase was: A) H2O+0.1% formic acid and B) acetonitrile+0.1% formic acid. Desalting of the samples was performed online using a reversed-phase SYMMETRY C18™ trapping column (180 μm internal diameter, 20 mm length, 5 μm particle size; WATERS®, Milford, Mass., USA). The peptides were then separated using a T3 HSS™ nano-column (75 μm internal diameter, 250 mm length, 1.8 μm particle size; WATERS®, Milford, Mass., USA) at 0.35 μL/minutes. Peptides were eluted from the column into the mass spectrometer using the following gradient: 4% to 30% B in 155 minutes, 30% to 90% B in 5 minutes, maintained at 90% for 5 minutes and then back to initial conditions. The nanoUPLC™ was coupled online through a nanoESI™ emitter (10 μm tip; NEW OBJECTIVE™: Woburn, Mass., USA) to a quadrupole orbitrap mass spectrometer (Q EXACTIVE PLUS™, THERMOFISHER SCIENTIFIC™) using a FLEX-ION™ nanospray apparatus (PROXEON™). Data were acquired in data dependent acquisition (DDA) mode, using a Top10 method. MS1 resolution was set to 70,000 (at 200m/z), mass range of 300-1650m/z, AGC of 3e6 and maximum injection time was set to 60 msec. MS2 resolution was set to 17,500, quadrupole isolation 1.7m/z, AGC of 1e5, dynamic exclusion of 60 sec and maximum injection time of 60 msec. Raw data were processed with MaxQuant v1.6.0.16. The data were searched with the Andromeda search engine against the SwissProt N. benthamiana or G. max proteome database appended with the seven cow's milk proteins and common lab protein contaminants and the following modifications: carbamidomethyl on C and oxidation of M. Quantification was based on the label-free quantification (LFQ) method, based on unique peptides.

### Example 1: Construction of Binary Expression Vectors with DNA Associated with Prominent Cow's Milk Proteins

To examine whether plants can express seven of the most prominent cow's milk proteins, seven DNA binary vectors were constructed. TABLE 3 shows the cDNA sequences encoding the cow's milk proteins (TABLE 4).

TABLE 3
DNA sequences encoding he seven cow's milk genes.

| Gene Name | Protein Name | cDNA Sequence |
|---|---|---|
| ALB | Serum albumin | ATGAAGTGGGTGACTTTTATTTCTCTTCTCCTT |
| | | CTCTTCAGCTCTGCTTATTCCAGGGGTGTGTTT |
| | | CGTCGAGATACACACAAGAGTGAGATTGCTCA |
| | | TCGGTTTAAAGATTTGGGAGAAGAACATTTTA |
| | | AAGGCCTGGTACTGATTGCCTITTCTCAGTATC |
| | | TCCAGCAGTGTCCATTTGATGAGCATGTAAAA |
| | | TTAGTGAACGAACTAACTGAGTTTGCAAAAAC |
| | | ATGTGTTGCTGATGAGTCCCATGCCGGCTGTG |
| | | AAAAGTCACTTCACACTCTCTTTGGAGATGAA |
| | | TTGTGTAAAGTTGCATCCCTTCGTGAAACCTAT |
| | | GGTGACATGGCTGACTGCTGTGCGAAACAAGA |
| | | GCCTGAAAGAAATGAATGCTTCCTGAGCCACA |
| | | AAGATGATAGCCCAGACCTCCCTAAATTGAAA |
| | | CCAGACCCCAATACTTTGTGTGATGAGTTTAA |
| | | GGCAGATGAAAAGAAGTTTTGGGGAAAATACC |
| | | TATACGAAATTGCTAGAAGACATCCCTACTTTT |
| | | ATGCACCAGAACTCGTTACTATGCTAATAAAT |
| | | ATAATGGAGTTTTTCAAGAATGCTGCCAAGCT |
| | | GAAGATAAAGGTGCCTGCCTGCTACCAAAGAT |
| | | TGAAACTATGAGAGAAAAAGTACTGACTTCAT |
| | | CTGCCAGACAGAGACTCAGGTGTGCCAGTATT |
| | | CAAAAATTTGGAGAAAGAGCTTTAAAAGCATG |
| | | GTCAGTAGCTCGCCTGAGCCAGAAATTTCCCA |
| | | AGGCTGAGTTTGTAGAAGTTACCAAGCTAGTG |
| | | ACAGATCTTCACAAAAGTCCACAAGGAATGCTG |
| | | CCATGGTGACCTACTTGAATGCGCAGATGACA |
| | | GGGCAGATCTTGCCAAGTACATATGTGATAAT |
| | | CAAGATACAATCTTCCAGTAAACTGAAGGAATG |
| | | CTGTGATAAGCCTTTGTTGGAAAAATCCCACT |
| | | GCATTGCTGAGGTGGAAAAAGATGCCATACCT |
| | | GAAAACCTGCCCCCATTAACTGCTGACTTTGCT |
| | | GAAGATAAGGATGTTTGCAAAAACTATCAGGA |
| | | AGCAAAAGATGCCTTCCTGGGCTCGTTTTTGTA |
| | | TTGAATATTTCAAGAAGGCATCCTGAATTATGCTG |
| | | TCTCAGTGCTATTGAGACTTGCCAAGGAATAT |
| | | GAAGCCACACTGGAGGAATGCTGTGCCAAAGA |
| | | TGATCCACATGCATGCTATTCCACAGTGTTTGA |
| | | CAAACTTAAGCATCTTGTGGATGAGCCTCAGA |
| | | ATTTAATCAAACAAAACTGTGACCAATTCGAA |
| | | AAACTTGGAGAGTATGGATTCCAAAATGAGCT |
| | | CATAGTTCGTTTACACCAGGAAAGTACCCCAAG |
| | | TGTCAACTCCAACTCTCGTGGAGGTTTCAAGA |
| | | AGCCTAGGAAAAGTGGGTACTAGGTGTTGTAC |
| | | AAAGCCGGAATCAGAAAGAATGCCCTGTGCTG |
| | | AAGACTTATCTGAGCTTGATCCTGAACCGGTTG |
| | | TGCGTGCTGCATGAGAAGACACCAGTGAGTGA |
| | | AAAAGTCACCAAGTGCTGCACAGAGTCATTGG |
| | | TGAACAGACGGCCATGTTTCTCTGCTCTGACAC |
| | | CTGATGAAACATATGTACCCAAAGCCTTTGAT |
| | | GAGAAATTGTTCACCTTCCATTGAGATATATG |
| | | CACACTTCCCGATACTGAGAAACAAATCAAGA |
| | | AACAAACTGCACTTGTTGAGCTGTTGAAACAC |
| | | AAGCCCAAGGCAACAGAGGAACAACTTGAAAA |
| | | CCGTCATGGAGAATTTTGTGGCTTTTGTAGGCA |
| | | AGTGCTGTGCAGCTGATGACAAAGAGGCCTGC |
| | | TTTGCTGTGGAGGGTCCAAAACTTGTTGTTTCA |
| | | ACTCAAACAGCCTTAGCCTAA (SEQ ID NO: 29) |
| CSN1S1 | α-S1-Casein | ATGAAACTTCTCATCCTTACCTGTCTTGTGGCT |
| | (alpha-S1-Casien) | GTTGCTCTTGCCAGGCCTAAACATCCTATCAAG |
| | | CACCAAGGACFCCCFCAAGAATTCCTTCAATTGA |
| | | AAATTTACTCAGGTTTTTTGTGGCACCTTTTCC |
| | | AGAAGTGTTTGGAAAGGAGAAGGTCAATGAA |
| | | CTGAGCAAGGATATTGGGAGTGAATCAACTGA |
| | | GGATCAAGCCATGGAAGATATTAAGCAAATGG |
| | | AAGCTGAAAGCATTTTGTCAAGTGAGGAAATT |
| | | GTTCCCAATAGTGTTGAGCAGAAGCACATTCA |
| | | AAAGGAAGATGTGCCCTCTGAGCGTTACCTGG |
| | | GTTATCTGGAACAGCTTCTCAGACTGAAAAAA |
| | | TACAAAGTACCCCAGCTGGAAATTGTTCCCAA |
| | | TAGTGCTGAGGAACGACTTCACAGTATGAAAG |
| | | AGGGAATCCATGCCCAACAGAAAGAACCTATG |
| | | ATAGGAGTGAATCAGGAACTGGCCTACTTCTA |
| | | CCCTGAGCTTTTCAGACAATTCTACCAGCTGGA. |
| | | TGCCTATCCATCTGGTGCCTGGTATTACGTTCC |
| | | ACTAGGCACACAATACACTGATGCCCCATCAT |
| | | TTTCTGACATCCCTAATCCCATTGGCTCTGAGA |
| | | ACAGTGAAAAGACTACTATGCCACTGTGGTGA |
| | | (SEQ ID NO: 30) |
| CSN1S2 | α-S2-Casein | ATGAAACTTCTCATCCTTACCTGTCTTGTGGCT |
| | (alpha-S2-Casein) | GTTGCTCTTGCCAGGCCTAAACATCCTATCAAG |
| | | CACCAAGGACTCCCTCAAGAAGTCCTCAATGA |
| | | AAATTTACTCAGGTTTTTTGTGGCACCTTTTCC |
| | | AGAAGTGTTTTTGGAAAGGAGAAGGTCAATGAA |
| | | CTGAGCAAGGATATTGGGAGTGAATCAACTGA |
| | | GGATCAAGCCATGGAAGATATTAAGCAAATGG |
| | | AAGCTTGAAAGCATTTTCTTCAAGTGAGGAAATT |
| | | GTTCCCAATAGTGTTGAGCAGAAGCACATTCA |
| | | AAAGGAAGATGTGCCCFCTGAGCGTTACCTGG |
| | | GTTATCTGGAACAGCTTCTCAGACTGAAAAAA |
| | | TACAAAGTACCCCAGCTGGAAATTGTTCCCAA |
| | | TAGTGCTGAGGAACGACTTCACAGTATGAAAG |
| | | AGGGAATCCATGCCCAACAGAAAGAACCTATG |
| | | ATAGGAGTGAATCAGGAACTGGCCTACTTCTA |
| | | CCCTGAGCTTFTCAGACAATTCTACCAGCTGGA |
| | | TGCCTATCCATCTGGTGCCTGGTATTACGTTCC |
| | | ACTAGGCACACAATACACTGATGCCCCATCAT |
| | | TCTCTGACATCCCTAATCCCATTGGCTCTGAGA |
| | | ACAGTGAAAAGACTACTATGCCACTGTGGTGA |
| | | (SEQ ID NO: 31) |
| CSN2 | β-Casein | ATGAAGGTCCTCATCCTTGCCTGCCTGGTGGCT |
| | (beta-Casein) | CTGGCCCTTGGAAGAGAGCTGGAAGAACTCAA |
| | | TGTACCTGGTGAGATTGTGGAAAGCCTTTCAA |
| | | GCAGTGAGGAATCTTTATTACACGATCAATAAG |
| | | AAAATTGAGAAGTTTCAGAGTGAGGAACAGCA |
| | | GCAAACAGAGGATGAACTCCAGGATAAAATCC |
| | | ACCCCFTTGCCCAGACACATCTCTAGTCTATC |
| | | CCTTCCCTGGGCCCATCCATAACAGCCTCCCAC |
| | | AAAACATTCCTCCTCTTACTCAAACCCCFGTGG |
| | | TGGTGCCGCCTTTCCTTCAGCCTGAAGTAATGG |
| | | GAGTCTCCAAAGTGAAGGAGGCTATGGCTCCT |
| | | AAGCACAAAGAAATGCCCTTCCCTAAATATCC |
| | | AGTTGAGCCCTTTACTGAAAGGCAGAGCCTGA |
| | | CTCTCACTGATGTTGAAAATCTGCACCTTCCTC |
| | | TGCCTCTGCTCCAGTCTTGGATGCACCAGCCTC |
| | | ACCAGCCTCTICCTCCAACTGTCATGTTTCCTC |
| | | CTCAGTCCGTGCTGTCCCTTTGTCAGTCCAAAG |
| | | TCCTGCCTGTTCCCCAGAAAGCAGTGCCCTATC |
| | | CCCAGAGAGATATGCCCATTCAGGCCTTTCTG |
| | | CTGTACCAGGAGCCTGTACTCGGTCCTGTCCG |
| | | GGGACCCTTCCCTATTATTGTCTAA |
| | | (SEQ ID NO: 32) |
| CSN3 | κ-Casein | ATGATGAAGAGTTTTTTCCTAGTTGTGACTATC |
| | (kappa-Casein) | GTGGCATTAACCCTGCCATTTTTGGGTGCCCAG |
| | | GAGCAAAACCAAGAACAACCAATACGCTGTG |
| | | AGAAAGATGAAAGATTCTTCAGTGACAAAATA |
| | | CTCCAAATATATCCCAATTCAGTATCTTGCTGAG |
| | | TAGGTATCCTAGTTATGGACTCAATTACTACCA |
| | | ACAGAAACCAGTTGCACTAATTAATAATCAAT |
| | | TTCTGCCATACCCATATTATGCAAAGCCAGCTG |
| | | CAGTTAGGTCACCTGCCCAAATTCTTCAATGGC |
| | | AAGTTTTGTCAAATACTGIGCCTTGCCAAGTCCT |
| | | GCCAAGCCCAGCCAACTACCATGCACGTCAC |
| | | CCACACCCACATTTATCATTTATGGCCATTCCA |
| | | CCAAAGAAAAATCAGGATAAAACAGAAATCC |
| | | CTACCATCAATACCATTGCTAGTGGTGAGCCT |
| | | ACAAGTACACCTACCATCGAAGCAGTAGAGAG |
| | | CACTGTAGCTACTCTAGAAGCTTCTCCAGAAG |
| | | TTATTGAGAGCCGACCTGAGATCAACACAGTC |
| | | CAAGTTACTTCAACTGCGGTCTAA |
| | | (SEQ ID NO: 33) |
| LGB, LACB | β-Lactoglobulin | ATGAAGTGCCTCCTGCTTGCCCTGGGCCCTCACT |
| | (beta- | TGTGGCGCCCAGGCCCTCATTGTCACCCAGAC |
| | Lactoglobulin) | CATGAAGGGCCTGGATATCCAGAAGGTGGCGG |
| | | GGACTTGGTACTCCTTGGCCATGGCGGCCAGC |
| | | GACATCTCCCTGCTGGACCCCCACACTGCCCC |
| | | CCTGAGAGTGTATGTGGAGGAGCTGAAGCCCA |
| | | CCCCTGAGGGCGACCTGGAGATCCTGCTGCAG |
| | | AAATGGGAGAACGGTGAGTGTGCTCAGAAGA |
| | | AGATCATTGCAGAAAAAACCAAGATCCCTGCG |
| | | GTGTTCAAGATCGATGCCTTGAATGAGAACAA |
| | | AGTCCTTGTGCTGGACACCGACTACAAAAAGT |
| | | ACCTGCTCTTCTGCATGGAGAACAGTGCTGAG |
| | | CCCGAGCAAAGCCTGGCCTGCCAGTGCCTGGT |
| | | CAGGACCCCGGAGGTGGACGACGAGGCCCTG |
| | | GAGAAATTCGACAAAGCCCTCAAGGCCCTGCC |
| | | CATGCACATCCGGCTGTCCTTCAACCCAACCC |
| | | AGCTGGAGGACCAGTGCCACATCTAG |
| | | SEQ ID NO: 34) |
| LALBA | α-Lactalbumin | ATGATGTCCTTTGTCTCTCTGCTCCTGGTAGGC |
| | (alpha- | ATCCTATTCCATGCCACCCAGGCTGAACAGTT |
| | Lactalbumin) | AACAAAATGTGAGGTGTTCCGGGAGCTGAAAG |
| | | ACTTGAAGGGCTACGGAGGTGTCAGTTTGCCT |
| | | GAATGGEACTGTACCACGTTTCATACCAGTGG |
| | | TTATGACACACACAAGCCATAGTACAAAACAATG |
| | | ACAGCACAGAATATGGACTCTTCCAGATAAAT |
| | | AATAAAATTTGGTGCAAAGACGACCAGAACCC |
| | | TCACTCAAGCAACATCTGTAACATCTCCTGTGA |
| | | CAAGTTCCTGGATGATGATCTTACTGATGACAT |
| | | TATGTGTGTCAAGAAGATTCTGGATAAAGTAG |
| | | GAATTAACTACTGGTTGGCCCATAAAGCACTC |
| | | TGTTCTGAGAAGCTGGATCAGTGGCTCTGTGA |
| | | GAAGTTGTGA (SEQ ID NO: 35) |

TABLE 4
Amino acid sequences of the cow's milk genes.

| Gene Name | Protein Name | Amino Acid Sequence |
|---|---|---|
| ALB | Serum albumin | MKWVTFISLLLLFSSAYSRGVFRRDTHKSEIAHR |
| | | FKDLGEEIIFKGLNLIAFSQYLOQCPFDEHVKLVN |
| | | ELTEFAKTCVADESHAGCEKSLHTLFGDELCKV |
| | | ASLRETYGDMADCCAKQEPERNECFLSHKDDSP |
| | | DLPKLKPDPNTLCDEFKADEXKFWGKYLYEIAR |
| | | RHPYFYAPELLYYANKYNGVFQECCQAEDKGA |
| | | CLLPKIETNIREKVLTSSARQRLRCASIQKFGERA |
| | | LKAWSVARLSOKFPKAEFVEVTKLVTDLTKVHK |
| | | ECCHGDLLECADDRADLAKYICDNQDTISSKLKE |
| | | CCDKPPLLEKSHCIAEVEKDAIPENLPPLTADFAED |
| | | KDVCKNYQEAKDAFLGSFLYEYSRREPEYAVSV |
| | | LLRLAKEYEATLEECCAKDDPHACYSTVFDKLK |
| | | HLVDEPQNLIKQNCDQFEKLGEYGFQNELIVRYT |
| | | RKVPQVSTPTLVEVSRSLGKVGTRCCTKPESERM |
| | | PCAEDYLSLILNRLCVLHEKTPVSEKVTKCCTES |
| | | LVNRRPCFSALTPDETYVPKAFDEKLFHADIC |
| | | TLPDTEKQIKKQTALVELLKHKHKPKATEEQLKTV |
| | | MENFVAFVGKCCAADDKEACFAVEGPKLVVST |
| | | QTALA* (SEQ ID NO: 36) |
| CSN1S1 | α-S1-Casein | MKLLILTCLVAVALARPKHPIKIHQGLPQEVLNEN |
| | (alpha-S1-Casein) | LLRFFVAPFPEVFGKEKVNELSKDIGSESTEDQA |
| | | MEDIKQMEAESISSSEEIVPNSVEQKHIQKEDVPS |
| | | ERYLGYLEQLLRLKKYKVPQLEIVPNSAEERLHS |
| | | MKEGEIHAQQKEPMIGVNQELAYFYPELFRQFYQ |
| | | LDAYPSGAWYYVPLGTQYTDAPSFSDIPNPIGSE |
| | | NSEKITMPLW* (SEQ ID NO: 37) |
| CSN1S2 | α-S2-Casein | MKFFIFTCLLAVALAKNTMEHVSSSEESIISQETY |
| | (alpha-S2-Casein) | KQEKNMDINPSKENLCSTFCKEVVRNANEEEYSI |
| | | GSSSEESAEVATEEVKITVDDKHYQKALNEINQF |
| | | YRKFPQYLQYLYQGPIVLNPWDQVKRNAVPITP |
| | | TLNREQLSTSEENSKKTVDMESTEVFTKKTKLTE |
| | | EEKNRLNFLKKISQRYQKFALPQYLKTVYQHQK |
| | | AMKPWIQPKTKVIPYVRYL* (SEQ ID NO: 38) |
| CSN2 | β-Casein | MKVLILACLVALALARELEELNVPGEIVESLSSSE |
| | (beta-Casein) | ESITRINKKIEKFQSEEQQQTEDELQDKIHPFAQT |
| | | QSLVYPFPGRIHINSLPQNIPPLTQTPVVVPPFLQPE |
| | | VMGVSKVKEAMAPKHKEMPFPKYPVEPFTERQS |
| | | LTLTDVENLEILPLPLLOSWNIHOPH.QPLPHYMI-P |
| | | PQSVLSLSQSKVLPVPQKAVPYPQRDMPIQAFLL |
| | | YQEPVLGPVRGPFPIIV* (SEQ ID NO: 39) |
| CSN3 | κ-Casein | MMKSFFINVIILALTLPFLGAQEQNQEQPIRCEK |
| | (kappa-Casein) | DERFFSDKIAKYIPIQYVLSRYPSYGLNYYQQKP |
| | | VALINNQFLPYPYYAKPAAVRSPAQILQWQVLS |
| | | NTVPAKSCQAQPTTMARHPHPHLSFMAIPPKN |
| | | QDKTEIPTINTIASGEPTSTPTIEAVESTVATLEASP |
| | | EVIESPPEINTVQVTSTAV* (SEQ ID NO: 40) |
| LGB, LACB | β-Lactoglobulin | MKCLLLALALTCGAQALIVTQTMKGLDIQKVAG |
| | (beta- | TWYSLAMAASDISLLDAQSARIRVYYEELKPTPE |
| | Lactoglobulin) | GDLEILLOKWENGECAOKKIIAEKTKIPAVFKIDA |
| | | LNENKVLVLDTDYKKYLLFCMENSAEPEQSLAC |
| | | QCLVRTPEVDDEALEKFDKALKALPMHERLSFNP |
| | | TQLEEQCHI* (SEQ ID NO: 41) |
| LALBA | α-Lactalbumin | MMSFVSLLLVGILFHATOAEOLTKCEVFRELKDL |
| | (alpha- | KGYGCWSLPEWVCTTFHSGYDNAIVQNNDST |
| | Lactalbumin) | EYGLFQINNKIWCKDDQNPHSSNICNISCDKFLD |
| | | DDLTDDIMCVKKILDKVGINYANLAHKMLCSEKL |
| | | DQWLCEKL* (SEQ ID NO: 42) |

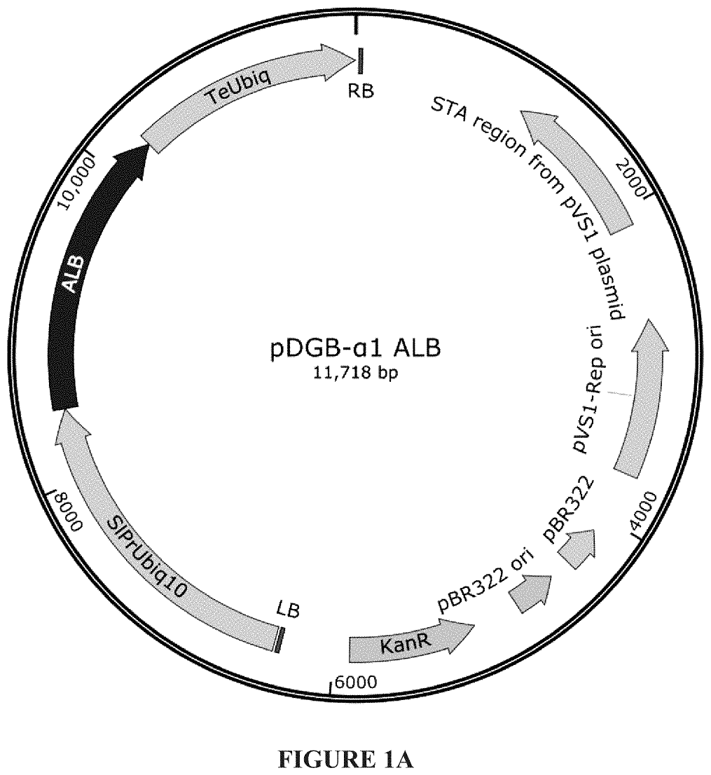

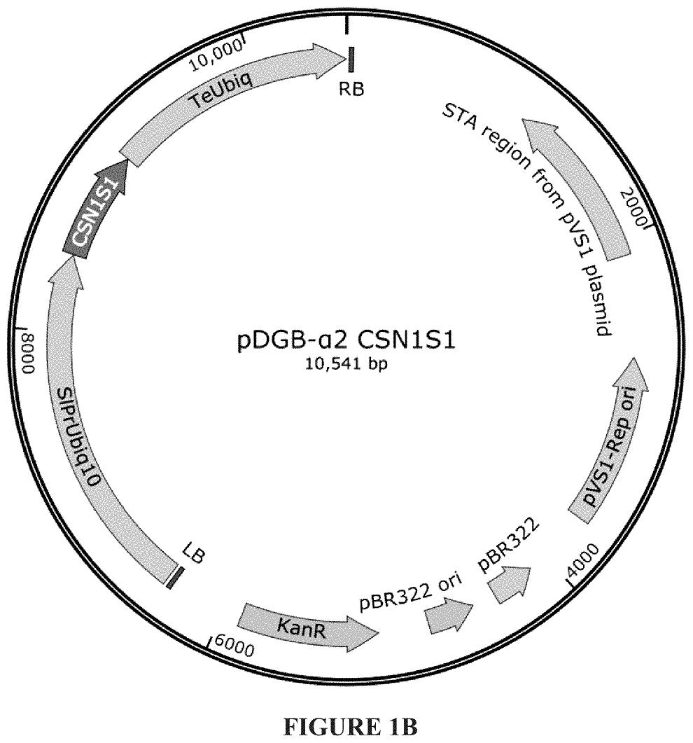

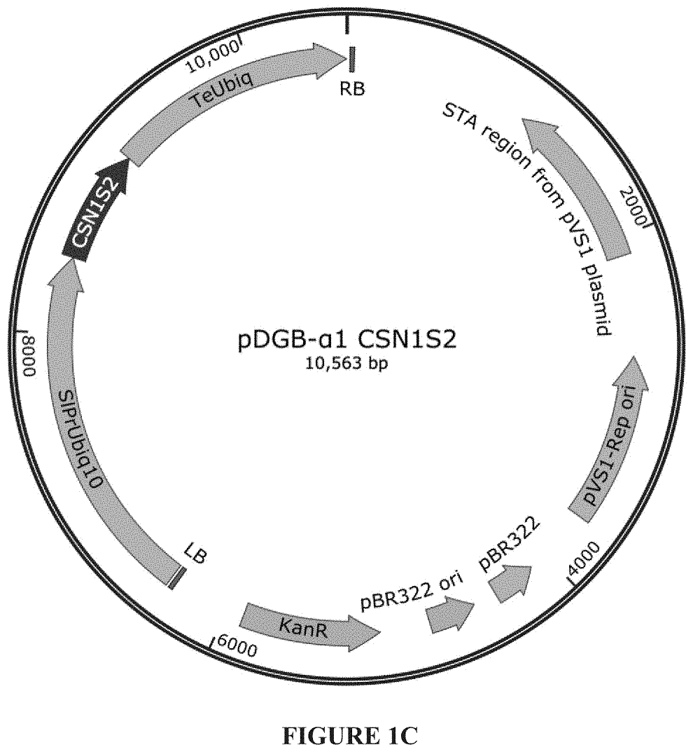

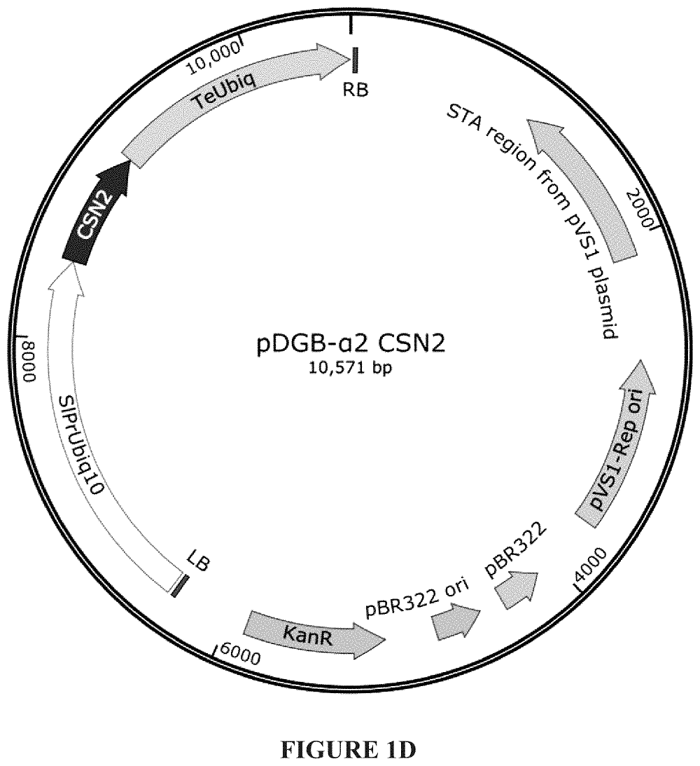

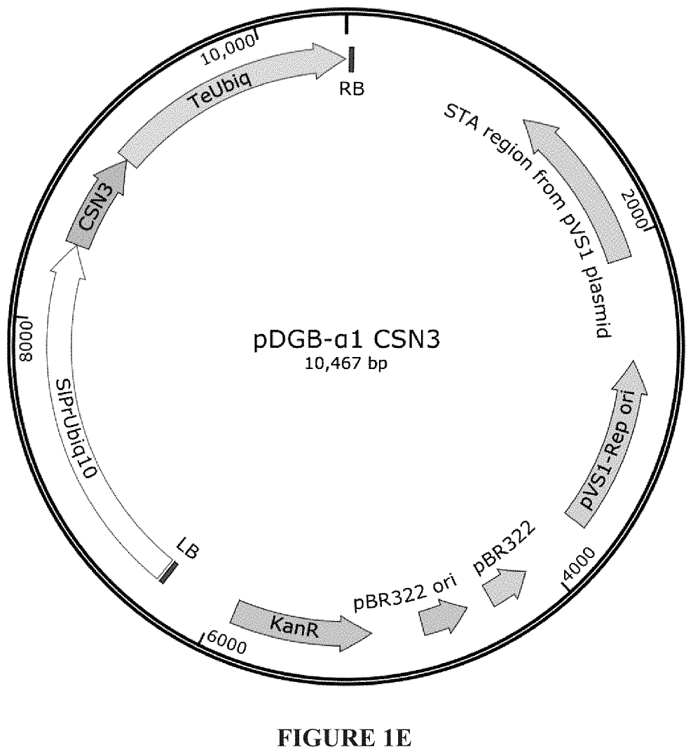

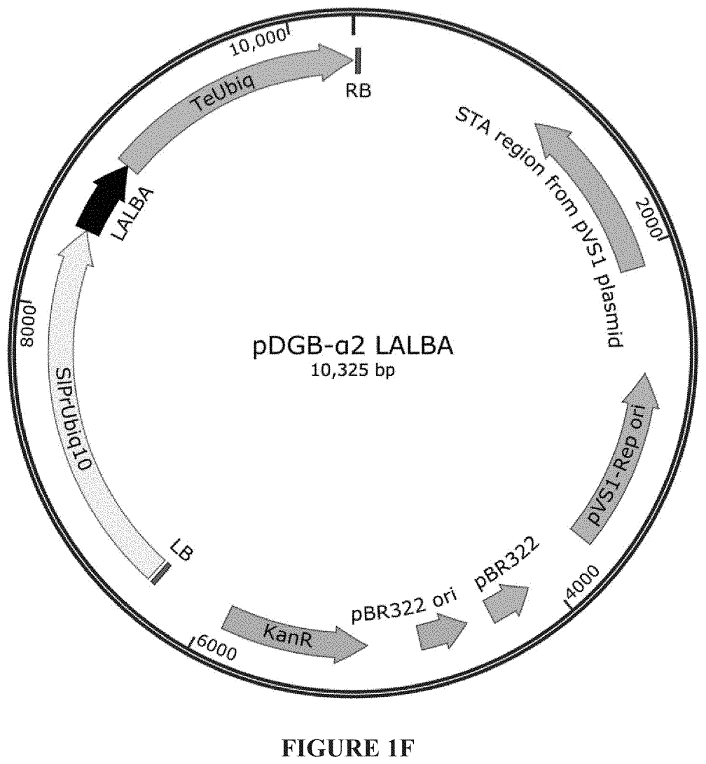

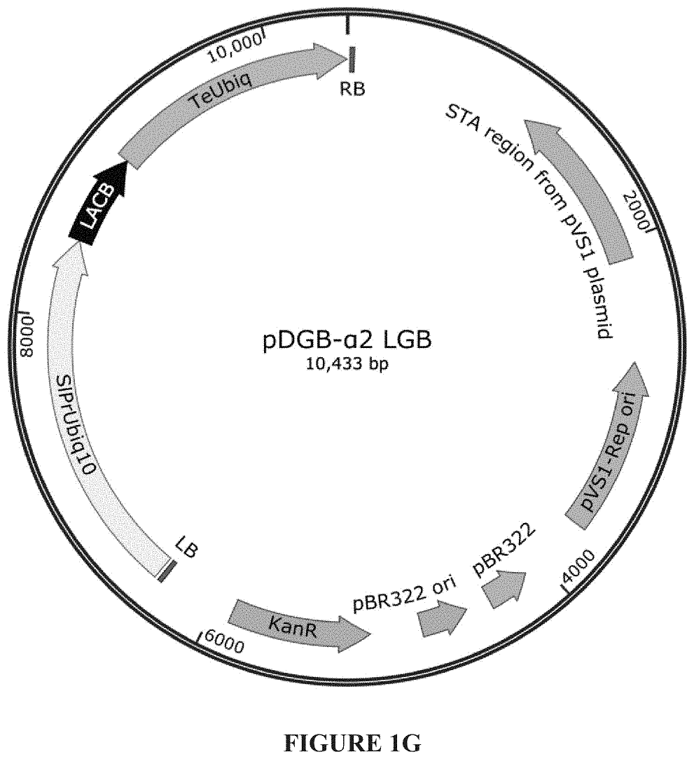

Seven T-DNA binary vectors were constructed, each expressing one of the seven prominent cow's milk proteins. These vectors code for each of the cow's milk seven proteins under the control of constitutive Solanum lycopersicum Ubiquitin promoter 10 (SlPrUbiq10) (FIGS. 1A-1G, TABLE 5).

TABLE 5
Sequences of the seven T-DNA binary vectors for the
expression of cow's milk genes.

>pDGB-α1 ALB (pDGB-alpha1 ALB) (Serum Albumin)
CGCTGTCATGAGACGAATTCTGACAGGATATATTGGCGGGTAAACCTAAGAGAAA
AGAGCGTTTATFAGAATAATCGGATATTTAAAAGGGCGTGAAAAGGTTTATCCGT
TCGTCCATTTGTATGTGCATGCCAACCACAGGGTTCCCCTCGGGATCAAAGTACTT
TGATCCAACCCCTCCGCTGCTATAGTGCAGTCGGCTTCTGACGTTCAGTGCAGCCG
TCATCTGAAAACGACATGTCGCACAAGTCCTAAGTTACGCGACAGGCTGCCGCCC
TGCCCTTTTCCTGGCGTTTTCTTGTCGCGTGTTTTAGTCGCATAAAGTAGAATACTT
GCGACTAGAACCGGAGACATTACGCCATGAAGAAGAGCGCCGCCGCTGGCCTGCT
GGGCTATGCCCGCGTCAGCACCGACGACCAGGACTTGACCAACCAACGGGCCGA
ACTGCACGCGGCCGGCTGCACCAAGCGTTTTCCGAGAAGATCACCGGCACCAGG
CGCGACCGCCCGGAGCTGGCCAGGATGCTTGACCACCTACGCCCTGGCGACGTTG
TGACAGTGACCAGGCTAGACCGCCTGGCCCGCAGCACCCGCGACCTACTGGACAT
TGCCGAGCGCATCCAGGAGGCCGGCGCGGGCCTGCGTAGCCTGGCAGAGCCGTG
GGCCGACACCACCACGCCGGCCGGCCGCATGGTGTTGACCGTGTTCGCCGGCATT
GCCGAGTTCGAGCGTTCCCTAATCATCGACCGCACCCGGAGCGGGCGCGAGGCCG
CCAAGGCCCGAGGCGTGAAGTTTGGCCCCCGCCCTACCCTCACCCCGGCACAGAT
CGCGCACGCCCGCGAGCTGATCGACCAGGAAGGCCGCACCGTGAAAGAGGCGGC
TGCACTGCTIGGCGTGCATCGCTCGACCCTGTACCGCGCACTTGAGCGCAGCGAG
GAAGTGACGCCCACCGAGGCCAGGCGGCGCGGTGCCTTCCGTGAGGACGCATTG
ACCGAGGCCGACGCCCTGGCGGCCGCCGAGAATGAACGCCAAGAGGAACAAGCA
TGAAACCGCACCAGGACGGCCAGGACGAACCGTTTTTCATTACCGAAGAGATCGA
GGCGGAGATGATCGCGGCCGGGTACGTGTTCGAGCCGCCCGCGCACCTCTCAACC
GTGCGGCTGCATGAAATCCTGGCCGGTITGTCTGATGCCAAGGTGGCGGCCTGGC
CGGCCAGCTTGGCCGCTGAAGAAACCGAGCGCCGCCGTCTAAAAAGGTGATGTGT
ATTTGAGTAAAACAGCTTGCGTCATGCGGTCGCTGCGTATATGATCCGATGAGTA
AATAAACAAATACGCAAGGGGAACGCATGAAGGTTATCGCTGTACTTAACCAGA
AAGGCGGGTCAGGCAAGACGACCATCGGNACCGATCTAGCCCGCGCCCTGGAACT
CGCCGGGGCCGATGTTCTGTTAGTCGATTCCGATCCCCAGGGCAGTGCCCGCGAT
TGGGCGGCCGTGCGGGAAGATCAACCGCTAACCGTTGTCGGCATCGACCGCCCGA
CGATTGACCGCGACGTGAAGGCCATCGGCCGGCGCGACTTCGTAGTGATCGACGG
AGCGCCCCAGGCGGCGGACTTGGCTGTGTCCGCGATCAAGGCAGCCGACTTCGTG
CTGATTCCGGTGCAGCCAAGCCCTTACGACATATGGGCCACCGCCGACCTGGTGG
AGCTGGTTAAGCAGCGCATIGAGGTCACGGATGGATGGCTACAAGCGGCCTTTGT
CGTGTCGCGGGCGATCAAAGGCACGCGCATCGGCGGTGAGGTTGCCGAGGCGCT
GGCCGGGTACGAGCTGCCCATTCTTGAGTCCCGTATCACGCAGCGCGTGAGCTAC
CCAGGCACTGCCGCCGCCGGCACAACCGTTCTTGAATCAGAACCCGAGGGCGACG
CTGCCCGCGAGGTCCAGGCGCTGGCCGCTGAAATTAAATCAAAACTCATTTGAGT
TAATGAGGTNAAGAGAAAATGAGCAAAAGCACTAACACGCTAAGTGCCGGCCGT
CCGAGCGCACGCAGCAGCAAGGCTGCAACGTTGGCCAGCCTGGCAGACACGCCA
GCCATGAAGCGGGTCAACTTTCAGITGCCGGCGGAGGATCACACCAAGCTGAAGA
TGTACGCGGTACGCCAAGGCAAGACCATTACCGAGCTGCTATCTGAATAGATCGC
GCAGGTACCAGAGTAAATGAGCAAATGAATAAATGAGTAGATGAATTITAGCGGC
TAAAGGAGGCGGCATGGAAAATCAAGAACAACCAGGCACCGACGCCGTGGAATG
CCCCATGTGTGGAGGAACGGGCGGTTGGCCAGGCGTAAGCGGCTGGGTTGTCTGC
CGGCCCIGCAATGGCACTGGAACCCCCAAGCCCGAGGAATCGGCGTGACGGTCGC
AAACCATCCGGCCCGGTACANATCGGCGCGGCGCTGGGTGATGACCTGGTGGAG
AAGTTGAAGGCCGCGCAGGCCGCCCAGCGGCAACGCATCGAGGCAGAAGCACGC
CCCGGTGAATCGTGGCAAGCGGCCGCTGATCGAATCCGCAAAGAATCCCGGCAAC
CGCCGGCAGCCGGTGCGCCGTCGATTAGGAAGCCGCCCAAGGGCGACGAGCAAC
CAGATTTTTTCGTTCCGATGCTCTATGACGTGGGCACCCGCGATAGTCGCAGCATC
ATGGACGTGGCCGTTTTTCCGTCTGTCGAAGCGTGACCGACGAGCTGGCGAGGTGA
TCCGCTACGAGCTTTCCAGACGGGCACGTAGAGGTTTCCGCAGGGCCGGCCGGCAT
GGCCAGTGTGTGGGATTACGACCTGGTACTGATGGCGGTTTCCCATCTAACCGAA
TCCATGAACCGATACCGGGAAGGGAAGGGAGACAAGCCCGGCCGCGTGTTCCGT
CCACACGTTGCGGACGTACTCAAGTTCTGCCGGCGAGCCGATGGCGGAAAGCAGA
AAGACGACCTGGTAGAAACCTGCATTCGGTTAAACACCACGCACGTTGCCATGCA
GCGTACGAAGAAGGCCAAGAACGGCCGCCTGGTGACGGTATCCGAGGGTGAAGC
CTTGATTAGCCGCTACAAGATCGTAAAGAGCGAAACCGGGCGGCCGGAGTACATC
GAGATCGAGCTAGCTGATTGGATGTACCGCGAGATCACAGAAGGCAAGAACCCG
GACGTGCTGACGGTTCACCCCGATTACTTTTTGATCGATCCCGGCATCGGCCGTTT
TCTCTACCGCCTGGCACGCCGCGCCGCAGGCAAGGCAGAAGCCAGATGGTTGTTC
AAGACGATCTACGAACGCAGTGGCAGCGCCGGAGAGTTCAAGAAGTTCTGTTTCA
CCGTGCGCAAGCTGATCGGGTCAAATGACCTGCCGGAGTACGATTTGAAGGAGGA
GGCGGGGCAGGCTGGCCCGATCCTAGTCATGCGCTACCGCAACCTGATCGAGGGC
GAAGCATCCGCCGGTTCCTAATGTACGGAGCAGATGCTAGGGCAAATTGCCCTAG
CAGGGGAAAAAGGTCGAAAAGGACTCTTTCCTGTGGATAGCACGTACATTGGGAA
CCCAAAGCCGTACATTGGGAACCGGAACCCGTACATTGGGAACCCAAAGCCGTAC
ATTGGGAACCGGTCACACATGTAAGTGACTGATATAAAAGAGAAAAAAGGCGAT
TTTTCCGCCTAAAACTCTTTAAAACTTATTAAAACTCTTAAAACCCGCCTGGCCTG
TGCATAACTGTCTGGCCAGCGCACAGCCGAAGAGCTGCAAAAAGCGCCTACCCTT
CGGTCGCTGCGCTCCCTACGCCCCGCCGCTTCGCGTCGGCCTATCGCGGCCGCTGG
CCGCTCAAAAATGGCTGGCCTACGGCCAGGCAATCTACCAGGGCGCGGACAAGC
CGCGCCGTCGCCACTCGACCGCCGGCGCCCACATCAAGGCACCCTGCCTCGCGCG
TTTCGGTGATGACGGTGAAAACCTCTGACACATGCAGCTCCCGGTGACGGTCACA
GCTTGTCTGTAAGCGGATGCCGGGAGCAGACAAGCCCGTCAGGGCGCGTCAGCG
GGTGTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCACCAT
ATGCGGTGTGAAATACCGCACAGATGCGTAAGGAGAAAATACCGCATCAGGCGC
TCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAA
GGCCGCGTTGGTGGCGTTTTTCCATAGGCTCCGCCCCCCTGACGAGCATCACAAA
AATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACT
ACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACGT
TAAGGGATTTTGGTCATGCATTCTAGGTGATTAGAAAAACTCATCGAGCATCAAA
TGAAACTGCAATTTATTCATATCAGGATTATCAATACCATATTTTTGAAAAAGCCG
TTTCTGTAATGAAGGAGAAAACTCACCGAGGCAGTTCCATAGGATGGCAAGATCC
TGGTATCGGTCTGCGATTCCGACTCGTCCAACATCAATACAACCTATTAATTTCCC
CTCGTCAAAAATAAGGTTATCAAGTGAGAAATCACCATGAGTGACGACTGAATCC
GGTGAGAATGGCAAAAGTTTATGCATTTCTTTCCAGATTGTTCAACAGGCCAGCC
ATTACGCTCGTCATCAAAATCACTCGCATCAACCAAACCGTTATTCATTCGTGATT
GCGCCTGAGCGAGTCGAAATACGCGATCGCTGTTAAAAGGACAATTACAAACAG
GAATCGAATGCAACCGGCGCAGGAACACTGCCAGCGCATCAACAATATTTTCACC
TGAATCAGGATATTCTTCTAATACCTGGAATGCTGTTTTCCCTGGGATCGCAGTGG
TGAGTAACCATGCATCATCAGGAGTACGGATAAAATGCTTGATGGTCGGAAGAGG
CATAAATTCCGTCAGCCAGTTTAGTCTGACCATCTCATCTGTAACATCATTGGCAA
CGCTACCTTTGCCATGTTTCAGAAACAACTCTGGCGCATCGGGCTTCCCATACAAT
CGGTAGATTGTCGCACCTGATTGCCCGACATTATCGCGAGCCCATTTATACCCATA
TAAATCAGCATCCATGTTGGAATTTAATCGCGGCCTTGAGCAAGACGTTTCCCGTT
GAATATGGCTCATAACAGAACTTATTATTTCCTTCCTCTTTTCTACAGTATTTAAAG
ATACCCCAAGAAGCTAATTATAACAAGACGAACTCCAATTCACTGTTCCTTGCATT
CTAAAACCTTAAATACCAGAAAACAGCTTTTTCAAAGTTGTTTTCAAAGTTGGCGT
ATAACATAGTATCGACGGAGCCGATTTTGAAACCGCGGTGATCACAGGCAGCAAC
GCTCTGTGATCGTTACAATCAACATGCTACCCTCCGCGAGATGATCCGTGTTTCAA
ACCCGGCAGCTTAGTTGCCGTTCTTCCGAATAGCATCGGTAACATGAGCAAAGTC
TGCCGCCTTACAACGGCTCTCCCGCTGACGCCGTCCCGGACTGATGGGCTGCCTGT
ATCGAGTGGTGATTTTGTGCCGAGCTGCCGGTCGGGGAGCTGTTGGCTGGCTGGT
GGCAGGATATATTGTGGTGTAAACATAACGAATTCGTCTCAGGAGGTCAACTACC
CCAATTTAAATTTTATTTGATTAAGATATTTTTATTGGACCTACTTTATAATTAAAA
TATTTTCTATTTGAAAAGGAAGGACAAAAATCATACAATTTTGGTCCAACTACTCC
TCTCTTTTTTTTTTTGGCTTTATAAAAAAGGAAAGTGATTAGTAATAAATAATTAA
ATAATGAAAAAAGGAGGAAATAAAATTTTCGAATTAAAATGTAAAAGAGAAAAA
GGAGAGGGAGTAATCATTGTTTAACTTTATCTAAAGTACCCCAATTCGATTTTACA
TGTATATCAAATTATACAAATATTTTATTAAAATATAGATATTGAATAATTTTATT
ATTCTTGAACATGTAAATAAAAATTATCTATTATTTCAATTTTTATATAAACTATTA
TTTGAAATCTCAATTATGATTTTTTAATATCACTITCTATCCATGATAATTTCAGCT
TAAAAAGTTTGTCAATAATTACATTAATTTTGTTGATGAGGATGACAAGATTTCG
GTCATCAATTACATATACACAAATTGAAATAGTAAGCAACTTGATTTTTTTTCTCA
TAATGATAATGACAAAGACACGAAAAGACAATTCAATATTCACATTGATTTATTT
TTATATGATAATAATTACAATAATAATATTCTTATAAAGAAAGAGATCAATTTTGA
CTGATCCAAAAATTTATTTATTTTTACTATACCAACGTCACTAATTATATCTAATA
ATGTAAAACAATTCAATCTTACTTAAATATTAATTTGAAATAAACTATTTTTATAA
CGAAATTACTAAATTTATCCALATAACAANAAGGTCTTAAGAAGACATAAATTCTT
TTTTTGTAATGCTCAAATAAATTTGAGTAAAAAAGAATGAAATTGAGTGATTTTTT
TTTAATCATAAGAAAATAAATAATTAATTTCAATATAATAAAACAGTAATATAAT
TTCATAAATGGNATTCAATACTTACCTCTTAGATATAAAAAATNAATATAAAAAT
AAAGTGTTTCTAATAAACCCGCAATTTAAATAAAATATTTAATATTTTCAATCAAA
TTTAAATAATTATATTAAAATATCGTAGAAAAAGAGCAATATATAATACAAGAAA
GAAGATTTAAGTACAATTATCAACTATTATTATACTCTAATTTTGTTATATTTAATT
TCTTACGGTTAAGGTCATGTTTACGATAAACTCAAAATACGCTGIATGAGGACAT
ATTTTAAATTTTAACCAATAATAAAACTAAGTTATTTTTAGTATATTTTTTTGTTTA
ACGTGACTTAATTTTTCTTTTCTAGAGGAGCGTGTAAGTGTCAACCTCATTCTCCT
AATTTTCCCAACCACATAAAAAAAAAATAAAGGTAGCTTTTGCGTGTTGATTTGGT
ACACTACACGTCATTATTACACGTGTTTTCGTATGATTGGTTAATCCATGAGGCGG
TTTCCTCTAGAGTCGGCCATACCATCTATAAAATAAAGCTITCTGCAGCTCATTTT
TTCATCTTCTATCTGATTTCTATTATAATTTCTCTGAATTGCCTTCAAATTTCTCTTT
CAAGGTTAGAATTTTTCTCTATTTTTTGGTTTTTGTTTGTTTAGATTCTGAGTTTAGT
TAATCAGGTGCTGTTAAAGCCCTAAATTTTGAGTTTTTTTCGGTTGTTTTGATGGA
AAATACCTAACAATTGAGTTTTTTCATGTTGTTTTGTCGGAGAATGCCTACAATTG
GAGTTCCTTTCGTTGTTTTGATGAGAAAGCCCCTAATTTGAGTGTTTTTCCGTCGAT
TTGATTTTAAAGG (SEQ ID NO: 43)
>pDGB-α2 CSN1S1 (pDGB-alpha CSN1S1) (α-S1-Casein; alpha-S1-
Casein)
CGCTTGAGACGAAGCTITGACAGGATATATTGGCGGGTAAACCTAAGAGAAAAG
AGCGTTTATTAGAATAATCGGATATTTAAAAGGGCGTGAAAAGGTTTATCCGTTC
GTCCATTTGTATGTGCATGCCAACCACAGGGTTCCCCTCGGGATCAAAGTACTTTG
ATCCAACCCGICCGCTGCTATAGTGCAGTCGGCTTCTGACCTTTCAGTGCAGCCGTC
ATCTGAAAACGACATGTCGCACAAGTCCTAAGTTACGCGACAGGCTGCCGCCCTG
CCCTTTTCCTGGCGTTTTCTTGTCGCGTGTTTTAGTCGCATAAAGTAGAATACTTGC
GACTAGAACCGGAGACATTACGCCATGAACAAGAGCGCCGCCGCTGGCCTGCTG
GGCTATGCCCGCGTCAGCACCGACGACCAGGACTTGACCAACCAACGGGCCGAA
CTGCACGCGGCCGGCTGCACCAAGCTGTTTTCCGAGAAGATCACCGGCACCAGGC
GCGACCGCCCGGAGCTGGCCAGGATGCTTGACCACCTACGCCCTGGCGACGTTGT
GACAGTGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCCGTGG
GCCGACACCACCACGCCGGCCGGCCGCATGGTGTTGACCGTGTTCGCCGGCATTG
CCGAGTTCGAGCGTTCCCTAATCATCGACCGCACCCGGAGCGGGCGCGAGGCCGC
CAAGGCCCGAGGCGTGAAGTTTGGCCCCCGCCCTACCCTCACCCCGGCACAGATC
GCGCACGCCCGCGACGCTGATCGACCAGGAAGGCCGCACCGTGAAAGAGGCGGCT
GCACTGCTTGGCGTGCATCGCTCGACCCTGTACCGCGCACTTGAGCGCAGCGAGG
AAGTGACGCCCACCGAGGCCAGGCGGCGCGGTGCCTTCCGTGAGGACGCATTGAC
CGAGGCCGACGCCCTGGCGGCCGCCGAGAATGAACGCCAAGAGGAACAAGCATG
AAACCGCACCAGGACGGCCAGGACGAACCGTTTTTCATTACCGAAGAGATCGAG
GCGGAGATGATCGCGGCCGGGTACGTGTTCGAGCCGCCCGCGCACCTCTCAACCG
TGCGGCTGCATGAAATCCTGGCCGGTTTGTCTGATGCCAAGCTGGCGGCCTGGCC
GGCCGCTTGGCCGCTGAAGAAACCGAGCGCCGCCGTCTAAAAAGGTGATGTGTA
TTTGAGTAAAACAGCTTGCGTCATGCGGTCGCTGCGTATATGATCCGATGAGTAA
ATAAACAAATACGCAAGGGGAACGCATGAAGGTTATCGCTGTACTTAACCAGAA
AGGCGGGTCAGGCAAGACGACCATCGGAACCCATCTAGCCCGCGCCCTGCAACTC
GCCGGGGCCGATGTTCTGTTAGTCGATTCCGATCCCCAGGGCAGTGCCCGCGATT
GGGCGGCCGTGCGGGAAGATCAACCGCTAACCGTTGTCGGCATCGACCGCCCGAC
GATTGACCGCGACGTGAAGGCCATCGGCCGGCGCGACTTCGTAGTGATCGACGA
GCGCCCCAGGCGGCGGACTTGGCTGTGTCCGCGATCAAGGCAGCCGACTTCGTGC
TGATTCCGGTGCAGCCAAGCCCTTACGAGATATGGGCCACCGCCGACCTGGTGGA
GCTGGTTAAGCAGCGCATTGAGGTCACGGATGGAAGGCTACAAGCGGCCTFTGTC
GTGTCGCGGGCGATCAAAGGCACGCGCATCGGCGGIGAGGTTGCCGAGGCGCTG
GCCGGGTACGAGCTGCCCATTCTTGAGTCCCGTATCACGCAGCGCGTGAGCTACC
CAGGCACTGCCGCCGCCGGCACAACCGTTCTTGAATCAGAACCCGAGGGCGACGC
TGCCCGCGAGGTCCAGGCGCTGGCCGCTGAAATTAAATCAAAACTCATTTGAGTT
AATGAGGTAAAGAGAAAATGAGCAAAAGCACAAACACGCTAAGTGCCGGCCGTC
CGAGCGCACGCAGCAGCAAGGCTGCAACGTTGGCCAGCCTGGCAGACACGCCAG
CCATGAAGCGGGTCAACTTTCAGTTGCCGGCGGAGGATCACACCAAGCTGAAGAT
GTACGCGGTACGCCAAGGCAAGACCATTACCGAGCTGCTATCTGAATAGATCGCG
CAGCTACCAGAGTAAATGAGCAAATGAATAAATGAGTAGATGAATTTTAGCGGCT
AAAGGAGGCGGCATGGAAAATCAAGAACAACCAGGCACCGACGCCGTGGAATGC
CCCATGTGTGGAGGAACGGGCGGTTGGCCAGGCGTAAGCGGCTGGGTTGTCTGCC
GGCCCTGCAATGGCACTGGAACCCCCAAGCCCGAGGAATCGGCGTGACGGTCGC
AAACCATCCGGCCCGGTACAAATCGGCGCGGCGCTGGGTGATGACCTGGTGGAG
AAGTTGAAGGCCGCGCAGGCCGCCCAGCGGCAACGCATCGAGGCAGAAGCACGC
CCCGGTGAATCGTGGCAAGCGGCCGCTGATCGAATCCGCAAAGAATCCCGGCAAC
CGCCGGCAGCCGGTGCGCCGTCGATTAGGAAGCCGCCCAAGGGCGACGAGCAAC
CAGATTTTTTCGTTCCGATGCTCTATGACGTGGGCACCCGCGATAGTCGCAGCATC
ATGGACGTGGCCGTTTTCCGTCTGTCGAAGCGTGACCGACGAGCTGGCGAGGTGA
TCCGCTACGAGCTTCCAGACGGGCACGTAGAGGTTTCCGCAGGGCCGGCCGGCAT
GGCCAGTGTGTGGGATTACGACCTGGTACTGATGGCGGTTTCCCATCTAACCGAA
TCCATGAACCGATACCGGGAAGGGAAGGGAGACAAGCCCGGCCGCGTGTTCCGT
CCACACGTTGCGGACGTACTCAAGTTCTGCCGGCGAGCCGATGGCGGAAAGCAGA
AAGACGACCTGGTAGAAACCTGCATTCGGTTAAACACCACGCACGTTGCCATGCA
GCGTACGAAGAAGGCCAAGAACGGCCGCCTGGTGACGGTATCCGAGGGTGAAGC
CTTGATTAGCCGCTACAAGATCGTAAAGAGCGAAACCGGGCGGCCGGAGTACATC
GAGATCGAGCTAGCTGATTGGATGTACCGCGAGATCACAGAAGGCAAGAACCCG
GACGTGCTGACGGITCACCCCGATTACTTTTTGATCGATCCCGGCATCGGCCGTTT
TCTCTACCGCCTGGCACGCCGCGCCGCAGGCAAGGCAGAAGCCAGATGGTTGTTC
AAGACGATCTACGAACGCAGTGGCAGCGCCGGAGAGTTCAAGAAGTTCTGTTTCA
CCGTGCGCAAGCTGATCGGGTCAAATGACCTGCCGGAGTACGATTTGAAGGAGGA
GGCGGGGCAGGCTGGCCCGATCCTAGTCATGCGCTACCGCAACCTGATCGAGGGC
GAAGCATCCGCCGGTTCCTAATGTACGGAGCAGATGCTAGGGCAAATTGCCCTAG
CAGGGGAAAAAGGTCGAAAAGGACTCTTTCCTGTGGATAGCACGTAGATTGGGAA
CCCAAAGCCGTACATTGGGAACCGGAACCCGTACATTGGGAACCCAAAGCCGTAC
ATTGGGAACCGGTCACACATGTAAGTGACTGATATAAAAGAGAAAAAAGGCGAT
TTTTCCGCCTAAAACTCTTTAAAACTTATTAAAACTCTTAAAACCCGCCTGGCCTG
TGCATAACTGTCTGGCCAGCGCACAGCCGAAGAGCTGCAAAAAGCGCCTACCCTT
CGGTCGCTGCGCTCCCTACGCCCCGCCGCTTCGCGTCGGCCTATCGCGGCCGCTGG
CCGCTCAAAAATGGCTGGCCTACGGCCAGGCAATCTACCAGGGCGCGGACAAGC
CGCGCCGTCGCCACTCGACCGCCGGCGCCCACATCAAGGCACCCTGCCTTTCGCGCG
TTTCGGTGATGACGGTGAAAACCTCTGACACATGCAGCTCCCGCGTGACGCGTCACA
GCTTGTCTGTAAGCGGATGCCGGGAGCAGACAAGCCCGTCAGGGCGCGTCAGCG
GGTGTTGGCGGGTGTCGGGGCGCAGCCCTGACCCAGFCACGTAGCGATAGCGGAG
TGTATACTGGCTTAACTATGCGGCATCAGAGCAGATTGTACTGAGAGTGCACCAT
ATGCGGTGTGAAATACCGCACAGATGCGTAAGGAGAAAATACCGCATCAGGCGC
TCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAG
GCGTTTCCCCCFGGAAGGTCCCTCGTGCGCTCTCCTTTGTTCCGACCCTGCCGCTTAC
CGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCAC
GAACCCCCCGTTCAGCCCGACCGCTGCGCCTTNTTCCGGTAACTATCGTCTTGAGTC
CAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACT
ACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGC
GGTGGTTTTTTTGTTTGCAAGCAGCAGATTTTACGCGCAGAAAAAAAGGATCTCAAG
AAGATCCTTTGATCTTTTCTACGGGGTCTGACGCTCAGTGGAACGAAAACTCACGT
TAAGGGATITTGGTCATGCMTVTAGGTGATTAGAAAAACTCATCGAGCATCAAA
TGAAACTGCAATTTATTCATATCAGGATTATCAATACCATATTTTTGAAAAAGCCG
TTTCTGTAATGAAGGAGAAAACTCACCGAGGCAGTTCCATAGGATGGCAAGATCC
TGGTATCGGTCTGCGATTCCGACTCGTCAACATCAATACAACCTATTAATTTCCC
CTCGTCAAAAATNAGGTIATCAAGTGAGAAATGACCATGAGTGACGACTGAATCC
GGTGAGAATGGCAAAAGTTTATGCATTTCTTTCCAGACTTGTTCAACAGGCCAGCC
ATTACGCTCGTCATCAAAATCACTCGCATCAACCAAACCGTTATTTTCATTCGTGATT
GCGCCTGAGCGAGTCGAAATACGCGATCGCTGTTAAAAGGACAATTACAAACAG
GAATCGAATGCAACCGGCGCAGGAACACTGCCAGCGCATCAACAATATTTTCACC
TGAATCAGGATATTCTTCTAATACCTGGAATGCTGTTTTCCCTOGGATCGCAGTGG
TGAGTAACCATGCATCATCAGGAGTACGGATAAAATGCTTGATGGTCGGAAGAGG
CATAAATTCCGTCAGCCAGTTTAGTCTGACCATCTCATCTGTAACATCATITGGCAA
CGCTACCTTTGCCATGTTTCAGAAACAACTCTGGCGCATCGGGCTTCCCATACCCCATA
CGGTAGATTGTCGCACCTGATTGCCCGACATTATCGCGAGCCCATTTATACCCATA
TAAATCAGCATCCATGTTGGAATTTAATCGCGGCCTTGAGCAAGACGTTTCCCGTT
GAATATGGCTCATAACAGAACTTATTATTTCCTTCCTCTTTTCTAGAGTATTTAAAG
ATACCCCAAGAAGCTAATTATAACAAGACGAACTCCAATTCACTGTTCCTTGCATT
CTAAAACCTTAAATACCAGAAAACAGCTTTTTCAAAGTTGTTTTCAAAGTTGGCGT
ATAACATAGTATCGACGGAGCCGATTTTGAAACCGCGGTGATCACAGGCAGCAAC
GCTCTGTCATCGTTACAATCAACATGCTACCCTCCGCGAGATCATCCGTGTTTCAA
ACCCGGCAGCTTAGTTGCCGTTCTTCCGAATAGCATCGGTAACATGAGCAAAGTC
TGCCGCCTTACAACGGCTCTCCCGCTGACGCCGTCCCGGACTGATGGGCTGCCTGT
ATCGAGTGGTGATTTTGTGCCGAGCTGCCGGTCGGGGAGCTTTGTTGGCTGGCTGGT
GGCAGGATATATTGTGGTGTNAACATAACAAGCTTCGTCTCAGTCAGGAGGTCAA
CTACCCCAATTTAAATTTTATTTGATTAAGATATTTTTATGGACCTACTTTATAATT
AAAAATATTTTCTATTTGAAAAGGAAGGACAAAAATCATACAATTTTGGTCCAAC
TACTCCTCTCTTTTTTTTTTTGGCTTTATAAAAAAGGAAAGTGATTAGTAATAAAT
AATTAAATAATGAAAAAAGGAGGAAATAAAATTTTCGAATTAAAATGTAAAAGA
GAAANAGGAGAGGGAGTAATCATTGTTTNACTTTATCTAAAGTACCCCAATTCGA
TTTTACATGTATATCAAATTATACAAATATTTTATTAAAATATAGATATTGAATAA
TTTTATTATTCTTGAACATGTAAATAAAAATTATCTATTATTTCAATTTTTATATAA
ACTATTATTTGAAATCTCAATTATGATTTTTTAATATCACTTTCTATCCATGATAAT
TTCAGCTFAAAAAGTTTTGTCAATAATTACATTAATTTTGTTGATGAGGATGACAA
GATTTCGGTCATCAATTACATATACACAAATTGAAATAGTAAGCAACTTGATTTTT
TTTCTCATAATGATAATGACAAAGACACGAAAAGACAATTCAATATTCACATTGA
TTTATTTTTATATGATAATAATTACAATAATAATATTCTTATAAAGAAAGAGATCA
ATTTTGACTGATCCAAANATTTATTTATTTTTACTATACCAACGTCACTAATTATAT
CTAATAATGTAAAACAATTCAATCTTACTTAAATATTAATTTGAAATAAACTATTT
TTATAACGAAATTACTAAATTTATCCAATAACAAAAAGGTCTTAAGAAGACATAA
ATTCTTTTTTTGTAATGCTCAAATAAATTTGAGTAAANAAGNATGAAATTGAGTGA
TTTTTTTTTAATCATAAGAAAATAAATAATTAATTTCAATATAATAAAACAGTAAT
ATAATTTCATAAATGGAATTCAATACTTACCTCTTAGATATAAAAAATAAATTTATAA
ANATAAAGTGTTTCTAATAAACCCGCAATTTAAATAAAATATTTAATATTTTGAAT
CAAATTTAAATAATTATATTAAAATATCGTAGAAAAAGAGCAATATATAATACAA
GAAAGAAGATTTAAGTACAATTATCAACTATTATTATACTCTAATTTTGTTATATT
TAATTTCTTACGGTTAAGGTCATGTTCACGATAAACTCAAAATACGCTGTATGAGG
ACATATTTTAAATTTTAACCAATAATAAAACTAAGTTAITTTTAGTATATTTTTTTG
TTTNACGTGACTTAATTTTTCTTTCTAGAGGAGCGTGTAAGTGTCAACCTCATTCT
CCTAATTTTCCCAACCACATAAAAAAAAAATAAAGGTAGCTTTTGCGTGTTGATTT
GGTACACTACACGTCATTATTACACGTGTTTTCGTATGATTGGTTAATCCATGAGG
CGGTTTCCTCTAGAGTCGGCCATACCATCTATAANATAAAGCTTTCTGCAGCTCAT
TTFFTCATCTTCTATCTGATFFCTATTATAATTTCTCTGAATTGCCTTCAAATTTCTC
TTTCAAGGTTAGAATTTTTCTCTATTTTTTGGTTTTTGTTTGTTTAGATTCTGAGTTT
AGTTAATCAGGTGCTGTTAAAGCCCTAAATTTTGAGTTTTTTTCGGTTGTTTTGATG
GAAAATACCTAACAATTGAGTTTTTTCATGTTGTTTTGTCGGAGAATGCCTACAAT
TGGAGTTCCTTTCGTTGTTTTGATGAGAAAGCCCCTAATTTGAGTGTTTTTCCGTCG
ATTTGATTTTAAA (SEQ ID NO: 44)
>pDGB-α1 CSN1S2 (pDGB-alpha1 CSN1S2) (α-S2-Casein, alpha-S2-
Casein)
CGCTGTCATGAGACGAATTCTGACAGGATATATTGGCGGGTAAACCTAAGAGAAA
AGAGCGTTTATTAGAATAATCGGATATTTAAAAGGGCGTGAAAAGGTTTATCCGT
TCGTCCATTTGTATGTGCATGCCAACCACCAGGGTTCCCCTCGGGATCAAAGTACTT
TGATCCAACCCCTCCGCTGCTATAGTGCAGTCGGCTTCTGACGTTCAGTGCAGCCG
TCATCTGAAAACGACATGTCGCACAAGTCCTAAGTTACGCGAAGGCTGCCGCCC
TGCCCTTTTCCTGGCGTTTTCTTGTCGCGTGTTTTAGTCGCATAAAGTAGAATACTT
GCGACTAGAACCGGAGACATTACGCCATGAACAAGAGCGCCGCCGCTGGCCTGCT
GGGCTATGCCCGCGTCAGCACCGACGACCAGGACTTGACCAACCAACGGGCCGA
ACTGCACGCGGCCGGCTGCACCAAGCTGTTTTCCGAGAAGATCACCGGCACCAGG
CGCGACCGCCCGGAGCTGGCCAGGATGCTTGACCACCTACGCCCTGGCGACGTTG
TGACAGTGACCAGGCTAGACCGCCTGGCCCGCAGCACCCGCGACCTACTGGACAT
TGCCGAGCGCATCCAGGAGGCCGGCGCGGGCCTGCGTAGCCTGGCAGAGCCGTG
GGCCGACACCACCACGCCGGCCGGCCGCATGGTGTTGACCGTGTTCGCCGGCATT
GCCGAGTTCGAGCGTTCCCTAATCATCGACCGCACCCGGAGCGGGCGCGAGGCCG
CCAAGGCCCGAGGCGTGAAGTTTGGCCCCCGCCCTACCCTCACCCCGGCACAGAT
CGCGCACGCCCGCGAGCTGATCGACCAGGAAGGCCGCACCGTGAAAGAGGCGGC
TGCACTGCTFGGCGTGCATCGCTCGACCCTGTACCGCGCACTTGAGCGCAGCGAG
GAAGTGACGCCCACCGAGGCCAGGCGGCGCGGTGCCTTCCGTGAGGACGCATTG
ACCGAGGCCGACGCCCTGGCGGCCGCCGAGAATGAACGCCAAGAGGAACAAGCA
TGAAACCGCACCAGGACGGCCAGGACGAACCGTTTTTCATTACCGAAGAGATCGA
GGCGGAGATGATCGCGGCCGGGTACGTGTTCGAGCCGCCCGCGCACCTCTCAACC
GTGCGGCTGCATGAAATCCTGGCCGGTTTGTCTGATTGCCAAGCTAGCGGCCTGGC
CGGCCAGCTTGGCCGCTGAAGAAACCGAGCGCCGCCGTCTAAAAAGGTGATGTGT
ATTTGAGTAAAACAGCTTGCGTCATGCGGTCGCTGCGTATATGATCCGATGAGTA
AATAAACAAATACGCAAGGGGAACGCATGAAGGTTATCGCTGTACTTTAACCAGA
AAGGCGGGTCAGGCAAGACGACCATCGGAACCCATCTAGCCCGCGCCCTGCAACT
CGCCGGGGCCGATGTTCTGTTAGTCGATTCCGATCCCCAGGGCAGTGCCCGCGAT
TGGGCGGCCGTGCGGGAAGATCAACCGCTAACCGTTGTCGGCATCGACCGCCCGA
CGATTGACCGCGACGTGAAGGCCATCGGCCGGCGCGACTTCGTAGTGATCGACGG
AGCGCCCCAGGCGGCGGACTTGGCTGTGTCCGCGATCAAGGCAGCCGACTTCGTG
CTGATTCCGGTGCAGCCAAGCCCTTACGACATAIGGGCCACCGCCGACCTGGTGG
AGCTGGTTAAGCAGCGCATTGAGGTCACGGATCGAAGGCTACAAGCGGCCTTTGT
CGTGTCGCGGGCGATCAAAGGCACGCGCATCGGCGGTGAGGTTGCCGAGGCGCT
GGCCGGGTACGAGCTGCCGATTCTTGAGTCCCGTATCACGCAGCGCGTGAGCTAC
CCAGGCACTGCCGCCGCCGGCACAACCGTTCTTGAATCAGAACCCGAGGGCGACG
CTGCCCGCGAGGTCCAGGCGCTGGCCGCTGAAATTAAATCAAAACTCATTTGAGT
TAATGAGGTAAAGAGAAAATGAGCAAAAGCACAAACACGCTAAGTGCCGGCCGT
CCGAGCGCACGCAGCAGCAAGGCTGCAACGTTGGCCAGCCTGGCAGACACGCCA
GCCATGAAGCGGGTCAACTTTCAGTTGCCGGCGGAGGATCACACCAAGCTGAAGA
TGTACGCGGTACGCCAAGGCAAGACCATTACCGAGCTGCTATCTGAATAGATCGC
GCAGCTACCAGAGTAAATGAGCAAATGAATAAATGAGTAGATGAATTTTAGCGGC
TAAAGGAGGCGGCATGGAAAATCAAGAACAACCAGGCACCGACGCCGTGGAATG
CCCCATGTGTGGAGGAACGGGCGGTTGGCCAGGCGTAAGCGGCTGGGTTGTCTGC
CGGCCCTGCAATGGCACTGGAACCCCCAAGCCCGAGGAATCGGCGTGACGGTCGC
AAACCATCCGGCCCGGTACAAATCGGCGCGGCGCTGGGTGATGACCTGGTGGAG
AAGTTGAAGGCCGCGCAGGCCGCCCAGCGGCAACGCATCGAGGCAGAAGCACGC
CCCGGTGAATCGIGGCAAGCGGCCGCTGATCGAATCCGCAAAGAATCCCGGCAAC
CGCCGGCAGCCGGTGCGCCGTCGATTAGGAAGCCGCCCAAGGGCGACGAGCAAC
CAGATTTTTTCGTTCCGATGCTCTATTGACGTGGGCACCCGCGATAGTCGCAGCATC
ATGGACGTGGCCGTTTTCCGTCTGTCGAAGCGTGACCGACGAGCTGGCGAGGTGA
TCCGCTACGAGCTTCCAGACGGGCACGTAGAGGTTTCCGCAGGGCCGGCCGGCAT
GGCCAGTGTGTGGGATTACGACCTGGTACTGATGGCGGTTTCCCATCTAACCGAA
TCCATGAACCGATACCGGGAAGGGAAGGGAGACAAGCCCGGCCGCGTGTTCCGT
CCACACGTTGCGGACGTACTCAAGTTCTGCCGGCGAGCCGATGGCGGAAAGCAGA
AAGACGACCTGGTAGAAACCTTGCATTCGGTTAAACACCACGCACGTTGCCATGCA
GCGTACGAAGAAGGCCAAGAACGGCCGCCTGGTGACGGTATCCGAGGGTGAAGC
CTTGATTAGCCGCTACAAGATCGTAAAGAGCGAAACCGGGCGGCCGGAGTACATC
GAGATCGAGCTAGCTGATTGGATGTACCGCGAGATCACAGAAGGCAAGAACCCG
GACGTGCTGACGGTTCACCCCGATTACTTTTTGATCGATCCCGGCATCGGCCGTTT
TCTCTACCGCCTGGCACGCCGCGCCGCAGGCAAGGCAGAAGCCAGATGGTTGTTC
AAGACGATCTACGAACGCAGTGGCAGCGCCGGAGAGTTCAAGAAGTTCTGTTTCA
CCGTGCGCAAGCTGATCGGGTCAAATGACCTGCCGGAGTACGATTTGAAGGAGGA
GGCGGGGCAGGCTGGCCCGATCCTAGTCATGCGCTACCGCAACCTGATCGAGGGC
GAAGCATCCGCCGGTTCCTAATGTACGGAGCAGATGCTAGGGCAAATTGCCCTAG
CAGGGGAAAAAGGTCGAAAAGGACTCTTTCCTGTGGATAGCACGTACATTGGGAA
CCCAAAGCCGTACATTGGGAACCGGAACCCGTACATTGGGAACCCAAAGCCGTAC
ATTGGGAACCGGTCACACATGTAAGTGACTGATATAAAAGAGAAAAAAGGCGAT
TTTTCCGCCTAAAACTCTTTAAAACTTTATTAAAACTCTTAAAACCCGCCTGGCCTG
TGCATAACTGTCTGGCCAGCGCACAGCCGAAGAGCTGCAAAAAGCGCCTACCCTT
CGGTCGCTGCGCTCCCTACGCCCCGCCGCTTCGCTTCGGCCTATCGCGGCCGCTGG
CCGCTCAAAAATGGCTGGCCTACGGCCAGGCAATCTACCAGGGCGCGGACAAGC
CGCGCCGTCGCCACTCGACCGCCGGCGCCCACATCAAGGCACCCTGCCTCGCGCG
TTTCGGTGATGACGGTGAAAACCTCTGACACATGCAGCTCCCGGTGACGGTCACA
GCTTGTCTGTAAGCGGATGCCGGGAGCAGACAAGCCCGTCAGGGCGCGTCAGCG
GGTGTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCACCAT
ATGCGGTGTGAAATACCGCACAGATGCGTAAGGAGAAAATACCGCATCAGGCGC
TCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCCTCACAAA
AATCGACGCTCAAGTCAGAGGTGGCGAAACCCGACAGGACTATAAAGATACCAG
GCGTTTCCCCCTGGAAGCTCCCTCGTGCGCTGTCCATTCCGACCCTGCCGCTTAC
CGGATACCTGTCCGCCTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCAC
GAACCCCCCGTTCAGCCCGACCGCTGCGCTAACGGTAACTATCGTCTTGAGTC
CAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACT
ACGGCTACACTAGAAGGACAGTATTTGGTATCTGCGGTCTGCTGAAGCCAGTTAC
CTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAG
AAGATCCTTTGATTCTTTTCTACGGGGTCTGACGCTCAGTGGAACGAAAACTCACGT
TAAGGGATTTTGGTCATGCATTCTAGGTATTAGAAAAACTCATCGAGCATCAAA
TGAAACTGCAATTTATTCATATCAGGATTATCAATACCATATTTTTGAAAAAGCCG
TTTCTGTAATGAAGGAGAAAACTCACCGAGGCAGTTCCATAGGATGGCAAGATCC
TGGTATCGGTCTGCGATTCCGACTCGTCCAACATCAATACAACCTATTAATTTCCC
CTCGTCAAAAATAAGGTTATCAAGTGAGAAATCACCATGAGTGACGACTGAATCC
GGTGAGAATGGCAAAAGTTTATGCATTTCTTTCCAGACTTGTTCAACAGGCCAGCC
ATTACGCTCGTCATCAAAATCACTCGCATCAACCAAACCGTTATTCATTCGTGATT
GCGCCTGAGCGAGTCGAAATACGCGATCGCTGTTAAAAGGACAATTACAAACAG
GALATCGAATGCAACCGGCGCAGGAACACTGCCAGCGCATCAACAATATTTTCACC
TGAATCAGGATATTCTTCTAATACCTGGAATGCTGTTTTCCCTGGGATCGCAGTGG
TGAGTAACCATGCATCATCAGGAGTACGGATAAAATTGTTGATGGTCGGAAGAGG
CATAAATTCCGTGAGCCAGTTTAGTCTGACGATCTCATCTGTAACATCATTGGCAA
CGCTACCTTTGCCATTTTTCAGAAACAACTCTGGCGCATCGGGCTTCCCATACAAT
CGGTAGATTGTCGCACCTGATTGCCCGACATTATCGCGAGCCCATTTATACCCATA
TAAATCAGCATCCATGTTGGAATTTAATCGCGGCCTTGAGCAAGACGTTTCCCGTT
GAATATGGCTCATAACAGAACTTATTATTTCCTTCCTCTTTTCTACAGTATTTAAAG
ATACCCCAAGAAGCTAATTATAACAAGACGAACTCCAATTCACTGTTCCTTGCATT
CTAAAACCTTAAATACCAGAAAACAGCTTTTTCAAAGTTGTTTTCAAAGTTGGCGT
ATAACATAGTATCGACGGAGCCGATTTTGAAACCGCGGTGATCACAGGCAGCAAC
GCTCTGTCATCGTTACAATCAACATGCTACCCTCCGCGAGATCATCCGTGTTTCAA
ACCCGGCAGCTTAGTTGCCGTTCTTCCGAATAGCATCGGTAACATGAGCAAAGTC
TGCCGCCTTACAACGGCTCTCCCGCTGACGCCGTCCCGGACTGATGGGCTGCCTGT
ATCGAGTGGTGATTTTGTGCCGAGCTGCCGGTCGGGGAGCTGTTGGCTGGCTGGT
GGCAGGATATATTGTGGTGTAAACATAACGAATTCGTCTCAGGAGGTCACTACC
CCAATTTAAATTTTATTTGATTAAGATATTTTTATGGACCTACTITATNATTAAAAA
TATTTTCTATTTGAAAAGGAAGGACAAAAATCATACAATTTTGGTCCAACTACTCC
TCTCTTTTTTIFFTIGGCTTTATAAAAAAGGAAAGTCTATTAGTAATAAATAATTAA
ATAATGAAAAAAGGAGGAAATAAAATTTTCGAATTAAAATGTAAAAGAGAAAAA
GGAGAGGGAGTAATCATTGTTTAACTTTATCTAAAGTACCCCAATTCGATTTTACA
TGTATATCAAATTATACAAATATTTTATTAAAATATAGATATTGAATAATTTTATT
ATTCTTGAACATGTAAATAAAAATTATCTATTATTTCAATTTTTATATAAACTATTA
TTTGAAATCTCAATTATGATTTTTTAATATCACTTTCTATCCATGATAATTTCAGCT
TAAAAAGTTTTGTCAATAATTACATTAATTTTGTTGATGAGGATGACAAGATTTCG
GTCATCAATTACATATACACAAATTGAAATAGTAAGCAACTTGATTTTTTTTCTCA
TAATGATAATGACAAAGACACGAAAAGACAATTCAATATTCACATTGATTTATTT
TTATATGATAATAATTACAATAATAATATTCTTATAAAGAAAGAGATCAATTTTGA
CTGATCCAAAAATTTATTTATTTTTACTATACCAACGTCACTAATTATATCTAATA
ATGTAAAACAATTCAATCTTACTTAAATATTAATTTGAAATAAACTATTTTTATAA
CGAAATTACTAAATTTATCCAATAACAAAAAGGTCTTAAGAAGACATAAATTCTT
TTTTTGTAATGCTCAAATAAATTTGAGTAAAAAAGAATGAAATTGAGTGATTTTTT
TTTAATGATAAGAAAATAAATAATTAATTTCAATATAATAAAACAGTAATATAAT
TTCATAAATGGAATFCAATACTTACCTCTTAGATATAAAAAATAAATATAAAAAT
AAAGTGTTTCTAATAAACCCGCAATTTAAATAAAATATTTAATATTTTCAATCAAA
TTTAAATAATTATATTAAAATATCGTAGAAAAAGAGCAATATATAATACAAGAAA
GAAGATITAAGTACAATTATCAACTATTATTATACTCTAATTTTTTTATATITAATT
TCTTACGTTTTAAGGTCATGTTCACGATAAACTCAAAATACGCYGTGTGAGGACGT
ATTTTAAATTTTAACCAATAATAAAACTAAGTTATTTTTAGTATATTTTTTTGTTTA
ACGTGACTTAATTTTTCTTTTCTAGAGGAGCGTGTAAGTGTCAACCTCATTCTCCT
AATTTTCCCAACCACATAAAAAAAAAATANAGGTAGCTTTTGCGTGTTGATTTGGT
ACACTACACGTCATTATTACACGTGTTTTCGTATGATTGGTTAATCCATGAGGCGG
TTTCCTCTAGAGTCGGCCATACCATCTATAAAATAAAGCTTTCTGCAGCTCATTTT
TTCATCTTCTATCTGATTTCTATTATAATTTCTCTGAATTGCCTTCAAATTTCTCTTT
CAAGGTTAGAATTTTTCTCTATTTTTTGGTTTTTGTTTGTTTAGATTCTGAGTTTAGT
TAATCAGGTGCTGTTAAAGCCCTAAATTTTGAGTTTTTTTCGGTTGCGTTTGATGGA
AAATACCTAACAATTGAGTTTTTTCATGTTGTTTTGTCGGAGAATGCCTACAATTG
GAGTTCCTTTCGTTGTTTTGATGAGAAAGCCCCTAATTTGAGTCTTTTTTCCGTCGAT
TTGATTTTAAAGG (SEQ ID NO: 45)
>pDGB-α2 CSN2 (pDGB-alpha2 CSN2) (β-Casein; beta-Casein)
CGCTTGAGACGAAGCTTTGACAGGATATATTGGCGGGTAAACCTAAGAGAAAAG
AGCGTTTATTAGAATAATCGGATATTTANAAGGGCGTGAAAAGGTTTATCCGTTC
GTCCATTTGTATGTGCATGCCAACCACAGGGTTCCCCTCGGGATCAAAGTACTTTG
ATCCAACCCCTCCGCTGCTATAGTGCAGTCGGCTTCTGACGTTCAGTGCAGCCGTC
ATCTGAAAACGACATGTCGCACAAGTCCTAAGTTACGCGACAGGCTGCCGCCCTG
CCCTTTTCCTGGCGTTTTCTTGTCGCGTGTTTTAGTCGCATAAAGTAGAATACTTGC
GACTAGNACCGGAGACATTACGCCATGAACAAGAGCGCCGCCGCTGGCCTGCTG
GGCTATGCCCGCGTCAGCACCGACGACCAGGACTTGACCAACCAACGGGCCGAA
CTGCACGCGGCCGGCTGCACCAAGCTGTTTTCCGAGAAGATCACCGGCACCATTGC
GCGACCGCCCGGAGCTGGCGAGGATGCTTGACCACCTACGCCCIGGCGACGTTGT
GACAGTGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCCCAGG
GCCGACACCACCACGCCGGCCGGCCGCATGGTGTTGACCGTGTTCGCCGGCATTG
CCGAGTTCGAGCGTTCCCTAATCATCGACCGCACCCGGAGCGGGCGCGAGGCCGC
CAAGGCCCGAGGCGTGAAGTTTGGCCCCCGCCCTACCCTCACCCCGGCACAGATC
GCGCACGCCCGCGAGCTGATCGACCAGGAAGGCCGCACCGTGAAAGAGGCGGCT
GCACTGCTTGGCGTGCATCGCTCGACCCTGTACCGCGCACTTGAGCGCAGCGAGG
AAGTGACGCCCACCGAGGCCAGGCGGCGCGGTGCCTTCCGTGAGGACGCATTGAC
CGAGGCCGACGCCCTGGCGGCCGCCGAGAATGAACGCCAAGAGGAACAAGCATG
AAACCGCACCAGGACGGCCAGGACGAACCGTTTTTCATTACCGAAGAGATCGAG
GCGGAGATGATCGCGGCCGGGTACGTGTTCGAGCCGCCCGCGCACCTCTCAACCG
TGCGGCTGCATGAAATCCTGGCCGGTTTGTCTGATGCCAAGCTGGCGGCCTGGCC
GGCCAGCTTGGCCGCTGAAGAAACCGAGCGCCGCCGATAAAAAGGTGATCTTGTA
TTTGAGTAAAACAGCTTGCGTCATGCGGTCGCTGCGTATATGATCCGATGAGTAA
ATAAACAAATACGCAAGGGGAACGCATGAAGGTTATCGCTGTACTTAACCCAGAA
AGGCGGGTCAGGCAAGACGACCATCGGAACCCATCTAGCCCGCGCCCTGCAACK
GCCGGGGCCGATGTTCTGTTAGTCGATTCCGATCCCCAGGGCAGTGCCCGCGATT
GGGCGGCCGTTGCGGGAAGATCAACCGCTAACCGTTGTCGGCATCGACCGCCCGAC
GATTGACCGCGACGTGAAGGCCATCGGCCGGCGCGACTTCGTAGTGATCGACGGA
GCGCCCCAGGCGGCGGACTTGGCTGTGTCCGCGATCAAGGCAGCCGACTTCGTGC
TGATTCCGGTGCAGCCAAGCCCTTACGACATATGGGCCACCGCCGACCTGGTGGA
GCTGGTTAAGCAGCGCATTGAGGTCACGGATGGAAGGCTACAAGCGGCCTTTGTC
GTGTCGCGGGCGATCAAAGGCACGCGCATVGGCGGTGAGGTTGCCGAGGCGCTG
GCCGGGTACGAGCTGCCCATTCTTGAGTCCCGTATCACGCAGCGCGTGAGCTACC
CAGGCACTGCCGCCGCCGGCACAACCGTTCTTGAATCAGAACCCGAGGGCGACGC
TGCCCGCGAGGTCCAGGCGCTGGCCGCTGAAATTAAATCAAAACTCATTTGAGTT
AATGAGGTAAAGAGAAAATGAGCAAAAGCACAAACACGCTAAGTGCCGGCCGTC
CGAGCGCACGCAGCAGCAAGGCTGCAACGTTGGCCAGCCTGGCAGACACGCCAG
CCATGAAGCGGGTCAACTTTCAGTTGCCGGCGGAGGATCACACCAAGCTGAAGAT
GTACGCGGTACGCCAAGGCAAGACCATTACCGAGCTGCTATCTGAATAGATCGCG
CAGCTACCAGAGTAAATGAGCAAATGAATAAATGAGTAGATGAATTTTAGCGGCT
AAAGGAGGCGGCATGGAAAATCAAGAACAACCAGGCACCGACGCCGTGGAATGC
CCCATGTGTGGAGGAACGGGCGGTTGGCCAGGCGTAAGCGGCTGGGTTGTCTGCC
GGCCCTGCAATGGCACTGGAACCCCCAAGCCCGAGGAATCGGCGTGACGGTCGC
AAACCATCCGGCCCGGTACAAATCGGCGCGGCGCTGGGTGATGACCTGGTGGAG
AAGTTGAAGGCCGCGCAGGCCGCCCAGCGGCAACGCATCGAGGCAGAAGCACGC
CCCGGTGAATCGTGGCAAGCGGCCGCTGATCGAATCCGCAAAGAATCCCGGCAAC
CGCCGGCAGCCGGTGCGCCGTCGATTAGGAAGCCGCCGAAGGGCGACGAGCAAC
CAGATTTTTTCGTTCCGATGCTCTATGACGTGGGCACCCGCGATAGTCGCAGCATC
ATGGACGTGGCCGTTTTCCGTCTGTCGAAGCGTGACCGACGAGCTGGCGAGGTGA
TCCGCTACGAGCTTCCAGACGGGCACGTAGAGGTTTCCGCAGGGCCGGCCGGCAT
GGCCAGTGTGTGGGATTACGACCTGGTACTGATGGCGGTTTCCCATCTAACCGAA
TCCATGAACCGATACCGGGAAGGGAAGGGAGACAAGCCCGGCCGCGTGTTCCGTT
CCACACGTTGCGGACGTACTCAAGTTCTGCCGGCGAGCCGATGGCGGAAAGCAGA
AAGACGACCTGGTAGAAACCTGCATTCGGTTAAACACCACGCACGTTGCCATGCA
GCGTACGAAGAAGGCCAAGAACGGCCGCCTGGTGACGGTATCCGAGGGTGAAGC
CTTGATTAGCCGCTACAAGATCGTAAAGAGCGAAACCGGGCGGCCGGAGTACATC
GAGATCGAGCTAGCTGATTGGATGTACCGCGAGATCACAGAAGGCAAGAACCCG
GACGTGCTGACGGTTCACCCCGATTACTTTTTGATCGATCCCGGCATCGGCCGITT
TCTCTACCGCCTGGCACGCCGCGCCGCAGGCAACCGCAGAAGCCAGATGGTTGTTC
AAGACGATCTACGAACGCAGTGGCAGCGCCGGAGAGTTCAAGAAGTTCTGTTTCA
CCGTGCGCAAGCTGATCGGGTCAAATGACCTGCCGGAGTACGATTTGAAGGAGGA
GGCGGGGCAGGCTGGCCCGATCCTAGTCATGCGCTACCCAACCTGATCGAGGGC
GAAGCATCCGCCGGTTCCTAATGTACGGAGCAGATGCTAGGGCAAATTGCCCTAG
CAGGGGAAAAAGGTCGAAAAGGACTCTTTCCTGTGGATAGCACGTACATTGGGAA
CCCAAAGCCGTACATTGGGAACCGGAACCCGTCATTGGGAACCCAAACCCGTAC
ATTGGGAACCGGTCACACATGTAAGTGACTGATATAAAAGAGAAAAAAGGCGAT
TTTTCCGCCTAAAACTCTTTAAAACTTATTAAAACTCTTAAAACCCGCCTGGCCTG
TGCATAACTGTCTGGCCAGCCCACAGCCGAAGAGCTGCAAAAAGCGCCTACCCTT
CGGTCGCTGCGCTCCCTACGCCCCGCCGCTTCGCGTCGGCCTATCGCGGCCGCTGG
CCGCTCAAAAATGGCTGGCCTACGGCCAGGCAATCTACCAGGGCGCGGACAAGC
CGCGCCGTCGCCACTCGACCGCCGGCGCCCACATCAAGGCACCCTGCCTCGCGCG
TTTCGGTGATGACGGTGAAAACCTCTGACACATGCAGCTCCCGGTGACGGTCACA
GCTFGTCTGTAAGCGGATGCCGTGAGCAGACAAGCCCGTCAGGGCCCGTCAGCG
GGTGTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCTTCCAT
ATGCGGTGTGAAATACCGCACAGATGCGTAAGGAGAAAATACCGCATCAGGCGC
TCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCAC
GCTGTAGGTATCTCAGTTCGGTGTAGTTCGTTCGCTCCAAGCTGGGCFGTGTTGCAC
GAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACT
ACGGCTACACTAGAAGGACAGTATTTGGTATCTGCGCTCTGCTGAAGCCAGTTAC
CFTCGGAAAAAGAGTTGGTAGCTCTITATCCGGCAAACAAACCACCGCTGGTAGC
GGIGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACGT
TAAGGGATTTTGGTCATGCATTCTAGGTGATTAGAAAAACTCATCGAGCGTCAAA
TGAAACTGCAATTTATTCATATCAGGATTATCAATACCATATTTTTGAAAAAGCCG
TTTCTGTAATGAAGGAGAAAACTCACCGAGGCAGTTCCATAGGATGGCAAGATCC
TGGTATCGGTCTGCGATTCCGACTCGTCCAACATCAATACAACCTATTAATTTCCC
CTCGTCAAAAATAAGGTTATCAAGTGAGAAATCACCATGAGTGACGACTGAATCC
GGTGAGAATGGCAAAAGTTTATGCATTTCTTTCCAGACTTGTTCAACAGGCCAGCC
ATTACGCTCGTCATCAAAATGACTCGCATCAACCAAACCGTTATTCATTCGTGATT
GCGCCTGAGCGAGTCGAAATACGCGATCGCTGTTAAAAGGACAATTACAACAG
GAATCGAATGCAACCGGCGCAGGAACACTGCCAGCGCATCAACAATATTTTCACC
TGAATCAGGATATTCTTCTAATACCTGGAATGCTGTTTTCCCTGGGATCGCAGTGG
TGAGTAACCATGCATCATCAGGAGTACGGATAAAATGCTTGATGGTCGGAAGAGG
CATAAATTTCCCTTCAGCCAGTTTAGTCTGACCATCTCATTCTGTAACATCATTGGCAA
CGCTACCTITGCCAIGTTTCAGAAACAACTCTGGCGCATCGGGCTTCCCATACAAT
CGGTAGATTGTCGCACCTGATTGCCCGACATTATVGCGAGCCCATTTATACCCATA
TAAATCAGCATCCATGTTGGAATTTAATCGCGGCCTTGAGCAAGACGTTTCCCGTT
GAATATGGCTCATAACAGAACTTATTATTTCCTTCCTCTTTTCTACAGTATTTAAAG
ATACCCCAAGAAGCTAATTATAACAAGACGAACTCCAATTCACTGTTCCTTGCATT
CTAAAACCTTAAATACCAGAAAACAGCTTTTTCAAAGTTGTTTTCAAAGTTGGCGT
ATAACATAGTATCGACGGAGCCGATTTTGAAACCGCGGTGATCACAGGCAGCAAC
GCTCTGTCATCGTTACAATCAAGATGCTACCCTCCGCGAGATCATCCGTGTTTCAA
ACCCGGCAGCTTAGTTGCCGTTCTTCCGAATAGCATCGGTAACATGAGCAAAGTC
TGCCGCCTTACAACGGCTCTCCCGCTGACGCCGTCCCGGACTGATGGGCTGCCTGT
ATCGAGTGGTGATTTTGTGCCGAGCTGCCGGTCGGGGAGCTGTTGGCTGGCTGGT
GGCAGGATATATTGTGGTGTAAACATAACAAGCTTCGTCTCAGTCAGGAGGTCAA
CTACCCCAATTTAAATTTTATTTGATTAAGATATTTTATGGACCTACTTTATAATT
AANAATATTTTCTATTTGAAAAGGAAGGACAAAAATCATACAATTTTGGTCCAAC
TACTCCTCTCTTTTTTTTTTTGGCTTTATAAAAAAGGAAAGTGATFAGTAATAAAT
AATTAAGTAATGAAAAAAGGAGGAAATAAAATTTTCGAATTAAAATGTAAAAGA
GAAAAAGGAGAGGGAGTAATCATTGTTTAACTTTATCTAAAGTACCCCAATTCGA
TTTTACATGTATATCAAATTATMAAATATTTTATTAAAATATAGATATTGAATAA
TTTTATTATTCTTGAACATGTAAATAAAAATTATCTATTATTTCAATTTTTATATAA
ACTATTATTTGAAATCTCAATTATGATTTTTTAATATCACTTTCTATCCATGATAAT
TTCAGCTTAAAAAGTTTTGTCAATAATTACATTAATTTTGTTGATGAGGATGACAA
GATTTCGGTCATCAATTACATATACACAAATTGAAATAGTAAGGAACTTGATTTTT
TTTCTCATAATGATAATGACAAAGACACGAAAAGACAATTCAATATTCACATTGA
TTTATTTTTATATGATAATAATTACAATAATAATATTCTTATAAAGAAAGAGATCA
ATTTTGACTGATCCAAAAATTTATTTATTTTTACTATACCAACGTCACTAATTATAT
CTAATAATGTAAAACAATTCAATCTTACTTAAATATTAATTTGAAATAAACTATFF
TGATAACGAAATTACTAAATTTATCCAATAACAAAAAGGTCTTAAGAAGACATAA
ATTCTTTTTTTGTAATGCTCAAATAAATTTGAGTAAAAAAGAATGAAATTGAGTGA
TTTITTTTTAATCATAAGAAAATAAATAATTAATTTCAATATAATAAAACAGTAAT
ATAATTTCATAAATGGAATTCAATACTTACCTCTTAGATATAAAAAATANATATAA
AAATAAAGTGTTTCTAATAAACCCGCAATTTAAATAAAATATTTAATATTTTCAAT
CAAATTTAAATAATTATATTAAAATATCGTAGAAAAAGAGCAATATMAATACAA
GAAAGAAGATTTAAGTACAATTATCTAACTATTATTATACTCTAATTTTGTTATATT
TAATTTCTTACGGTTAAGGTCATGTTCACGATAAACTCAAAATACGCTGTATGAGG
ACATATITTAAATTTTAACCAATAATAAAACTAAGTTATTTTTAGTATATTTTTTG
TTTAACGTGACTTAATTTTTCTTTTCTAGAGGAGCGTGTAAGTGTCAACCTCATTCT
CCTAATTTTCCCAACCACATAAAAAAAAAATAAAGGTAGCTTTTGCGTGTTGATTT
GGTACACTACACGTCATTATTACACGTGTTTTCGTATGATTGGTTAATCCATGAGG
CGGTTTCCTCTAGAGTCGGCCATACCATCTATAAAATAAAGCTTTCTGCAGCTCAT
TTTTTCATCTTCTATCTGATTTCTATTATAATTTCTCTGAATTGCCTTCAAATTTCTC
TTTCAAGGTTAGAATTTTTCTCTATTTTTTGGTTTTTGTTTGTTTAGATTCTGAGTTT
AGTTAATCAGGTGCTGTTAAAGCCCTAAATTTTGAGTTTITTTCGGTTGTTTTGATG
GAAAATACCTAACAATTGAGTTTTTTCATGTTGTTTTGTCGGAGAATGCCTACAAT
TGGAGTTCCTTTCGTTGTTTTGATGAGAAAGCCCCTAATTTGAGTGTTTTTCCGTCG
ATTTGATITTAAA (SEQ ID NO: 46)
>pDGB-α1 CSN3 (pDGB-alpha1 CSN3) (κ-Casein; kappa-Casein)
CGCTGTCATGAGACGAATTCTGACAGGATATATTGGCGGGTAAACCTAAGAGAAA
AGAGCGTTTATTAGAATAATCGGATATTTAAAAGGGCUTTAAAAGGTTTATCCTE
TCGTCCATTTGTATGTGCATGCGAACCAGAGGGTTCCCCTCGGGATCAAAGTACTT
TGATCCAACCCCTCCGCTGCTATAGTGCAGTCGGCTTGTCTGACGTTCAGTGCAGCCG
TCATCTGAAAACGACATGTCGCACAAGTCCTAAGTTACGCGACAGGCTGCCGCCC
TGCCCTTTTCCTGGCGTTTTCTTGTCGCGTGTTTTAGTCGCATAAAGTAGAATACTT
GCGACTAGAACCGGAGACATTACGCCATGAACAAGAGCGCCGCCGCTGGCCTGCT
GGGCTATGCCCGCGTCAGCACCGACGACCAGGACTTGACCAACCAACGGGCCGA
ACTGCACGCGGCCGGCTGCACCAAGCTGTTTTCCGAGAAGATCACCGGCACCAGG
CGCGACCGCCCGGAGCTGGCCAGGATGCTTGACCACCTACGCCCTGGCGACGTTG
TGACAGTGACCAGGCTAGACCGCCTGGCCCGCAGCACCCGCGACCTACTGGACAT
TGCCGAGCGCATCCAGGAGGCCGGCGCGGGCCTGCGTAGCCTGGCAGAGCCGTG
GGCCGACACCACCACGCCGGCCGGCCGCATGGTGTTGACCGTGTTCGCCGGCATT
GCCGAGTTCGAGCGTTCCCTAATCATCGACCGCACCCGGAGCGGGCGCGAGGCCG
CCAAGGCCCGAGGCGTGAAGTTTGGCCCCCGCCCTACCCTCACCCCGGCACAGAT
CGCGCACGCCCGCGAGCTGATCGACCAGGAAGGCCGCACCGTGAAAGAGGCGGC
TGCACTGCTTGGCGTGCATCGCTCGACCCTGTACCGCGCACTTGAGCGCAGCGAG
GAAGTGACGCCCACCGAGGCCAGGCGGCGCGGTGCCTTCCGTGAGGACGCATTG
ACCGAGGCCGACGCCTGGCGGCCGCCGAGAATGAACGCCAAGAGGAACAAGCA
TGAAACCGCACCAGGACGGCCAGGACGAACCGTTTTTCATTACCGAAGAGATCGA
GGCGGAGATGATCGCGGCCGGGTACGTGTTCGAGCCGCCCGCGCACCTCTCAACC
GTGCGGCTGCATGAAATCCTGGCCGGTTTGTCTGATGCCAAGCTGGCGGCCTGGC
CGGCCAGCTTGGCCGCTGAAGAAACCGAGCGCCGCCGTCTAAAAAGGTGATGTGT
ATTTGAGTAAAACAGCTTGCGTCATGCGGTCGCTGCGTATATGATCCGATGAGTA
AATAAACAAATACGCAAGGGGAACGCATGAAGGTTATCGCTGTACTTAACCAGA
AAGGCGGGTCAGGCAAGACGACCATCGGAACCCATCTAGCCCGCGCCCTGCAACT
CGCCGGGGCCGATGTTCTGTTAGTCGATTCCGATCCCCAGGGCAGTGCCCGCGAT
TGGGCGGCCGTGCGGGAAGATCAACCGCTAACCGTTGTCGGCATCGACCGCCCGA
CGATTGACCGCGACGTGAAGGCCATCGGCCGGCGCGACTTCGTAGTGATCGACGG
AGCGCCCCAGGCGGCGGACTTGGCTGTGTCCGCGATCAAGGCAGCCGACTTCGTG
CTGATTCCGGTGCAGCCAAGCCCTTACGACATATGGGCCACCGCCGACCTGGTGG
AGCTGGTTAAGCAGCGCATTGAGTTTACGGATGGAAGGCTACAAGCGGCCTTTGT
CGTGTCGCGGGCGATCAAAGGCACGCGCATCGGCGGTGAGGTTGCCGAGGCGCT
GGCCGGGTACGAGCTGCCCATTCTTGAGTCCCGTATCACGCAGCGCGTGAGCTAC
CCAGGCACTGCCGCCGCCGGCACAACCGTTCTTGAATCAGAACCCGAGGGCGACG
CTGCCCGCGAGGTCCAGGCGCTGGCCGCTGAAATTAAATCAAAACTCATITGAGT
TAATGAGGTAAAGAGAAAATGAGCAAAAGCACAAACACGCTAAGTGCCGGCCGT
CCGAGCGCACGCAGCAGCAAGGCTGCAACGTTGGCCAGCCTGGCAGACACGCCA
GCCATGAAGCGGGTCAACTTTCAGTTGCCGGCGGAGGATCACACCAAGCTGAAGA
TGTACGCGGTACGCCAAGGCAAGACCATTACCGAGCTGCTATCTGAATAGATCGC
GCAGCTACCAGAGTAAATGAGCAAATGAATAAATGAGTAGATGAATTTTAGCGGC
TAAAGGAGGCGGCATGGAAAATCAAGAACAACCAGGCACCGACGCCGTGGAATG
CCCCATGTGTGGAGGAACGGGCGGTTGGCCAGGCGTAAGCGGCTGGGTTGTCTGC
CGGCCCTGCAATGGCACTGGAACCCCCAAGCCCGAGGAATCGGCGTGACGGTCGC
AAACCATCCGGCCCGGTACAAATCGGCGCGGCGCTGGGTGATGACGTGGTGGAG
AAGTTGAAGGCCGCGCAGGCCGCCCAGCGGCAACGCATCGAGGCAGAAGCACGC
CCCGGTGAATCGTGGCAAGCGGCCGCTGATCGAATCCGCAAAGAATCCCGGCAAC
CGCCGGCAGCCGGTGCGCCGTCGATTAGGAAGCCGCCCAAGGGCGACGAGCAAC
CAGATTTTTTCGTTCCGATGCTCTATGACGTGGGCACCCGCGATAGTCGCAGCATC
ATGGACGTGGCCGTTTTCCGTCTGTCGAAGCGTGACCGACGAGCTGGCGAGGTGA
TCCGCTACGAGCTTCCAGACGGGCACGTAGAGGTTTCCGCAGGGCCGGCCGGCAT
GGCCAGTGTGTGGGATTACGACCTGGTACTGATGGCGGTTTCCCATCTAACCGAA
TCCATGAACCGATACCGGGAAGGGAAGGGAGACAAGCCCGGCCGCGTGTTCCGT
CCACACGTTGCGGACGTACTCAAGTTCTGCCGGCGAGCCGATGGCGGAAAGCAGA
AAGACGACCTGGTAGAAACCTGCATTCGGTTAAACACCACGCACGTTGCCATGCA
GCGTACGAAGAAGGCCAAGAACGGCCGCCTGGTGACGGTATCCGAGGGTGAAGC
CTTGATTAGCCGCTACAAGATCGTAAAGAGCGAAACCGGGCGGCCGGAGTACATC
GAGATCGAGCTAGCTGATTGGATGTACCGCGAGATCACAGAAGGCAAGAACCCG
GACGTGCTGACGGTTCACCCCGATTACTTTTTGATCGATCCCGGCATCGGCCGTTT
TCTCTACCGCCTGGCACGCCGCGCCGCAGGCAAGGCAGNAGCCAGATGGTTGITC
AAGACGATCTACGAACGCAGTGGCAGCGCCGGAGAGTTCAAGAAGTTCTGTTTCA
CCGTGCGCAAGCTGATCGGGTCAAATGACCTGCCGGAGTACGATTTGAAGGAGGA
GGCGGGGCAGGCTGGCCCGATCCTAGTCATGCGCTACCGCAACCTGATCGAGGGC
GAAGCATCCGCCGGTTCCTAATGTACGGAGCAGATGCTACCGCAAATTGCCCTAG
CAGGGGAAAAAGGTCGAAAAGGACTCTTTCCTGTGGATAGCACGTACATTGGGAA
CCCAAAGCCGTACATTGGGAACCGGAACCCGTACATTGGGAACCCAAAGCCGTAC
ATTGGGAACCGGTCACACATGTAAGTGACTGATATAAAAGAGAAAAAAGGCGAT
TTTTCCGCCTAAAACTCTTIAAAACITATTAAAACTCTTANAACCCGCCTGGCCTG
TGCATAACTGTCTGGCCAGCGCACAGCCGAAGAGCTGCAAAAAGCGCCTACCCTT
CGGTCGCTGCGCTCCCTACGCCCCGCCGCTTCGCGTCGGCCTATCGCGGCCGCTGG
CCGCTCAAAAATGGCTGGCCTACGGCCAGGCAATCTACCAGGGCGCGGACAAGC
CGCGCCGTCGCCACTCGACCGCCGGCGCCCACATCAAGGCACCCTGCCTCGCGCG
TTTCGGTGATGACGGTGAAAACCTCTGACACATGCAGCTCCCGGTGACGGTCACA
GCTTGTCTGTAAGCGGATGCCGGGAGCAGACAAGCCCGTCAGGGCGCGTCAGCG
GGTMTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCACCAT
ATGCGGTGTGAAATACCGCACAGATGCGTAAGGAGAAAATACCGCATCAGGCGC
TCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACT
ACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTGACGT
TAAGGGATTTTGGTCATGCATTCTAGGTGATTAGAAAAACTCATCGAGCATCAAA
TGAAACTGCAATTTATTCATATCAGGATTATCAATACCATATTTTTGAAAAAGCCG
TTTCTGTAATGAAGGAGAAAACTCACCGAGGCAGTTCCATAGGATGGCAAGATCC
TGGTATCGGTCTGCGATTCCGACTCGTCCAACATCAATACATACCTATTAATTTCCC
CTCGTCAAAAATAAGGTTATCAAGTGAGAAATCACCATGAGTGACGACTGAATCC
GGTGAGAATGGCAAAAGTTTATGCATTTCTTTCCAGACTTGTTCAACAGGCCAGCC
ATTACGCTCGTCATCAAAATCACTCGCATCAACCTAAACCGTATTCATTCGTGATT
GCGCCTGAGCGAGTCGAAATACGCGATCGCTGTTAAAAGGACAATTACAAACAG
GAATCGAATGCAACCGGCGCAGGAACACTGCCAGCGCATCAACAATATTTTCACC
TGAATCAGGATATTCTTCTAATACCTGGAATGCTGTTTTCCCTGGGATCGCAGTGG
TGAGTAACCATGCATGATCAGGAGTACGGATAAAATGCTTGATGGTCGGAAGAGG
CATAAATTCCGTCAGCCAGTTTAGTCTGACCATCTCATCTGTAACATCATTGGCAA
CGCTACCTTTGCCATGTTTCAGAAACAACTCTGGCGCATCGGGCTTCCCATACAAT
CGGTAGATTGTCGCACCTGATTGCCCGACATTATCGCGAGCCCATTTATACCCATA
TAAATCAGCATCCATGTTGGAATTTAATCGCGGCCTTGAGCAAGACGTTTCCCGTT
GAATATGGCTCATAACAGAACTTATTATTTCCTTCCTCTTTTCTACAGTATTTAAAG
ATACCCCAAGAAGCTAATTATAACAAGACGAACTCCAATTCACTGTTCCTTGCATT
CTAAAACCTTAAATACCAGAAAACAGCTTTTTCAAAGTTGTTTTCAAAGTTGGCGT
ATAAGATAGTATCGACGGAGCCGATTTTGAAACCGCGGTGATCACAGGCAGGAAC
GCTCTGTCATCGTTACAATCAACATGCTACCCTCCGCGAGATCATCCGTGTTTCAA
ACCCGGCAGCTTAGTTGCCGTTCTTCCGAATAGCATCGGTAACATGAGCAAAGTC
TGCCGCCTTACAACGGCTCTCCCGCTGACGCCGTCCCGGACTGATGGGCTGCCTGT
ATCGAGTGGTGATTTTGTGCCGAGCTGCCGGTCGGGGAGCTGTTGGCTGGCTGGT
GGCAGGATATATTGTGGTGTAAACATAACGAATTCGTCTCAGGAGGTCAACTACC
CCAATTTAAATTTTATTTGATTAAGATATTTTTATGGACCTACTTTATAATTAAAAA
TATTTTCTATTTGAAAAGGAAGGACAAAAATCATACAATTTTGGTCCAACTACTCC
TCTCTTTTTITTTITGGCTTTATAAAAAAGGAAAGTGATTAGTAATAAATAATTAA
ATAATGAAAAAAGGAGGAAATAAAATTTTCGAATTAAAATGTAAAAGAGAAAAA
GGAGAGGGAGTAATCATTGTTTAACTTTATCTAAAGTACCCCAATTCGATTTTACA
TGTATATCAAATTATACAAATATTTTATTAAAATATAGATATTGAATAATTTTATT
ATTCTTGAACATGTAAATAAAAATTATCTATTATTTCAATTTTTATATAAACTATTA
TTTGAAATCTCAATTATGATTTTTTAATATCACTTTCTATCCATGATAATTTCAGCT
TAAAAAGTTTTGTCAATAATTACATTAATTTTGTTGATGAGGATGACAAGATTTCG
GTCATCAATTACATATACACAAATTGAAATAGTAAGCAACTTGATTTTTTTTCTCA
TAATGATAATGACAAAGACACGAAAAGACAATTCAATATTCACATTGATTTATTT
TTATATGATAATAATTACAATAATAATATTATTATAAAGAAAGAGATCAATTITGA
CTGATCCAAAAATTTATTTATTTTTACTATACCAACGTCACTAATTATATCTAATA
ATGTAAAACAATTCAATCTTACTTAAATATTAATTTGAAATAAACTATTTTTATAA
CGAAATTACTAAATTTATCCAATAACAAAAAGGTCTTAAGAAGACATAAATTCTT
TTTTTGTAATGCTGAAATAAATTTGAGTAAAAAAGAATGAAATTGAGTGATTTTTT
TTTAATCATAAGAAAATAAATAATTAATTTCAATATAATAAAACAGTAATATAAT
TTCATAAATGGAATTCAATACTTACCTCTAGATATAAAAAATAAATATAAAAATTT
AAAGTGTTTCTAATAAACCCGCAATTTAAATAANATATTTNATATTTTCAATCAAA
TTTAAATAATTATATTAAAATATCGTAGAAAAAGAGCAATATATAATACAAGAAA
GAAGATTTAAGTACAATTATCAACTATTATTATACTCTAATTTTGTTATATTTAATT
TCTTACGGTTAAGGTCATGTTCACGATAAACTCAAAATACGCTGTATGAGGACAT
ATTTTAAATTTTAACCAATAATAAAACTAAGTTATTTTTAGTATATTTTTTTGTTTA
ACGTGACTTAATTTTTCTTTTCTAGAGGAGCGTGTAAGTGTTCAACCTCATTCTCCT
AATTTTCCCAACCACATAAAAAAAAAATAAAGGTAGCTTTTGCGTGTTGATTTGGT
ACACTACACGTCATTATTACACGTGTTTTCGTATGATTGGTTAATCCATGAGGCGG
TTCCTCTAGAGTCGGCGATACCATCTATAAAATAAAGCTTTCTGGAGCTCATTTT
TTCATCTTCTATCTGATTTCTATTATAATTTCTCTGAATTGCCTTCAAATTTCTCTTT
CAAGGTTAGAATTTTTCTCTATTTTATGGTTTTTGTTTGTTTAGATTCTGAGTTTAGT
TAATCAGGTGCTGITAAAGCCCTAAATTTTGAGTTTTTTTCGGTTGTTTGATGGA
AAATACTAACAATTGAGTTTTTTCATGTTGTTTTGTCGGAGAATGCCTACAATTG
GAGTTCCTTTCGTTGTTTTGATGAGAAAGCCCCTTAATTTGAGTGTTTTCCGTCGAT
TTGATTTTAAAGG (SEQ ID NO: 47)
>pDGB-α2 LALABA (pDGB-alpha2 LALABA) (α-lactalbumin; alpha-
lactalbumin; LALBA)
CGCTTGAGACGAAGCTTTGACAGGATATATTGGCGGGTAAACCTAAGAGAAAAG
AGCGTTTATTAGAATAATCGGATATTTAAAAGGGCGTGAAAAGGTTTATCCGTTC
GTCCATTTGTATGTGCATGCCAACCACAGGGTTCCCCTCGGGATCAAAGTACTTTG
ATCCAACCCCTCCGCTGCTATAGTGCAGTCGGCTTCTGACGTTCAGTGCAGCCGTC
ATCTGAAAACGACATGTCGCACAAGTCCTAAGTTACGCGAGAGGCTGCCGCCCTG
CCCTTTTCCTGGCGTTTTCTTGTCGCGTGTTTTAGTCGCATAAAGTAGAATACTTGC
GACTAGAACCGGAGACATTACGCCATGAACAAGAGCGCCGCCGCTGGCCTGCTG
GGCTATGCCCGCGTCAGCACCGACGACCAGGACTTGACCAACCAACGGGCCGAA
CTGCACGCGGCCGGCTGCACCAAGCTGTTTTCCGAGAAGATCACCGGCACCATTGC
GCGACCGCCCGGAGCTGGCCAGGATGCTTGACCACCTACGCCCTGGCGACGTTGT
GACAGTGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCCGTGG
GCCGACACCACCACGCCGGCCGGCCGCATGGTTTTGACCGTGTTCGCCGGCATTG
CCGAGTTCGAGCGTTCCCTAATCATCGACCGCACCCGGAGCGGGCGCGAGGCCGC
CAAGGCCCGAGGCGTGAAGTTTGGCCCCCGCCCTACCCTCACCCCGGCACAGATC
GCGCACGCCCGCGAGCTGATCGACCAGGAAGGCCGCACCGTGAAAGAGGCGGCT
GCACTGCTTGGCGTGCATCGCTCGACCCTGTACCGCGCACTTGAGCGCAGCGAGG
AAGTGACGCCCACCGAGGCCAGGCGGCGCGGTGCCTTCCGTGAGGACGCATTGAC
CGAGGCCGACGCCCTGGCGGCCGCCGAGAATGAACGCCAAGAGGAACAAGCATG
AAACCGCACCAGGACGGCCAGGACGAACCGTTTTTCATTACCGAAGAGATCGAG
GCGGAGATGATCGCGGCCGGGTACGTGTTCGAGCCGCCCGCGCACCTCTCACCCG
TGCGGCTGCATGAAATCCTGGCCGGTTTGTCTGATGCCAAGCTGGCGGCCTGGCC
GGCCAGCTTGGCCGCTGAAGAAACCGAGCGCCGCCGTCTAAAAAGGTGATGTGTA
TTTGAGTAAAACAGCTTGCGTCATGCGGTCGCTGCGTATATGATCCGATGAGTAA
ATAAACAAATACGCAAGGGGAACGCATGAAGGTTATCGCTGTACTTAACCAGAA
AGGCGGGTCAGGCAAGACGACCATCGGAACCCATCTAGCCCGCGCCCTGCAACTC
GCCGGGGCCGATGTTCTGTTAGTCGATTCCGATCCCCACGGCAGTGCCCGCGATT
GGGCGGCCGTGCGGGAAGATCAACCGCTAACCGTTGTCGGCATCGACCGCCCGAC
GATTGACCGCGACGTGAAGGCCATCGGCCGGCGCGACTTCGTAGTGATCGACGGA
GCGCCCGAGGCGGCGGACTTGGCTGTGTCCGCGATCAAGGCAGCCGACTTCGTGC
TGATTCCGGTGCAGCCAAGCCCTTACGACATATGGGCCACCGCCGACCTGGTGGA
GCTGGTTAAGCAGCGCATTGAGGTCACGGATGGAAGGCTACAAGCGGCCTTTGTC
GTGTCGCGGGCGATCAAAGGCACGCGCATCGGCGGTGAGGTTGCCGAGGCGCTG
GCCGGGTACGAGCTGCCCATTCTTGAGTCCCGTATCACGCAGCGCGTGAGCTACC
CAGGCACTGCCGCCGCCGGCACAACCGTTCTTGAATCAGAACCCGAGGGCGACGC
TGCCCGCGAGGTCCAGGCGCTGGCCGCTGAAATTAAATCAAAACTGATTTGAGTT
AATGAGGTAAAGAGAAAATGAGCAAAAGCACAAACACGCTAAGTGCCGGCCGTC
CGAGCGCACGCAGCAGCAAGGCTGCAACGTTGGCCAGCCTGGCAGACACGCCAG
CCATGAAGCGGGTCAACTTTCAGTTGCCGGCGGAGGATCACACCAAGCTGAAGAT
GTACGCGGTACGCCAAGGCAAGACCATTACCGAGCTGCTATCTGAATAGATCGCG
CAGCTACCAGAGTAAATGAGCAAATGAATAAATGAGTAGATGAATTTTAGCGGCT
AAAGGAGGCGGCATGGAAAATCAAGAACAACCAGGCACCGACGCCGTGGAATGC
CCCATGTGTGGAGGAACGGGCGGTTGGCCAGGCGTAAGCGCCTGGGTTGTCTGCC
GGCCCTGCAATGGCACTGGAACCCCGAAGCCCGAGGAATCGGCGTGACGGTCGC
AAACCATCCGGCCCGGTACAAATCGGCGCGGCGCTGGGTGATGACCTGGTGGAG
AAGTTGAAGGCCGCGCAGGCCGCCCAGCGGCAACGCATCGAGGCAGAAGCACGC
CCCGGTGAATCGTGGCAAGCGGCCGCTGATCGAATCCGCAAAGAATCCCGGCAAC
CGCCGGCAGCCGGTGCGCCGTCGATTAGGAAGCCGCCCAAGGGCGACGAGCAAC
CAGATTTTTTCGTTCCGATGCTCTATGACGTGGGCACCCGCGATAGTCGCAGCATC
ATGGACGTGGCCGTTTTCCGTCTGTCGAAGCGTGACCGACGAGCTGGCGAGGTGA
TCCGCTACGAGCTTCCAGACGGGCACGTAGAGGTTTCCGCAGGGCCGGCCGGCAT
GGCCAGTGTGTGGGATTACGACCTGGTACTGATGGCGGTTTCCCATCTAACCGAA
TCCATGAACCGATACCGGGAAGGGAAGGGAGACAAGCCCGGCCGCGTGTTCCGT
CCACACGTTGCGGACGTACTCAAGTTCTGCCGGCGAGCCGATGGCGGAAAGCAGA
AAGACGACCTGGTAGAAACCTGCATTCGGTTAAACACCACGCACGTTGCCATGCA
GCGTACGAAGAAGGCCAAGAACGGCCGCCTGGTGACGGTATCCGAGGGTGAAGC
CTTGATTAGCCGCTACAAGATCGTAAAGAGCGAAACCGGGCGGCCGGAGTACATC
GAGATCGAGCTAGCTGATTGGATGTACCGCGAGATCACAGAAGGCAAGAACCCG
GACGTGCTGACGGTTCACCCCGATTACTTTTTGATCGATCCGGCATCGGCCGTTT
TCTCTACCGCCTGGCACGCCGCGCCGCAGGCAAGGCAGAAGCCAGATGGTTGTTC
AAGACGATCTACGAACGCAGTGGCAGCGCCGGAGAGTTCAAGAAGTTCTGTTTCA
CCGTGCGCAAGCTGATCGGGTCAAATGACCTGCCGGAGTACGATTTGAAGGAGGA
GGCGGGGCAGGCTGGCCCGATCCTAGTCATGCGCTACCGCAACCTGATCGAGGGC
GAAGCATCCGCCGGTTCCTAATGTACGGAGCAGATGCTAGGGCAAATTGCCCTAG
CAGGGGAAAAAGGTCGAAAAGGACTCTTTCCTGTGGATAGCACGTACATTGGGAA
CCCAAAGCCGTACATTGGGAACCGGAACCCGTACATTGGGAACCCAAAGCCGTAC
ATTGGGAACCGGTCACACATGTAAGTGACTGATATAAAAGAGAAAAAAGGCGAT
TTTTCCGCCTAAAACTCTTTAAAACITATTAAAACTCTTAAAACCCGCCTGGCCTG
TGCATAACTGTCTGGCCAGCGCACAGCCGAAGAGCTGCAAAAAGCGCCTACCCAT
CGGTCGCTGCGCTCCCTACGCCCCGCCGCTTTCGCGTCGGCCTATTCGGGCCGCTGG
CCGCTCAAAAATGGCTGGCCTACGGCCAGGCAATCTACCAGGGCGCGGACAAGC
CGCGCCGTCGCCACTCGACCGCCGGCGCCCACATCAAGGCACCCTGCCTCGCGCG
TTTCGGTGATGACGGTGAAAACCTCTGACACATGCAGCTCCCGGTGACGGTCACA
GCTTGTCTGTAAGCGGATGCCGGGAGCAGACAAGCCCGTCAGGGCGCGTCAGCG
GGTGTTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCACCAT
ATGCGGTGTGAAATACCGCACAGATGCGTAAGGAGAAAATACCGCATCAGGCGC
TCTTCCGCTTCCTCGCTCACTGACTCGCIGCGCTCGGTCGTTCGGCTGCGGCGAGC
GGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAG
GCGTFTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACT
ACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGC
GGTGGTTITTTTGTTTGCAAGCAGCAGATTACGCGCAGAAAAAAAGGATCTCAAG
AAGATCCTTTGATCTTTTCTACGGGGICTGACGCTCAGTGGAACGAAAACTGACGT
TAAGGGATTTTGGTCATGCATTCTAGGTGATTAGAAAAACTCATCGAGCATCAAA
TGAAACTGCAATTTATTCATATCAGGATTATCAATACCATATTTTTGAAAAAGCCG
TTTCTGTAATGAAGGAGAAAACTCACCGAGGCAGTTCGATAGGATGGCAAGATCC
TGGTATCGGTCTGCGATTCCGACTCGTCCAACATCAATACAACCTATTAATTTCCC
CTCGTCAAAAATAAGGTTATCAAGTGAGAAATCACCATCTAGTGACGACTGAATCC
GGTGAGAATGGCAAAAGTTTATGCATTTCTTTCCAGACTTGTTCAACAGGCCAGCC
ATTACGCTCGTCATCAAAATCACTCGCATCAACCAAACCGTATTCATTCGTGATT
GCGCCTGAGCGAGTCGAAATACGCGATCGCTGTTAAAAGGACAATTACAAACAG
GAATCGAATGCAACCGGCGCAGGAACACTGCCAGCGCATCAACAATATTTTCACC
TGAATCAGGATATTCTTCTAATACCTGGAATGCTGTTTTCCCTGGGATCGCAGTGG
TGAGTAACCATGCATGATCAGGAGTACGGATAAAATGCTTGATGGTCGGAAGAGG
CATAAATTCCGTCAGCCAGTTTAGTCTGACCATCTCATCTGTAACATCATTGGCAA
CGCTACCTTTGCCATGTTTCAGAAACAACTTGGCGCATCGGGCTTCCCATACAAT
CGGTAGATTGTCGCACCTGATTGCCCGACATTATCGCGAGCCCATTTATACCCATA
TAAATCAGCATCCATGTTGGAATTTAATCGCGGCCTTGAGCAAGACATTTCCCCTT
GAATATGGCTCATAACAGAACTTATTATTTCCTTCCTCTTTTCTACAGTATTTAAAG
ATACCCCAAGAAGCTAATTATAACAAGACGAACTCCAATTCACTGTTCCTTGCATT
CTAAAACCTTAAATACCAGAAAACAGCTTTTTCAAAGTTGTTTTCAAAGTTGGCGT
ATAACATAGTATCGACGGAGCCGATTTTGANACCGCGGTGATCACAGGCAGCAAC
GCTCTGTCATCGTTACAATCAACATGCTACCCTCCGCGAGATCATCCGTGTTTCAA
ACCCGGCAGCTTAGTTGCCGTTCTTCCGAATAGCATCGGTAACATGAGCAAAGTC
TGCCGCCTTACAACGGCTCTCCCGCTGACGCCGTCCCGGACTGATGGGCTGCCTGT
ATCGAGTGGTGATTTTGTGCCGAGCTGCCGGTCGGGGAGCTGTTGGCTGGCTGGT
GGCAGGATATATTGTGGTGTAAACATAACAAGCTTCGTCTCAGTCAGGAGGTCAA
CTACCCCAATTTAAATTTTATTTGATTAAGATATTTTTATGGACCTACTTTATAATT
AAAAATATTTTCTTTTTGAAAAGGAAGGACAAAAATCATACAATTTTGGTCCAAC
TACTCCTCTCTTTTTTTTTTTGGCTTTATAAAAAAGGAAAGTGATTAGTAATAAAT
AATTAAATAATGAAAAAAGGAGGAAATAAAATTTTCGAATTAAAATGTAAAAGA
CTAAAAAGGAGAGGGAGTAATCATTGTTTAACTTTATCTAAAGTACCCCAATTCGA
TTTTACATGTATATCAAATTATACAAATATTTTATTAAAATATAGATATTGAATAA
TTTTATTATTCTTGAACATGTAAATAAAAATTATCTATTATTTCAATTTTTATATAA
ACTATTATTTGAAATCTCAATTATGATTTTTTAATATCACTTTCTATCCATGATAAT
TTCAGCTTAAAAAGTTTTGTCAATAATTACATTAATTTTGTTGATGAGGATGACAA
GATITCGGTCATCAATTACATATACACAAATTGAAATAGTAAGCAACTTGATTTTT
TTICTCATAATGATAATGACAAAGACACGAAAAGACAATTCNATATTCACATTGA
TTTATTTTTATATGATAATAATTACAATAATAATATTCTTATAAAGAAAGAGATCA
ATTTTGACTGATCCAAAAATTTATTTATTTTTACTATACCAACGTCACTAATTATAT
CTAATAATGTAAAACAATTCAATCTTACTTAAATATTAATTTGAAATAAACTATTT
TTATAACGAAATTACTAAATTTATCCAATAACAAAAAGGTCTTAAGAAGACATAA
ATTCTTTTITTGTAATGCTCAAATAAATTTGAGTAAAAAAGAATGAAATTGAGTGA
TTTTTTTTTAATCATAAGAAAATAAATAATTAATTTCAATATAATAAAACAGTAAT
ATAATTTCATAAATGGAATTCAATACTTACCTCTTAGGATAAAAAATAAATATAA
AAATAAAGTGTTTCTAATAAACCCGCAATTTAAATAAAATATTTAATATTTTCAAT
CAAATTTAAATAATTATATTAAAATATCGTAGAAAAAGAGCAATATATAATACAA
GAAAGAAGATTTAAGTACAATTATCAACTATTATTATACTCTAATTTTGTTATATT
TAATTTCTTACGGTTAAGGTCATGTTGACGATAAACTCAAAATACGCTGTATGAGG
ACATATTTTAAATTTTAACCAATAATAAAACTAAGTTATTTTTAGTATATTTTTTTG
TTTAACGTGACTTAATTTTTCTTTTCTAGAGGAGCGTGTAAGTGTCAACCTCATTCT
CCTAATTTTCCCAACCACATAAAAAAAAAATAAAGGTAGCTTTTGCGTGTTGATTT
GGTACACTACACGTCATTATTACACGTGTTTTCGTATGATTGGTTAATCCATGAGG
CGGTTTCCTCTAGAGTCGGCCATACCATCTATAAAATAAAGCTTTCTGCAGCTCAT
TTTTTCATCTTCTATCTGATTTCTATTATAATTTCTCTGAATTGCCTTCAAATTTCTC
TTTCAAGGTTAGAATTTTTCTCTATTTTTTGGTTTTTGTTTGTTTAGATTCTGAGTTT
AGTTAATCAGGTGCTGTTAAAGCCCTAAATTTTGAGTTTTTTTCGGTTGTTTTGATG
GAAAATACCTAACAATTGAGTTTTTTCATGTTGTTTTGTCGGAGAATGCCTACAAT
TGGAGTTCCTTTCGTTGTTTTGATGAGAAAGCCCCTAATTTGAGTGTTTTTCCGTCG
ATTTGATTTTAAA (SEQ ID NO: 48)
>pDGB-α2 LGB (pDGB-alpha2 LCB) (β-lactoglobulin; beta-
lactoglobulin; LACB)
CGCTTGAGACGAAGCITTGACAGGATATATTGGCGGGTAAACCTAAGAGAAAAG
AGCGTTTATTAGAATAATCGGATATTTAAAAGGGCGTGAAAAGGTTTATCCGTTC
GTCCATTTGTATCTTGCATGCCAACCACAGGGTTCCCCTCGGGATCAAAGTACTTTG
ATCCAACCCCTCCGCTGCTATAGTGCAGTCGGCTTCTGACGTTCAGTGCAGCCGTC
ATCTGAAAACGACATGTCGCACAAGTCCTAAGTTACGCGACAGGCTGCCGCCGTG
CCCTTTTCCTGGCGTTTTCTTGTCGCGTGTTTTAGTCGCATAAAGTAGAATACTTGC
GACTAGAACCGGAGACATTACGCCATGAACAAGAGCGCCGCCGCTGGCCTGCTG
GGCTATGCCCGCGTCAGCACCGACGACCAGGACTGACCAACCAACGGGCCGAA
CTGCACGCGGCCGGCTGCACCAAGCTGTTTTCCGAGAAGATCACCGGCACCAGGC
GCGACCGCCCGGAGCTGGCCAGGATGCTTGACCACCTACGCCCTGGCGACGTTGT
GACAGTGACCAGGCTAGACCGCCTGGCCCGCAGCACCCGCGACCTACTGGACATT
GCCGAGCGCATCCAGGAGGCCGGCGCGGGCCTGCGTAGCCTGGCAGAGCCGTGG
GCTCGACACCACCACGCCGGCCGGCCGCATGGTGTTGACCGTGTTCGCCGGCATTG
CCGAGTTCGAGCGTTCCCTAATCATCGACCGCACCCGGAGCGGGCGCGAGGCCGC
CAAGGCCCGAGGCGTGAAGTTTGGCCCCCGCCCTACCCTCACCCCGGCACAGATC
GCGCACGCCCGCGAGCTGATCGACCAGGAAGGCCGCACCGTGAAAGAGGCGGCT
GCACTGCTTGGCGTGCATCGCTCGACCCTGTACCGCGCACTTGAGCGCAGCGAGG
AAGTGACGCCCACCGAGGCCAGGCGGCGCGGTGCCTTCCGTGAGGACGCATTGAC
CGAGGCCGACGCCCTGGCGGCCGCCGAGAATGAACGCCAAGAGGAACAAGCATG
AAACCGCACCAGGACGGCCAGGACGAACCGTTTTTCATTACCGAAGAGATCGAG
GCGGAGATGATCGCGGCCGGGTACGTGTTCGAGCCGCCCGCGCACCTCTCAACCG
TCTCGGCTGCATGAAATCCTGGCCGGTTTGTCTGATGCCAAGCTGGCGGCCTGGCC
GGCCAGCTTGGCCGCTGAAGAAACCGAGCGCCGCCGTCTAAAAAGGTGATGTGTA
TTTGAGTAAAACAGCTTGCGTCATGCGGTCGCTGCGTATATGATCCGATGAGTAA
ATAAACAAATACGCAAGGGGAACGCATGAAGGTTATCGCTGTACTTAACCAGAA
AGGCGGGTCAGGCAAGACGACCATCGGAACCCATCTAGCCCGCGCCCTGCAACTC
GCCGGGGCCGATGTTCTGTTAGTCGATTCCGATCCCCAGGGCAGTGCCCGCGATT
GGGCGGCCGTGCGGGAAGATCAACCGCTAACCGTTGTCGGCATCGACCGCCCGAC
GATTGACCGCGACGTGAAGGCCATCGGCCGGCGCGACTTCGTAGTGATCGACGGA
GCGCCCCAGGCGGCGGACTTGGCTGTGTCCGCGATCAAGGCAGCCGACTTCGTGC
TGATTCCGGTGCAGCCAAGCCCTTACGACATATGGGCCACCGCCGACCTGGTGGA
GCTGGTTAAGCAGCGCATTGAGGTCACGGATGGAAGGCTACAAGCGGCCTTTGTC
GTGTCGCGGGCGATCAAAGGCACGCGCATCGGCGGTGAGGTTGCCGAGGCGCTG
GCCGGGTACGAGCTGCCGATTCTTGAGTCCCGTATCACGCAGCGCGTGAGCTACC
CAGGCACTGCCGCCGCCGGCACAACCGTTCTTGAATCAGAACCCGAGGGCGACGC
TGCCCGCGAGGTCCAGGCGCTGGCCGCTGAAATTAAATCAAAACTCATTTGAGTT
AATGAGGTAAAGAGAAAATGAGCAAAAGCACAAACACGCTAAGTGCCGGCCGTC
CGAGCGCACGCAGCAGCAAGGCTGCAACGTTGGCCAGCCTGGCAGACACGCCAG
CCATGAAGCGGGTCAACTTTCAGTTGCCGGCGGAGGATCACACCAAGCTGAAGAT
GTACGCGGTACGCCAAGGCAAGACCATTACCGAGCTGCTATCTGAATAGATCGCG
CAGCTACCAGAGTAAATGAGCAAATGAATAAATGAGTAGATGAATTTTAGCGGCT
AAAGGAGGCGGCATGGAAAATCAAGAACAACCAGGCACCGACGCCGTGGAATGC
CCCATGTGTGGAGGAACGGGCGGTTGGCCAGGCGTAAGCGGCTGGGTTGTCTGCC
GGCCCTGCAATGGCACTGGAACCCCCAAGCCCGAGGAATCGGCGTGACGGTCGC
AAACCATCCGGCCCGGTACAAATCGGCGCGGCGCTGGGTGATGACCTGGTGGAG
AAGTTGAAGGCCGCGCAGGCCGCCCAGCGGCAACGCATCGAGGCAGAAGCACGC
CCCGGTGAATCGTGGCAAGCGGCCGCTGATCGAATCCGCAAAGAATCCCGGCAAC
CGCCGGCAGCCGGTGCGCCGTCGATTAGGAAGCCGCCCAAGGGCGACGAGCAAC
CAGATTTTTTCGTTCCGATGCTCTATGACGTGGGCACCCGCGATAGTCGCAGCATA
ATGGACGTGGCCGTTTTCCGTCTGTCGAAGCGTGACCGACGAGCTGGCGAGGTGA
TCCGCTACGAGCTTCCAGACGGGCACGTAGAGGTTTCCGCAGGGCCGGCCGGCAT
GGCCAGTGTGTGGGATTACGACCTGGTACTGATGGCGGTTTCCCATCTAACCGAA
TCCATGAACCGATACCGGGAAGGGAAGGGAGACAAGCCCGGCCGCGTGTTCCGT
CCACACGTTGCGGACGTACTCAAGTTCTGCCGGCGAGCCGATGGCGGAAAGCAGA
AAGACGACCTGGTAGAAACCTGCATTCGGTTAAACACCACGCACGTTGCCATGCA
GCGTACGAAGAAGGCCAAGAACGGCCGCCTGGTGACGGTATCCGAGGGTGAAGC
CTTGATTAGCCGCTACAAGATCGTAAAGAGCGAAACCGGGCGGCCGGAGTACATC
GAGATCGAGCTAGCTGATTGGATGTACCGCGAGATCACAGAAGGCAAGAACCCG
GACGTGCTGACGGTTCACCCCGATTACTTTTTGATCGATCCCGGCATCGGCCGTTT
TCTCTACCGCCTGGCACGCCGCGCCGCAGGCAAGGCAGAAGCCAGATGGTTGTTC
AAGACGATCTACGAACGCAGTGGCAGCGCCGGAGAGTTCAAGAAGTTCTGTTTCA
CCGTGCGCAAGCTGATCGGGTCAAATGACCTGCCGGAGTACGATTTGAAGGAGGA
GGCGGGGCAGGCTGGCCCGATCCTAGTCATGCGCTACCGCAACCTGATCGAGGGC
GAAGCATCCGCCGGTTCCTAATGTACGGAGCAGATGCTAGGGCAAATTGCCCTAG
CAGGGGAAAAAGGTCGAAAAGGACTCTTTCCTGTGGATAGCACGTACATTGGGAA
CCCAAAGCCGTACATTGGGAACCGGAACCCGTACATTGGGAACCCAAAGCCGTAC
ATTGGGAACCGGTCACACATGTAAGTGACTGATATAAAAGAGAAAAAAGGCGAT
TTTTCCGCCTAAAACTCTTTAAAACTTATTAAAACTCTTAAAACCCGCCTGGCCTG
TGCATAACTGTCTGGCCAGCGCACAGCCGAAGAGCTGCAANAAGCGCCTACCCTT
CGGTCGCTGCGCTCCCTACGCCCCGCCGCTTCGCGTCGGCCTATCGCGGCCGCTGG
CCGCTCAAAAATGGCTGGCCTACGGCCAGGCAATCTACCAGGGCGCGGACAAGC
CGCGCCGTCGCCACTCGACCGCCGGCGCCCACATCAAGGCACCCTGCCTCGCGCG
TTTCGGTGATGACGGTGAAAACCTCTGACACATGCAGCTCCCGGTGACGGTCACA
GCTTGTCTGTAAGCGGATGCCGGGAGCAGACAAGCCCGTCAGGGCGCGTCAGCG
GGTGTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCACCAT
ATGCGGTGTGAAATACCGCACAGATGCGTAAGGAGAAAATACCGGATCAGGCGC
TCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAAC
GCAGGAAAGAACATGTGAGCAAAAGGCCAGCAAAAGGCCAGGAACCGTAAAAA
GGCCGCGTTGGTGGCGTTTTTCCATAGGCTCCGCCCCCCTGACGAGCATCACAAA
AATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCAC
GAACCCCCCGTTCAGCCCGACCGCTGCGCTTATCCGGTAACTATCGTCTTGAGTC
CAACCCGGTAAGACACGACTTATCGCCACTGGCAGCAGCCACRGGTAACAGGATT
AGCAGAGCGAGGTATGTAGGCGGTGCTACAGAGTTCTTGAAGTGGTGGCCTAACT
ACGGCTACACTAGAAGGACAGTATTTGGTATCTGCGCTCTGCTGAAGCCAGTTAC
CTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAG
AAGATCCITTGATCTTTCTACGGGGTCTGACGCTCAGTGGAACGAAAACTCACGT
TAAGGGATTTTGGTCATGCATTCTAGGTGATTAGAAAAACTCATCGAGCATCAAA
TGAAACTGCAATTTATTCATATCAGGATTATCAATACCATATTTTTGAAAAAGCCG
TTTCTGTAATGAAGGAGAAAACTCACCGAGGCAGTTCCATAGGATGGCAAGATCC
TGGTATCGGTCTGCGATTCCGACTCGTCGAACATCAATACAACCTATTAATTTCCC
CTCGTCAAAAATAAGGTTATCAAGTGAGAAATCACCATGAGTGACGACTGAATCC
GGTGAGAATGGCAAAAGTTTATGCATTTCTATCCAGATTGTTCAACAGGCCAGCC
ATTACGCTCGTCATCAAAATCACTCGCATCAACCAAACCGTTATTCATTCGTGATT
GCGCCTGAGCGAGTCGAAATACGCGATCGGTGTTAAAAGGACAATTACAAACAG
GAATCGAATGCAACCGGCGCAGGAACACTGCCAGCGCATCAACAATATTTTCACC
TGAATCAGGATATTCTTCTAATACCTGGAATGCTGTTTTCCCTGGGATCGCAGTGG
TTAGTAACCATGCATCATCAGGAGTACGGATAAAATGCTTGATGGTCGGAAGAGG
CATAAATTCCGTCAGCCAGTTTAGTCTGACCATCTCATCTGTAACATCATTGGCAA
CGCTACCTTTGCCATGTTTCAGAAACAACTCTGGCGCATCGGGCTTCCCATACAAT
CGGTAGATTGTCGCACCTGATTGCCCGACATTATCGCGAGCCCATTTATACCCATA
TAAATCAGCATCCATGTTGGAATTTAATCGCGGCCTTGAGCAAGACGTTTCCCGTT
GAATATGGGTCATAACAGAACTTATTATTTCCTTCCTCTTTTCTACAGTATTTAAAG
ATACCCCAAGAAGCTAATTATAACAAGACAACTCCAATTCACTGTTCCTTGCATT
CTAAAACCTTAAATACCAGAAAACAGCTTTTTCAAAGTTGTTTTCAAAGTTGGCGT
ATAACATAGTATCGACGGAGCCGATTTTGAAACCGCGGTGATCACAGGCAGCAAC
GCTCTGTCATCGTTACAATCAAGATGCTACCCTCCGCGAGATCATCCGTGTTTCAA
ACCCGGCAGGTTAGTTGCCGTTCTTCCGAATAGCATCGGTAACATGAGCAAAGTC
TGCCGCCTTACAACGGCTCTCCCGCTGACGCCGTCCCGGACTGATGGGCTGCCTGT
ATCGAGTGGTGATTTTGTGCCGAGCTGCCGGTCGGGGAGCTGTTGGCTGGCTGGT
CTGCAGGATATATTGTGTTTGTAAACATAACAAGCTTCGTCTCAGTCAGGAGGTGAA
CTACCCCAATTTAAATTTTATTTGATTAAGATATTTTTATGGACCTACTTTATAATT
AAAAATATTTTCTATTTGAAAAGGAAGGACAAAAATCATACAATTTTGGTCCAAC
TAGTCCTCTCTTTTTTTTTTTTGGCTTTATAAAAAAGGAAAGTGATTAGTAATAAAT
AATTAAATAATGAAAAAAGGAGGAAATAAAATTTTCGAATTAANATCTAAAAGA
GAAAAAGGAGAGGGAGTAATCATTGTTTAACTTTATCTAAAGTACCCCAATTCGA
TTTTACATGTATATCAAATTATACAAATATTTTATTAAAATAAGATATTGAATAA
TTTTATTATTCTTGAACATGTAAATAAAAATTATCTATTATTTCAATTTTTATATAA
ACTATTATTTGAAATCTCAATTATGATTTTTTAATATCACTTTCTATCCATGATAAT
TTCAGCTTAAAAAGTTTTGTGAATAATTACATTAATTTTGTTGATGAGGATGACAA
GATTTCGGTCATCAATTACATATACACAAATTGAAATAGTAAGCAACTTGATTTTT
TTTCTCATAATGATAATGACAAAGACACGAAAAGACAATTCAATATTCACATTGA
TTTATTTTTATATGATAATAATTACAATNATAATATTCTTAIAAAGAAAGAGATCA
ATTTTGACTGATCCAAAAATTTATTTATTTTTACTATACCAACGTCACTAATTATAT
CTAATAATGTAAAACAATTCAATCTTACTTAAATATTAATTTGAAATAAACTATTT
TTATAACGAAATTACTAAATTTATCCAATAACAAAAAGGTCTTAAGAAGACATAA
ATTCTTTTTTTGTAATGCTCAAATAAATTTGAGTAAAAAAGAATGAAATTGAGTGA
TTTTTTTTTAATCATAAGAAAATAAATAATTAATTTCAATATAATAAAACAGTAAT
ATAATTTCATAAATGGAATTCAATACTTACCTCTTAGATATAAAAAATAAATATAA
AAATAAAGTGTTTCTAATAAACCCGCAATTTAAATAAAATATTTAATATTTTCAAT
CAAATTTAAATAATTATATTAAAATATCGTAGAAAAAGAGGAATATATAATACAA
GAAAGAAGATTTAAGTACAATTATCAACTATTATTATACTCTAATTTTGTTATATT
TAATTTCTTACGGTTAAGGTCATGTTCACGATAAACTCAAAATACGCTGTATGACG
ACATATTTTAAATTTTAACCAATAATAAAACTAAGTTATTTTTAGTATATTTTTTTG
TTTAACGTGACTTAATTTTTCTTTTCTAGAGGAGCGTGTAAGTGTCAACCTCATTCT
CCTAATTTTCCCAACCACATAAAAAAAAAATAAAGGTACATTTTGCGTGTTGATTT
GGTACACTACACGTCATTATTACACGTGTTTTCGTATGATTGGTTAATCCATGAGG
CGGTTTCGTCTAGAGTCGGCCATACCATCTATAAAATAAAGCTTTCTGCAGCTCAT
TTTTTCATCTTCTATCTGATTTCTATTATAATTTCTCTGAATTGCCTTCAAATTTCTC
TTTCAAGGTTAGAATTTTTCTCTATTTTTTGGTTTTTGTTTGTTTAGATTCTGAGTTT
AGTTAATCAGGTGCTTTAAAGCCCTAAATTTTGAGTTTTTTTAGGTTCTTTTGAT
GAAAATACCTAACAATTGAGTTTTTTGATGTTGTTTTGTCGGAGAATGCCTACAAT
TGGAGTTCCTTTCGTTGTTTTGATGAGAAAGCCCCTAATTTGAGTGTTTTTCCGTCG
ATTTGATTTTAAA (SEQ ID NO: 49)

### Example 2: Transfection of Nicotiana benthamiana Plant Leaves with Binary Expression Vectors and Expression of mRNA Transcripts of Cow's Milk Genes

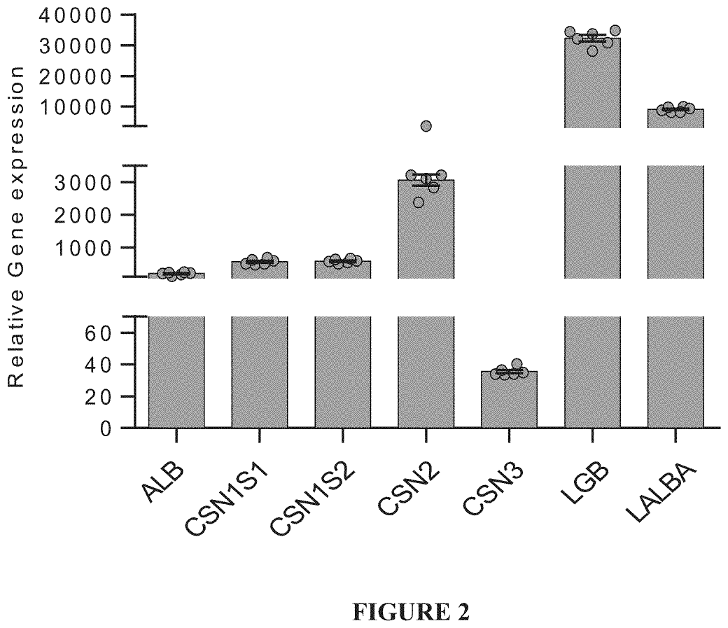

Next, four-week old Nicotiana benthamiana (N. benthamiana) plant leaves were transformed with Agrobacterium tumefaciens, each carrying one of these seven constructs. Analysis of gene expression using quantitative real-time polymerase chain reaction (qRT-PCR), showed high expression levels of mRNA transcripts of all seven genes compared with non-transformed leaves (control) (FIG. 2). Gene expression is presented as fold change compared with non-transformed leaves and normalized to the house keeping gene F-BOX.

### Example 3: Protein Expression of Cow's Milk Genes in Nicotiana benthamiana Plant Leaves

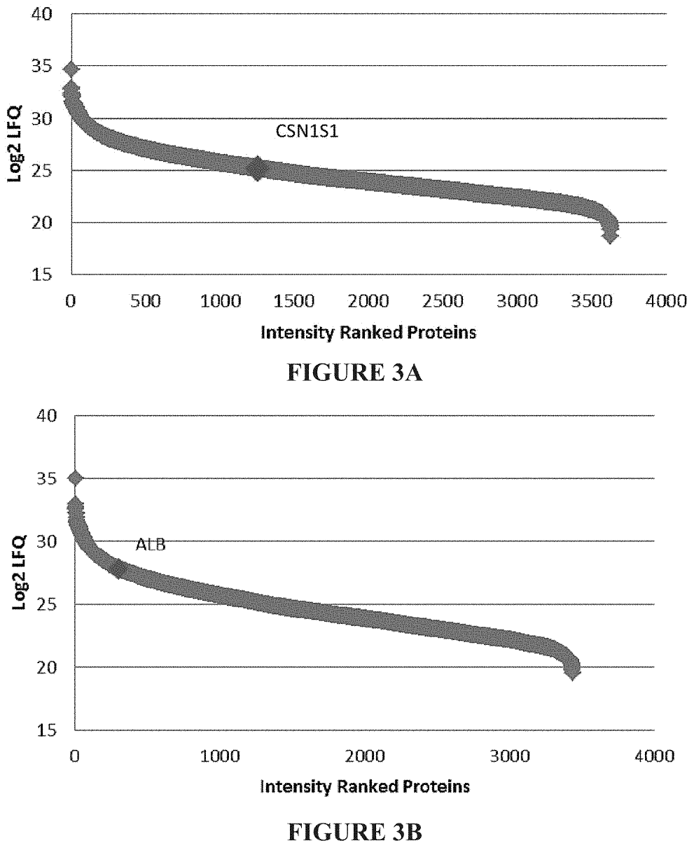

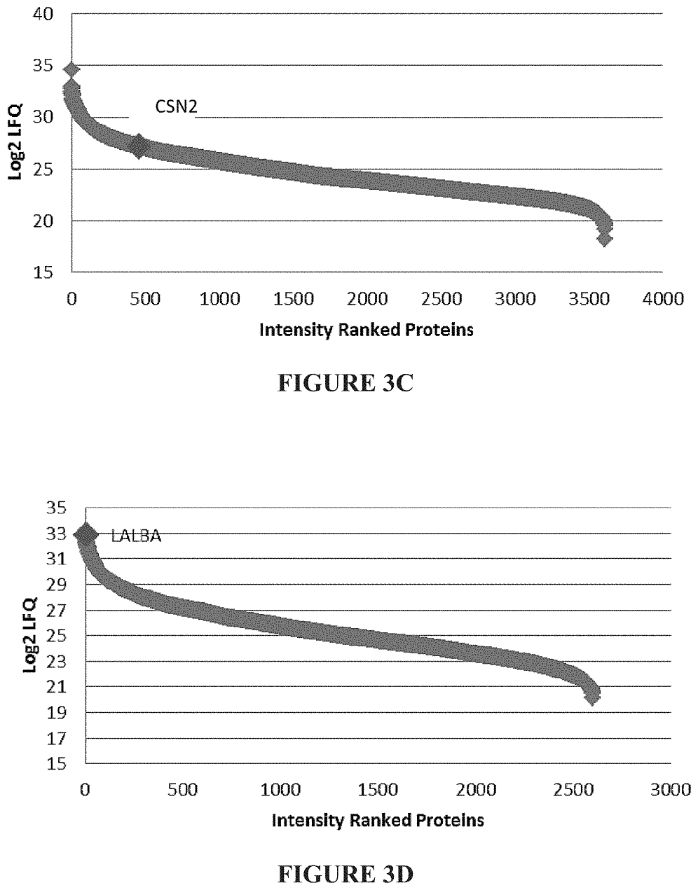

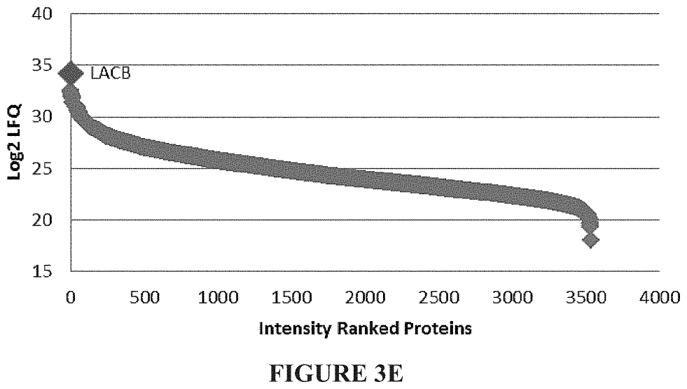

To confirm the protein expression of the cow's milk genes in the transformed N. benthamiana leaves, LC-MS/MS proteomic analysis was utilized and successfully identified high expression of five of the seven expressed cow's milk proteins (FIGS. 3A-3E), demonstrating that these proteins can be expressed in plants. These five proteins are: (FIG. 3A) CSN1S1 (α-S1-casein; alpha-S2-casein), (FIG. 3B) ALB (serum albumin), (FIG. 3C) CSN2 (β casein; beta casein), (FIG. 3D) LALBA (α-lactalbumin; alpha-lactalbumin), and (FIG. 3E) LGB (LACB) (β-lactoglobulin; beta-lactoglobulin).

Therefore, cow's milk proteins could be expressed in plants. The expression of these genes did not result in gross morphological abnormalities in the leaves of Nicotiana benthamiana.

### Example 4: Vector for Co-Expression of Cow's Milk Genes Simultaneously in a Single Plant

To express all seven genes simultaneously in a single plant (e.g., Nicotiana benthamiana plant leaf, rice plant or seed, soy plant or seed/soybean), the T-DNA binary vector (plasmid), pDGB-Ω1 Seven bovine milk genes (pDGB-Ω1 Seven milk genes, pDGB-Ω1 Seven genes; pDGB-omega1 Seven bovine milk genes, pDGB-omega1 Seven genes; pDGB-Seven genes), carrying all the seven cow's milk proteins under the control of constitutive SlPUbiq10 promoters as well as the BASTA resistance gene, was constructed, as pDGB-Ω1 has been transfected in N. benthamiana (FIG. 4, TABLE 6).

The pDGB-S1 Seven bovine milk genes (pDGB-omega1 Seven bovine milk genes) plasmid was co-transfected with an Agrobacterium plasmid encoding integration genes. Transformed plants included Nicotiana benthamiana, Oryza sativa (rice), and Glycine mar (soybean). Where integration takes place, the integration region lies substantially between the LB and RB sequences (FIG. 4). Gene-edited plants can also be produced according to standard methodology.

TABLE 6
Sequence of T-DNA plasmid coding for seven cow's milk genes
and BASTA resistance gene.

>pDGB-Ω1 (pDGB-omega1) Seven Bovine Milk Genes
TTTTGATGTCGCTTTGGTTCTCAAGGCCTAAGATCTGAGTTTCTCCGGTTGTTTTGA
TGAAAAAGCCCTAAAATTGGAGTTTTTATATTGTGTTTTAGGTTGTTTTAATCCTTA
TAATTTGAGTTTTTTCGTTGTTCTGATTGTTGTTTTTATGAATTTTGCAGAATGAAG
TGGGTGACTTTTATTTCTCTTCTCCTTCTCTTCAGCTCTGCTTATTCCAGGGGTGTG
TTTCTCGAGATACACACAAGAGTGAGATTGCTCATCGGTTTAAAGATTTGGGAG
AAGAACATTTTAAAGGCCTGGTACTGATTGCCTTTTGTCAGTATCTCCAGCAGTGT
CCATTTGATGAGCATGTAAAATTAGTGAACGAACTAACTGAGTTTGCAAAAACAT
GTGTTGCTGATGAGTCCCATGCCGGCTGTGAAAAGTCACTTCACACTCTCTTTGGA
GATGAATTGTGTAAAGTTGCATCCCTTCGTGAAACCTATGGTGACATGGCTGACTG
CTGTGCGAAACAAGAGCCTGAAAGAAATGAATGCTTCCTGAGCCACAAAGATGAT
AGCCCAGACCTCCCTAAATTGAAACCAGACCCCAATACTTTGTGTGATGAGTTTA
AGGCAGATGAAAAGAAGTTTTGGGGAAAATACCTATACGAAATGCTAGAAGAC
ATCCCTACTTTTATGCACCAGAACTCCTTTACTATGCTAATAAATATAATGGAGTT
TTTCAAGAATGCTGCCAAGCTGAAGATAAAGGTGCCTGCCTGCTACCAAAGATTG
AAACTATGAGAGAAAAAGTACTGACTTCATCTGCCAGACAGAGACTCAGGTGTGC
CAGTATTCAAAAATTTGGAGAAAGAGCTTTAAAAGCATGGTCAGTAGCTCGCCTG
AGCCAGAAATTTCCCAAGGCTGAGTTTGTAGAAGTTACCAAGCTAGTGACAGATC
TCACAAAAGTCCACAAGGAATGCTGCCATGGTGACCTACTTGAATGCGCAGATGA
CAGGGCAGATCTTGCCAAGTACATATGTGATAATCAAGATACAATCTCCAGTAAA
CTGAAGGAATGGTGTGATAAGCCTTTGTTGGAAAAATCCCACTGCATTGCTGAGG
TGGAAAAAGATGCCATACCTGAAAACCTGCCCCCATTAACTGCTGACTTTGCTGA
AGATAAGGATGTTTGCAAAAACTATCAGGAAGCAAAAGATGCCTTCCTGGGCTCG
TTTTTGTATGAATATTCAAGAAGGCATCCTGAATATGCTGTCTCAGTGCTATTGAG
ACTTGCCAAGGAATATGAAGCCACACTGGAGGAATGCTGTGCCAAAGATGATCCA
CATGCATGCTATTCCACAGTGTTTGACAAACTTAAGCATCTTGTGGATGAGCCTCA
GAATTTAATCAAACAAAACTGTGACCAATTCGAAAAACTTGGAGAGTATGGATTC
CAAAATGAGCTCATAGTTCGTTACACCAGGAAAGTACCCCAAGTGTCAACTCCAA
CTCTCGTGGAGGTTTCAAGAAGCCTAGGAAAAGTGGGTACTAGGTGATGTACAAA
GCCGGAATCAGAAAGAATGCCCTGTGCTGAAGACTATCTGAGCTTGATCCTGAAC
CGGTTGTGCGTGCTGCATGAGAAGACACCAGTGAGTGAAAAAGTCACCAAGTGCT
GCACAGAGTCATTGGTGAACAGACGGCCATGTTTCTCTGGTCTGACACCTGATGA
AACATATGTACCCAAAGCCTTTGATGAGAAATTGTTCACCTTCCATGCAGATATAT
GCACACTTCCCGATACTGAGAAACAAATCAAGAAACAAACTGCACTTGTTGAGCT
GTTGAAACACAAGCCCAAGGCAACAGAGGAACAACTGAAAACCATATGGAGAA
TTTTGTGGCTITTGTAGGCAAGTGCTGTGCAGCTGATGACAAAGAGGCCTGCTTTG
CTGTGGAGGGTCCAAAACTTGTTGTTTCAACTCAAACAGCCTTAGCCTAAGCTTGT
TGTGGTTGTCTGGTTGCGTCTGTTGCCCGTTGTCTGTTGCCCATTGTGGTGGTTGTG
TTTGTATGATGGTCGTTAAGGATCATCAATGTGTTTTCGCTTTTTGTTCCATTCTGT
TTCTCATTTGTGAATAATAATGGTATCTTTATATTATGCAGTTTGTGGTTTCTTTT
CTGATTGCAGTTCTGAGCATTTTGTTTTTGCTTCCGITTACTATACCACTTACAGTT
TGCACTAATTTAGTTGATATGCGAGCCATCTGATGTTTGATGATTCAAATGGCGTT
TATGTAACTCGTACCCGAGTGGATGGAGAAGAGCTCCATTGCCGGTTTGTTTCATG
GGTGGCGGAGGGCAACTCCTGGGAAGGAACAAAAGAAAAACCGTGATACGAGTT
CATGGGTGAGAGCTCCAGCTTGATCCCTTCTCTGTCGATCAAATTTGAATTTTTGG
ATCACGGCAGGCTCACAAGATAATCCAAAGTAAAACATAATGAATAGTACTTCTC
AATGATCACTTATTTTTAGCAAATCAGCAATTGTGCATGTCAAATGATTTCGGTGT
AAGAGAAAGAGTTGATGAATCAAAATATCTGTAGCTGGATCAAGAATCTGAGGC
AGTTGTATGTATCAATGATCTTTCCGCTACAATGATGTTAGCTATCCGAGTCAAAT
TGTTGTAGAATTGCATACTTCGGCATCACATTCTGGATGACATAATAAATAGGAA
GICTTCAGATCCCTAAAAAATTGAGAGCTAATAACATTAGTCCTAGATGTAACTG
GGTGACAACCAAGAAAGAGACATGCAAATACTACTTTTGTTTGAAGGAGCATCCC
TGGTTTGACATATTTTTTCTGAATATCAAACTTTGAAACTCTACCTAGTCTAATGTC
TAACGACAGATCTTAGTGGTTTAACTGCAGTGATATCTACTATCTTTTGGAATGTT
TTCTCCTTCAGTTATACATCAAGTTCCAAGATGCAGGTGTGCTTGATTGATGTACA
TGGCTGTGAGAAGTGCATCCTGATGTTCAGATGATGGTTCATTCTAATGTCTTTTC
CTTCAATCAGTTTTCTCAGTCTGACTTAGCTTGTTTCATCTGCATGTTTGAATGTTC
GTTTACTCATAGTAATTGCATTTTTGTAGCAGAACATATCATTGGTCATGGTTTCA
ACTGTGCGCGAGTCTTATGCTIATTCAAACTAGGAAAGCCTCCGTCTAGAGGGTA
CACGAGTTGTTGCTCTGTGTGCGTCAGTCCATAGTATTAATCTTGCTAGTTGTAGT
ATATTGTTTATATGGACTCGGAATTCATCATATGCTCCTTCTTTGCATCAAGTAAG
GCAAGGTNATGTATAGAAGCTTTTTAACTCTTTCATGGAAGCTGGCCTTTGCCAGC
ATACCATCCAGAAGATATCAACCCTGCATCTTGGCTGCCGCGCTGTCAGGAGGTC
AACTACCCCAATTTAAATTTTATTTGATTAAGATATTTTTATGGACCTACTTTATAA
TTAAAAATATTTTCTATTTGAAAAGGAAGGACAANAATGATAGAATTTTGGTCCA
ACTACTCCTCTCTTTTTTTTTTTGGCTTTATAAAAAAGGAAAGTGATTAGTAATAA
ATAATTAAATAATGAAAAAAGGAGGAAATAAAATTTTCGAATTAAAATGTAAAA
GAGAAAAAGGAGAGGGAGTAATCATTGTTTAACTTTATCTAAAGTACCCCAATTC
GATTTTACATGTATATCAAATTATACAAATATTTTATTAAAATATAGATATTGAAT
AATTTTATTATTCTTGAACATGTAAATAAAAATTATCTATTATTTCAATTTTTATAT
AAACTATTATTTGAAATCTCAATTATGATTTTTTAATATCACTTTCTATCCATGATA
ATTTCAGCTTAAAAAGTTTTGTCAATAATTACATTAATTTTGTTGATGAGGATGAC
AAGATTTCGGTCATCAATTACATATACACAAATTGAAATAGTAAGGAACTTGATTT
TTTTTCTCATAATGATAATGACAAAGACACGAAAAGACAATTCAATATTCACATT
GATTTATTTTTATATGATAATAATTACAATAATAATATTCTTATAAAGAAAGAGAT
CAATTTTGACTGATCCAAAAATTTATTTATTTTTACTATACCAACGTCACTAATTAT
ATCTAATAATGTAAAACAATTCAATCTTACTTAAATATTAATTTGAAATAAACTAT
TITTATAACGAAATTACTAAATTTATCCAATAACAAAAAGGTCTTAAGAAGACAT
AAATTCTTTTTTTGTAATGCTCAAATAAATTTGAGTAAAAAAGAATGAAATTGAGT
GATTTTTTTTTAATCATAAGAAAATAAATAATTAATTTCAATATAATAAAACAGTA
ATATAATTTCATAAATGGAATTCAATACTTACCTCTTAGATATAAAAAATAAATAT
AAAAATAAAGTATTTCTAATAAACCCGCAATTTAAATAAAATATTTAATATTTTCA
ATCAAATTTAAATAATTATATTAAAATATCGTAGAAAAAGAGCAATGATAATAC
AAGAAAGAAGATTTAAGTAGAATTATCAACTATTATTATACTCTNATTTTGTTATA
TTTAATTTCTTACGGTTAAGGTCATGTTCACGATAAACTCAAAATACGCTGTATGA
GGACATTTTAAATTTTAACCAATAATAAAACTAAGTTATTTTTAGTATATTTTTT
TGTTTAACGTGACTTAATTTTTCTTTTCTAGAGGAGCGTGTAAGTGTCAACCTCATT
CTCCTAATTTTCCCAACCACATAAAAAAAAAATAAAGGTAGCTTTTGCGTGTTGAT
TTGGTACACTACACGTCATTATTACACGTGTTTTCGTATGATTGGTTAATCCATGA
GGCGGTTTCCTCTAGAGTCGGCCATACCATCTATAAAATAAAGCTTTCTGCAGCTC
ATTTTTTCATCTTCTATCTGATTTCTATTATAATTTCTCTGAATTGCCTTCAAATTTC
TCTTTCAAGGTTAGAATTTTTCTCTATTTTTTGGTTTTTGTTTGTTTAGATTCTGAGT
TTAGTTAATCAGGTGCTGTTAAAGCCCTAAATTTTGAGTTTTTTTCGGTTGTTTTGA
TGGAAAATACCTAACAATTGAGTTTTTTCATGTTGTTTTGTCGGAGAATGCCTACA
ATTGGAGTTCCTTTCGTTGTTTTGATGAGAAAGCCCCTAATTTGAGTGTTTTTCCGT
CGATTTGATTTTAAAGGTTTATATTCGAGTTTTTTTCGTCGGTTTAATGAGAAGGC
CTAAAATAGGAGTTTTTCTGGTTGATTTGACTAAAAAAGCGATGGAATTTGTGTT
TTTGATGTCGCTTTGGTTCTCAAGGCCTAAGATCTGAGTTTCTCCGGTTGTTTTGAT
GAAAAAGCCCTAAAATTGGAGTTTTTATTGTGTTTTAGGTTGTTTTAATCCTTAT
AATTTGAGTTTTTTCGTTGTTCTGATTGTTGTTTTTATGAATTTTGCAGAATGAAAC
TTCTCATCCTTACCTGTCTTGTGGCTGTTGCTCTTGCCAGGCCTAAACATCCTATCA
AGCACCAAGGACTCCCTCAAGAAGTCCTCAATGAAATTTTACTCAGGTTTTTTGTG
GCACCTTTTCCAGAAGTGTTTGGAAAGGAGAAGGTCAATGAACTGAGCAAGGATA
TTGGGAGTGAATCAACTGAGGATCAAGCCATGGAAGATATTAAGCAAATGGAAG
CTGAAAGCATTTCGTCAAGTGAGGAAATTCGCCCAATAGTGTTGAGCAGAAGCA
CATTCAAAAGGAAGATGTGCCCTCTGAGCGTTACCTGGGTTATCTGGAACAGCIT
CTCAGACTGAAAAAATACAAAGTACCCCAGCTGGAAATTGTTCCCAATAGTGCTG
AGGAACGACTTCACAGTATGAAAGAGGGAATCCATGCCCAACAGAAAGAACCTA
TGATAGGAGTGAATCAGGAACTGGCCTACTTTACCCTGAGCTTTTCAGACAATTC
TACCAGCTGGATGCCTATCCATCTGGTGCCTGGTATTACGTTCCACTAGGCACACA
ATACACTGATGCCCCATCATTCTCTGACATCCCTAATCCCATTGGCTCTGAGAACA
GTGAAAAGACTACTATGCCACTGTGGTGAGCTTGTTGTGGTTATCTGGTTGCGTCT
GTTGCCCGTTGTCTGTTGCCCATTGTGGTGGTTGTGTTTGTATGATGGTCGTTAAG
GATCATCAATGTGTTTTCGCTTTTTGTTCCATTCTGTTTCTCATTTGTGAATAATAA
TGGTATCTTTATGAATATGCAGTTTGTGGTTTCTTTTCTGATTGCAGTTCTGAGCAT
TTTGTTTTTGCTTCCGTTTACTATACCACTTACAGTTTGCACTAATTTAGTTGATAT
GCGAGCCATCTGATGTTTGATGATTCAAATGGCGTTTATGTAACTCGTACCCGAGT
GGATGGAGAAGAGCTCCATTGCCGGTTTGTTTCATGGGTGGCGGAGGGCAACTCC
TGGGAAGGAACAAAAGAAAAACCGTGATACGAGTTCATGGGTGAGAGCTCCAGC
TTGATCCCTTCTCTGTCGATCAAATTAAATTTTTGGATCACGGCAGGCTCACAAG
ATAATCCAAAGTAAAACATAATGAATAGTACTTCTCAATGATCACTTATTTTTAGC
AAATCAGCAATTGTGCATGTCAAATGATTTCGGTGTAAGAGAAAGAGTTGATGAA
TCAAAATATCTGTAGCTGGATCAAGAATCTGAGGCAGTTGTATGTATCAATGATCT
TTCCGCTACAATGATGTTAGCTATCCGAGTCAAATTGTTGTAGAATTGGATACTTC
GGCATCACATTCTGGATGACATAATAAATAGGAAGTCTTCAGATCCCTAAAAAAT
TGAGAGCTAATAACATTAGTCCTAGATGTAACTGGGTGACAACCAAGAAAGAGAC
ATGCAAATACTACTTTTGTTTGAAGGAGCATCCCTGGTTTGACATATTTTTTCTGA
ATATCAAACTTTGAAACTCTACCTAGTCTAATGTCTAACGACAGATCTTACTGGTT
TAACTGCAGTGATATCTACTATCTTTTGGAATGTTTTCTCCTTCAGTTATACATCAA
GTTCCAAGATGCAGGTGTGCTTGATTGATGTACATGGCTGTGAGAAGTGCATCCT
GATGTTCAGATGATGGTTCATTCTAATGTCTTTTCCTTCAATCAGTGTCTCAGTCT
GACTTAGCTTGTTTCATCTGCATGTTTGAATGTTCGTTTACTCATAGTAATTGCATT
TTTGTAGCAGAACATATCAATGGTCATGGTTTCAACTGTGCGCGAGTCTTATGCTT
ATTCAAACTAGGAAAGCCTCCGTCTAGAGGGTACACGAGTTGTTGCTCTGTGTGC
GTCAGTCCATAGTATTAATCTTGCTAGTTGTAGTATATTGTTTATGTGGACTCGGA
ATTCATCATATGCTCCTTCCTTTGCATCAAGTAAGGCAAGGTAATGTATAGAAGCTT
TTTAACTCTTTCATGGAAGCTGGCCTTTGCCAGCATACCATCCAGAAGATATCAAC
CCTGCATCTTGGCTGCCGCGCTGTCAGGAGGTCAACTACCCCAATTTAAATTTTAT
TTGATTAAGATATTTTTATGGACCTACTTTATAATTAAAAATATTTTCTATTTGAAA
AGGAAGGACAAAAATCATACAATTTTGGTCCAACTACTCATATCTTTTTTTTTTTG
GCTTTATAAAAAAGGAAAGTGATTAGTAATAAATAATTAAATAATGAAAAAAGG
AGGAAATAAAATTTTCGAATTAAAATGTAAAAGAGAAAAAGGAGAGGGAGTAAT
CATTGTTTAACTTTATCTAAAGTACCCCAATTCGATTTTACATGTATATCAAATTAT
ACAAATATTTTATTAAAATATAGATATTGAATAATTTTATTATTGCTTGAACATGTA
AATAAAAATTATCTATTATTTCAATTATTATATAAACTATTATTTGAAATCTCAATT
ATGATTTTTTAATATCACTTTCTATCCATGATAATTTCAGCTTAAAAAGTTTTGTCA
ATAATTACGTAATTTTGTTGATGAGGATGACAAGATTTCGGTCATCAATTACATA
TAGACAAATTGAAATAGTAAGCAACTTGATTTTTTTTCTCATAATGATAATGACAA
AGACACGAAAAGACAATTCAATATTCACATTGATTTATTTTTATATGATAATAATT
ACAATAATAATATTCTTATAAAGAAAGAGATCAATTTTGACTGATCCAAAAATTT
ATTTATTTTTACTATACCAACGTCACTAATTATATCTAATAATGTAAAACAATTCA
ATCTTACTTAAATATTAATTTGAAATAAACTATTTTTATAACGAAATTACTAAATT
TATCCAATAACAAAAAGGTCTTAAGAAGACATAAATTCTTTTTTTGTAATGCTCAA
ATAAATTTGAGTAAAAAAGAATGANATTGAGTGATTTTTTTTTAATCATAAGAAA
ATAAATAATTAATTTCAATATAATAAAACAGTAATATAATTTCATAAATGGAATTC
AATACTTACCTCTTAGMATAAAAAATAAATATAAAAAATAAAGTGTTTCTAATAA
ACCCGCAATTTAAATAAAATATTTAATATTTTCAATCAAATTTAAATAATTATATT
AAAATATCGTAGAAAAAGAGCAATATATAATACAAGAAAGAAGATTTATACA
ATTATCAACTATTATTATACTCTAATTTTGTTATATTTAATTTCTTACGGTTNAGGT
CATGTTCACGATAAACTCAAAATACGCTGTATGAGGACATATTTTAAATTTTAACC
AATAATAAAACTAAGTTATTTTTAGTATATTTTTTTGTTTAACGTGACTTAATTTTT
CTTTTCTAGAGGAGCGTGTAAGTGTCAACCTCATTCTCCTAATTTTCCCAACCACA
TAAAAAAAAAATAAAGGTAGCTTTTGCGTGTTGATTTGGTACACTACACGTCATT
ATTACACGTGTTTTCGTATGATTGGTTAATCCATGAGGCGGTTTCCTCTAGAGTCG
GCCATACCATCTATAAAATAAAGCTTTCTGCAGCTCATTTTTTCATCTTCTATCTGA
TTTCTATTATAATTTCTCTGAATTGCCTTCAAATTTCTCTTTCAAGGTTAGAATTT
TCTCTATTTTTTGGTTTTTGTTTGTTTAGATTCTGAGTTTAGTTNATCAGGTGCTGTT
AAAGCCCTAAATTTTGAGTTTTTTTCGGTTGTTTTGATGGAAAATACCTAACAATT
GAGTTTTTTTCATGTTGGCTTTTGTCGGAGAATGCCTACAATTGGAGTTCCTACGTTGT
TTTGATGAGAAAGCCCCTAATTTGAGTGTTTTTCCGTCGATTTGATTTTAAAGGTTT
ATATTCGAGTTTTTTTCGTCGGTTTAATGAGAAGGCCTAAAATAGGAGTTTTTATG
GTTGATTTGACTAAAAAAGCGATGGAATTTTGTGTTTTTGATGTCGCTTTGGTTCTC
AAGGCCTAAGATCTGAGTTTCTCCGGTTGTTTTGATGAAAAAGCCCTAAAATTGG
AGTTTTTATCTTGTGTTTTAGGTTGTTTTAATCCTTATAATTTGAGTTTTTTCGTTGT
TCTGATTGTTGTTTTTATGAATTTTGCAGAATGAAGTTCTTCATCTTTACCTGCCTT
TTGGCTGTTGCCGTGCAAAGAATACGATGGAACATGTCTCCTCCAGTGAGGAAT
CTATGTATCTCCCAGGAAACATATAAGCAGGAAAAGAATATGGACATTAATCCCAG
CAAGGAGAACCTTTGCTCCACATTCTGCAAGGAAGTTGTAAGGAACGCAAATGAA
GAGGAATATTCTATCGGCTCATCTAGTGAGGAATCTGCTGAAGTTGCCACAGAGG
AAGTTAAGATTACTGTGGACGATAAGCACTACCAGAAAGCACTGAATGAAATCAA
TCAGTTTTATCGGAAGTTCCCCCAGTATCTCCAGTATCTGTATCAAGGTCCAATTG
TTTTGAACCCATGGGATCAGGTTAAGAGAAATGCTGTTCCCATTACTCCCACTCTG
AACAGAGAGCAGCTCTCCACCAGTGAGGAAAATTCAAAGAAGACCGTTGACATG
GAATCAACAGAAGTATTCACTAAGAAAACTAAACTGACTGAAGAAGAAAAGAAT
CGCCTAAATTTTCTGAAAAAAATCAGCCAGCGTTACCAGAAATTCGCCTTGCCCC
AGTATCTCAAAACTGTTTATCAGGATCAGAAAGCTATGAAGCCATGGATTCAACC
TAACTACAAAGGTTATTCCCTATGTGAGGTACCTTTAAGCTTGTTGTGGTTGTCTGG
TTGCGTCTGTTGCCCGTTGTCTGTTGCCCATTGTGGTGGTTGTGTTTGTATGATGGT
CGTTAAGGATCATCAATGTGTTTTCGCTTTTTGTTCCATTCTGTTTCTCATTTGTGA
ATAATAATGGTATCTTTATGAATATGCAGTTTGTGGTTTCTTTTCTGATTGCAGTTC
TGAGCATTTTGTTTTTGCTTCCGTITACTATACCACTTACAGTTTGCACTAATTIAG
TTGATATGCGAGCCATCTGATGTTTGATGATTCAAATGGCGTTTATGTAACTCGTA
CCCGAGTGGATGGAGAAGAGCTCCATTGCCGGTTTGTTTCATGGGTGGCGGAGGG
CAACTCCTGGGAAGGAACAAAAGAAAAACCGTGATACGAGTTCATGGGTGAGAG
CTCCAGCTTGATCCCTTCTCTGTCGATCAAATTTGAATTTTTGGATCACGGCAGGC
TCACAAGATAATCCAAAGTAAAACATAATGAATAGTACTTCTCAATGATCAATA
TTTTTAGCAAATCAGCAATTGTGCATGTCAAATGATTTCGGTGTAAGAGAAAGAG
TTGATGAATCAAAATATCTGTAGCTGGATCAAGAATCTGAGGCAGTTGTATGTAT
CAATGATCTTTCCGCTACAATGATGTTAGCTATCCGAGTCAAATTGTTGTAGAATT
GCATACTTCGGCATCACATTCTGGATGACATAATAAATAGGAAGTCTTCAGATCC
CTAAAAAATTGAGAGCTAATAACATTAGTCCTAGATGTAACTGGGTGACAACCAA
GAAAGAGACATGCAAATACTACTTTTGTTTGAAGGAGCATCCCTGGTTTGACATA
TTTTTTCTGAATATCAAACTTTGAAACTCTACCTAGTCTAATGTCTAACGACAGAT
CTTACTGGTTTAACTGCAGTGATATCTACTATCTTTTGGAGTGTTTTCTCCTTCAGT
TATACATCAAGTTCCAAGATGCAGGTGTGCTTGATTGATGTACATGGCTGTGAGA
AGTGCATCCTGATGTTCAGATGATGGTTCATRTAATGTCTTTTCCTTCAATCAGTT
TTCTCAGTCTGACTTAGCTTGTTTCATCTGATGTTTGAATGTTCGTTTACTCATAG
TAATTGCATTTTTGTAGCAGAACATATCATTGGTCATGGTTTCAACTGTGCGCGAG
TCTTATGCTTATTCAAACTAGGAAAGCCTCCGTCTAGAGGGTACACGAGTTGTTGC
TCTGTGTGCGTGAGTCCATAGTATTAATCTTGCTAGTTGTAGTATATTGTTTATGTG
GACTCGGAATTCATCATATGCTCCTTCTTTGCATCAAGTAAGGCAAGGTAATGTAT
AGNAGCTTTTTAACTCTTTCATGGAAGCTGGCCTTTGCCAGCATACCATCCAGAAG
ATATCAACCCTGCATCTTGGCTGCCGCGCTGTCAGGAGGTCAACTACCCCAATTTA
AATTTTATTTGATTAAGATATTTTTATGGACCTACTTTATAATTAAAAATATTTTCT
ATTTGAAAAGGAAGGACAAAAATCATACAATTTTGGTCCAACTACTCCTCTCTTTT
TTTTTTTGGCTTTATAAAAAAGGAAAGTGATTAGTAATAAATAATTAAATAATGA
AAAAAGGAGGAAATAAAATTTTCGAATTAAAATGTAAAAGAGAAAAAGGAGAGG
GAGTAATCATTGTTTAACTTTATCTAAAGTACCCCAATTCGATTTTACATGTATAT
CAAATTATACAAATATTTTATTAAAATATAGATATTGAATAATTTTATTATTCTTG
AACATGTAAATAAAAATTATCTATTATTTCAATTTTTATATAAACTATTATTTGAA
ATCTCAATTATGATTTTTTAATATCACTTTCTATCCATGATAATTTCAGCTTAAAAA
GTTTTGTCAATAATTACATTAATTTTGTMATGAGGATGACAAGATTTCGGTCATC
AATTACATATACACAAATTGAAATAGTAAGCAACTTGATTTTTTTTCTCATAATGA
TAATGACAAAGACACGAAAAGACAATTCAATATTCACATTGATTTATTTTTATATG
ATAATAATTACAATAATAATATTCTTATAAAGAAAGAGATCAATTTTGACTGATCC
AAAAATTTATTTATTTTTACTATACCAACGTCACTAATTATATCTAATAATGTAAA
ACAATTCAATCTTACTTAAATATTAATTTGAAATAAACTATTTTTATAACGAAATT
ACTAAATTTATCCAATAACAAAAAGGTCTTAAGAAGACATAAATTCTTTTTTTGTA
ATGCTCAAATAAATTTGAGTAAAAAAGAATGAAATTGAGTGATTTTTTTTTAATCA
TAAGAAAATAAATAATTAATTTCAATATAATAAAACAGTAATATAATTTCATAAA
TGGAATTCAATACTTACCTCTTAGATATAAAAAATAAATATAAAAATAAAGTGTT
TCTAATAAACCCGCAATTTAAATAAAATATTTAATATTTTCAATCAAATTTAAATA
ATTATATTAAAATATCGTAGAAAAAGAGCAATATATAATACAAGAAAGAAGATTT
AAGTACAATTATCAACTATTATTATACTCTAATTTTGTTATATTTAATTTCTTACGG
TTAAGGTCATGTTCACGATAAACTCAAAATACGCTGTATGAGGACATATTTTAAAT
TITAACCAATAATAAAACTAAGTTATTTTTAGTATATTTTTTTGTTTAACGTGACTT
AATTTTTCTTTTCTAGAGGAGCGTGTAAGTGTCAACCTCATTCTCCTAATTTTCCCA
ACCACATAAAAAAAAAATAAAGGTAGCTTTTGCGTGTTGATTTGGTACACTACAC
GTCATTATTACACGTGTTTTCGTATGATTGGTTAATCCATGAGGCGGTTTCCTCTA
GAGTCGGCCATACCATCTATAAAATAAAGCTTTCTGCAGCTCATTTTTTCATCTTC
TATCTGATTTCTATTATAATTTCTCTGAATTGCCTTCAAATTTCTCTTTCAAGGTTA
GAATTTTTCTCTATTTTTTGGTTTTTGTTTGTTTAGATTCTGAGTTTAGTTAATCAGG
TGCTGTTAAAGCCCTAAATTTTGAGTTTTTTTCGGTTGTTTTGATGGAAAATACCTA
ACAATTGAGTTTTTTCATGTTGTTTTGTCGGAGAATGCCTACAATTGGAGTTCCTTT
CGTTGTTTTGATGAGAAAGCCCCTAATTTGAGTGTTTTTCCGTCGATTTGATTTTAA
AGGTTTATATTCGAGTTTTTTTCGTCGGTTTAATGAGAAGGCCTAAAATAGGAGTT
TTTCTGGTTGATTTGACTAAAAAAGCCATGGAATTTTGTGTTTTTGATGTCGCTTTG
GTTCTCAAGGCCTAAGATCTGAGTTTCTCCGGTTGTTTTGATGAAAAAGCCCTAAA
ATTGGAGTTTTTATCTTGTGTTTTAGGTTGTTTTAATCCTTATAATTTGAGTTTTTTC
GTTGTTCTGATTGTTGTTTTTATGAATTTTGCAGAATGAAGGTCCTCATCCTTGCCT
GCCTGGTGGCTCTGGCCCTTGCAAGAGAGCTGGAAGAACTCAATGTACCTGGTGA
GATTGTGGAAAGCCTTTCAAGCAGTGAGGAATCTATTACACGATCAATAAGAAA
ATTGAGAAGTTTCAGAGTGAGGAACAGCAGCAAACAGAGGATGAACTCCAGGAT
AAAATCCACCCCTTTGCCCAGACACAGTCTCTAGTCTATCCCTTCCCTGGGCCCAT
CCATAACAGCCTCCCACAAAACATCCCTCCTCTTACTCAAACCCCTGTGGTGGTGC
CGCCTTTCCTTCAGCCTGAAGTAATGGGAGTCTCCAAAGTGAAGGAGGCTATGGC
TCCTAAGCACAAAGAAATGCCCTTCCCTAAATATCCAGTTGACCGTTACTGAAA
GGCAGAGCCTGACTCTCACTGATGTTGAAAATCTGCACCTTCCTCTGCCTCTGCTC
CAGTCTTGGATGCACCAGCCTCACCAGCCTCTTCCTCCAACTGTCATGTTTCCTCC
TCAGTCCGTGCTGTCCCTTTCTCAGTCCAAAGTCCTGCGTCTTTCCCCAGAAAGCAG
TGCCCTATCCCCAGAGAGATATGCCCATTCAGGCCTTTCTGCTGTACCAGGAGCCT
GTACTCGGTCCTGTCCGGGGACCCTTCCCTATTATTGTCTAAGCTTGTTGTGTGGTGT
CTGGTTGCGTCTGTTGCCCGTTGTCTGTTGCCCATTGTGGTGGTTGTGTTTGTATGA
TGGTCGTTAAGGATCATCAATGTGTTTTCGCTTTTTGTTCCATTCTGTTTCTCATTT
GTGAATAATAATGGTATCITTATGAATATGCAGTTTGTGGTTTCTTTTCTGATTGCA
GTTCTGAGCATTTTGTTTTTGCTTCCGTTTACTATACCACTTACAGTTTGCACTAAT
TTAGTTGATATGCGAGCCATCTGATGTTTGATGATTCAAATGGCGTTTATGTAACT
CGTACCCGAGTGGATGGAGAAGAGCTCCATTGCCGGTTTGTTTCATGGGTGGCGG
AGGGCAACTCCTGGGAAGGAACAAAAGAAAAACCGTGATACGAGTTCATGGGTG
AGAGCTCCAGCTTGATCCCTTCTGTCGATCAAATTTGAATTTTTGGATCACGGC
AGGCTCACAAGATAATCCAAAGTAAAACATAATGAATAGTACTTCTCAATGATTCA
CTTATTTTTAGCAAATCAGCAATTGTGCATGTCAAATGATTTCGGTGTAAGAGAAA
GAGTTGATGAATCAAAATATCTGTAGCTGGATCAAGAATCTGAGGCAGTTGTATG
TATCAATGATCTTTCCGCTACAATGATGTTAGCTATCCGAGTCAAATTGTTGTAGA
ATTGCATACTTCGGCATCACATTCTGGATGACATAATAAATAGGAAGTCTTCAGAT
CCCTAAAAAATTGAGAGCTAATAACATTAGTCCTAGATGTAACTGGGTGACAACC
AAGAAAGAGACATGCAAATACTACTTTTGTTTGAAGGAGCATCCCTGGTTTGACA
TATTTTTTCTGAATATCAAACTTTGAACTCTACCTAGTCTAATGTCTAACGACAG
ATCTTACTGGTTTAACTGCAGTGATATCTACTATCTTTTGGAATGTTTTCTCCTTCA
GTTATACATCAAGTTCCAAGATGCAGGTGTGCTTGATTGATGTACATGGCTGTGAG
AAGTGCATCCTGATGTTCAGATGATGGTTCATTCTAATGTCTTTTCCTTCAATCAGT
TTTCTCAGTCTGACTTAGCTTGTTTGATCTGCATGTTTGAATGTTCGTTTACTCATA
GTAATTGCATTTTTGTAGCAGAACATATCATTGGTCATGGTTTCAACTGTGCGCGA
GTCTTATGCTTATTCAAACTAGGAAAGCCTCCGTCTAGAGGGTACACGAGTTGTTG
CTCTGTGTGCGTCAGTCCATAGTATTAATCTTGCTAGTTGTAGTATATTGTTTATGT
GGACTCGGAATTCATCATATGCTCCTTCTTGCATCAAGTAAGGCAAGGTAATGTA
TAGAAGCTTTTTAACTCTTTCATGGAAGCTGGCCTTTGCCAGCATACCATCCAGAA
GATATCAACCCTGCATCTTGGCTGCCGCGCTGTGATGAGACCGGATCCTGACAGG
ATATATTGGCGGGTAAACCTAAGAGAAAAGAGCGTTTATTAGAATAATCGGATAT
TTAAAAGGGCGTGAAAAGGTTTATCCGTTCGTCCATTTGTATGTTGCATGCCAACCA
CAGGGTTCCCCTCGGGATCAAAGTACTTTGATCCAACCCCTCCGCTGCTATAGTGC
AGTCGGCTTCTGACGTTCAGTGCAGCCGTCATCTGAAAACGACATGTCGCACAAG
TCCTAAGTTACGCGACAGGCTGCCGCCCTGCCCTTTTCCTGGCGTTTTCTTGTCGC
GTGTTTTAGTCGCATAAAGTAGAATACTTGCGACTAGAACCGGAGACATTACGCC
ATGAACAAGAGCGCCGCCGCTGGCCTGCTGGGCTATGCCCGCGTCAGCACCGACG
ACCAGGACTTGACCAACCAACGGGCCGAACTGCACGCGGCCGGCTGCACCAAGC
TGTTTTCCGAGAAGATCACCGGCACCAGGCGCGACCGCCCGGAGCTGGCCAGGAT
GCTTGACCACCTACGCCCTGGCGACGTTGTGACAGTGACCAGGCTAGACCGCCTG
GCCCGCAGCACCCGCGACCTACTGGACATTGCCGAGCGCATCCAGGAGGCCGGCG
CGGGCCTGCGTAGCCTGGCAGAGCCGTGGGCCGACACCACCACGCCGGCCGGCC
GCATGGTGTTGACCGTGTTCGCCGGCATTGCCGAGTTCGAGCGTTCCCTAATCATC
GACCGCACCCGGAGCGGGCGCGAGGCCGCCAAGGCCCGAGGCGTGAAGTTTGGC
CCCCGCCCTACCCTCACCCCGGCACAGATCGCGCACGCCCGCGAGCTGATCGACC
AGGNAGGCCGCACCGTGAAAGAGGCGGCTGCACTGCTTGGCGTGCATCGCTCGAC
CCTGTACCGCGCACTTGAGCGCAGCGAGGAAGTGACGCCCACCGAGGCCAGGCG
GCGCGGTGCCTTCCGTGAGGACGCATTGACCGAGGCCGACGCCCTGGCGGCCGCC
GAGAATGAACGCCAAGAGGAACAAGCATGAAACCGCACCAGGACGGCCAGGAC
GAACCGTTTTTCATTACCGAAGAGATCGAGGCGGAGATGATCGCGGCCGGGTACG
TGTTCGAGCCGCCCGCGCACCTCTCAACCGTGCGGCTGCATGAAATCCTGGCCGG
TTTGTCTGATGCCAAGCTGGCGGCCTGGCCGGCCAGCTTGGCCGCTGAAGAAACC
GAGCGCCGCCGTCTAAAAAGGTGATGTGTATTTGAGTAAAACAGCTTGCGTCATG
CGGTCGCTGCGTATATGATCCGATGAGTAAATAAACAAATAGCAAGGGGAACGC
ATGAAGGTTATCGCTGTACTTAACCAGAAAGGCGGGTCAGGCAAGACGACCATCG
GAACCCATCTAGCCCGCGCCCTGCAACTCGCCGGGGCCGATGTTCTGTTAGTCGA
TTCCGATCCCCAGGGCAGTGCCCGCGATTGGGCGGCCGTGCGGGAAGATCAACCG
CTAACCGTTGTCGGCATCGACCGCCCGACGATTGACCGCGACGTGAAGGCCATCG
GCCGGCGCGACTTCGTAGTGATCGACGGAGCGCCCCAGGCGGCGGACTTGGCTGT
GTCCGCGATCAAGGCAGCCGACTTCGTGCTGATTCCGGTGCAGCCAAGCCCTTAC
GACATATGGGCCACCGCCGACCTGGTGGAGCTGGTTAAGCACGGCATTGAGGTCA
CGGATGGAAGGCTACAAGCGGCCTTTGTCGTGTCGCGGGCGATCAAAGGCACGCG
CATCGGCGGTGAGGTTGCCGAGGCGCTGGCCGGGTACGAGCTGCCCATTCTTGAG
TCCCGTATCACGCAGCGCGTGAGCTACCCAGGCACTGCCGCCGCCGGCACAACCG
TTCTTGAATCAGAACCCGAGGGCGACGCTGCCCGCGAGGTCCAGGCGCTGGCCGC
TGAAATTAAATCAAAACTCATTTGAGTTAATGAGGTAAAGAGAAAATGAGCAAA
AGCACAAACACGCTAAGTGCCGGCCGTCCGAGCGCACGCAGCAGCAAGGCTGCA
ACGTTGGCCAGCCTGGCAGACACGCCAGCCATGAAGCGGGTCAACTTTCAGTTGC
CGGCGGAGGATCACACCAAGCTGAAGATGATGTACGCGGTACGCCAAGGCAAGACCA
TTACCGAGCTGCTATCTGAATAGATCGCGCAGCTACCAGAGTAAATGAGCAAATG
AATAAATGAGTAGATGAATTTTAGCGGCTAAAGGAGGCGGCATGGAAAATCAAG
AACAACCAGGCACCGACGCCGTGGAATGCCCCATGTGTGGAGGAACGGGCGGTT
GGCCAGGCGTAAGCGGCTGGGTTGTCTGCCGGCCCTGCAATGGCACTGGAACCCC
CAAGCCCGAGGAATCGGCGTGACGGTCGCAAACCATCCGGCCCGGTACAAATCG
GCGCGGCGCTGGGTGATGACCTGGTGGAGAAGTTGAAGGCCGCGCAGGCCGCCC
AGCGGCAACGCATCGAGGCAGAAGCACGCCCCGGTGAATCGTGGCAAGCGGCCG
CTGATCGAATCCGCAAAGAATCCCGGCAACCGCCGGCAGCCGGTGCGCCGTCGAT
TAGGAAGCCGCCCAAGGGCGACGAGCAACCAGATTTTTTCGTTCCGATGCTCTAT
GACGTGGGCACCCGCGATAGTCGCAGCATCATGGACGTGGCCGTTTTCCGTCTGT
CGAAGCGTGACCGACGAGCTGGCGAGGTGATCCGCTACGAGCTTCCAGACGGGC
ACGTAGAGGTTTCCGCAGGGCCGGCCGGCATGGCCAGTGTGTGGGATTACGACCT
GGTACTGATGGCGGTTTCCCATCTAACCGAATCCATGAACCGATACCGGGAAGGG
AAGGGAGACAAGCCCGGCCGCGTGTTCCGTCCACACGTTGCGGACGTACTCAAGT
TCTGCCGGCGAGCCGATGGCGGAAAGCAGAAAGACGACCTGGTAGAAACCTGCA
TTCGGTTAAACACCACGCACGTTCCATTCCGAGCGTACGAAGAAGGCCAAGAACG
GCCGCCTGGTGACGGTATCCGAGGGTGAAGCCTTGATTAGCCGCTACAAGATCGT
AAAGAGCGAAACCGGGCGGCCGGAGTACATCGAGATCGAGCTAGCTGATTGGAT
GTACCGCGAGATCACAGAAGGCAAGAACCCGGACGTGCTGACGGTTCACCCCGA
TTACTTTTTGATCGATCCCGGCATCGGCCGTTTTCTCTACCGCCTGGCACGCCGCG
CCGCAGGCAAGGCAGAAGCCAGATGGTTGTTCAAGACGATCTACGAACGCAGTG
GCAGCGCCGGAGAGTTCAAGAAGTTCTGTTTCACCGTGCGCAAGCTGATGGGTC
AAATGACCTGCCGGAGTACGATTTGAAGGAGGAGGCGGGGCAGGCTGGCCCGAT
CCTAGTCATGCGCTACCGCAACCTGATCGAGGGCGAAGCATCCGCCGGTTCCTAA
TGTACGGAGCAGATGCTAGGGCAAATTGCCCTAGCAGGGGAAAAAGGTCGAAAA
GGACTCTTTCCTTGTGGATAGCACGTACATTGGGAACCCAAAGCCGTACATTGGGA
ACCGGAACCCGTACATTTGGGAACCCAAAGCCGTACATTGGGAACCGGTCACACT
GTAAGTGACTGATATAAAAGAGAAAAAAGGCGATTTTTCCGCCTAAAACTCTTTA
AAACTTATTAAAACTCTTAAAACCCGCCTGGCCTGTGCATAACTGTCTGGCCAGCG
CCATAACAGCCTCCCACAAAACATCCCTCCTCTTACTCAAACCCCTGTGGTGGTGC
CGCCTTTCCTTCAGCCTGAAGTAATGGGAGTCTCCAAAGTGAAGGAGGCTATGGC
TACGGCCAGGCAATCTACCAGGGCGCGGACAAGCCGCGCCGTCGCCACTCGACCG
CCGGCGCCCACATCAAGGCACCCTGCCTCGCGCGTTTCGGTGATGACGGTGAAAA
CCTCTGACACATGCAGCTCCCGGTGACGGTCACAGCTTGTCTGTAAGCGGATGCC
GGGAGCAGACAAGCCCGTCAGGGCGCGTCAGCGGGTGTTGGCGGGTGTCGGGGC
GCAGCCATGACCCAGTCACGTAGCGATAGCGGAGTGTATACTGGCTTAACTATGC
GGCATCAGAGCAGATTGTACTGAGAGTGCACCATATGCGGTTTGAAATACCGCAC
AGATGCGTAAGGAGAAAATACCGCATCAGGCGCTCTTCCGCTTCCTCGCTCACTG
ACTCGCTGCGCTCGGTCGTTCGGCTGCGGCGAGCGGTATCAGCTCACTCAAAGGC
GGTAATACGGTTATCCACAGAATTCAGGGGATTAACGCAGGAAAGAACATGTGAGC
AAAAGGCCAGCAAAAGGCGAGGNACCGTAAAAAGGCCGCGTTGCTGGCGTTTTTC
CATAGGCTCCGCCCCCCTGACGAGCATCACAAAAATCGACGCTCAAGTCAGAGGT
GGCGAAACCCGACAGGACTATAAAGATACCAGGCGTTTTCCCCCTGGAAGCTCCCT
CGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGT
AGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGGACAGT
ATTTGGTATCTGCGCTCTGCTGAAGCCAGTTACCTTCGGAAAAAGAGTTGGTAGCT
CTTGATCCGGCAAACAAACCACCGCGGAGCGGTGGTTTTTTTGTTTGCAAGCAG
CAGATTACGCGCAGAAAANAAGGATCTCAAGAAGATCCTTTGATCTTTTCTACGG
GGTCTGACGCTCAGTGGAACGAAAACTCACGTTAAGGGATTTTGGTCATGCATTC
TAGGTGATTATTTGCCGACTACCTTGGTGATCTCGCCTTTCAGTAGTGGACAAAT
TCTTCCAACTGATCTGCGCGCGAGGCCAAGCGATCTTCTTCTTGTCCAAGATAAGC
CTGTCTAGCTTCAAGTATGACGGGCTGATACTGGGCCGGCAGGCGCTCCATTGCC
CAGTCGGCAGCGACATCCTTCGGCGCGATTTTGCCGGTTACTGCGCTGTACCAAAT
GCGGGACAACGTAAGCACTACATTTCGCTCATCACCAGCCCAGTCGGGCGGCGAG
TTCCATAGCGTTAAGGTTTCATTTAGCGCCTCAAATACATCCTGTTCAGGAACCGG
ATCAAAGAGTTCCTCCGCCGCTGGACCTACCAAGGCAACGCTATGTTCTCTTGCTT
TTGTCAGCAAGATAGCCAGATCAATGTCGATCGTGGGTGGGTCGAAGATACCGC
AAGAATGTCATTGCGCTGCCATTCTCCAAATTGCAGTTCGCGCTTACGTGGATAAC
GCCACGGAATGATGTCGTCGTGCACAACAATGGTGACTTCTACAGCGCGGAGAAT
CTCGCTCTCTCCAGGGGAAGCCGAAGTTTCCAAAAGGTCGTTGATCAAAGCTCGC
CGCGTTGTTTCATCAAGCCTTACGGTCACCGTAACCAGCAAATCAATATCACTGTG
TGGCTTCAGGCCGCCATCCACTGCGGAGCCGTACAAATGTACGGCCAGCAACGTC
GGTTCGAGATGGCGCTCGATGACGCCAACTACCTCTGATAGTTGAGTCGATACTTC
GGCGATCACCGCTTCCCTCATAATGTTTAACTTTGTTTTAGGGCGACTGCCCTGCT
GCGTAACATCGTTGCTGCTCCATAACATCAAACATCGACCCACGGCGTAACGCGC
TTGCTGCTTGGATGCCCGAGGCATAGACTGTACCCCAAAAAAACAGTCATAACAA
GCCATGAAAACCGCCACTGCGCCGTTACCACCGCTGCGTTCGGTCAAGGTTCTGG
ACCAGTGTCGTGAGCGCATACGCTACTTGCATTACAGCTTACGAACCGAACAGGC
TTATGTCCACTGGGTTCCAGCCTTCATCCGTTTCCACGGTGTGCGTTCACCCGGCAA
CCTTGGGTAGCAGCGAAGTCGAGGCATTTCTGTCCTGGCTGGAACAGAACTTATT
ATTTCCTTCCTCTTTTCTACAGTATTTAAAGATACCCCAAGAAGCTAATTATAACA
AGACGAACTCCAATTCACTGTTCCTTGCATTCTAAAACCTTAAATACCAGAAAAC
AGCTTTTTCAAAGTTGTTTTCAAAGTTGGCGTATAACATAGTATCGACGGAGCCGA
TTTTGAAACCGCGGTGATCACAGGCAGCAACGCTCTGTCATCGTTACATTRAACA
TGCTACCCTCCGCGAGATCATCCGTGTTTCAAACCCGGCAGCTTAGTTGCCGTTCT
TCCGAATAGCATCGGTAACATGAGCAAAGTCTGCCGCCTTACAACGGCTCTCCCG
CTGACGCCGTCCCGGACTGATGGGCTGCCTGTATCGAGTGGTGATTTTGTGCCGAG
CTGCCGGTCGGGGSGCTGTTGGCTGGCTGGTGGCAGGATATATTGTGGTGTAAAC
ATAACGGATCCGGTCTCAGGAGAGCGATCAGCTTGCATGCCGGTCGATCTAGTAA
CATAGTAGATGACACCGCGCGCGATAATTTATCCTAGTTTGCGCGCTATATTTTGT
TTTCTATCGCGTATTAAATGTATAATTGCGGGACTCTAATCATAAAAACCCATCTC
ATAAATAACGTCATGCATTACATGTTAATTATTACATGCTTAACGTAATTCAACAG
AAATTATATGATAATCATCGCAAGACCGGCAACAGGATTCAATCTTAAGAAACTT
TATTGCCAAATGTTTGAACGATCTGCTTGACTCTAGGGGTCATCAGATTTCGGTGA
CGGGCAGGACCGGACGGGGCGGCACCGGCAGGCTGAAGTCCAGCTGCCAGAAAC
CCACGTCATGCCAGTTCCCGTGCTTGAAGCCGGCCGCCCGCAGCATGCCGCGGGG
GGCATATCCGAGCGCCTCGTGCATGCGCACGCTCGGGTCGTTGGGCAGCCCGATG
ACAGCGACCACGCTCTTGAAGCCCTGTGCCTCCAGGGACTTCAGCAGGTGGGTGT
AGAGCGTGGAGCCCAGTCCCGTCCGCTGGTGGCGGGGGGATACGTACACGGTCG
ACTCGGCCGTCCAGTCGTAGGCGTTGCGTGCCTTCCAGGGACCCGCGTAGGCGAT
GCCGGCGACCTCGCCGTCCACCTCGGCGACGAGCCAGGGATAGCGCTCCCGCAGA
CGGACGGTCGTCCGTCCACTCCTGCGTTCCTGCGGTTCCTGCGGCTCGGTACGGAAGTT
GACCGTGCTTGTCTCGATGTAGTGGTTGACGATGGTGCAGACCGCCGGCATGTCCGCC
TCGGTGGCACGGCGGATGTCGGCCGGGCGTCGTTCTGGGCTCATGGTAGATCCCC
TCGATCGACTTTGAGAGTGAATATGAGACTCTAATTGGATACCGAGGGGAGTTTAT
GGAACGTCAGTGGAGCATTTTTGAGAAGAAATATTTGCTAGCTGATAGTGACCTT
AGGCGACTTTTGAACGCGCAATAATGGTTTCTGACGTATGTGCTTAGCTCATTAAA
CTCCAGAAACCCGCGGCTCACTTGGCTCCTTCAACGTTGCGCTTTCTCTTCAGTTCCAA
ACGTAAAACGGCTTGTCCCGCGTCATCGGCGGGGGTCATAACGTGACTCCCTTAA
TTCTCATGTATGATACTCCGTCAGGAGGTCAACTACCCCAATTTAAATTTTATTTG
ATTAAGATATTTTTATGGACCTACTTTATAATTAAAAATATTTTCTATTTGAAAACT
GAAGGACAAAAATCATACAATTTTGGTCCAACTACTCCTCTCTTTTTTTTTTTGGCT
TTATAAAAAAGGAAAGTGATTAGTAATAAATAATTAAATAATGAAAAAAGGAGG
ANATAAAATTTTCGAATTANAATGTAAAAGAGAAAAAGGAGAGGGAGTAATCAT
TGTTTAACTTTATCTAAAGTACCCCAATTCGATTTTACATGTATATCAAATTATAC
AAATATITTATTAAAATATAGATATTGAATAATTTTATTATTCTTGAACATGTAAA
TAAAAATTATCTATTATTTCAATTTTTATATAAACTATTATTTGAAATCTCAATTAT
GATTTTTTAATATCACTTTCTATCCATGATAATTTCAGCTTAAAAACTTTTTGTCAAT
AATTACATTAATTTTGTTGATGAGGATGACAAGATTTCGGTCATCAATTACATATA
CACAAATTGAAATAGTAAGCAACTTGATTTTTTTTCTGATAATGATAATGACAAAG
ACACGAAAAGACAATTCAATATTCACATTGATTTATTTTTATATGATAATAATTAC
AATAATAATATTCTTATAAAGAAAGAGATCAATTTTGACTGATCCAAAAATTTATT
TATTTTTACTATACCAACGTCACTAATTATATCTAATAATGTAAAACAATTCAATC
TTACTTAAATATTAATTTGAAATAAACTATITTTATAACGAAATTACTAAATTTAT
CCAATAACAAAAAGGTCTTAAGAAGACATAAATTCTTTTTTTGTAATGCTCAAATA
AATTTGAGTAAAAAAGAATGAAATTGAGTGATTTTTTTTTAATCATAAGAAAATA
AATAATTAATTTCAATATAATAAAACAGTAATATAATTTCATAAATGGAATTCAAT
ACTTACCTCTTAGATATAAAAAATAAATATAAAAATAAAGTGTTTCTAATAAACC
CGCAATTTAAATAAAATATTTAATATTTTCAATCAAATTTAAATAATTATATTAAA
ATATCGTAGAAAAAGAGCAATATATAATACAAGAAACTAAGATTTAAGTACAATT
ATCAACTATTATTATACTCTAATTTTGTTATATTTAATTTCTTACGGTTAAGGTCAT
GTTCACGATAAACTCAAAATACGCTGTATGAGGACATATTTTAAATTTTAACCAAT
AATAAAACTAAGTTATTTTTAGTATATTTTTTTGTTTAACGTGACTTAATTTTTCTT
TTCTAGAGGAGCGTGTAAGTGTCAACCTCATTCTCCTAATTTTCCCAACCACATAA
AAAAAAAATAAAGGTAGCTTTTGCGTGTTGATTTGGTACACTACACGTCATTATTA
CACGTGTTTTCGTATGATTGGTTAATCCATGAGGCGGTTTCCTCTAGAGTCGGCCA
TACCATCTATAAAATAAAGCTTTCTGCAGCTCATTTTTTCATCTTCTATCTGATTTC
TATTATAATTTCTCTGAATTGCCTTCAAATTTCTCTTTCAAGGTTAGAATTTTTCTC
TATTTTTTGGTTTTTGTTTGTTTAGATTCTGAGTTTAGTTAATCAGGTGCTGTTAAA
GCCCTAAATTTTGAGTTTTTTTCGGTTGTTTTGATGGAAAATACCTAACAATTGAG
TTTTTTCATGTTTGTTTTGTCCTGAGAATGCCTACAATTGGAGTTCCTTTCGTTGTTTT
GATGAGAAAGCCCCTAATTTGAGTGTTTTTCCGTCGATTTGATTTTAAAGGTTTAT
ATTCGAGTTTTTTTCCTTCGCTTTTAATGAGAACTGCCTAAAATAGGAGTTTTTCTGGT
TGATTTGACTAAAAAAGCCATGGAATTTTGTGTTTTTGATGTCGCTTTGGTTCTCA
AGGCCTAAGATCTGAGTTTCTCCGGTTGTTTTGATGAAAAAGCCCTAAAATTGGA
GTTTTTATCTTGTGTTTTAGGTTGTTTTAATCCTTATAATTTGAGTTTTTTCGTTGTT
CTGATTGTTGTTTTTATGAATTTTGCAGAATGATGTCCTTTGTCTCTCTGCTCCTGG
TAGGCATCCTATTCCATGCCACCCAGGCTGAACAGTTAACAAAATGTGAGGTGTT
CCGGGAGCTGAAAGACTTGAAGGGCTACGGAGGTGTCAGTTTGCCTGAATGGGTC
TGTACCACGTTTCATACCAGTGGTTATGACACACAAGCCATAGTACAAAACAATG
ACAGCACAGAATATGGACTCTTCCAGATAAATAATAAAATTTGGTGCAAAGACGA
CCAGAACCCTCACTCAAGCAACATCTGINACATCTCCTGIGACAAGTTCCTGGAT
GATGATCTTACTGATGACATTATGTGTGTCAAGAAGATTCTGGATAAAGTAGGAA
TTAACTACTGGTTGGCCCATAAAGCACTCTGTTCTGAGAAGCTGGATCAGTGGCTC
TGTGAGAAGTTGTGAGCTTGTTGTGGTTGTCTGGTTGCGTCTGTTGCCCGTTGTCT
GTTGCCCATTGTGGTGGTTGTGTTTGTATGATGGTCGTTAAGGATCATCAATGTGT
TTTCGCTTTTTGTTCCATTCTGTTTCTCATTTGTGAATAATAATGGTATCTTTATGA
ATATGCAGTTTGTGGTTTCTTTTCTGATTGCAGTTCTGAGCATTTTGTTTTTGCTTC
CGTTTACTATACCACTTACAGTTTGCACTAATTTAGTTGATATGCGAGCCATCTGA
TGTTTGATGATTCAAATGGCGTTTATGTAACTCGTACCCGAGTGGATGGAGAAGA
GCTCCATTGCCGGTTTGTTTCATGGGTGGCGGAGGGCAACTCCTGGGAAGGAACA
AAAGAAAAACCGTGATACGAGTTCATGGGTGAGAGCTCCAGCTTGATCCCTTCTC
TGTCGATCAAATTTGAATTTTTGGATCACGGCAGGCTCACAAGATAATCCAAAGT
AAAACATAATGAATAGTACTTCTCAATGATCACTTATTTTTAGCAAATCAGCAATT
GTGCATGTCAAATGATTTCGGTGTAAGAGAAAGAGTTGATGAATCAAAATATCTG
TAGCTGGATCAAGAATCTGAGGCAGTTGTATGTATCAATGATCTTTCCGCTACAAT
GATGTTAGCTATCCGAGTCAAATTGTTGTAGATTGCGGCACTTCGGCGTCACATTC
TGGATGACATAATAAATAGGAAGTCTTCAGATCCCTAAAAAATTGAGAGCTAATA
ACATTAGTCCTAGATGTAACTGGGTGACAACCAAGAAAGAGACATGCAAATACTA
CTTTTGTTTGAAGGAGCATCCCTGGTTTGACATATTTTTTCTGAATATCAAACTTTG
AAACTCTACCTAGTCTAATGTCTAACGACAGATCTTACTGGTTTNACTGCAGTGAT
ATCTACTATCTTTTGGAATGTTTTCTCCTTCAGTTATACATCAAGTTCCAAGATGCA
GGTGTGCTTGATTGATGTACATGGCTGTGAGAAGTGCATCCTGATGTTCAGATGAT
GGTTCATTCTAATGTCTTTTCCTTCAATCAGTTTTCTCAGTCTGACTTAGCTTGTTT
CATCTGCATCTTTTGAATGTTCGTTTACTCATAGTAATTGCATTTTTGTAGCAGAAC
ATATCATTGGTGATGGTTTCAACTGTGCGCGAGTCTTATGCTTATTCAAACTAGGA
AAGCCTCCGTCTAGAGGGTACACGAGTTGTTGCTCTGTGTGCGTCAGTCCATAGTA
TTAATCTTGCTAGTTGTAGTATATTGTTTATGTGGACTCGGAATTCATCATATGCTC
CTTCTTTGCATCAAGTAAGGCAAGGTAATGTATAGAAGCTTTTTAACTCTTTCATG
GAAGCTGGCCTTTGCCAGCATACCATCCAGAAGATATCAACCCTGCATCTTGGCT
GCCGCGCTGTCAGGAGGTCAACTACCCCAATTTAAATTTTATTTGATTAAGATATT
TTTATGGACCTACTTTATAATTAAAAATATTTTCTATTTGAAAAGGAAGGACAAAA
ATCATACAATTTTGGTCCAACTACTCCTCTCTTTTTTTTTTTGGCTTTATAAAAAAG
GAAAGTGATTAGTAATAAATNATTAAATAATGAAAAAAGGAGGAAATAAAATTT
TCGAATTAAAATGTAAAAGAGAAAAAGGAGAGGGAGTAATCATTGTTTAACTTTA
TCTAAAGTACCCCAATTCGATTTTACATGTATATCAAATTATACAAATATTTTATT
AAAATATAGATATTGAATAATTTTATTATTCTTGAAGATGTAAATAAAAATTATCT
ATTATTTCAATTTTTATATAAACTATTATTTGAAATCTCAATTATGATTTTTTAATA
TCACTTTCTATCCATGATAATTTCAGCTTAAAAAGTTTTGTCAATAATTACATTAAT
TTTGTTGATGAGGATGACAAGATTTCGGTCATCAATTACATATACACAAATTGAA
ATAGTAAGCAACTTGATTTTTTTTCTCATAATGATAATGACAAAGACACGAAAAG
ACAATTCAATATTCACATTGATTTATTTTTATATGATAATAATTACAATAATAATA
TTCTTATAAAGAAAGAGATCAATTTTGACTGATCCAAAAATTTATTTATTTTTACT
ATACCAACGTCACTAATAATATCTAATAATGTAAAACAATTCAATCTTACTTAAAT
ATTAATTTGAAATAAACTATTTTTATAACGAAATTACTAAATTTATCCAATAACAA
AAAGGTCTTAAGAAGACATAAATTGTTTTTTTGTAATGCTCAAATAAATTTGAGTA
AAAAAGAATGAAATTGAGTGATTTTTTTTTAATCATAAGAAAATAAATAATTAAT
TTCAATATAATAAAACAGTAATATAATTTCATAAATGGAATTCAATACTTACCTCT
TAGATATAAAAAATAAATATAAAAATAAAGTGTTTCTAATAAACCCGCAATTTAA
ATAAAATATTTAATATTTTCAATCAAATTTAAATAATTATATTAAAATATCGTAGA
AAAAGAGCAATATATAATACAAGAAAGAAGATTTAAGTACAATTATCAACTATTA
TTATACTCTAATTTTGTTATATTTAATTTCTTACGGTTAAGGTCATGTTCACGATAA
ACTCAAAATACGCTGTATGAGGACATATTTTAAATTTTAACCAATAATAAAACTA
AGTTATTTTTAGTATATTTTTTTGTTTAACGTGACTTAATTTTTCTTTTCTAGAGGA
GCGTGTAAGTGTCAACCTCATTCTCCTAATTTTCCCAACCACATAAAAAAAAAATA
AAGGTAGCTTTTGCGTGTTGATTTGGTACACTACACGTCATTATTACACGTGTTTT
CGTATGATTGGTTAATCCATGAGGCGGTTTCCTCTAGAGTCGGCCATACCATCTAT
AAAATAAAGCTTTCTGCAGCTCATTTTTTCATCTTCTATCTGATTTCTATTATAATT
TCTCTGAATTGCCTTCAAATTTCTCTTTCAAGGTTAGAATTTTTCTCTATTTTTTGGT
TTTTGTTTGTTTAGATTCTGAGTTTAGTTAATCAGGTGCTGTTAAAGCCTAAATTT
TGAGTTTTTTTCGGTTGTTTTGATGGAAAATACCTAACAATTGAGTTTTTTCATGTT
CTTTTTGTCGGAGAATGCCTACAATTGGAGTTCCTTTCGTTGTTTTGATGAGAAAGC
CCCTAATTTGAGTGTTTTTCCGTCGATTTGATTTTAAAGGTTTATATTCGAGTTTTT
TTCGTCGGTTTAATGAGAAGGCCTAAAATAGGAGTTTTTCTGGTTGATTTGACTAA
AAAAGCCATGGAATTTTGTGTTTTTGATGTCGCTTTGGTTCTCAAGGCCTAAGATC
TGAGTTTCTCCGGTTGTTTTGATGAAAAAGCCCTAAAATTGGAGTTTTTATCTTGT
GTTTTAGGTTGTTTTAATCCTTATAATTTGAGTTTTTTCGTTGTTCTGATTGTTGTTT
TTATGAATTTTGCAGAATGATGAAGAGTTTTTTCCTAGTTGTGACTATCCTGGCAT
TAACCCTGCCATTTTTGGGTGCCCAGGAGCAAAACCAAGAACAACCAATACGCTG
TGAGAAAGATGAAAGATTCTTCAGTGACAAAATAGCCAAATATATCCCAATTCAG
TATGTGCTGAGTAGGTATCCTAGTTATGGACTCAATTACTACCAACAGAAACCAG
TTGCACTAATTAATAATCAATTTCTGCCATACCCATATTATGCAAAGCCAGCTGCA
GTTAGGTCACCTGCCCAAATTCTTCAATGGCAAGTTTTGTCAAATACTGTGCCTGC
CAAGTCCTGCCAAGCCCAGCCAACTACCATGGCACGTCACCCACACCCACATTTA
TCATTTATGGCCATTCCACCAAAGAAAAATCAGGATAAAACAGAAATCCCTACCA
TCAATACCATTAGTGGTGAGCCTACAAGTACACCTACCATCGAAGCAGTAGA
GAGCACTGTAGCTACTCTAGAAGCTTCTCCAGAAGTTATTGAGAGCCCACCTGAG
ATCAACACAGTCCAAGTTACTTCAACTGCGGTCTAAGCTTGTTGTGGTTGTCTGGT
TGCGTCTGTTGCCCGTTGTCTGTTGCCCATTGTGGTGGTTGTGTTTGTATGATGGTC
GTTAAGGATCATCAATGTGTTTTCGCTTTTTGTTCCATTCTGTTTCTCATTTGTGAA
TAATAATGGTATCTTTATGAATATGCAGTTTGTGGTTTCTTTTCTGATTGCAGTTCT
GAGCATTTTGTTTTTGCTTCCGTTTACTTATACCACTACAGTTTGCACTAATTTAGT
TGATATGCGAGCCATCTGATGTTTGATGATTCAAATGGCGTTTATGTAACTCGTAC
CCGAGTGGATGGAGAAGAGCTCCATTGCCGGTTTGTTTCATGGGTGGCGGAGGGC
AACTCCTGGGAAGGAACAAAAGAAAAACCGTGATACGAGTICATGGGTGAGAGC
TCCAGCTTGATCCCTTCTCTGTCGATCAAATTTGAATTTTTGGATCACGGCAGGCT
CACAAGATAATCCAAAGTAAAACATAATGAATAGTACTTCTCAATGATCACTTAT
TTTTAGCAAATCAGCAATTGTGCATGTCAAATGATTTCGGTGTAAGAGAAAGAGT
TGATGAATCAAAATATCTGTAGCTGGATCAAGAATCTGAGGCAGTTGTATGTATC
AATGATCTTTCCGCTACAATGATGTAGCTATCCGAGTCAAATTTTGTAGAATTG
CATACTTCGGCATCACATTCTGGATGACATAATAAATAGGAAGTCTTCAGATCCCT
AAAAAATTGAGAGCTAATAACATTAGTCCTAGATGTAACTGGGTGACAACCAAGA
AAGAGACATGCAAATACTACTTTTGTTTGAAGGAGCATCCCTGGTTTGACATATTT
TTTCTGAATATCAAACTTTGAAACTCTACCTAGTCTAATGTCTAACGACAGATCTT
ACTGGTTTAACTGCAGTGATATCTACTATCTTTTGGAATGTTTTCTCCTTCAGTTAT
ACATCAAGTTCCAAGATGCAGGTGTGCTTGATTGATGTACATGGCTGTGAGAAGT
GCATCCTGATGTTCAGATGATGGTTCATTCTAATGTCTTTTCCTTCAATCAGTTTTC
TCAGTCTGACTTAGCTTGTTTCATCTGCATGTTTGAATGTTCGTTTACTCATAGTAA
TTGCATTTTTGTAGCAGAACATATCATTGGTCATGGTTTCAACTGTGCGCGAGTCT
TATGCTTATTCAAACTAGGAAAGCCTCCGTCTAGAGGGTACACGAGTTGTTGCTCT
GTGTGCGTCAGTCCATAGTATTAATCTTGCTAGTTGTAGTATATTGTTTATGTGGA
CTCGGAATTCATCATATGCTCCTTCTTTGCATCAAGTAAGGCAAGGTAATGTATAG
AAGCTTTTTAACTCTTTCATGGAAGCTGGCCTTTGCCAGCATACCATCCAGAAGAT
ATCAACCCTGCATCTTGGCTGCCGCGCTGTCAGGAGGTCAACTACCCCAATTTAA
ATTTTATTTGATTAAGATATTTTTATGGACCTACTTTATAATTAAAAATATTTTCTA
TTTGAAAAGGAAGGACAAAAATCATACAATTTTGGTCCAACTACTCCTCTCTTTTTT
TTTTTTGGCTTTATAAAAAAGGAAAGTGATTAGTAATAAATAATTAAATAATGAA
AAAAGGAGGAAATAAAATTTTCGAATTAAAATGTAAAAGAGAAAAAGGAGAGGG
AGTAATCATTGTTTAACTTTATCTAAAGTACCCCAATTCGATTTTACATGTATATC
AAATTATACAAATATTTTATTAAAATATAGATATTGAATAATTTTATTATTCTTGA
ACATGTAAATAAAAATTATCTATTATTTCAATTTTTATATAAACTATTATTTGAAA
TCTCAATTATGATTTTTTAATATCACTTTCTATCCATGATAATTTCAGCTTAAAAAG
TTTTGTCAATAATTACATTAATTTTGTTGATGAGGATGACAAGATTTCGGTCATCA
ATTACATATACACAAATTGAAATAGTAAGCAACTTGATTTTTTTTCTCATAATGAT
AATGACAAAGACACGAAAAGACAATTCAATATTCACATTGATTTATTTTTATATG
ATAATAATTACAATAATAATATTCTTATAAAGAAAGAGATCAATTTTGACTGATCC
AAANATTTATTTATTTTTACTATACCAACGTCACTAATTATATCTAATAATGTAAA
ACAATTCAATCTTACTTAAATATTAATTTGAAATAAACTATTTTTATAACGAAATT
ACTAAATTTATCCAATAACAAAAAGGTCTTAAGAAGACATAAATTCTTTTTTTGTA
ATGCTCAAATAAATTTGAGIAAAAAAGAATGAAATTGAGTGATTTTTTTTTAATCA
TAAGAAAATAAATAATTAATTTCAATATAATAAAACAGTAATATAATTTCATAAA
TGGAATTCAATACTTACCTCTTAGATATAAAAAATAAATATAAAAATAAAGTGTT
TCTAATAAACCCGCAATTTAAATAAAATATTTAATATTTTCAATCAAATTTAAATA
ATTATATTAAAATATCGTAGAAAAAGAGCAATATATAATACAAGAAAGAAGATTT
AAGTACAATTATCAACTATTATTATACTCTAATTTTGTTATATTTAATTTCTTACGG
TTAAGGTCATGTTCACGATAAACTCAAAATACGCTGTATGAGGACATATTTTAAAT
TTTAACCAATAATAAAACTAAGTTATTTTTAGTATATTTTTTTGTTTAACGTGACTT
AATTTTTCTTTTCTAGAGGAGCGTGTAAGTGTCAACCTCATTCTCCTAATTTTCCCA
ACCACATAAAAAAAAAATAAAGGTAGCTTTTGCGTGTTGATTTGGTACACTACAC
GTCATTATTACACGTGTTTTCGTATGATTGGTTAATCCATGAGGCGGTTTCCTCTA
GAGTCGGCCATACCATCTATAAAATAAAGCTTTCTGCAGCTCATTTTTTCATCTTC
TATCTGATTTCTATTATAATTTCTCTGAATTGCCTTCAAATTTCTCTTTCAAGGTTA
GAATTTTTCTCTATTTTTTGGTTTTTGTTTGTTTAGATTCTGAGTTTAGTTAATCAGG
TGCTGTTAAAGCCCTAAATTTTGAGTTTTTTTCGGTTGTTTTGATGGAAAATACCTA
ACAATTGAGTTTTTTTATGTTGTTTTGTCGGAGAATGCCTACAATTGGAGTTCCTTT
CGTTGTTTTGATGAGAAAGCCCCTAATTTGAGTGTTTTTCCGTCGATTTGATTTTAA
AGGTTTATATTCGAGTTTTTTTCGTCGGTTTAATGAGAAGGCCTAAAATAGGAGTT
TTTCTGGTTGATTTGACTAAAAAAGCCATGGAATTTTGTGTTTTTGATGTCGCTTTG
GTTCTCAAGGCCTAAGATCTGAGTTTCTCCGGTTGTTTTGATGAAAAAGCCCTAAA
ATTGGAGTTTTTATCTTGTGTTTTAGGTTGTTTTAATCCTTATAATTTGAGTTTTTTC
GTTGTTCTGATTGTTGTTTTTATGAATTTTGCAGAATGAAGTGCCTCCTGCTTGCCC
TGGCCCTCACTTGIGGCGCCCAGGCCCTCATTGTCACCCAGACCATGAAGGGCCT
GGATATCCAGAAGGTGGCGGGGACTTGGTACTCCTTGGCCATGGCGGCCAGCGAC
ATCTCCCTGCTGGACGCCCAGAGTGCCCCCCTGAGAGTGTATGTGGAGGAGCTGA
AGCCCACCCCTGAGGGCGACCTGGAGATCCTGCTGCAGAAATGGGAGAACGGTG
AGTGTGCTCAGAAGAAGATCATTGCAGAAAAAACCAAGATCCCTGCGGTGTTCAA
GATCGATGCCTTGAATGAGAACAAAGTCCTTGTGCTGGACACCGACTACAAAAAG
TACCTGCTCTTCTGCATGGAGAACAGTGCTGAGCCCGAGCAAAGCCTGGCCTGCC
AGTGCCTGGTCAGGACCCGGAGGTGGACGACGAGGCCCTGGAGAAATTCGACA
AAGCCCTCAAGGCCCTGCCCATGCACATCCGGCTGTCCTTCAACCCAACCCAGCT
GGAGGAGCAGTGCCACATCTAGGCTTGTTGTGGTTGTCTGGTTGCGTCTGTTGCCC
GTTGTCTGTTGCCCATTGTGGTGGTTGTGTTTGTATGATGGTCGTTAAGGATCATC
AATGTGTTTTCGCTTTTTGTTCCATTCTGTTTCTCATTTGTGAATAATAATGGTATC
TTTATGAATATGCAGTTTGTGGTTTCTTTTCTGATTGCAGTTCTGAGCATTTTGTTT
TTGCTTCCGTTTACTATACCACTTACAGTTTGCACTAATTTAGTTGATATGCGAGCC
ATCTGATUTTTGATGATTCAAATGGCGTTTATGTAACTCGTACCCGAGTGGATGGA
GAAGAGCTCCATTGCCGGTTTGTTTCATGGGTGGCGGAGGGCAACTCCTGGGAAG
GAACAAAAGAAAAACCGTGATACGAGTTCATGGGTGAGAGCTCCAGCTTGATCCC
TTTTCTGTCGATCAAATTTGAATTTTTGGATCACGGCAGGCTCACAAGATAATCCA
AAGTAAAACATAATGAATAGTACTTCTCAATGATCACTTATTTTTAGCAAATCAGC
AATTGTGCATGTCAAATGATTTCGGTGTAAGAGAAAGAGTTGATGAATCAAAATA
TCTGTAGCTGGATCAAGAATCTGAGGCAGTTGTATGTATCAATGATCTTTCCGCTA
CAATGATGTTAGCTATCCGAGTCAAATTGTTGTAGAATTGCATACTTCGGCATCAC
ATTCTGGATGACATAATAAATAGGAAGTCTTCAGATCCCTAAAAAATTGAGAGCT
AATAACATTAGTCCTAGATGTAACTGGGTGACAACCAAGAAAGAGACATGCAAAT
ACTACTTTTGTTTGAAGGAGCATCCCTGGTTTGACATATTTTTTCTGAATATCAAA
CTTTGAAACTCTACCTAGTCTAATGTCTAACGACAGATCTTACTGGTTTAACTGCA
GTGATATCTACTATCTTTTGGAATGTTTTCTCCTTCAGTTATACATCAAGTTCCAAG
ATGCAGGTGTGCTTGATTGATGTACATGGCTGTGAGAAGTGCATCCTGATGTTCAG
ATGATGGTTCATTCTAATGTCTTTTCCTTCAATCAGTTTTCTCAGTCTGACTTAGCT
TGTTTCATCTGCATGTTTGAATGTTCGTTTACTGATAGTAATTGCATTTTTGTAGCA
GAACATATCATTGGTCATGGTTTCATTCTGTGCGCGAGTCTTATGCTTATTCAAACT
AGGAAAGCCTCCGTCTAGAGGGTACACGAGTTGTTGCTCTGTGTGCGTCAGTCCA
TAGTATTAATCTTGCTAGTTGTAGTATATTGTTTATGTGGACTCGGAATTCATCAT
ATGCTCCTTCTTTGCATCAAGTAAGGCAAGGTAATGTATAGAAGCTTTTTAACTCT
TTTCATGGAAGCTGGCCTTTGCCAGCATACCATCCAGAAGATATCAACCCTGCATCT
TGGCTGCCGCGCTGTCAGGAGGTCAACTACCCCAATTTAAATTTTATTTGATTAAG
ATATTTTTATGGACCTACTTTATAATTAAAAATATTTTCTATTTGAAAAGGAAGGA
CAAAAATCATACAATTTTGGTCCAACTACTCCTCTCTTTTTTTTTTTGGCTTTATAA
AAAAGGAAAGTGATTAGTAATAAATAATTAAATAATGAAAAAAGGAGGAAATAA
AATTACGAATTAAAATGTAAAAGAGAAAAAGGAGAGGCAGTAATCATTGTTTAA
CTTTATCTAAAGTACCCCAATTCGATTTTACATGTATATCAAATTATACAAATATT
TTATTAAAATATAGATATTGAATAATTTTATTATTCTTGAACATGTAAATAAAAAT
TATCTATTATTTCAATTTTTATATAAACTATTATTTGAAATCTCAATTATGATTTTT
TAATATCACTTTCTATCCATGATAATTTCAGCTTAAAAAGTTTTGTCAATAATTAC
ATTAATTTTGTTGATGAGGATGACAAGATTTCGGTCATCAATTACATATACACAAA
TTGAAATAGTAAGCAACTTGATTTTTTTTTTCATAATGATAATGACAAAGACACGA
AAAGACAATTCAATATTCACATTGATTTATTTTTATATGATAATAATTACAATAAT
AATATTCTTATAAAGAAAGAGATCAATTTTGACTGATCCAAAAATTTATTTATTTT
TACTATACCAACGTCACTAATTATATCTAATAATGTAAAACAATTCAATCTTACTT
AAATATTAATTTGAAATAAACTATTTTTATAACGAAATTACTAAATTTATCCAATA
ACAAAAAGGTCTTAAGAAGACATAAATTCTTTTTTTGTAATGCTCAAATAAATTTG
AGTAAAAAAGAATGAAATTGAGTGATTTTTTTTTAATCATAAGAAAATAAATAAT
TAATTTCAATATAATAAAACAGTAATATAATTTCATAAATGGAATTCAATACTTAC
CTCTTAGATATAAAAAATAAATATAAAAATAAAGGTTTTCTAATAAACCCGCAAT
TTAAATAAAATATTTAATATTTTCAATCAAATTTAAATAATTATATTAAAATATCG
TAGAAAAAGAGCAATATATAATACAAGAAAGAAGATTTAAGTACAATTATCAACT
ATTATTATACTCTAATTTTGTTATATTTAATTTCTTACGGTTAAGGTCATGTTCACG
ATAAACTCAAAATACGCTGTATGAGGACATATTTTAAATTTTAACCAATAATAAA
ACTAAGTTATTTTTAGTATATTTTTTTGTTTAACGTGACTTAATTTTTCTTTTCTAGA
GGAGCGTGTAAGTGTCAACCTCATTCTCCTAATTTTCCCAACCACATAAAAAAAA
AATAAAGGTAGCTTTTGCGTGTTGATTTGGTACACTACACGTCATTATTACACGTG
TTTTCGTATGATTGGTTAATCCATGAGGCGGTTTCCTCTAGAGTCGGCGATACCAT
CTATAAAATAAAGCTTTCTGCAGCTCATTTTTTCATCTTCTATCTGATTTCTATTAT
AATTTCTCTGAATTGCCTTCAAATTTCTCTTCAAGGTTAGAATTTTTCTCTATTTIT
TGGTTTTTGTTTGTTTAGATTCTGAGTTTAGTTAATCAGGTGCTGTTAAAGCCCTAA
ATTTTGAGTTTTTTTCGGTTGTTTTGATGGAAAATACCTAACAATTGAGTTTTTTCA
TGTTGTTTTGTCGGAGAATGCCTACAATTGGAGTTCCTTTCGTTGTTTTGATGAGA
AAGCCCCTAATTTGAGTGTTTTTCCGTCGATTTGATTTTAAAGGTTTATATTCGAGT
TTTTTTCGTCGGTTTAATGAGAAGGCCTAAAATAGGAGTTTTTCTGGTTGATTTGA
CTAAAAAAGCCATGGAATTTTGTGT (SEQ ID NO: 50)

### Example 5: Transfection of Nicotiana benthamiana with a Vector for Co-Expression of Cow's Milk Genes Simultaneously in a Single Nicotiana benthamiana Plant Leaf

To express all seven genes simultaneously in a Nicotiana benthamiana plant leaf, the T-DNA binary vector (plasmid), pDGB-Ω1 Seven bovine milk genes (pDGB-Ω1 Seven milk genes, pDGB-S21 Seven genes; pDGB-omega1 Seven milk genes, pDGB-omega1 Seven genes), carrying all the seven cow's milk proteins under the control of constitutive SlPUbiq10 promoters as well as the BASTA resistance gene, was constructed as pDGB-Ω1 (pDGB-omega1) as described above (FIG. 4, TABLE 6). N. benthamiana has been transfected with the pDGB-Ω1 (pDGB-omega1) Seven bovine milk genes promoter, and resistance to BASTA has been demonstrated.

### Example 6: Transfection of Rice Plants with a Vector for Co-Expression of Cow's Milk Genes Simultaneously in a Rice Seed

To express all seven genes simultaneously in a single rice plant or seed, the T-DNA binary vector (plasmid), pDGB-omega1 Seven milk genes, carrying all the seven cow's milk proteins under the control of constitutive SlPUbiq10 promoters as well as the BASTA resistance gene, was constructed as described above (FIG. 4, TABLE 6). Rice plants have been transfected with the pDGB-omega1 Seven bovine milk genes plasmid.

### Example 7: Transfection of Soy Plants with a Vector for Co-Expression of Cow's Milk Genes Simultaneously in Soybeans

To express all seven genes simultaneously in a single soy plant or seed (soybean), the T-DNA binary vector (plasmid), pDGB-omega1 Seven milk genes, carrying all the seven cow's milk proteins under the control of constitutive SlPUbiq10 promoters as well as the BASTA resistance gene, was constructed as described above (FIG. 4, TABLE 6). Soy plants were transfected with the pDGB-omega1 Seven bovine milk genes plasmid.

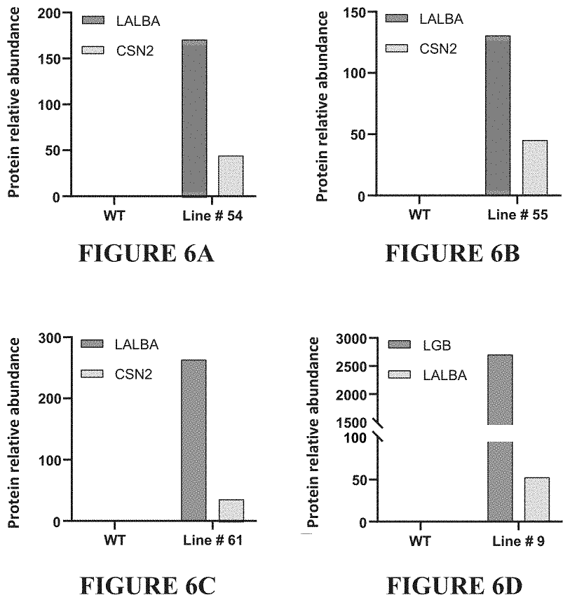

Protein expression of the cow milk genes in the transformed soy plants was confirmed by employing untargeted LC-MS/MS proteomic analysis. In brief, soy leaves were ground in liquid N, total protein was extracted and quantified. Similar amounts of leaf protein were subjected to tryptic digestion, followed by peptide recovery and desalting. The peptides obtained were analyzed using nano-UPLC coupled to a quadrupole orbitrap mass spectrometer. The data analysis revealed the production of three milk proteins in transformed soy leaves (FIGS. 6A-D). The milk proteins include CSN2 (β casein), LALBA (α-lactalbumin), and LGB (β-lactoglobulin). Approximately 40 independent soybean transgenic lines were generated. The results of 4 of them are shown in FIGS. 6A-D. Lines #54 (FIG. 6A), #55 (FIG. 6B) and #61 (FIG. 6C) produce LALBA and CSN2 while line #9 (FIG. 6D) produces LGB and LALBA.

Following the identification of milk proteins present in the soybean leaves of transgenic lines, protein expression of the cow milk genes in the transformed soy plants was also confirmed in the soybean seeds. Seeds from greenhouse-grown transgenic soy plants were collected and dried and subjected to proteomic analysis. The seeds were ground to fine powder in liquid nitrogen and total protein was extracted. After protein concentration measurements and normalization, 50 μg of total protein from the seeds of each transgenic line were subjected to trypsin digestion. The resulting peptides were desalted, acidified, dried, and analyzed by means of nano-UPLC, coupled through a nano-ESI emitter to a quadrupole orbitrap mass spectrometer. Protein identification and label-free quantification was carried out using the Proteome Discoverer software. A modified target database of soy proteins with supplemented 8 milk protein sequences was used to analyze the obtained data.

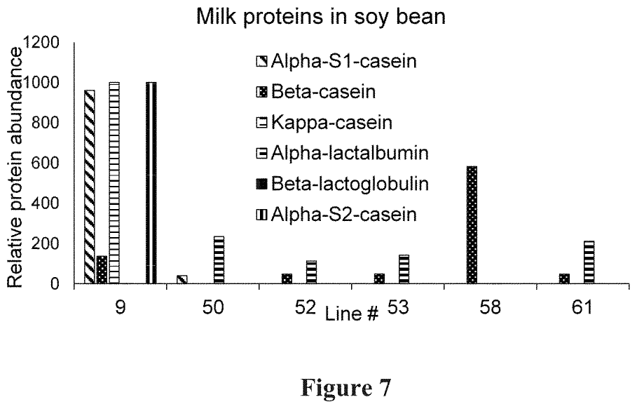

Five out of seven milk proteins were detected in the analyzed soybean seed samples: alpha-S1-casein, beta-casein, kappa-casein, alpha-lactalbumin, and alpha-S2-casein. FIG. 7 presents the results of representative seed samples from different transgenic lines. Transgenic lines where the presence of more than one protein was identified in the seeds with high confidence are shown in the bar graph of FIG. 7 and included lines 9, 50, 52, 53, 58, and 61. As seen in FIG. 7, five independent transgenic lines expressed and produced beta-casein (9, 52, 53, 58, and 61), six lines expressed and produced alpha-lactalbumin (9, 50, 52, 53, 58, and 61), while two lines showed a significant expression of alpha-S1-casein (9 and 50). Notably, soybean seeds from transgenic line #9 had a significant expression and production of four milk proteins (alpha-S1-casein, beta-casein, kappa-casein, and alpha S2-casein); and low expression of a fifth protein (alpha-lactalbumin).

### Example 8: Vector for Co-Expression of Cow's Milk Genes in Soybean and Having a Content Profile Reflecting the Content Profile of Cow's Milk

In cow's milk the major seven proteins are found in different proportions extending from 1% to 34% out of the total protein content (TABLE 7). Therefore, to achieve similar content profile in our animal-free milk requires differential expression of each of the proteins in the soybeans. To this end, we used a set of seed-specific promoters (Gunadi et al. (2016) Plant Cell. Tissue Organ Cult. 127(1): 145-160 [“Gunadi 2016”]) that are predicted to express the seven cow's milk proteins in similar proportions to those found in milk (Soy Online Database [available: https://soybase.org/; accessed: 29 Nov. 2018] [“Soybase”]) (TABLE 7). The sequences of these promoters are found in TABLE 8.

TABLE 7
Promoter assignments to the seven cow's milk proteins in the T-DNA
expression vector.

| | | Relative | Relative predicted |
|---|---|---|---|
| | | abundance in | abundance in |
| Promoter Name | Controlled gene | cow's milk | soybeans |
| Seed 1 | beta-Casein | 34% | 30% |
| Seed 2 | kappa-Casein | 9% | 13% |
| | beta-Lactoglobulin | 9% | 13% |
| Seed 3 | alpha-S2-Casein | 14% | 13% |
| Seed 4 | alpha-S1-Casein | 28% | 25% |
| Seed 5 | Serum Albumin | 1% | 1% |
| Seed 6 | alpha-Lactalbumin | 5% | 4% |

TABLE 8
Seed promotor sequences used for the expression of the cow's
milk genes.

| | |
|---|---|
| Seed 1 | AACACAAGCTTCAAGTTTTAAAAGGAAAAATGTCAGCCAAAAACTTTAA |
| | ATAAAATGGTAACAAGGAAATTATTCAAAAATTACAAACCTCATCAAA |
| | ATAGGAAAGAAAAAAAGTTTAGGGATTTAGAAAAAACATCAATCTAGT |
| | TCCACCTTATTTTATAGAGAGAAGAAACTAATATATAAGAACTAAAAAA |
| | CAGAAGAATAGAAAAAAAAAGTATTGACAGGAAAGAAAAAGTAGCTGT |
| | ATGCTTATAAGTACTTTGAGGATTTGAATTCTCTCTTATAAAACACAAAC |
| | ACAATTTTTAGATTTTATTTAAATAATCATCAACTCGATTATAATTATTT |
| | ATATATTTTTCTATTTTCAAAGAAGTAAATCATGAGCTTTTCCAACTCAA |
| | CTCTTTTTTTCTCTCAACCTTTTTCACATCTTAAGTAGTCTCACCCT |
| | TTATATATATAACTTATTTCTTACCTTTTACATTATGTAACTTTTATCACC |
| | AAAACCAACAACTTTAAAATTTTATTAAATAGACTCCACAAGTAACTTG |
| | ACACTCTTACATTCATCGACATTAACTTTTATCTGTTTTATAAATATTATT |
| | GTGATATAATTTAATCAAAATAACCACAAACTTTCATAAAAGGTTCTTA |
| | TTAAGCATGGCATTTAATAAGCAAAAACAACTCAATCACTTTCATATAG |
| | GAGGTAGCCTAAGTACCTACAAAATGCCTACAAATAAAAAAAAAGT |
| | TGCTTTAATAATGCCAAAACAAATTAATAAAACACTTACAACACCGGAT |
| | TTTTTTTAATTTAAAATGTGCCATTTAGGATAAATAGTTTAATATTTTTTAA |
| | TAATTGTTTAAAAAGCCGTATCTACTAAAATGATTTTTATTTGGTTGAAAA |
| | TATTAATATGTTTAAATCAACACAATCTATCAAAATTAAACTAAAAAAA |
| | AAATAAGTGTACGTGGTTTAACATTAGTACAGTAATATAAGAGGAAAAT |
| | GAGAAATTAAGAAATTGAAAGCGAGTCTAATTTTTAAATTATGAACCTG |
| | CATATATAAAAGGAAAGAAAGAATCCAGGAAGAAAAGAAATGAAACC |
| | ATGCATGGTCCCCTCGTCATCACGAGTTTCTGCCATTTGCAATAGAAAC |
| | ACTGAAACACCTTTCTCTTTGTCACTTAATTGAGATGCCGAAGCCACCTC |
| | ACACCATGAACTTCATGAGGTGTAGCACCCAAGGCTTCCATAGCCATGC |
| | ATACTGAAGAATGTCTCAAGCTCAGCACCCTACTTCTGTGACGTGTCCCT |
| | CATTCACCTTCCTCTCTTCCCTATAAATAACCACGCCTCAGGTTCTCCGC |
| | TTCACAACTCAAACATACACTCCATTGGTCCTTAAACACTCATCAGTCAT |
| | CACCATGGCCAAGCTA (SEQ ID NO: 51) |
| Seed 2 | TAGATTTTGAGTTGTTTGAGGTTCCATTGCCTTATTGCTAAAACTCCAAC |
| | TAAAATAACAAATAGCACATGCGGTGCAAACAACACGTTACTCTGATG |
| | AAGGTAGATCTAGCCTCTAGCAGTCTAGCTTATGAGGCTCGCTGCTTATCA |
| | ACGATTCATCATTCCCCAAGACGTGTACGCAGATTAAACAATGGACAAA |
| | ACTTCAATCGATTATAGAATAATAATTTTAACAGTGCCGAGTTTTTTCTG |
| | TAAACAAAAGGCCAGAATCATATCGCACATCATCTTGAATGCAGTGTCG |
| | AGTTTGGACCATTTGAGTACAAAGCCAATATTGAATGATTTTTCGATTTT |
| | ACATGTGTGAATCAGACAAAAGTGCATGCAATCACTTGCAAGTAAATTA |
| | AGGATACTAATCTATTCCTTTCATTTTATATGCTCCACTTTTATATAAAA |
| | AAATATACATTATTATATATGCATTATTAATTATTGCAGTATTATGCTAT |
| | TGGTTTTATGGCCCTGCTAAATAACCTAAATGAGTCTAACTATTGCATAT |
| | GAATCAAATGAAGGAAGAATCATGATCTAAACCTGAGTACCCAATGCA |
| | ATAAAATGCGTCCTATAACCTAAACTTCAAACACACATTGCCATCGGAC |
| | GTATAAATTAATGCATATAGATTATTTTGAGAAAAGAAAACATCAAAAG |
| | CTCTAAAACTTCTTTTAACTTTGAAATAAGCTGATAAAAATACGCTTTAA |
| | ATCAACTGTGTGCTGTATATAAGCTGCAATTTCACATTTTACCAAACCGA |
| | AACAAGAATGGTAACAGTGAGGCAAAAATTTGAAAAATGTCCTACTTC |
| | ACATTCACATCAAATTAATTACAACTAAATAAATAAACATCGTGATTCA |
| | AGCAGTAATGAAAGTCGAAATCAGATAGAATATACACGTTTAACATCA |
| | ATTGAATTTTTTTTTAAATGGATATATAGAAGTTTACTATTTTATATATA |
| | ATGAAAATTCATTTTGTGTTAGCACAAAACTTACAGAAAGAGATAAATT |
| | TTAAATAAAGAGAATTATATCCAATTTTATAATCCAAAATAATCAAATE |
| | AAAGNATATTGGCTAGATAGACCGGCTTTTTGACTGCCCCTGCTGGATA |
| | ATGAAAATTCATATCAAAACAATACAGAAGTTCTAGTTTAATAATAAAA |
| | AAGTTGGCAAACTAGTCATTCCCTGTTGGTTTTTAAGCCAAATCACAATTC |
| | AATTACGTATCAGAAATTAATTTAAACCAAATATATAGCTACGAGGGAA |
| | CTTCTTCAGTCATTACTAGCTAGCTCACTAATCACTATATATACGACATG |
| | CTACAAGTGAAGTGACCATATCTTAATTTCAAATCATAAAATTCTTCCAC |
| | CAAGTTATGGGTTTCCT (SEQ ID NO: 52) |
| Seed 3 | ATTATTTCTGTTAGTACATAGCTAATACTCAATCAACGGAATTAGTATAT |
| | GGTTCTTCATATAGGAGAGTACTTATTTATTCTATTGAATTTTAACATAT |
| | AAGCATAATAAAATACTTTTGGACTCTCGTATAAAGTTCGATTTTAATCT |
| | TTTTAATAATTCAATCTAAATGTTTAATTCCCTCTTAAATGCAAAATTCA |
| | GTTTTTCGTTCCTTTAATGTGACACCATTAGGTCACATGAACCGGAAATG |
| | ACGTGGTGATCGAATTATGACTTGAATCCATTGACCACATTAGCATTTC |
| | ACCTATGGTCACTAGTATGAAGGATGAAAACAAGTCTATTTCTCAAATT |
| | ATAAATGAAAACCTTTTAAGTTAAACCTGAGGATCCAAAAACGAATTTT |
| | ACTAAATTTTGAAGAACTAAAAAATATTTAATCTAGTAAAACGCGTGTC |
| | TATCTAATATAACATGCACGCTCGTCATGTAATCAATTAGGCATAAAAA |
| | TAGTGTTTGATTTTTTGACACATTATTAAGTGTTTTATTTTTAAGTTTAAA |
| | AGCATTGGTATCCTTTCATAAAAGGAGGTAATCTTATTTAAGTCAAGGA |
| | GAATTATTATGGGAAATAAAACCTTTTTTTTTAAAGTGTTTAATATAATT |
| | ATATACTCAAAATTCGATTTATGATTAAATCTAAGTGACATTTAAAAAA |
| | AATTAGTGTGAAAATAATTTATATATAATTTTGAAAAATTTATCATTAAT |
| | TTTTTTTTATAAATAAATGTTAATTTATTAGTTTTTATTATAAATGTGAAT |
| | AGAATGGATTCGAAGCAGCAATTTCTCTCTTTCTCCTTTTCCATGCCAAC |
| | CTTATATATGGTGACGAACTGCATATACAGTAAAACAGTTCAAATTGAG |
| | AAAGATTTTAAACATCATAGTATTTGATATATATCTTTTACAGAGACAAT |
| | TATGCTGCAGGAGTTAGATAAGATTATTGTGGATGTCATTTTCTTTTTTA |
| | ATATTTAACGCATTATATAAAAGATGATATAGTATGGTTATAAAAAAAT |
| | TATTTAACAGTTTATAAAACCTTTTTTTTTATCTTTTACAGTAATATTATT |
| | TATTTTATTTCATCATTTTTTTCATATCCTTATCTCATTTATAAAGGAAATT |
| | AATTGTATAAAAAAAATATGATGCACTGAATAGAATGCTGATCTTATTG |
| | TATAAGGAGGATAGAATTTGAGACGCGGAGAATCTGTAGAGGGGGACC |
| | ATTCAGGGTGCCTGCAATTTTGGTGTTGTTCATGTACGGTTGCAGATATA |
| | AACGAAGCATAGCTTATGTATGAGGTGTAACAAAATTGGAAACAATAG |
| | CCATGCAAGGTGAAGAATGTCACCAACTCAGAAACCCTTCTTCATTGAC |
| | GTGTCCCTCACTCACTCTCCTTCTCTTCACTGTAAATCGCCACTCTTCGTGT |
| | TCTCCACTTCACCAACTCCTTCAAACTTATTAACACTTTCCTTAGTTCAA |
| | TATGGGGAAGC (SEQ. ID NO: 53) |
| Seed 4 | AACTTAATCGTATATAAAAAATTCAATATATGAATAATTCTAAGTGAGT |
| | TTTTAAGAAAAAATAAAATTAGTAACGAAGTAATTTATATATAATTTTG |
| | AAAAATTATCACTAAATTTGTGATCCACTGTTAACATTAATTTATTCCTC |
| | TTGTATTGAATAAAATAGTTCAGACATGGTCCCAGTCTTTAATCAATTAT |
| | TCATGCTTCTCTCTCTTCTTCACTTATGTAATCCTTTAATCCAAACATTACT |
| | CAGATAGCTAGATCCACCGATCAATCGTATATATATACGCATAAAATCG |
| | ACGCGTCTGTATTTTTAGACTGTAGCCCAAATTCACTATCCGAATAAAA |
| | TAAGGGAGGCACGTGTACGTAATTTATATCATATGATAGCCATGCATAT |
| | GCACACGTGCAGAAGAGCTGTTACCCTCTATACGTGTACTCACCTTCTC |
| | ATCCTCTCTGAATATTITGAGTGCTCTTCCTAGTTATCTAGTAATGCATG |
| | AAATTAAACTTACTAAATGTTTCTTCAATTTAAAGAAATAATTGTTTATC |
| | TGTTTCAATTTTTTTAAGAGAATTTTAAAAAGATAATTGTTTCGGGGAGA |
| | GAGATATAAAAAAGAAAAGGGAGAAATATTAAAATGTACTAAATAATA |
| | TGATAAGAAAAGAGAGAAAAATAAAAGAGAAAATTTGTATATAGITAT |
| | AATTATTCATGTAATAAGGATTCATCTCTCAACTGAAAATATACTTAATG |
| | CAGAAGAAAAAATCATTATTTACAAACGTTGAGTCTTGAGTGGGAAAA |
| | GAGGAGGCGCCGTTACTATACAATATAAGATCATAGTACTGACAAAATG |
| | CACAGTAAAACAGTTCAAATTGAGAAGGATTCTTAACACACCATAGTAT |
| | TTAATATATATCTTTACAGAGACAATTATGCTGGAGGATTCAGGCAAAG |
| | ATTATATATTGTGGATTTGTTTTTTAATAATTAACGCATCATATGAAAGA |
| | TCGATGATATATACTAATGGTTATAAGAAAAATATTTAACAGTTTCTAT |
| | AACCTTTTTCTTTTATCTTTTACTGTAATATTATTTATTTTATTTCACATTT |
| | TTAATCAGCTTATCTCATTTATAAACGAAATTGTATAAAAATATACATG |
| | ATGAACTGAATAGAACAATATTGATCTGATATTCTCATATTGTATAAGA |
| | GGATAGACTTTGAGACGCGGAGAATCTGTAGGAGGGGACCATTCAGAG |
| | TGCCTCCAATTTTGGTGTTGTTCATTGTACCATTGCAAATATAAACGAAG |
| | CATGCATGCTTATGTATGAGGTGTAACAAAATTGGAAACAATAGCCATG |
| | CAAGGTGAAGAATGTCACAAACTCAGCAACCCTTATTCATTGACGTGTC |
| | CCTCAGTCACTCTCCTCTCATACCTATAAATCACCACTCCTCATGTTCTTT |
| | CCAATTACCAACTCCTTCAAACTTAATTATTAACACTTCCTTAGTTCAAT |
| | ATGGGGAAGCC (SEQ ID NO: 54) |
| Seed 5 | ATAATTATAAAATTGTCACTGCGTTCAAAACGACAATGGTTTTGGGACA |
| | ACTATCATTAATCGTGCATTGTAAAAAGGTGTGTTTTTAGTAGTGGACCC |
| | TCGATAAATTGACTGTGATGATTGTTACATGTTGTTAAGTCTCACCTATA |
| | AGAAAAAAACTAAACATATATATAGATCCCAATTTTGGGGTCAGGTGTA |
| | TAGATGAAAAAAAGAAACAAATAGACAAATAAAAAAATAAAAGAAAA |
| | AAAATTGATAGATGTGAGAAATGATGAGAAGAGAAGTGCAAATAACAC |
| | ACTCTTTCTAACATTATTTTACTATTGATTAAAATTTATTGAAAATTACT |
| | ATATAATATAAAAAGTGAAACTAGTTAAACTATAGTCAATAATTGAGAA |
| | TATTTAAAAATTTAGAAAATACATTACTTATATTTCTTAAAATAAAAAAT |
| | ATAAATAAAAATAGAAAAAATGGAGTAAAATGAGATAGAAGAGAAGTT |
| | AGGTTTATAAATACATTAGTTCCGCCTACAATATATTTAAATTAGCTAGA |
| | TTAATGCAGTAAATTTTTGGCATTTACTTGATTTTATTTTCTTTAAAAGC |
| | ATTCATTGTATTCTCACGTATGCTTTTTTTCTTCATCTGCATTATGAAT |
| | TAAATCATTTACTTTGTGTCACAATTGCATTTAGCGAGGTCATGCATTGG |
| | TTAGACCGACGGTGTATTATGTCATGACTTAGGTCTTGAAGGTTGTTGGT |
| | TACTTATTATGGTCCATGGGTACACGCGTTGGTTAGATTCGATAGGCAA |
| | ATTTTGTGAACGATAGAAATTTATCTTTATTAAATAAACCACACTATATA |
| | TATATATATATATATATATATATATATATATATATATATTAATTCGTAAT |
| | TTCTTTTCTGTCTTTCATTTTGATTTTCTTTTATGGCTTTTATCTTTAAAAA |
| | TTTTCCCGTTCTTTAAAATTTACAACACTTTATAATCACAATAAAATAAA |
| | ATATTTAAAATATTACATAAATAATAACACAAATATTTATAAATCTGA |
| | AATGACATAAAATAACATTATAATCACAAAAAGTATTTAATAAAAATAA |
| | AATTACATAAATAAAATATGTGAAAACTAAGTAAAAGGTATCATGCAC |
| | GTAATCATATGAAAATAGCTTTAGAAAAAATATCAAGGCAAGTACCGC |
| | ACGTACGATAAATGAAAAAAGATTAAAAAGAAATATAATAAATAATAA |
| | TACTAAATTAATGGTGAATAAAATACTAAAAAAATAAATTTATAATTAA |
| | ATAATATGTATTACAAACACAAATAAGAAATAATAGTACATAATATTAT |
| | AATAAATAGTAGTATATAACATATCATAAATATGTTTAAAATAATGATA |
| | AAATATTGACTTTCTTTTAGTGGAACTATTTGTCAAAATGTGAACACCTG |
| | GATATGAAAAGGCATCTTAGGTAGATGATATGATGCGATAGAACGTAA |
| | AAGAAAAATGAGAAATGTGATGAGAGGTTAAAAATACCCTTCATAAC |
| | AAGCACACATCTATAAGTCTTATTCACCCAACAACGTTGCTTATTCA |
| | CGCAACTAAATAAGAAATGAAGAGTACTAT (SEQ ID NO: 55) |
| Seed 6 | TATAAACACCACTTTAATTTGACTCGGATACATGCATCCATAAAGACTA |
| | CAAAAGGCAAAAAGAGAAGGAAATGAGATACGAATATATGTCATAAGT |
| | ATATATAGGTGACAAGGGCAAATTAAATAGGTTGGTATTTAAATGCAAA |
| | ATCCTATGTTTGATAAAGAATGGTATGAAAAACAGGCAAAGTTAATTGC |
| | AATTCAAAGGTGAACAAAGCATTTCTTTGTCTACACTAATGGCATGTCT |
| | AAGTAAATTATTAGTCTTGTATCTATATGTCCACAAGTTATTAATTAGTC |
| | TTATACTATCAAAAACAAGTTAAGTTGCAAATCAAACATGAACAAAGCA |
| | TTTGTGTTGTAACCTACGAAAAAATACCCTAACATMGTGATACGAATAA |
| | TGTGGCCTAAATTGATCGTTTACCAAATTACGGTGCTGGAAAAAAAAAT |
| | TGCTCCTTTACCAACAAAATTAAGAACTGATACATCTTGTTTTTTGTCAC |
| | TGAAGATAAACACGTGATCTTTGGCAAAACGTAAAGGCCAACAAAACA |
| | AACTTGTCTCATCCCTGAATGATTCGAATGCCATCGTATGCGTGTCACAA |
| | AGTGGAATACAGCAATGAACAAATGCTATCCTCTTGAGAAAAGTGAAT |
| | GCAGCAGCAGCAGCAGACTAGAGTGCTACAATGCTTGTCCTCTTGAGA |
| | AAAGTGAATGCAGCGGCAGCAGACCTGAGTGCTATATACAATTAGACA |
| | CAGGGTCTATTAATTGAAATTGTCTTATTATTAAATATTTCGTTTTATATT |
| | AATTTTTTAAATTTTAATTAAATTTATATATATTATATTTAAGACAGATA |
| | TATTTATTTGTGATTATAAATGTGTCACTTTTTCTTTTAGTCCATGTATTC |
| | TTCTATTTTTTCAATTTAACTTTTTATTTTATTTTTAAGTCCACTCTTGATC |
| | AAGAAAACATTGTTGACATAAAACTATTAACATAAAATTATGTTAACAT |
| | GTGATAACATCATATTTTACTAATATAACGTCGCATTTTAACGTTTTTTT |
| | AACAAATATCGACTGTAAGAGTAAAAATGAAATGTTTGAAAAGGTTAA |
| | TTGCATACTAACTATTTTTTTTCCTATAAGTAATCTTTTTTGGGATCAATT |
| | GTATATCATTGAGATACGATATTAAATATGGGTACCTTTTCACAAAACC |
| | TAACCCTTGTTAGTCAAACCACACATAAGAGAGGATGGATTTAAACCAG |
| | TCAGCACCGTAAGTATATAGTGAAGAAGGCTGATAACACACTCTATTAT |
| | TGTTAGTACGTACGTATTTCCTTTTTTGTTTAGTTTTTGAATTTAATTAAT |
| | TAAAATATATATGGAACAACATTAAATTTTAAAATTACGTCTAATTATA |
| | TATTGTGATGTATAATAAATTGTCAACCTTTAAAAATTATAAAAGAAAT |
| | ATTAATTTTGATAAACAACTTTTGAAAAGTACCCAATAATGCTAGTATA |
| | AATAGGGGCATGACTCCCCATGCATCTCACTTGCAATTTAGCTGAAGCAA |
| | AGCAATGGCTACTT (SEQ ID NO: 56) |

Soybeans are highly enriched with proteins, however only eight genes code for 80% of the total protein content (Takahashi et al. Planta (August 2003) 217(4): 577-586 [“Takahashi 2003”]). In addition, the proteins coded by these genes are mostly responsible for soybean allergic response in humans (Takahashi 2003). It is important to mention that loss of these genes in soybeans, does not affect the growth rate or fertility of the plants (Takahashi 2003) and is compensated by general increased production of proteins in the seed (Takahashi 2003).

Therefore, one objective was to deplete the expression of these genes, by CRISPR/Cas9 mediated gene knock out in order to reduce the allergenic potential of the soybean and to allow increase production of the cow's milk proteins at the same time (Takahashi 2003).

TABLE 9
List of guide RNA sequences designed to target the 11S and 7S globulin genes.

| Complex name | Gene name | Accession numbers | Guide 1 sequence | Guide 2 sequence |
|---|---|---|---|---|
| glycinin (11S) | GY1 | NM_001248898.3 | TATACGGTTATC | AGAGGGCAAC |
| | GY2 | NM_001248881.1 | CGGTTTGA | ACCGGCACAC |
| | GY3 | NM_001249911.2 | (SEQ ID NO: 57) | (SEQ ID NO: 58) |
| | GY4 | NM_001251079.2 | GGCTTCCCCAT | CACCGCGTTGA |
| | GY5 | NM_001249747.3 | ATTGAACTA | GTCCGAAGG |
| | | | (SEQ ID NO: 59) | (SEQ ID NO: 60) |
| β- | alpha- | NM_001249927.2 | CGGTTCCCATTA | TCGTTGCAACC |
| conglycinin | conglycinin | | CTGTTGCT | TCCTTAAGG |
| (7S) (beta- | alpha-prime | NM_001250387.2 | (SEQ ID NO: 61) | (SEQ ID NO: 62) |
| conglycinin | conglycinin | | | |
| | beta- | NM_001249943.2 | TTAGAGCTTCTC | TGGGGGAGAA |
| | Conglycinin | | AAGTAGAA | GGATTGTGTT |
| | | | (SEQ ID NO: 63) | (SEQ ID NO: 64) |

In soybeans, deletions of FAD2-1A and FAD2-1B genes increased oleic acid production (Haun 2014), and deletion of SACPD-C was shown to increase the production of stearic acid (Carrero-Colón et al. (May 2014) PLoS One 9(5): e97891 [“Carrero-Colon 2014”]). Increased content of oleic and stearic fatty acids in soybeans is considered favorable and desired by the public as it is beneficial for human health (Bodkowski 2016; Zsogon 2017; Carrero-Colon 2014).

Therefore, one focus is to redirect the fatty acid biosynthetic pathway of the soybeans from production of linoleic, linolenic and palmitic fatty acids towards increased production of oleic and stearic fatty acid by depleting the above-mentioned genes. To this end, the same CRISPR system with an additional 2 pairs of guide RNAs that target the two fatty acid desaturase genes (FAD2-1A and FAD2-1B), and delta-9-stearoyl-acyl-carrier protein desaturase enzyme (SACPD-C) is used (TABLE 10).

TABLE 10
List of guide RNA sequences designed to target FAD2-1A, FAD2-1B
and SACPD-C genes.

| Gene name | Accession number | Guide 1 sequence | Guide 2 sequence |
|---|---|---|---|
| FAD2-1A | NM_001251413.1 | TTGAGTTGGCCAACA | AATAGATTGGCCAT |
| FAD2-1B | NN_001354936.1 | GTGAA | GCAATG |
| | | SEQ ID NO: 65) | (SEQ ID NO: 66) |
| SACPD-C | NM_001249462.2 | AGTGCTAGCGGCGTA | GAAGTTTATGCGAA |
| | | AGGAA | TTTATG |
| | | (SEQ ID NO: 67) | (SEQ ID NO: 68) |

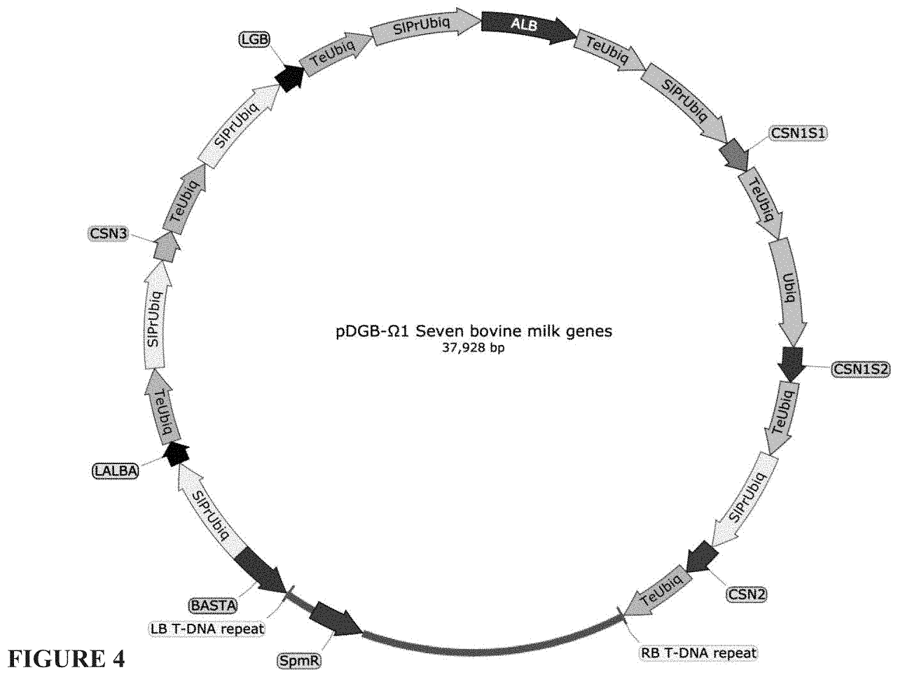

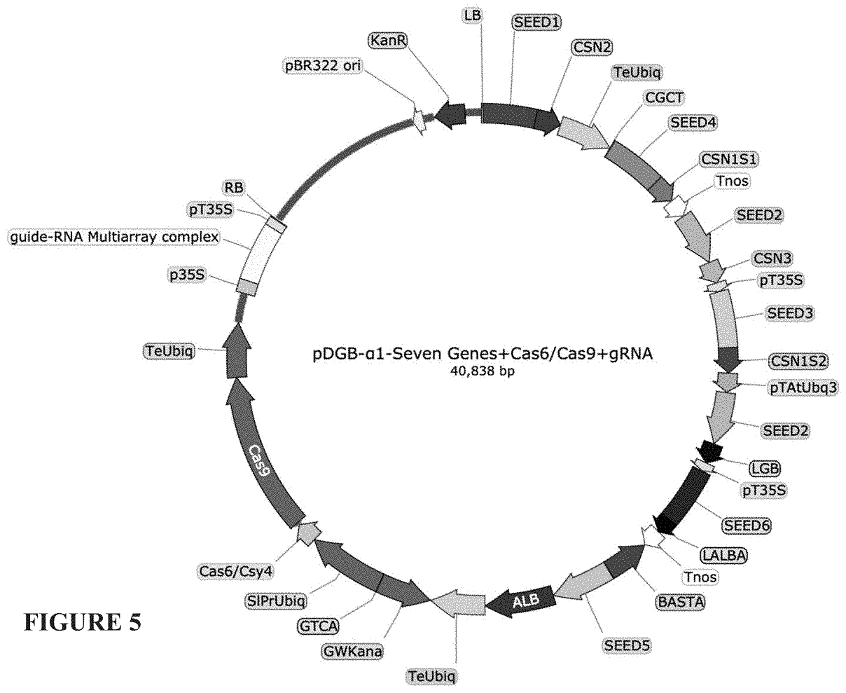

To this end, a DNA binary vector that expresses CRISPR/Cas9 and CRISPR/CSY4 together with a guide-RNA multiarray complex was designed (FIG. 5). This guide-RNA array expression is controlled by the cauliflower mosaic virus Pol-III promoter, CaMV-35S-promoter (p35s), that allows expression of long RNA molecules. The guide-RNA complex will be processed into single guide-RNAs by the CRISPR/CSY4 RNA endonuclease (see, e.g., Takahashi 2003). Four pairs of guide-RNAs to target these eight genes to induce deletion in their 5′ prime translated region that will most likely result in their silencing were designed (TABLE 9). The vector could be co-transfected with, e.g., an Agrobacterium vector encoding integration genes. The integration region lies substantially between the LB and RB sequences (FIG. 5). The vector carries the seven cow's milk genes under seed-specific promoters, and a CRISPR/Cas9 system to knock out the 11S and 7S complexes coding genes, together with knocking out the 3 fatty acid desaturases (FIG. 5, TABLE 11).

TABLE 11
pDGB-α1-Seven Genes + CSY4/Cas9 + gRNA (pDGB-alpha1-Seven
Genes + CSY4/Cas9 + gRNA)

TAACGAATTCGTCTCAGGAGAACACAAGCTTCAAGTTTTAAAAGGAAAAATGTCA
GCCAAAAACTTTAAATAAAATGGTAACAAGGAAATTATTCAAAAATTACAAACCT
CGTCAAAATAGGAAAGAAAAAAAGTTTAGGGATTTAGAAAAAACATCAATCTAG
TTCCACCTTATTTTATAGAGAGAAGAAACTAATATATAAGAACTAAAAAACAGAA
GAATAGAAAAAAAAAGTATTGACAGGAAAGAAAAAGTAGCTGTATGCTTATAAG
TACTTTGAGGATTTGAATTCTCTCTTATAAAACACAAACACAATTTTTAGATTTTA
TTTAAATAATCATCAATCCGATTATAATTATTTATATATTTTTCTATTTTCAAAGAA
GTAAATCATGAGCTTTTCCAACTCAACATCTATTTTTTTTCTCTCAACCTTTTTCAC
ATCTTAAGTAGTCTCACCCTTTATATATATAACTTATTTCTTACCTTTTACATTATG
TAACTTTTATCACCAAAACCAACAACTTTAAAATTTTATTAAATAGACTCCACAAG
TAACTTGACACTCTTACATTCATCGACATTAACTTTTATCTGTTTTATAAATATTAT
TGTGATATATTTAATCAAAATAACCACAAACTTTTCATAAAAGGTTCTTATTAAGC
ATGGCATTTAATAAGCAAAAACAACTCAATCACTTTCATATAGGAGGTAGCCTAA
GTACGTACTCAAAATGCCAACAAATAAAAAAAAAGTTGCTTTAATAATGCCAAAA
CAAATTAATAAAACACTTACAACACCGGATTTTTTTTAATTAAAATGTGCCATTTA
GGATAAATAGTTAATATTTTTAATAATTATTTAAAAAGCCGTATCTACTAAAATGA
TTTTTATTTGGTTGAAAATATTAATATGTTTAAATCAACACAATCTATCAAAATTA
AACTAAAAAAAAAATAAGTGTACGTGGTTAACATTAGTACAGTAATATAAGAGG
AAAATGAGAAATTAAGAAATTGAAAGCGAGTCTAATTTTTAAATTATGAACCTGC
ATATATAAAAGGAAAGAAAGAATCCAGGAAGAAAAGAAATGAAACCATGCATGG
TCCCCTCGTCATCACGAGTTTCTGCCATTTGCAATAGAAACACTGAAACACCTTTC
TCTTTGTCACTTAATTGAGATGCCGAAGCCACCTCACACCATGAACTTCATGAGGT
GTAGCACCCAAGGCTTCCATAGCCATGCATACTGAAGAATGTCTCAAGCTCAGCA
CCCTACTTCTGTGACGTGTCCCTCATTCACCTTCCTCTCTTCCCTATAAATAACCAC
GCCTCAGGTTCTCCGCTTCACAACTCAAACATTCTCTCCATTGGTCCTTAAACACT
CATCAGTCATCACCATGGCCAAGCTAAATGAAGGTCCTCATCCTTTGCCTGCCTGT
GGCTCTGGCCCTTGCAAGAGAGCTGGAAGAACTCAATGTACCTGGTGAGATTGTG
GAAAGCCTTTCAAGCAGTGAGGAATCTATTACACGCATCAATAAGAAAATGAGA
AGTTTCAGAGTGAGGAACAGCAGCAAACAGAGGATGAACTCCAGGATAAAATCC
ACCCCTTTGCCCAGACACAGTCTCTAGTCTATCCCTTCCCTGGGCCCATCCATAAC
AGCCTCCCACAAAACATCCCTCCTCTTACTCAAACCCCTGTGGTGGTGCCGCCTTT
CCTTCAGCCTGAAGTAATGGGAGTCTCCAAAGTGAAGGAGGCTATGGCTCCTAAG
CCAAAGAAATGCCCTTCCCTAAATATCCAGTTGAGCCCTTTACTGAAAGGCAGA
GCCTGACTCTCACTGATGTTGAAAATCTGCACCTTCCTCTGCCTCTGCTCCAGTCTT
GGATGCACCAGCCTCACCAGCCTCTTCCTCCAACTGTCATGTTTCCTCCTCAGTCC
CTTGCTGTCCCTTTCTCAGTCCAAACTTCCTGCCTGTTCCCCAGAAAGCAGTGCCCTA
TCCCCAGAGAGATATGCCCATTCAGGCCTTTCTGCTGTACCAGGAGCCTGTACTCG
GTCCTGTCCGGGGACCCTTCCCTATTATTGTCTAAGCTTGTTGTGGTTGTCTGGTTG
CGTCTGTTGCCCGTTGTCTGTTGCCCMTGTGGTGGTTGTGTTTGTATGATGGTCGT
TAAGGATCATCAATGTGTTTTCGCTTTTTGTTCCATTCTGTTTCTCATTTGTGAATA
ATAATGGTATCTTTATGAATATGCAGTTTGTGGTTTCTTTTCTGATTGCAGTTCTGA
GCATTTTGTTTTTGCATCCGTTTACTATACCACTTACAGTTTGCACTAATTTAGTTG
ATATGCGAGCCATCTGATGTTTGATGATTCAAATGGCGTTTATGTAACTCGTACCC
GAGTGGATGGAGAAGAGCTCCATTGCCGGTTTGTTTCATGGGTGGCGGAGGGCAA
CTCCTGGGAAGGAACAAAAGAAAAACCGTGATACGAGTTCATGGGTGAGAGCTC
CAGCTTGATCCCTTCTCTGTCGATCAAATTTGAATTTTTGGATCACGGCAGGCTCA
CAAGATAATCCAAAGTAAAACATAATGAATAGTACTTCTCAATGATCACTTATTTT
TAGCAAATCAGCAATTGTGCATGTCAAATGATTTCGGTGTAAGAGAAAGAGTTGA
TGAATCAAAATATCTGTAGCTGGATCAAGAATCTGAGGCAGTTGTATGTATCAAT
GATCTTTCCGCTACAATGATGTTAGCTATCCGAGTCAAATTGTTGTAGAATTGCAT
ACTTCGGCATCACATTCTGGATGACATAATAAATAGGAAGTCTTCAGATCCCTAA
AAAATTGAGAGCTAATAACCATTAGTCCTAGATGTAACTGGGTGACAACCAAGAAA
GAGACATGCAAATACTACTTTTGTTTGAAGGAGCATCCCTGGTTTGACATATTTTT
TCTGAATATCAAACTTTGAAACTCTACCTAGTCTAATGTCTAACGACAGATCTTAC
TGGTTTAACTGCAGTGATATACTAATCTTATTGGAATGTTTTCFCCTTCAGTTATAC
ATCAAGTTCCAAGATGCAGGTGTGCTTGATTGATGTACATGGCTGTGAGAAGTGC
ATCCTGATGTTCAGATGATGGTTAATTCTAATGTCTTTTTCTTAAATCAGTTTTCTC
AGTCTGACTTAGCTTGTTTCATCTGCATGTTTGAATGTTCCATTACTCATAGTAATT
GCATTTTTGTAGCAGAACATATCATTGGTCATGGTTTCAACTGTGCGCGAGTCTTA
TGCTTATTCAAACTAGGAAAGCCTCCGTCTAGAGGGTACACGAGTTGTTGCTCTGT
GTGCGTCAGTCCATAGTATTAATCTTGCTAGTTGTAGTATATTGTTTATGTGGACT
CGGAATTCATCATATGCTCCTTCTTTGCATCAAGTAAGGCAAGGTAATGTATAGAA
GCTTTTTAACTCTTTCATGGAAGCTGGCCTTTGCCAGCATACCATCCAGAAGATAT
CAACCCTGCATCTTGGCTGCCGCGCTGTCAGGAGAACTTAATCGTATATAAAAAA
TTCAATATATGAATAATTCTAAGTGAGTFTTTAAGAAAAAATAAAATTAGTAACG
AAGTAATTTATATATAAATTTGAAAAATTATCACTAAATTTGTGATCCACTTGTTAA
CATTAATTTATTCCTCTTGTATTGAATAAAATAGTTCAGACATGGTCCCAGTCTTT
AATCAATTATTCATGCTTCTCTGTCTCTCACTTATATAATCCTGTAATCCAAACATT
ACTCAGATAGCTAGATCCACCGATCAATCGTATATATATACGCATAAAATCGACG
CCTCTGTATTTTTTAGACTGTAGCCCAAATTCACTATCCGAATAAAATAAGGGAGG
CACGTGTACGTAATTTATATCATATGATAGCCATGCATATGCACACGTGCAGAAG
AGCTGTTACCCTCTATACGTGTACTCACCTTCTCATCCTCTCTGAATATTTTGAGTG
CTCTTCCTAGTTATCTAGTAATGCATGAAATTAAACTTACTAAATGTTCTTCAATT
TAAAGAAATAATTGTTTATCTGTTTCAATTTTTTTAAGAGAATTTTAAAAAGATAA
TTGTTTCGGGGAGAGAGATATAAAAAAGAAAAGGGAGAAATATTAAAATGTACT
AAATAATATGATAAGAAAAGAGAGAAAAATAAAAGAGAAAATTTGTATATAGTT
ATAATTATTCATGTAATAAGCATTCATCTCTCAACTGAAAATATACTTAATGCAGA
AGAAAAAATCATTATTTACAAACGTTGAGTCTTGAGTGGGAAAAGAGGAGGCGCC
GTTACTATACAATATAAGATCATAGTACTGACAAAATGCACAGTAAAACAGTTCA
AATTGAGAAGGATTCTTAACACACCATAGTATTTAATATATATCTTTACAGAGACA
ATTATGCTGGAGGATTCAGGCAAAGATTATATATTGTGGATTTGTTTTTTAATAAT
TAACGCATCATATGAAAGATCGATGATATATACTAATGGTTATAAGAAAAATATT
TAACAGTTTCTATAACCTTTTTCTTTTATCTTTTACTGTAATATTATTTATTTTATTT
CACATTTTTAATCAGCTTATCTCATTTATAAACGAAATTGTATAAAAATATACATG
ATGAACTGAATAGAACAATATTGATCTGATATTCTCATATTGTATAAGAGGATAG
ACTTTGAGGCGCGGAGAATCTGTAGGAGGGGACCATTCAGAGTGCCTCCAATTTT
GGTGTTGTTCATTGTACCATTGCAAATATAAACGAAGCATGCATGCTTATGTATGA
GGTGTAACAAAATTGGAAACAATAGCCATGCAAGGTGAAGAATGTCACAAACTC
AGCAACCCTTATTCATTGACGTGTCCCTCAGTCACTCTCCTCTCATACCTATAAAT
CACCACTCCTCATGTTCTTTCCAATTACCAACTCCTTCAAACTTAATTATTAACACT
TCCTTAGTTCAATATGGGGAAGCCAATGAAACTTCTCATCCTTACCTGTCTTGTGG
CTGTTGCTCTTGCCAGGCCTAAACATCCTATCAAGCACCAAGGACTCCCTCAAGA
AGTCCTCAATGAAAATTTACTCAGGTTTTTTGTGCACCTTTTCCAGAAGTGTTTG
GAAAGGAGAAGGTCAATGAACTGAGCAAGGATATTGGGAGTGAATCAACTGAGG
ATCAAGCCATGGAAGATATTAAGCAAATGGAAGCTGAAAGCATTTCCACAAGTGA
GGAAATTGTTCCCAATAGTGTTGAGCAGAAGCACATTCAAAAGGAAGATGTGCCC
TCTGAGCGTTACCTGGGTTATCTGGAACAGCTTCTCAGACTGAAAAAATACAAAG
TACCCAGCTGGAAATTGTTCCCAATAGTGCTGAGGAACGACTTCACAGTATGAA
AGAGGGAATCCATGCCGAACAGAAAGAACCTATGATAGGAGTGAATCAGGAACT
GGCCTACTTCTACCCTGAGCTTTTCAGACAATTCTACCAGCTGGATGCCTATCCAT
CTGGTGCCTGGTATTACGTTCCACTAGGCACACAATACACTGATGCCCCATCATTC
TCTGACATCCCTAATCCCATTGGCTCTGAGAACAGTGAAAAGACTACTATGCCACT
GTGGTGAGCTTGGAATGGATCTTCGATCCCGATCGTTCAAACATTGGCAATAAA
GTTTCTTAAGATTGAATCCTGTTGCCGGTCTTGCGACGATTATCATATAATTTCTGT
TGAATTACGTTAAGCATGTAATAATTAACATGTAATGCATGACGTTATTTATGAGA
TGGGTTTTTATGATTAGAGTCCCGCAATTATACATTTAATACGCGATAGAAAACAA
AATATAGCGCGCAAACTAGGATAAATTATCGCGCDCGGTGTCATCTATGTTACTA
GATCGGGAATTGCCAAGCTAATTCTTGAAGACGAAAGGGCCTCGTGATACGCCTA
TTTTTATAGGTTAATGTCATGATAATAATGGTTTCTTAGACGTCAGGTGGCACTTT
TCGGGGAAATGTGCGCGGAACCCCTATTTGTTTATTTTTCTAAATACATTCAAATA
TGTATCCGCTCATGAGACAATAACCCTGATAAATGCTTCAATATATGGGACCGACT
CGCGCTGTCAGGAGTACATTTTGAGTTGTTTCAGGTTCGATTGCCTTATTGCTAAA
ACTCCAACTAAAATAACAAATAGCACATGCAGGTGCAAACAACACGTTACTCTGA
TGAAGGTGATGTGCCTCTAGCAGTCTAGCTTATTGAGGCTCGCTGCTTATCAACGAT
TCATCATTCCCCAAGACGTGTACGCAGATTAAACAATGGACAAAACTTCAATCGA
TTATAGAATAATAATTTTAACAGTGCCGACTTTTTTATGTAAACAAAAGGCCAGAA
TCATATTCGTACATCATCTTGAATGCAGTGTCGAGTTTGGACCATTTGAGTACAAAG
CCAATATTGAATGATTTTTCGATTTTACATGTGTGAATCAGACAAAAGTGCATGCA
ATCACTTGCAAGTAAATTAAGGATACTAATCTATTCCTTTCATTTTATATGCTCCA
CTTTTATATAAAAAAATATACATTATTATATATGCATTATTAATTATTGCAGTATT
ATGCTATTGGTTTTATGGCCCTGCTAAATAACCTAAATGAGTCTAACTATTGCATA
TGAATCAAATGAAGGAAGAATCATGATCTAAACCTGAGTACCCAATGCAATAAAA
TGCGTCCTATTACCTAAACTTCAAACACACATTGCCATCGGACGTATAAATTAATG
CATATAGATTATTTTGAGAAAAGAAAACATCAAAAGCTCTAAAACTTCTTTTAACT
TTGAAATAAGCTGATAAAAATACGCTTTAAATCAACTGTGTGCTGTATATAAGCT
GCAATTTCAGATTTTACCAAACCGAAACAAGAATGGTAACAGTGAGGCAAAAATT
TGAAAAATGTCCTACTTCACATTCACATCAAATTAATTACAACTAAATAAATAAA
CATCGTGATTCAAGCAGTAATGAAAGTCGAAATCAGATAGAATATACACGTTTAA
CATCAATTGAATTTTTTTTTAAATGGATATATACAAGTTTACTATTTTATATATAAT
GAAAATTCATTTTGTGTTAGCACAAAACTTACAGAAAGAGATAAATTTAAATAA
AGAGAATTATATCCAATTTTATAATCCAAAATAATCAAATTAAAGAATATTGGCT
AGATAGACCGGCTTTTTCACTGCCCCTGCTGGATAATGAAAATTCATATCAAAAC
AATACAGAAGTTCTAGTTTAATAATAAAAAAGTTGGCAAACTGTCATTCCCTGTTG
GTTTTTAAGCCAAATCACAATTCAATTACGTATGAGAAATTAATTTAAACCAAATA
TATAGCTACGAGGGAACTTCTTCAGTCATTACTAGCTAGCTCACTAATCACTATAT
ATACGACATGCTACAAGTGAAGTGACCATATCTTAATTTCAAATCATAAAATTCTT
CCACCAAGTTATGGGTTTCCTAATGATGAAGAGTTTTTTCCTAGTTGTGACTATCC
TGGCATTAACCCTGCCATTTTTGGGTGCCCAGGAGCAAAACCAAGAACAACCAAT
ACGCTGTGAGAAAGATGAAAGATTCTTCAGTGACAAAATAGCCAAATATATCCCA
ATTCAGTATGTGCTGAGTAGGTATCCTAGTTATGGACTCAATTACTACCAACAGAA
ACCAGTTGCACTAATTAATAATCAATTTCTGCCATACCCATATTATGCAAAGCCAG
CTGCAGTTAGGTCACCTGCCCAAATTCTTCAATGGCAAGTTTTGTCAAATACTGTG
CCTGCCAAGTCCTGCCAAGCCCAGCCAACTACCATGGCACGTCACCCACACCCAC
ATTTATCATTTATGGCCATTCCACCAAAGAAAAATCAGGATAAAACAGAAATCCC
TACCATCAATACCATTGCTAGTGGTGAGCCTAGAAGTACACCTACCATCGAAGCA
GTAGAGAGCACTGTAGCTACTCTACAAGCTTCTCCAGAAGTTATTGAGAGCCCAC
CTGAGATCAACACAGTCCAAGTTACTTCAACTGCGGTCTAAGCTTCGGCCATGCTA
GAGTCCGCAAAAATCACCAGTCTCTCTCTACAAATCTATCTCTCTCTATTTTTCTCC
AGAATAATGTGTGAGTAGTTCCCAGATAAGGGAATTAGGGTTCTTATAGGGTTTC
GCTCATGTGTTGAGCATATAAGAAACCCTTAGTATGTATTTGTATTTGTAAAATAC
TTCTATCAATAAAATTTCTAATTCCTAAAACCAAAATCCAGTGACCTCGCTGTCAG
GAGATTATTTCTGTTAGTACATAGCTAATACTCAATCAACGGAATTAGTATATGGT
TCTTCATATAGGAGAGTACTTATTTATTCTATTGAATTTTAACATATAAGCATAAT
AAAATACTTTTGGACTCTCGTATAAAGTTCGATTTTAATCTTTTAATAATTCAATC
TAAATGTTTAATTCCCTCTTAAATGCAAAATTCAGTTTTCGTTCCTTTAATGTGACA
CCATTAGGTCACATGAACCGGAAATGACGTGGTGATCGAATTATGACTTGAATCC
ATTGACCACATTAGCATTTCACCTATGGTCACTAGTATGAAGGATGAAAACAAGT
CTATTTCTCAAATTATAAATGAAAACGTTTAACTTTAAACCTGAGGATCCAAAAAC
GAATTTTACTAAATTTTGAAGAACTAAAAAATATTTAATCTAGTAAAACGCGTGTC
TATCTAGTATAACATGCACGCTCGTCATGTATTCAATTAGGCATAAAAATAGTGTTT
TGATTTTTTGACACATTATTAAGTGTTTTATTTTTAAGTTTAAAAGCATTGGTATCC
TTTCATAAAAGGAGGTAATCTTATTTAAGTCAAGGAGAATTATTATGGGAAATAA
AACCTTTTTTTTTAAAGTGTTTAATATAATTATATACTCAAAATTCGATTTATGATT
AAATCTAAGTGACATTTAAAAAAAATTAGTGTGAAAATAATTTATATATAATTTTG
AAAAATCATTAATTTTTTTTTATAAATAAATGTTAATTTKTTAGTTTTTATTTA
TAAATGTGAATAGAATGGATTCGAAGCAGCAATTTCTCTCTTTCTCCTTTTCCATG
CCAACCTTATATATGGTGACGAACTGCATATACAGTAAAACAGTTCAAATTGAGA
AAGATTTTAAACATCATAGTATTTGATATATATCTTTTACAGAGACAATTATGCTG
CAGGAGTTAGATAAGATTATTGTGGATGTCATTTTCTTTTTTAATATTTAACGCATT
ATATAAAAGATGATATAGTATGGTTATAAAAAAATTATTTAACAGTTTATAAAAC
CTTTTTTTTTATCTTTTAGAGTAATATTATTTATTTTATTTCACATTTTTTTCATATC
CTTATCTCATTTATAAAGGAAATTAATTGTATAAAAAAAATATGATGCACTGAAT
AGAATGCTGATCTTATTGTATAAGGAGGATAGAATTTGAGACACGGAGAATCTGT
AGAGGGGGACCATTCAGGGTGCCTGCAATTTTGGTGTTGTTCATGTACGGTTGCA
GATATAAACGAAGCATAGCTTATGTATGAGGTGTAACAAAATTGGAAACAATAGC
CATGCAAGGTGAAGAATGTCACCAACTCAGAAACCCTTCTTCATTGACGTGTCCCT
CACTCACTCTCCTCTCTTCACTATAAATCGCCACTCTTCGTGTTCTCCACTTCACCA
ACTCCTTCAAACTTATTAACACTTTCCTTAGTTCAATATGGGGAAGCAATGAAGTT
CTTCATTCTTTACCTGCCTTTTGGCTGTTGCCCTTGCAAAGAATACGATGGAACATG
TCTCCTCCAGTGAGGAATCTATCATCTCCCAGGAAACATATAAGCAGGAAAAGAA
TATGGACATTAATCCCAGCAAGGAGAACCTTTGCTCCACATTCTGCAAGGAAGTT
GTAAGGAACGCAAATGAAGAGGAATATTCTATCGGCTCATCTAGTGAGGAATCTG
CTGAAGTTGCCACAGAGGAAGTTAAGATTACTGTGGACGATAAGCACTACCAGAA
AGCACTGAATGAAATCAATCAGTTTTATCGGAAGTTCCCCCAGTATCTCCAGTATC
TGTATCAAGGTCCAATTGTTTTGAACCCATGGGATCAGGTTAAGAGAAATGCTGTT
CCCATTACTCCCACTCTGAACAGAGAGCAGCTCTCCACCAGTGAGGAAAATTCAA
AGAAGACCGTTGACATGGAATCAACAGAAGTATTCACTAAGAAAACTAAACTGA
CTGAAGAAGAAAAGAATCGCCTAAATTTTCTGAAAAAAATCAGCCAGCGTTACCA
GAAATTCGCCTTGCCCCAGTATCTCAAAACTGTTTATCAGCATCAGAAAGCTATGA
AGCCATGGATTCAACCTAAGACAAAGGTTATTCCCTATGTGACGTACCTTTAAGCT
TAAGCTTTTTGTGATCTGATGATAAGTGGTTGGTTCGTGTCTCATGCACTTGGGAG
GGATCTATTTCACCTGGTGTAGTTTGTGTTTCCGTCAGTTGGAAAAACTTATCCCT
ATCGATTTCGTTTTCATTTTCTGCTTTTCTTTTATGTACCTTCGTTTGGGCTTGTAAC
GGGCCTTTGTATTTCAACTCTCAATAATAATCCAAGTGCATGTTAAACAATTTGTC
ATCTGTTTCGGCTTTGATATACTACTGGTGAAGATGGGCCGTACTACTGCATCACA
ACGAAAAATAATAATAAGATGAAAAACTTGAAGTGGAAAAAAAAAAAACTTGAA
TGTTCACTACTACTCATTGACCATAATGTTTAACATACATAGCTCAATAGTATTTTT
GTGAATATGGCAACACAAACAGTCCAAAACAATTGTCTCTTACTATACCAAACCA
AGGGCGCCGCTTGTTTGCCACTCTTTGTGTGCAATAGTGTGATTACCACACGCTGT
CAGGAGTACATTTTGAGTTGTTTCAGGTTCCATTGCCTTATTGCTAAAACTCCAAC
TAAAATAACAAATAGCACATGCAGGTGCAAACAACACGTTACTCTGATGAAGGTG
ATGTGCCTCTAGCAGTCTAGCTTATGAGGCTCGCTGCTTATCAACGATTCATCATT
CCCCAAGACGTGTACGCAGATTAAACAATGGACAAAACTTCAATCGATTATAGAA
TAATAATTTTAACAGTGCCGACTTTTTTCTGTAAACAAAAGGCCAGAATCATATCG
CACATCATCTTGAATGCAGTGTCGAGTTTGGACCATTTGAGTACAAAGCCAATATT
GAATGATTTTTCGATTTTACATGTGTGAATCAGACAAAAGTGCATGCAATCACTTG
CAAGTAAATTAAGGATACTAATCTATTCCTTTCATTTTATATGCTCCACTTTTATAT
AAAAAAATATACATTATTATATATGCATTATTAATTATTGCAGTATTATGCTATTG
GTTTTATGGCCCTGCTAAATAACCTAAATGAGTCTAACTATTGCATATGAATCAAA
TGAAGGAAGAATCATGATCTAAACCTGAGTACCGATATGCAATAAAATGCGTCCTA
TTACCTAAACTTCAAACACACATTGCCATCGGACGTATAAATTAATGCATGTAGAT
TATTTTGAGAAAAGAAAACATCAAAAGCTCTAAAACTTCTTTTAACTTTGAAATA
AGCTGATAAAAATACGCTTTAAATCAACTGTGTGCTGTATATAAGCGTCAATTTCA
CATTTTACCAAACCGAAACAAGAATGGTAACAGTGAGGCAAAAATTTGAAAAAT
GTCCTACTTCACATTCACATCAAATTAATTACAACTAAATAAATAAACATCGTGAT
TCAAGCAGTAATGAAAGTCGAAATCAGATAGAATATACACGTTTAACATCAATTG
AATTTTTTTTTAAATGGATATATACAAGTTTACTATTTTATATATAATGAAAATTCA
TTTTGTGTAGCACAAAACTTACAGAAAGAGATAAAFTTTAAATAAAGAGAATTA
TATCCAATTTTATAATCCAAAATAATCAAATTAAAGAATATTGGCTAGATAGACC
GGCTTTTTCACTGCCCCTGCTGGATAATGAAAATTCATATCAAAACAATACAGAA
GTTCTAGTTTAATAATAAAAAAGTTGGCAAACTGTCATTCCCTGTTGGTTTTTAAG
CCAAATCACAATTCAATTACGTATCAGAAATTAATTTAAACCAAATATATAGCTA
CGAGGGAACTTCTTCAGTCATTACTAGCTAGCTCACTAATCACTATATATACGACA
TGCTACAAGTGAAGTGACCATATCTTAATTGTAAATCATAAAATTCTTCCACCAAG
TTATGGGTTTCCTAATGAAGTGCCTCCTGCTTGCCCTGGCCCTCACTTGTGGCGCC
CAGGCCCTCATTGTCACCCAGACCATGAAGGGCCTGGATATCCAGAAGGTGGCGG
GGACTTGGTACTCCTTGGCCATGGCGGCCAGCGACATCTCCCTGCTGGACGCCCCCA
GAGTGCCCCCCTGAGAGTGTATGTGGAGGAGCTGAAGCCCACCCCTGAGGGCGAC
CTGGAGATCCTGCTGCAGAAATGGGAGAACGGTGAGTGTGCTCAGAAGAAGATC
ATTGCAGAAAAAACCAAGATCCCTGCGGTGTTCAAGATCGATGCCTTGAATGAGA
ACAAAGTCCTTGTGCTGGACACCGACTACAAAAAGTACCTGCTCTTCTGCATGGA
GAACAGTGCTGAGCCCGAGCAAAGCCTGGCCTGCCAGTGCCTGGTCAGGACCCCG
GAGGTGGACGACGAGGCCCTGGAGAAATTCGACAAAGCCCTCAAGGCCCTGCCC
ATGCACATCCGGCTGTCCTTCAACCCAACCCAGCTGGAGGAGCAGTGCCACATCT
AGGCTTCGGCCATGCTAGAGTCCGCAAAAATCACCAGTCTCTCTCTACAAATCTAT
CTCTCTCTATTTTTCTCCAGAATAATGTGTGAGTAGTTCCCAGATAAGGGAATTAG
GGTTATAGGGTTTCGCTCATGGTTTGAGCATATAAGAAACCCTTAGTATGTAT
TTGTATTTGTAAAATACTTCTATCAATAAAATTTCTAATTTCCTAAAACCAAAATCC
AGTGACCTCGCTGTCAGGAGTATAAACACCACTTTAATTTGACTCGGATACATGC
ATCCATAAAGACTACAAAAGGCAAAAAGAGAAGGAAATGAGATACGAATATATG
TCATAAGTATATATAGGTGACAAGGGCAAATTAAATAGGTTGGTATTTAAATGCA
AAATCCTATGTTTGATAAAGAATGGTATGAAAAACAGGCAAAGTTAATTGCAATT
CAAAGGTGAACAAAGCATTTCTTTGTCTACACTAATGGCATGTCTAAGTAAATTAT
TAGTCTTGTATCTATATGTCCACAAGTTATTAATTAGTCTTATACTATCAAAAACA
AGTTAAGTTGCAAATCAAACATGAACAAAGCATTTGTGTTGTAACCTACGAAAAA
ATACCCTAACATACTGATACGAATAATGTGGCCTAAATGTATCGTTTACCAAATTA
CGGTGCTGGAAAAAAAAATTGCTCCTTTACCAACAAAATTAAGAACTGATACATC
TTGTTFTTTGTCACTGAAGATAAACACGTGATCTTMGCAAAACATAAAGGCCAAC
AAAACAAACTTGTTCTTCATCCCTGAATGATTCGAATGCCATCGTATGCGTGTCACAA
AGTGGAATACAGCAATGAACAAATGCTATCCTCTTGAGAAAAGTGAATGCAGCAG
CAGCAGCAGACTAGAGTGCTACAAATGCTTATCCTCTGTAGAAAAGTGAATGCAG
CGGCAGCAGACCTGAGTGCTATATACAATTAGACACAGGGTCTATTAATTGAAAT
TGTCTTATTATTAAATATTTCGTTTTATATTAATTTTTTAAATTTTAATTAAATTTAT
ATATATTATATTTAAGACAGATATATTTATTTGTGATTATAAATGTGTCACTTTTTC
TTTTAGTCCATGTATTCTTCTATTTTTTCAATTTAACTTTTTATTTTTATTTTTAAGT
CACTCTTGATCAAGAAAACATTGTTGACATAAAACTATTAACATAAAATTATGTTA
ACATGTGATAACATCATATTTTACTAATATAACGTCGGATTTTAACGTTTTTTTAAC
AAATATCGACTGTAAGAGTAAAAATGAAATGTTTGAAAAGGTTAATTGCATACTA
ACTATTTTTTTTCCTATAAGTAATCTTTTTTGGGATCAATTGTATATCATTGAGATA
CGATATTAAATATGGGTACCTTTTCACAAAACCTAACCCTTGTTAGTCAAACCACA
CATAAGAGAGGATGGATTTAAACCAGTCAGCACCGTAAGTATATAGTGAAGAAG
GCTGATAACACACTCTATTATTGTTAGTACGTACGTATTTCCTTTTTTGTTTAGTTT
TTGAATTTAATTAATTAAAATATATATGCTAACAACATTAAATTTTAAATTTACGT
CTAATTATATATTGTGATGTATAATAAATTGTCAACGTTAAAAATTATAAAAGAA
ATATTAATTTTGATAAACAACTTTTGAAAAGTACCCAATAATGCTAGTATAAATAG
GGGCATGACTCCCCATGCATCACAGTGCAATTTAGCTGAAGCAAAGCAATGGCTA
CTTAATGATGTCCTTTGTCTCTCTGCTCCTGGTAGGCATCCTATTCCATGCCACCCA
GGCTGAACAGTTAACAAAATGTGAGGTGTTCCGGGAGCTGAAAGACTTGAAGGG
CTACGGAGGTGTCAGTTTCGCCTGAATGGGTCTGTACCACGTTTCATACCAGTGGTT
ATGACACACAAGCCATAGTACAAAACAATGACAGCACAGAATATGGACTCTTCCA
GATAAATAATAAAATTTGGTGCAAAGACGACCAGAACCCTCACTCAAGCAACATC
TGTAACATCTCCTGTGACAAGTTCCTGGATGATGATCTTACTGATGACATTATGTG
TGTCAAGAAGATTCTGGATAAAGTAGGAATTAACTACTGGTTGGCCCATAAAGCA
CTCTGTTCTGAGAAGCTGGATCAGTGGCTCTGTGAGAAGTTGTGAGCTTGGAATG
GATCTTCGATCCGATCGTTCAAACATTTGGCAATAAAGTTTCTTAAGATTGAATC
CTGTTGCCGGTCTTGCGACGATTATGATATAATTTCTGTTGAATTACGTTAAGCAT
GTAATAATTAACATGTAATGCATGACGTTATTTATGAGATGGGTTTTTATGATTAG
AGTCCCGCAATTATACATTTAATACGCGATAGAAAACAAAATATAGCGCGCAAAC
TAGGATAAATTATCGCGCDCGGTGTCATCTATGTTACTAGATCGGGAATTGCCAA
GCTAATTCTTGAAGACGAAAGGGCCTCGTGATACGCCTATTTTTATAGGTTAATGT
CATGATAATAATGGTTTCTTAGACGTCAGGTGGCACTTTTCGGGGAAATGTGCGC
GGAACCCCTATTTGTTTATTTTTCTAAATACATTCAAATATGTATCCGCTCATGAG
ACAATAACCCTGATAAATGCTTCAATAATGGGACCGACTCGCGCTGTCAGGAGAG
CGATCAGCTTGCATGCCGGTCGATCTAGTAACATAGTAGATGACACCGCGCGCGA
TAATTTATCCTAGTTTGCGCGCTATATTTTGTTTTCTATCGCGTATTAAATGTATAA
TTGCGGGACTCTAATCATAAAAACCCATCTCATAAATAACGTCATGCATTACATGT
TAATTATTACATGCTTAACGTAATTCAACAGAAATTATATGATAATCATCGCAAGA
CCGGCAACAGGATTCAATCTTAAGAAACTTTATTGCCAAATGTTTGAACGATCTGC
TTGACTCTAGGGGTCATCAGATTTCGTGACGGGCAGGACCGGACGGGGCGGCAC
CGGCAGGCTGAAGTCCAGCTGCCAGAAACCCACGTCATGCCAGTTCCCGTGCTTG
AAGCCGGCCGCCCGCAGCATGCCGCGGGGGGCATATCCGAGCGCCTCGTGCATGC
GCACGCTCGGGTCGTTGGGCAGCCCGATGACAGCGACCACGCTCTTGAAGCCCTG
TGCCTCCAGGGACTTCAGCAGGTGGGTGTAGAGCGTGGAGCCCAGTCCCGTCCGC
TGGTGGCGGGGGGATACGTACACGGTCGACTCGGCCGTCCAGTCGTAGGCGTTGC
GTGCCTTCCAGGGACCCGCGTAGGCGATGCCGGCGACCTCGCCGTCCACCTCGGC
GACGAGCCAGGGATAGCGCTCCCGCAGACGGACGAGGTCGTCCGTCCACTCCTGC
GGTTCCTGCGGCTCGGTACGGAAGTTGACCGTGCTTGTCTCGATGTAGTGGTTGAC
GATGGTGCAGACCGCCGGCATGTCCGCCTCGGTGGCACGGCGGATGTCGGCCGGG
CGTCGTTCTGGGCTCATGGTAGATCCCCTCGATCGAGTTGAGAGTGAATATGAGA
CTCTAATTGGATACCGAGGGGAATTTATGGAACGTCAGTGGAGCATTTTTGACAA
GAAATATTTGCTAGCTGATAGTGACCTTAGGCGACTTTTGAACGCGCAATAATGG
TTTCTGACGTATGTGCTTAGCTCATTAAACTCCAGAAACCCGCGGCTCAGTGGCTC
CTTCAACGTTGCGGTTCTGTCAGTTCCAAACGTAAAACGGCTTGTCCCGCGTCATC
GGCGGGGGTCATAACGTGACTCCCTTAATTCTCATGTATGATACTCCGTCAGGAG
ATAATTATAAAATTGTCACTGCGTTCAAAACGACAATGGTTTTGGGACAACTATC
ATTAATCGTGCATTGTAAAAAGGTGTGTTTTTAGTAGTGGACCCTCGATAAATTGA
CTGTGATGATTGTTACATGTTGTTTAAGTCTCACCTATAAGAAAAAAACTAAACATA
TATATAGATCCCAATTTTGGGGTCAGGTGTATAGATGAAAAAAAGAAACAAATAG
ACAAATAAAAAAATAAAAGAAAAAAAATTGATAGATGTGAGAAATGATGAGAAG
AGAAGTGCAAATAACACACTCTTTCTAACATTATTTTACTATTGATTAAAATTTAT
TGAAAATTACTATATAATATAAAAAGTGAAACTAGTTAAACTATAGTCAATAATT
GAGAATATTTAAAAATTTAGAAAATACATTACTTATATTTCTTAAAATAAAAAAT
ATAAATAAAAATAGAAAAAATGGAGTAAAATGAGATAGAAGAGAAGTTAGGTTT
ATAAATACATTAGTTCCGCCTACAATATATTTAAATTAGCTAGATTAATGCAGTAA
ATTTTTGGCATTTACTTGATTTTATTTTCTTTAAAAGCATTCTTTGTATTCTTCACTG
ATGGTTTTTTTTCTTCATCTGCATTATGAATTAAATCATTTACTTTGTGTCACAATT
GCATTTAGCGAGGTCATGCATTGGTTAGACCGACGGTGTATTATGTCATGACTTAG
GTCTTGAAGGTTGTTGGTTACTTATTATTGGTCCATGGGTACACGCTGTTGGTTAGAT
TCGATAGGCAAATTTTGTGAACGATAGAAATTTATCTTTATTAAATAAACCACACT
ATATATATATATATATATATATATATATATATATATATATATATTAATTCGTAATFT
CTTTTCTGTCTTTCATTTTGATTTTCTTTTATGGCTTTTATCTTTAAAAATTTTCCCC
TTCTTTAAAATTTACAACACTTTATAATCACAATAAAATAAAATAATTTAAAATAT
TACATAAATAATAACACAAATATTTATAAATCTGAAATGACATAAAATAACATTA
TAATCACAAAAAGTATTTAATAAAAATAAAATTACATAAATAAAATATTGTGAAA
ACTAAGTAAAAGGTATCATGCACGTAATCATATGAAAATAGCTTTAGAAAAAATA
TCAAGGCAACTTACCGCACGTACGATAAATGAAAAAAGATTAAAAAGAAATATAA
TAAATAATAATACTAAATTAATGGTGAATAAAATACTAAAAAAATAAATTTATAA
TTAAATAATATGTATTACAAACACAAATAAGAAATAATAGTACATAATATTATAA
TAAATAGTAGTATATAACATATCATAAATATGTTTAAAATAATGATAAAATATTG
AGTTTCTTTTAGTGGAACTATTTGTCAAAATGTGAACACCTGGATATGAAAAGGC
ATCTTAGGTAGATGATATGATGCGATAGAACGTAAAAGAAAAATGAGAAATGTTG
ATGAGAGGTTAAAAATACCCTTCATAACAAGCACACATCTATAAGTAGTCTTATT
CACCCAACAACGTTGCTTATTCACGCAACTAAATAAGAAATGAAGAGTACTATAA
TGAAGTGGGTGACTTTTATTTCTCTTCTCCTTCTCTTCAGCTCTGCTTATTCCAGGG
GTGTGTTTCGTCGAGATACACACAAGAGTGAGATTGCTCATCGGTTTAAAGATTTG
GGAGAAGAACATTTTAAAGGCCTGGTACTGATTGCCTTTTCTCAGTATCTCCAGCA
GTGTCCATTTGATGAGCATGTAAAATTAGTGAACGAACTAACTGAGTTTGCAAAA
ACATGTGTTGCTGATGAGTCCCATGCCGGCTGTGAAAAGTCACTTCACACTCTCTT
TGGAGATGAATTGTGTAAAGTTGCATCCCTTCGTGAAACCTATGGTGACATGGCT
GACTGCTGTGCGAAACAAGAGCCTGAAAGAAATGAATGCTTCCTGAGCCACAAA
GATGATAGCCCAGACCTCCCTAAATTGAAACCAGACCCCAATACTTTGTGTGATG
AGTTTAAGGCAGATGAAAAGAAGTTTTGGGGAAAATACCTATACGAAATTGCTAG
AAGACATCCCTACGTTATGCACCAGAACTCCTTTACTATGCTAATAAATATAATG
GAGTTTTTCAAGAATGCTGCCAAGCTGAAGATAAAGGTGCCTGCCTGCTACCAAA
GATTGAAACTATGAGAGAAAAAGTACTGACTTCATCTGCCAGACAGAGACTCAGG
TGTGCCAGTATTCAAAAATTTGGAGAAAGAGCTTTAAAAGCATGGTCAGTAGCTC
GCCTGAGCCAGAAATTTCCCAAGGCTGAGTTTGTAGAAGTTACCAAGCTAGTGAC
AGATCTCACAAAAGTCCACAAGGAATGCTGCCATGGTGACCTACTTGAATGCGCA
GATGACAGGGCAGATCTTGCCAAGTACATATGTGATAATCAAGATACAATCTCCA
GTAAACTGAAGGAATGCTGTGATAAGCCTTTGTTGGAAAAATCCCACTGCATTGC
TGAGGTGGAAAAAGATGCCATACCTGAAAACCTGCCCCCATTAACTGCTGACTTT
GCTGAAGATAAGGATGTTTGCAAAAACTATCAGGAAGCAAAAGATGCCTTCCTGG
GCTCGTTTTTGTATGAATATTCAAGAAGGCATCCTGAATATGCTGTCTCAGTGCTA
TTGAGACTTGCCAAGGAATATGAAGCCACACTGGAGGAATGCTGTGCCAAAGATG
ATCCACATGCATGCTATTCCACAGTGTTTGACAAACTTAAGCATCTTGTGGATGAG
CCTCAGAATTTAATCAAACAAAACTGTGACCAATTCGAAAAACTTGGAGAGTATG
GATTCCAAAATGAGCTCATAGTTCGTTACACCAGGAAAGTACCCCAAGTGTCAAC
TCCAACTCTCGTGGAGGTTTCAAGAAGCCTAGGAAAAGTGGGTACTAGGTGTTGT
ACAAAGCCGGAATCAGAAAGAATGCCCTGTGCTGAAGACTATCTGAGCTTGATCC
TGAACCGGTTGTGCGTGCTGCATGAGAAGACACCAGTGAGTGAAAAAGTCACCAA
GTGCTGCACAGAGTCATTGGTGAACAGACGGCCATGTTTCTCTGCTGACACCTG
ATGAAACATATGTACCCAAAGCCTTTGATGAGAAATTGTTCACCTTCCATGCAGAT
ATATGCACACTTCCCGATACTGAGAAACAAATCAAGAAACAAACTGCACTTGTTG
AGCTGTTGAAACACAAGCCCAAGCTTAACAGAGGAACAACTGAAAACCGTCATGG
AGAATTTTGTGGCTTTTGTAGGCAAGTGCTGTGCAGCTGATGACAAAGAGGCCTG
CTTTGCTGTGGAGGGTCCAAAACTTGTTGTTTCAACTCAAACAGCCTTAGCCTAAG
CTTGTTGTGGTTGTCTGGTTGCGTCTGTTGCCCGTTGTCTGTTGCCCATTGTGGTGG
TTGTGTTTGTATGATGGTCGTTAAGGATCATCAATGTGTTTTCGCTTTTTGTTCCAT
TCTGTTTCTCATTTGTGAATAATAATGGTATCTTTATGAATATGCAGTTTGTGGTTT
CTTTTCTGATTGCAGTTCTGAGCATTTTGTTTTTGCTTCCGTTTACTATACCACTTA
CAGTTTGCACTAATTTAGTTGATATGCGAGCCATCTGATGTTTGATGATTCAAATG
GCGTTTATGTAACTCGTACCCGAGTGGATGGAGAAGAGCTCCATTGCCGGTTTGTT
TCATGGGTGGCGGAGGGCAACTCCTGGGAAGGAACAAAAGAAAAACCGTGATAC
GAGTTCATGGGTGAGAGCTCCAGCTTGATCCCTTGTCTGTCGATCAAATTTGAATT
TTTGGATCACGGCAGGCTCACAAGATAATCCAAAGTAAAACATAATGAATAGTAC
TTCTCAATGATCACTTATTTTTAGCAAATCAGCAATTGTGCATGTCAAATGATTTC
GGTGTAAGAGAAAGAGTTGATGAATCAAAATATCTGTAGCTGGATCAAGAATCTG
AGGCAGTTGTATGTATCAATGATCTTTCCGCTACAATGATGTTAGCTATCCGAGTC
AAATTGTTGTAGAATTGCATACTTCGGCATCACATTCTGGATGACATAATAAATAG
GAAGTCTTCAGATCCCTAAAAAATTGAGAGCTAATAACATTAGTCCTAGATGTAA
CTGGGTGACAACCAAGAAAGAGACATGCAAATACTACTTTTGTTTGAAGGAGCAT
CCCTGGTTTGACATAGTTTTCTGAATATCAAACTTTGAAACTCTACCTAGTCTAAT
GTCTAACGACAGATCTTACTGGTTTAACTGCAGTGATATCTACTATCTTTTGGAAT
GTTTTCTCCTTCAGTTATACATCAAGTTCCAAGATGCAGGTGTGCTTGATTGATGT
ACATGGCTGTGAGAAGTGCATCCTGATGTTCAGATGATGGTTCATTCTAATGTCTT
TTCCTTCAATCAGTTTTCTCAGTCTGACTTAGCTTGTTTCATCTGCATGTTTGAATG
TTCGTTTACTCATAGTAATTGCATTTTTGTAGCAGAACATATCATTGGTCATGGTTT
CAACTGTGCGCGAGTCTTATGCTTATTCAAACTAGGAAAGCCTCCGTCTAGAGGG
TACACGAGTTGTTGCTCTGTGTGCGTCAGTCCATAGTATTAATCTTGCTAGTTGTA
GTATATTGTTTATGTGGACTCGGAATTCATCATATGCTCCTTCTTTGCATCAAGTA
AGGCAAGGTAATGTATAGAAGCTTTTAACTCTTTCATGGAAGCTGGCCTTTGCCA
GCATACCATCCAGAAGATATCAACCCTGCATCTTGGCTGCCGCGCTGTCAGGAGA
GCGATCAGCTTGCATGCCGGTCGATCTAGTAACATAGATGACACCGCGCGCGATA
ATTTATCCTAGTTTGCGCGCTATATTTTGTTTTCTATCGCGTATTAAATGTATAATT
GCGGGACTCTAATCATAAAAACCCATCTCATAAATAACGTCATGCATTACATGTT
AATTATTACATGCTTAACGTAATTCAACAGAAATTATATGATAATCATTGCAAGAC
CGGCAACAGGATTCAATCTTAAGAAACTTTATTGCCAAATGTTTGAACGATCTGCT
TGACTCTAGCTAGAGTCCGAACCCCAGAGTCCCGCTCAGAAGAACTCGTCAAGAA
GGCGATAGAAGGCTATGCGCTGCGAATCGGGAGCGGCGATACCGTAAAGCACGA
GGAAGCGGTCAGCCCATTCGCCGCCAAGCTCTTCAGCAATATCACGGGTAGCCAA
CGCTATGTCCTGATAGCGGTCCGCCACACCCAGCCGGCCACAGTCTCCAGATGAA
GAAAAGCGGCCATTTTCCACCATGATATTTCGGCAAGCAGGCGTGGCCGTGGGTCA
CGACGAGATCCTCGCCGTCGGGCATCCGCGCCTTGAGCCTGGCGAACAGTTCGGC
TGGCGCGAGCCCCTGATGCTCTTCGTCCAGATCATCCTGATCGACAAGACCGGCTT
CCATCCGAGTACGTGCTCGCTCGATTCGATGTTTCGCTTGGTGGTCGAATGGGCAG
GTAGCCGGATCAAGCGTATGCAGCCGCCGCATTGCATCAGCCATGATGGATACTT
TCTCGGCAGGAGCAAGGTGAGATGACAGGAGATCCTGCCCCGGCACTTCGCCCAA
TAGCAGCCAGTCCCTTCCCGCTTCAGTGACAACGTCGAGCACAGCTGCGCAAGGA
ACGCCCGTCGTGGCCAGCCACGATAGCCGCGCTGCCTCGTCTTGGAGTGTATTCA
GGGCACCGGACAGGTCGGTCTTGACAAAAAGAACCGGGCGCCCCTGCGCTGACA
GCCGGAACACGGCGGCATCAGAGCAGCCGATTGTCTGTTGTGCCCAGTCATAGCC
GAATAGCCTCTCCACCCAAGCGGCCGGAGAACCTGCGTGCAATCCATCTTGTTCA
ATCATGCCTCGATCGAGTTGAGAGTGAATATGAGACTCTAATTGGATACCGAGGG
GAATTTATGGAACGTCAGTGGAGCATTTTTGACAAGAAATATTTGCTAGCTGATA
GTGACCTTAGGCGACTTTTGAACGCGCAATANTTGGTTTCTGACGTATGTGCTTAGC
TCATTAAACTCCAGAAACCCGCGGCTGAGTGGCTCCTTCAACGTTGCGGTTCTGTC
AGTTCCAAACGTAAAACGGCTTGTCCCGCGTCATCGGCGGGGGTCATAACGTGAC
TCCCTTAATTCTCATGTATCTCCGTCAGGAGGTCAACTACCCCAATTTAAATTTTAT
TTGATTAAGATATTTTTATGGACCTACTTTATAATTAAAAATATTTTCTATTTGAAA
AGGAAGGACAAAAATCATACAATTTTGGTCCAACTACTCCTCTTCTTTTTTTTTTTG
GCTTTATAAAAAAGGAAAGTGATTAGTAATAAATAATTAAATAATGAAAAAAGG
AGGAAATAAAATTTTCGAATTAAAATGTAAAAGAGAAAAAGGAGAGGGAGTAAT
CATTGTTTAACTTTGTCTAAAGTACCCCAATTCGATTTTACATGTATATCAAATTAT
ACAAATATTTTATTAAAATATAGATATTGAATAATTTTATTATTCTTGAACATGTA
AATAAAAATTATCTATTATTTCAATTTTTATATAAACTATTATTTGAAATCTCAATT
ATGATTTTTTAATATCACTTTCTATCCATGATNATTTCAGCTTAAAAAGTTTTGTCA
ATAATTACATTAATTTTGTTGATGAGGATGACAAGATTTCGGTCATCAATTACATA
TACACAAATTGAAATAGTAAGCAACTTGATTTTTTTTCTCATAATGATAATGACAA
AGACACGAAAAGACAATTCAATATTCACATTGATTTATTTTTATATGATAATAATT
ACAATAATAATATTCTTATAAAGAAAGAGATCAATTTTGACTGATCCAAAAATTT
ATTTATTTTTACTATACCAACGTCACTAATTATATCTAATAATGTAAAACAATTCA
ATCTTACTTAAATATTAATTTGAAATAAACTATTTTTATAACGAAATTACTAAATT
TATCCAATAACAAAAAGGTCTFAAGAAGACATAAATTCTTTTTTTGTAATGCTCAA
ATAAATTTGAGTAAAAAAGAATGAAATTGAGTGATTTTTTTTTTAATCATAAGAAA
ATAAATAATTAATTTCAATATAATAAAACAGTAATATAATTTCATAAATGGAATTC
AATACTTACCTCTTAGATATAAAAAATAAATATAAAAATAAAGTGTTTCTAATAA
ACCCGCAATTTAAATAAAATATTTAATATTTTCAATCAAATTTAAATAATTATATT
AAAATATCGTAGAAAAAGAGCAATATATAATACAAGAAAGAAGATTTAAGTACA
ATTATCAACTATTATTATACTCTAATTTTGTTATATTTAATTCTTACGGTTAAGGT
CATGTTCACGATAAACTCAAAATACGCTGTATGAGGACATATTTTAAATTTTAACC
AATAATAAAACTAAGTTATTTTTAGTATATTTTTTTGTTTAACGTGACTTAATTTTT
CTTTTCTAGAGGAGCGTGTAAGTGTCAACCTCATTCTCCTAATTTTCCCAACCACA
TAAAAAAAAAATAAAGGTAGCTTTTGCGTGTTGATTTGGTACACTACACGTCATT
ATTACACGTGTTTTCGTATGATTGGTTAATCCATGAGGCGGTTTCCTCTAGAGTCG
GCCATACCATCTATAAAATAAAGCTTTCTGCAGCTCATTTTTTGATCTTCTATCTGA
TTTCTATTATAATTTCTCTGAATTGCCTTCAAATTTCTCTTTCAAGGTTAGAATTTT
TCTCTATTTTTTGGTTTTTGTTTGTTTAGATTCTGAGTTTAGTTAATCAGGTGCTGTT
AAAGCCCTAAATTTTGAGTTTTTTTCGGTTGTTTTGATGGAAAATACCTAACAATT
GAGTTTTTTCATGTTGTTTTGTCGGAGAATGCCTACAATTGGAGTTCCTTTCGTTGT
TTTGATGAGAAAGCCCCTAATTTGAGTGTTTTTCCGTCGATTTGATTTTAAAGGTTT
ATATTCGAGTTTTTTTCGTCGGTTTNATGAGAAGGCCTAAAATAGGAGTTTTTCTG
GTTGATTTGACTAAAAAAGCCATGGAATTTTGTGTTTTTGATGTCGCTTTGGTTCTC
AAGGCCTAAGATCTGAGTTTCTCCGGTTGTTTTGATGAAAAAGCCCTAAAATTGG
AGTTTTTATCTTGTGTTTTAGGTTGTTTTAATCCTTATAATTTGAGTTTTTTCGTTGT
TCTGATTGTTGTTTTTATGAATTTTGCAGAATGGATCATTATCTTGATATTAGACTT
AGACCTGATCCAGAATTTCCACCAGCTCAACTTATGTCTGTTCTTTTTGGAAAACT
TCATCAAGCTCTTGTTGCTCAAGGAGGAGATAGAATTGGAGYTTCTTTTCCTGATC
TTGATGAATCAAGATCAAGACTTGGAGAAAGACTTAGAATTCATGCTTCTGCTGA
TGATCTTAGAGCTTTGCTTGCTAGACCTTGGCTTGAAGGACTTAGAGATCATCTTC
AATTTGGAGAACCAGCTGTTGTTCCACATCCAACTCCTTATAGACAAGTTTCAAGA
GTTCAAGCTAAATCTAATCCAGAAAGACTTAGAAGAAGACTTATGAGAAGACATG
ATCTTTCTGAAGAAGAAGCTAGAAAAAGAATTCCTGATACTGTTGCTAGAGCTTT
GGATTTGCCTTTTGTTACACTTAGATCACAATCTACTGGACAACATTTTAGACTTTT
TATTAGACATGGACCACTTCAAGTTACTGCTGAAGAAGGAGGATTTACTTGTTATG
GACTTTCTAAGGGAGGTTTTGTTCCTTGGTTTGGATCTGGAGCTACTAATTTTTCTC
TTCTTAAGCAAGCTGGAGATGTTGAAGAAAATCCTGGACCCATGATGGATCCCCG
GGATCATCTACTTCTGAAGACTCAGACTCAGACTAAGCAGGTGACGAACGTCACC
AATCCCAATTCGATCTACATCGATAAGAAGTACTCTATCGGACTCGATATCGGAA
CTAACTCTGTGGGATGGGCTGTGATCACCGATGAGTACAAGGTGCCATCTAAGAA
GTTCAAGGTTCTCGGAAACACCGATAGGCACTCTATCAAGAAAAACCTTATCGGT
GCTCTCCTCTTAGATTCTGGTGAAACTGCTGAGGCTACCAGAGTCAAGAGAACCG
CTAGAAGAAGGTACACCAGAAGAAAGAACAGGATCTGCTACCTCCAAGAGATCT
TCTCTAACGAGATGGCTAAAGTGGATGATTCATTCTTCCACAGGCTCGAAGAGTC
ATTCCTCGTGGAAGAAGATAAGAAGCACGAGAGGCACCCTATCTTCGGAAACATTC
GTTGATGAGGTGGCATACCACGAGAAGTACCCTACTATCTACCACCTCAGAAAGA
AGCTCGTTGATTCTACTGATAAGGCTGATCTCAGGCTCATATACCTCGCTCTCGCT
CACATGATCAAGTTCAGAGGACACTTCCTCATCGAGGGTGATCTCAACCCTGATA
ACTCTGATGTGGATAAGTTGTTCATCCAGCTCGTGCAGACCTACAACCAGCTTTTC
GAAGAGAACCCTATCAACGCTTCAGGTGTGGATGCTAAGGCTATCCTCTCTGCTA
GGCTCTCTAAGTCAAGAAGGCTTGAGAACCTCATTGCTCAGCTCCCTGGTGAGAA
GAAGAACGGACTTTTCGGAAACTTGATCGCTCTCTCTCTCGGACTCACCCCTAACT
TCAAGTCTAACTTCGATCTCGCTGAGGATGCAAAGCTCCAGCTCTCAAAGGATAC
CTACGATGATGATCTCGATAACCTCCTCGCTCAGATCGGAGATCAGTACGCTGATT
TGTTCCTCGCTGCTAAGAACCTCTCTGATGCTATCCTCCTCAGTGATATCCTCAGA
GTGAACACCGAGATCACCAAGGCGCCACTCTCAGCTTCTATGATCAAGAGATACG
ATGAGCACCACCAGGATCTCACACTTCTCAAGGCTCTTGTTAGACAGCAGCTCCC
AGAGAAGTACAAAGAGATTTTCTTCGATCAGTCTAAGAACGGATACGCTGGTTAC
ATCGATGGTGGTGCATCTCAAGAAGAGTTCTACAAGTTCATCAAGCCTATCCTCG
AGAAGATGGATGGAACCGAGGAACTCCTCGTGAAGCTCAATAGAGAGGATCTTCT
CAGAAAGCAGAGGACCTTCGATAACGGATCTATCCCTCATCAGATCCACCTCGGA
GAGTTGCACGCTATCCTTAGAAGGCAAGAGGATTTCTACCGATTCCTCAAGGATA
ACAGGGAAAAGATTGAGAAGATTCTCACCTTCAGAATCCCTTACTACGTGGGACC
TCTCGCTAGAGGAAACTCAAGATTCGCTTGGATGACCAGAAAGTCTGAGGAAACC
ATCACCCCTTGGAACTTCGAAGAGGTGGTGGATAAGGGTGCTAGTGCTCAGTCTT
TCATCGAGAGGATGACCAACTTCGATAAGAACCTTCCAAACGAGAAGGTGCTCCC
TAAGGACTCTTTGCTCTACGAGTACTTGACCGTGTACAACGAGTTGACCAAGGTTA
AGTACGTGACCGAGGGAATGAGGAAGCCTGCTTTTTTGTCAGGTGAGCAAAAGAA
GGCTATCGTTGATCTCTTGTTCAAGACCAACAGAAAGGTGACCGTGAAGCAGCTC
AAAGAGGATTACTTCAAGAAAATCGAGTGCTTCGATTCAGTTGAGATTTCTGGTG
TTGAGGATAGGTTCAACGCATCTCTCGGAACCTACCACGATCTCCTCAAGATCATT
AAGGATAAGGATTTCTTGGATAACGAGGAAGAACGAGGATATCTTGGAGGATATCG
TTCTTACCCTGACCCTCTTTGAAGATAGAGAGATGATTGAAGAAAGGCTCAAGAC
CTACGCTCATGTCTTCGATGATAAGGTGATGAAGCAGTTGAAGAGAAGAAGATAC
ACTGGTTGGGGAAGGCTCTCAAGAAAGCTCATTAACGGAATCAGGGATAAGCAGT
CTGGAAAGACAATCCTTGATTTCCTCAAGTCTGATGGATTCGCTAACAGAAACTTC
ATGCAGCTCATCCACGATGATTCTCTCACCTTTAAAGAGGATATCCAGAAGGCTC
AGGTTTCAGGACAGGGTGATAGTCTCCATGAGCATATCGCTAACCTCGCTGGATC
TCCTGCAATCAAGAAGGGAATCCTCCAGACTGTGAAGGTTGTGGATGAGTTGGTG
AAGGTGATGGGAAGGCATAAGCCTGAGAACATCGTGATCGAAATGGCTAGAGAG
AACCAGACCACTCAGAAGGGACAGAAGAACTCTAGGGAAAGGATGAAGAGGATC
GAGGAAGGTATCAAAGAGCTTGGATCTCAGATCCTCAAAGAGCACCCTGTTGAGA
ACACTCAGCTCCAGAATGAGAAGCTCTACCTCTACTACCTCCAGAACGGAAGGGA
TATGTATGTGGATCAAGAGTTGGATATCAACAGGCTCTCTGATTACGATGTTGATC
ATATCGTGCCACAGTCATTCTTGAAGGATGATTCTATCGATAACAAGGTGCTCACC
AGGTCTGATAAGAACAGGGGTAAGAGTGATAACGTGCCAAGTGAAGAGGTTGTG
AAGAAAATGAAGAACTATTGGAGGCAGCTCCTCAACGCTAAGCTCATCACTCAGA
GAAAGTTCGATAACTTGACTAAGGCTGAGAGGGGAGGACTCTCTGAATTGGATAA
GGCAGGATTCATCAAGAGGCAGCTTGTGGAAACCAGGCAGATCACTAAGCACGTT
GCACAGATCCTCGATTCTAGGATGAACACCAAGTACGATGAGAACGATAAGTTGA
TCAGGGAAGTGAAGGTTATCACCCTCAAGTCAAAGCTCGTGTCTGATTTCAGAAA
GGATTTCCAATTCTACAAGGTGAGGGAAATCAACAACTACCACCACGCTCACGAT
GCTTACCTTAACGCTGTTGTTGGAACCGCTCTCATCAAGAAGTATCCTAAGCTCGA
GTCAGAGTTCGTGTACGGTGATTACAAGGTGTACGATGTGAGGAAGATGATCGCT
AAGTCTGAGCAAGAGATCGGAAAGGCTACCGCTAAGTATTTCTTCTACTCTATAACA
TCATGAATTTCTTCAAGACCGAGATTACCCTCGCTAACGGTGAGATCAGAAAGAG
GCCACTCATCGAGACAAACGGTGAAACAGGTGAGATCGTGTGGGATAAGGGAAG
GGATTTCGCTACCGTTAGAAAGGTGCTCTCTATGCCACAGGTGAACATCGTTAAG
AAAACCGAGGTGCAGACCGGTGGATTCTCTAAAGAGTCTATCCTCCCTAAGAGGA
ACTCTGATAAGCTCATTGCTAGGAAGAAGGATTGGGACCCTAAGAAATACGGTGG
TTTCGATTCTCCTACCGTGGCTTACTCTGTTTTCGTTGTGGCTAAGGTTGAGAAGG
GAAAGAGTAAGAAGCTCAAGTCTGTTAAGGAACTTCTCGGAATCACTATCATGGA
AAGGTCATCTTTCGAGAAGAACCCAATCGATTTCCTCGAGGCTAAGGGATACAAA
GAGGTTAAGAAGGATCTCATCATCAAGCTCCCAAAGTACTCACTCTTCGAACTCG
AGAACGGTAGAAAGAGGATGCTCGCTTCTGCTGGTGAGCTTCAAAAGGGAAACG
AGCTTGCTCTCCCATCTAAGTACGTTAACTTTCGTACCTCGCTTCTCACTACGAGA
AGFTGAAGGGATCTCCAGAAGATAACGAGCAGAAGCAACTATTCGTTGAGCAGCA
CAAGCACTACTTGGATGAGATCATCGAGCAGATCTCTGAGTTCTCTAAAAGGGTG
ATCCTCGCTGATGCAAACCTCGATAAGGTGTTGTCTGCTTACAACAAGCACAGAG
ATAAGCCTATCAGGGAACAGGCAGAGAACATCATCCATCTCTTCACCCTTACCAA
CCTCGGTGCTCCTGCTGCTTTCAAGTACTTCGATACAACCATCGATAGGAAGAGAT
ACACCTCTACCAAAGAATTGCTCGATGCTACCCTCATCCATCAGTCTATCACTGGA
CTCTACGAGACTAGGATCGATCTCTCACAGCTCGGTGGTGATTCAAGGGCTGATC
CTAAGAAGAAGAGGAAGGTTTGAGCTTGTTGTGGTTGTCTGGTTGCGTCTGTTGCC
CGTTGTCTGTTGCCCATTGTGGTGGTTGTGTTTGTATGATGGTCGTTAAGGATCAT
CAATGTGTTTTCGCTTTTTGTTCCATTCTGTTTCTCATTTGTGAATAATAATGGTAT
CTTTATGAATATGCAGTTTGTGGTTTCTTTTCTGATTGCAGTTCTGAGCATTTTGTT
TTTGCTTCCGTTTACTATACCACTTACAGTTTGCACTAATTTAGTTGATATGCGAGC
CATCTGATGTTTGATGATTCAAATGGCGTTTATGTAACTCGTACCCGAGTGGATGG
AGAAGAGCTCCATTGCCGGTTTGTTTCATGGGTGGCGGAGGGCAACTCCTGGGAA
GGAACAAAAGAAAAACCGTGATACGAGTTCATGGGTGAGAGCTCCAGCTTGATCC
CTTCTCTGTCGATCAAATTTGAATTTTTGGATCACGGCAGGCTCACAAGATAATCC
AAAGTAAAACATAATGAATAGTACTTCTCAATGATCACTTATTTTTAGCAAATCAG
CAATTGTGCATGTCAAATGATTTCGGTGTAAGAGAAAGAGTTGATGAATCAAAAT
ATCTGTAGCTGGATCAAGAATCTGAGGCAGTTGTATGTATCAATGATCTTTCCGCT
ACAATGATGTTAGCTATCCGAGTCAAATTGTTGTAGAATTGCGTACTTCGGCATCA
CATTCTGGATGACATAATAAATAGGAAGTCTTCAGATCCCTAAAAAATTGAGAGC
TAATAACATTAGTCCTAGATGTAACTGGGTGACAACCAAGAAAGAGACATGCAAA
TACTACTTTTGTTTGAAGGAGCATCCCTGGTTTGACATATTTTTTCTGAATATCAAA
CTTTGAAACTCTACCTAGTCTAATGTCTAACGCAGATCTTACTGGTTTAACTGCA
GTGATATCTACTATCTTTTGGAATGTTTTCTCCTTCAGTTATACATCAAGTTCCAAG
ATGCAGGTGTGCTTGATTGATGTACATGGCTGTGAGAAGTGCATCCTGATGTTCAG
ATGATGGTTCATTCTAATGTCTTTTCCTTCAATCAGTTTTCTCAGTCTGACTTAGCT
TGTTTCATCTGCATGTTTGAATGTTCGTTTACTCATAGTAATTGCATTTTTGTAGCA
GAACATATCATTGGTCATGGTTTCAACTGTGCGCGAGTCTTATGCTTATTCAAACT
AGGAAAGCCTCCGTCTAGAGGGTACACGAGTTGTTGCTCTGTGTGCGTCAGTCCA
TAGTATTAATCTTGCTAGTTGTAGTATATTGTTTATGTGGACTCGGAATTCATCAT
ATGCTCCTTCTTTGCATCAAGTAAGGCAAGGTAATGTATAGAAGCTTTTTAACTCT
TTCATGGAAGCTGGCCTTTGCCAGCATACCATCCAGAAGATATCACCCTGCATCT
TGGCTGCCGCGCTGTCAGGAGTCTCAATGGTAACTTTACTCTTTATTTAACCATAC
ATTTTTTTTTATTTTTTTCACTTTGTTCTTCATCCACTATTGTTCTTTGTTCATCTTGA
ACAAAAGCTCCCTCCTTCTTTGTTCTTCATCCACCATTGTTCTTCATCAATCATTTC
GCTGTCAGGAGACTAGAGCCAAGCTGATCTCCTTTGCCCCGGAGATCACCATGGA
CGACTTTCTCTATCTCTACGATCTAGGAAGAAAGTTCGACGGAGAAGGTGACGAT
ACCATGTTCACCACCGATAATGAGAAGATTAGCCTCTTCAATTTCAGAAAGAATG
CTGACCCACAGATGGTTAGAGAGGCCTACGCGGCAGGTATGATCAAGACGATCTA
CCCGAGTAATAATCTCCAGGAGATCAAATACCTTCCCAAGAAGGTTAAAGATGCA
GTCAAAAGATTCAGGACTAACTGCATCAAGAACACAGAGAAAGATATATTTCTCA
AGATCAGAAGTACTATTACAGTATGGACGATFCAAGGCTTGCTTCATAAACCAAG
GCAAGTAATAGAGATTGGAGTCTCTAAGAAAGTAGTTCCTACTGAATCAAAGGCC
ATGGAGTCAAAAATTCAGATCGAGGATCTAACAGAACTCGCCGTGAAGACTGGCG
AACAGTTCATACAGAGTCTATTACGACTCAATGACAAGAAGAAAATCTTCGTCAA
CATGGTGGAGCACGACACTCTCGTCTACTCCAAGAATATCAAAGATACAGTCTCA
GAAGACCAAAGGGCTATTGAGACTTTTCAACAAAGGGTAATATCGGGAAACCTCC
TCGGATTCCATTGCCCAGCTATCTGTCACTTCATCAAAAGGACAGTAGAAAAGGA
AGGTGGCACCTACAAATGCCATCACGCGATAAAGGAAAGGCTATCGTTCAAGAT
GCCCCTGCCGACAGTGGTCCCAAAGATGGACCCCCACCCACGAGGAGCATCGTGG
AAAAAGAAGACCTTTCCAACCACGTCTTCAAAGCAAGTGGATTGATGTGATATCTC
CACTGACGTAAGGGATGACGCACAATCCCACTATCCTTCGCAAGACCCTTCCTCT
ATATAAGGAAGTTCATTTCATTTGGAGAGGACTCCGGTATTTTTACAACAATTACC
ACAACAAAACAAACAACAAACAACATTACAATTTACTATTCTAGTCGAAATGGAT
CTGACTAGTCCTGCAGGTTCACTGCCGTATAGGCAGTATACGGTTATCCGGTTTGA
GTTTTAGAGCTAGAAATAGCAAGATAAAATAAGGCTAGTCCGTTATCAACTTGAA
AAAGTGGCACCGAGTCGGTGCGTTCACTGCCGTATAGGCAGCGACAAGAGTAGCA
AGCAAAGTTTTAGAGCTAGAAATAGCAAGTTAAAATAAGCTCTAGTCCGTTATCAA
CTTGAAAAAGTGGCACCGAGTCGGTGCGTTCACTGCCGTATAGGCAGCGGTTCCC
ATTACTGTTGCTGTTTTAGAGCTAGAAATAGCAAGTTAAAATAAGGCTAGTCCGTT
ATCAACTTGAAAAAGTGGCACCGAGTCGGTGCGTTCACTGCCGTATAGGCAGTTA
GAGCTTCTGAAGTAGAAGTTTTAGAGCTAGAAATAGCAAGTTAAAATAAGGCTAG
TCCGTTATCAACTTGAAAAAGTGGCACCGAGTCGGTGCGTTCACTGCCGTATAGG
CACTTTGAGTTGGCCAACAGTGAAGATTTAGAGCTAGAAATAGCAAGTTAAAATAA
GGCTAGTCCGTTATCAACTTGAAAAAGTGGCACCGAGTCGGTGCGTTCACTGCCG
TATAGGCAGAGTGCTAGCGGCGTAAGGAAGTTTTAGAGCTAGAAATAGCAAGTTA
AAATAAGGCTAGTCCGTTATCAACTTGAAAAAGTGGCACCGAGTCGGTGCGTTCA
CTGCCGTATAGGCAGAGAGGGCAACACCGGCACACGTTTTAGAGCTAGAAATAGC
AAGTTAAAATAAGGCTAGTCCGTTATCAACTTGAAAAAGTGGCACCGAGTCGGTG
CGTTCACTGCTTCGTATAGGCAGCACCGCGTTGAGTCCGAAGGGTTTTAGAGCTA
GAAATAGCAAGTTAAAATAAGGCTAGTCCGTTATCAACTTGAAAAAGTGGCACCG
AGTCGGTGCGTTCACTGCCGTATAGGCAGTCGTTGCAACCTCCTTAAGGGTTTTAG
AGCTAGNAATAGCAAGTTAAAATAAGGCTAGTCCGTTATCAACTTGAAAAAGTGG
CACCGAGTCGGTGCGTTCACTGCCGTATACGGCAGGTGGGGGAGAAGGATTGTGTT
GTTTTAGAGCTAGAAATAGCAAGTTAAAATAAGGCTAGTCCGTTATCAACTTGAA.
AAAGTGGCACCGAGTCGGTGCGTTCACTGCCGTATAGGCAGAATAGATTGGCCAT
GCAATGGTTTTAGAGCTAGAAATAGCAAGTTAAAATAAGGCTAGTCCGTTATCAA
CTTGAAAAAGTGGCACCGAGTCGGTGCGTTCACTGCCGTATAGGCAGGAAGTTTA
TGCGAATTTATGGTTTTAGAGCTAGAAATAGCAAGTTAAAATAAGGCTAGTCCGT
TATCAACTTGAAAAACTTGGCACCGAGTCGGTGCGTTCACTGCCGTATAGGCAGTC
GATCGACAAGGGTACCTAGGCTTCGGCCATGCTAGAGTCCGCAAAAATCACCAGT
CTCTCTCTACAAATCTATCTCTCTCTATTTTTCTCCAGAATAATGTGTGAGTAGTTC
CCAGATAAGGGAATTAGGGTTCTTATAGGGTTCGCTCATGTGTTGAGCATATAA
GAAACCCTTAGTATGTATTTGTATTTGTAAAATACTTCTATCAATAAAATTTCTAA
TTCCTAAAACCAAAATCCAGTGACCTCGCTGTCATGAGACGAATTCTGACAGGAT
ATATTGGCGGGTAAACCTAAGAGAAAAGAGCGTTTATTAGAATAATCGGATATTT
AAAAGGGCGTGAAAAGGTTTATCCGTTCGTCCATTTGTATGTGCATGCCAACCAC
AGGGTTCCCCTCGGGATCAAAGTACTTTGATCCAACCCCTCCGCGTCTATAGTGCA
GTCGGCTTCTGACGTTCAGTGCAGCCGTCATCTGAAAACGACATGTCGCACAAGT
CCTAAGTTACGCGACAGGCTGCCGCCCTGCCCTTTTCCTGGCGTTTTCTTGTCGCG
TGTTTTAGTCGCATAAAGTAGAATACTTGCGACTAGAACCGGAGACATTACGCCA
TGAACAAGAGCGCCGCCGCTGGCCTGCTGGGCTATGCCCGCGTCAGCACCGACGA
CCAGGACTTGACCAACCAACGGGCCGAACTGCACGCGGCCGGCTGCACCAAGCT
GTTTTCCGAGAAGATCACCGGCACCAGGCGCGACCGCCCGGAGCTGGCCAGGATG
CTTGACCACCTACGCCCTGGCGACGTTGTGACAGTGACCAGGCTAGACCGCCTGG
CCCGCAGCACCCGCGACCTACTGGACATTGCCGAGCGCATCCAGGAGGCCGGCGC
GGGCCTGCGTAGCCTGGCAGAGCCGTGGGCCGACACCACCACGCCGGCCGGCCG
CATGGTGTTGACCGTGTTCGCCGGCATTGCCGAGTTCGAGCGTTCCCTAATCATCG
ACCGCACCCGGAGCGGGCGCGAGGCCGCCAAGGCCCGAGGCGTGAAGTTTGGCC
CCCGCCCTACCCTCACCCCGGCACAGATCGCGCACGCCCGCGAGCTGATCGACCA
GGAAGGCCGCACCGTGAAAGAGGCGGCTGCACTGCTTGGCGTGCATCGCTCGACC
CTGTACCGCGCACTTGAGCGCAGCGAGGAAGTGACGCCCACCGAGGCCAGGCGG
CGCGGTGCCTTCCGTGAGGACGCATTGACCGAGGCCGACGCCCTGGCGGCCGCCG
AGAATGAACGCCAAGAGGAACAAGCATGAAACCGCACCAGGACGGCCAGGACG
AACCGTTTTTCATTACCGAAGAGATCGAGGCGGAGATGATCGCGGCCGGGTACGT
GTTCGAGCCGCCCGCGCACCTCTCAACCGTGCGGCTGCATGAAATCCTGGCCGGT
TTGTCTGATGCCAAGCTGGCGGCCTGGCCGGCCAGCTTGGCCGCTGAAGAAACCG
AGCGCCGCCGTCTAAAAAGGTGATGTGTATTTGAGTAAAACAGCTTGCGTCATGC
GGTCGCTGCGTATATGATCCGATGAGTAAATAAACAAATACGCAAGGGGAACGC
ATGAACTTTTATCGCTGTACTAACCAGAAAGGCGGGTCAGGCAAGACGACCAGCG
GAACCCATCTAGCCCGCGCCCTGCAACTCGCCGGGGCCGATGTTCTGTTAGTCGA
TTCCGATCCCCAGGGCAGTGCCCGCGATTGGGCGGCCGTGCGGGAAGATCAACCG
CTAACCGTTGTCGGCATCGACCGCCCGACGATTGACCGCGACGTGAAGGCCATCG
GCCGGCGCGACTTCGTAGTGATCGACGGAGCGCCCCAGGCGGCGGACTTGGCTGT
GTCCGCGATCAAGGCAGCCGACTTCGTGCTGATTCCGGTGCAGCCAAGCCCTTAC
GACATATGGGCCACCGCCGACCTGGTGGAGCTGGTTAAGCAGCGCATTGAGGTCA
CGGATGGAAGGCTACAAGCGGCCTTTGTCGTGTCGCGGGCGATCAAAGGCACGCG
CATCGGCGGTGAGGTTGCCGAGGCGCCGGCCGGGTACGAGCTGCCCATTCTTGAG
TCCCGTATCACGCAGCGCGTGAGCTACCCAGGCACTGCCGCCGCCGGCACAACCG
TTCTTGAATCAGAACCCGAGGGCGACGCTGCCCGCGAGGTCCAGGCGCTGGCCGC
TGAAATTAAATCAAAACTCATTTGAGTTAATGAGGTAAAGAGAAAAGTGAGCAAA
AGCACAAACACGCTAAGTGCCGGCCGTCCGAGCGCACGCAGCAGCAAGGCTGCA
ACGTTGGCCAGCCTGGCAGACACGCCAGCCATGAAGCGGGTCAACTTTCAGTTGC
CGGCGGAGGATCACACCAAGCTGAAGATGTACGCGGTACGCCAAGGCAAGACCA
TTACCGAGCTGCTATCTGAATAGATCGCGCAGCTACCAGAGTAAATGAGCAAATG
AATAAATGAGTAGATGAATTTTAGCGGCTAAAGGAGGCGGCATGGAAAATCAAG
AACAACCAGGCACCGACGCCGTGGAATGCCCCATGTGTGGAGGAACGGGCGGTT
GGCCAGGCGTAAGCGGCTGGGTTGTCTGCCGGCCCGCAATGGCACTGGAACCCC
CAAGCCCGAGGAATCGGCGTGACGGTCGCAAACCATCCGGCCCGGTACAAATCG
GCGCGGCGCTGGGTGATGACCTGGTGGAGAAGTTGAAGGCCGCGCAGGCCGCCC
AGCGGCAACGCATCGAGGCAGAAGCACGCCCCGGTGAATCGTGGCAAGCGGCCG
CTGATCGAATCCGCAAAGAATCCCGGCAACCGCCGGCAGCCGGTGCGCCGTCGAT
TAGGAAGCCGCCCAAGGGCGACGAGCAACCAGATTTTTTCGTTCCGATGCTCTAT
GACGTGGGCACCCGCGATAGTCGCAGCATCATGGACGTGGCCGTTTTCCGTCTGT
CGAAGCGTGACCGACGAGCTGGCGAGGTGATCCGCTACGAGCTTCCAGACGGGC
ACGTAGAGGTTTCCGCAGGGCCGGCCGGCATGGCCAGTGTGTGGGATTACGACCT
GGTACTGATGGCGGTTTCCCATCTAACCGAATCCATGAACCGATACCGGGAAGGG
AAGGGAGACAAGCCCGGCCGCGTGTTCCGTCCACACGTTGCGGACGTACTCAAGT
TCTGCCGGCGAGCCGATGGCGGAAAGCAGAAAGACGACCTGGTAGAAACCTGCA
TTCGGTTAAACACCACGCACGTTGCCATGCAGCGTACGAAGAAGGCCAAGAACG
GCCGCCTGGTGACGGTATCCGAGGGTGAAGCCTTGATTAGCCGCTACAAGATCGT
AAAGAGCGAAACCGGGCGGCCGGAGTACATCGAGGTCGAGCTAGCTGATTGGAT
GTACCGCGAGATCACAGAAGGCAAGAACCCGGACGTGCTGACGGTTCACCCCGA
TTACTTTTTGATCGATCCCGGCATCGGCCGTTTTCTCTACCGCCTGGCACGCCGCG
CCGCAGGCAAGGCAGAAGCCAGATGGTTGTTCAAGACGATCTACGAACGCAGTG
GCAGCGCCGGAGAGTTCAAGAAGTTCTGTTTCACCGTGCGCAAGCTGATCGGGTC
AAATGACCTGCCGGAGTACGATTTGAAGGAGGAGGCGGGGCAGGCTGGCCCGAT
CCTAGTCATGCGCTACCGCAACCTGATCGAGGGCGAAGCATCCGCCGGTTCCTAA
TGTACGGAGCAGATGCTAGGGCAAATTGCCCTAGCAGGGGAAAAAGGTCGAAAA
GGACTCTTTCCTGTGGATAGCACGTACATTGGGAACCCAAAGCCGTACATTGCGA
ACCGGAACCCGTACATTGGGAACCCAAAGCCGTACATTGGGAACCGGTCACACAT
GTAAGTGACTGATATAAAAGAGAAAAAAGGCGATTTTTCCGCCTAAAACTCTTTA
AAACTTATTAAAACTCTTAAAACCCGCCTGGCCTGTGCATAACTGTCTGGCCAGCG
CACAGCCGAAGAGCTGCAAAAAGCGCCTACCCTTCGGTCGCTGCGCTCCCTACGC
CCCGCCGCTTCGCGTCGGCCTATCGCGGCCGCTGGCCGCTCAAAAATGGCTGGCC
TACGGCCAGGCNATCTACCAGGGCGCGGACAAGCCGCGCCGTCGCCACTCGACCG
CCGGCGCCCACATCAAGGCACCCTGCCTCGCGCGTTTCGGTGATGACGGTGAAAA
CCTCTGACACATGCAGCTCCCGGTGACGGTCACAGCTTGTTCTTGTAAGCGGATGCC
GGGAGCAGACAAGCCCGTCAGGGCGCGTCAGCGGGTGTTGGCGGGTGTCGGGGC
GCAGCCATGACCCAGTCACGTAGCGATAGCGGAGTGTATACTGGCTTAACTATGC
GGCATCAGAGCAGATTGTACTGAGAGTGCACCATATGCGGTGTGAAATACCGCAC
AGATGCGTAAGGAGAAAATACCGCATCAGGCGCTCTTCCGCTTCCTCGCTCACTG
ACTCGCTGCGCTCGGTCGGCGCTGCGGCGAGCGGTATCAGCTCACTCAAAGGC
GGTAATACGGTTATCCACAGAATCAGGGGATAACGCAGGAAAGAACATGTGAGC
AAAAGGCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGT
AGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGGACAGT
ATTTGGTATCTGCGCTCTGCTGAAGCCAGTTACCTTCGGAAAAAGAGTTGGTAGCT
CTTGATCCGGCAAACAAACCACCGCTGGTAGCGGTGGTTTTTTTGTTTGCAAGCAG
CAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTGATGGATTC
TAGGTGATTAGAAAAACTCATCGAGCATCAAATGAAACTGCAATTTATTCATATC
AGGATTATCAATACCATATTTTTGAAAAAGCCGTTTCTCTAATGAAGGAGAAAAC
TCACCGAGGCAGTTCCATAGGATGGCAAGATCCTGGTATCGGTCTGCGATTCCGA
CTCGTCCAACATCAATACAACCTATTAATTTCCCCTCGTCAAAAATAAGGTTATCA
AGTGAGAAATCACCATGAGTGACGACTGAATCCGGTGAGAGGCAAAAGTTTAT
GCATTTCTTTCGAGACTTGTTCAACAGGCCAGCCATTACGCTCGTGATCAAAATCA
CTCGCATCAACCAAACCGTTATTCATTCGTGATTGCGCCTGACTCGACTTCGAAATAC
GCGATCGCTCTTTAAAAGGACAATTACAAACAGGAATCGAATGCAACCGGCGCAG
GAACACTGCCAGCGCATCAACAATATTTTCACCTGAATCAGGATATTCTTCTAATA
CGTGGAATGCTGTTTTCCCTGGTTATCGCACTTGTTTGAGTAACCATGCATCATCAGG
AGTACGGATAAAATGCTTGATGGTCGGAAGAGGCATAAATTCCGTGAGCCAGTTT
AGTCTGACCATCTCATCTGTAACATCATTGGCAACGCTACCTTTGCCATGTTTCAG
AAACAACTCTGGCGCATCGGGCTTCCCATACAATCGGTAGATTGTCGCACCTGATT
GCCCGACATTATCGCGAGCCCATTTATACCCATATAAATCAGGATCCATGTTGGAA
TTTAATCGCGGCCTTGAGCAAGACGTTTCCCGTTGAATATGGCTCATAACAGAACT
TATTATTTCCTTCCTCTTTTCTACAGTATTTAAAGATACCCCAAGAAGCTAATTATA
ACAAGACGAACTCCAATTCACTGTTCCTTGCATTCTAAAACCTTAAATACCAGAA
AACAGCTTTTTCAAAGTTGTTTTCAAAGTTGGCGTATAACATAGTATCGACGGAGC
CGATTTTGAAACCGCGGTGATCACAGGCAGCAACGCTCTGTCATCGTTACAATCA
ACATGCTACCCTCCGCGAGATCATCCGTGTTTCAAACCCGGCAGCTTAGTTGCCGT
TCTTCCGAATAGCATCGGTAACATGAGCAAAGTCTATCCGCCTTACAACGGCTCTC
CCGCTGACGCCGTCCCGGACTGATGGGCTGCCTGTATCGAGTGGTGATTTTGTGCC
GAGCTGCCGGTCGGGGAGCTGTTGGCTGGCTGGTGGCAGGATATATTGTGGTGTA
AACA (SEQ ID NO: 69)

### Discussion

Therefore, cow's milk proteins could be expressed in plants. As shown in Examples 1-3, the expression of these genes individually did not result in gross morphological abnormalities in the leaves of Nicotiana benthamiana nor did it result in robust changes in the protein expression profile of these plants.

In soybean plants, a vector is constructed to express these cow's milk proteins specifically in the soybean endosperm using a set of seed specific promotors, to avoid burdening vegetative tissues growth and preserve the crop yields. These promoters were selected to achieve similar proportions of protein expression of the seven cow's milk genes in soybean, as compared with cow's milk. Additionally, using CRISPR/CAS9, the expression of the eight allergenic proteins in the soybean will be knocked out, along with the three fatty acid desaturase genes to divert the fatty acid biosynthetic pathway of the soybean plant towards a more desirable fatty acid profile. By using these techniques, soybeans that produce mostly cow's milk proteins in a comparable proportion to that of cow's milk, with reduced allergenicity and with an improved fatty acid profile, can be engineered.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A genetically modified plant comprising at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source.

2. The genetically modified plant of claim 1, wherein the at least one cell further comprises:
(a) reduced expression of at least one globulin protein as compared to the expression thereof in a corresponding unmodified plant and wherein said at least one cell comprises a reduced content of the at least one globulin protein as compared to the content thereof in a corresponding unmodified plant; or
(b) reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant; or
(c) reduced expression of at least one seed storage protein as compared to the expression thereof in a corresponding unmodified plan and wherein said at least one cell comprises a reduced content of the at least one seed storage protein as compared to the content thereof in a corresponding unmodified plant; or
(d) a combination thereof.

3. The genetically modified plant of claim 2, wherein
a) said at least one globulin gene is selected from the group consisting of a gene encoding glycinin 1 (GY1), a gene encoding glycinin 2 (GY2), a gene encoding glycinin 3 (GY3), a gene encoding glycinin 4 (GLY4), a gene encoding glycinin 5 (GY5), a gene encoding alpha-conglycinin, a gene encoding alpha-prime-conglycinin, and a gene encoding beta-conglycinin; or
b) said at least one desaturase gene is selected from the group consisting of a gene encoding fatty acid desaturase 1A (FAD2-1A), a gene encoding fatty acid desaturase 1B (FAD2-1B), and a gene encoding delta-9-stearoyl-acyl-carrier protein desaturase (SACPD);
c) or a combination thereof.

4. The genetically modified plant of claim 2, wherein said at least one cell further comprises
(a) at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof;
(b) at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof; or
(c) at least one third series silencer targeted to a polynucleotide encoding at least one seed storage protein or a portion thereof; or
(d) or a combination thereof.

5. The genetically modified plant of claim 1, wherein the relative protein content of each of said at least two milk proteins is at least 70% of the relative protein content of the corresponding mammalian milk protein in the mammal's milk.

6. The genetically modified plant of claim 1, wherein said at least one cell comprises a seed, or a bean, grain, fruit, nut, legume, leaf, stem, or root cell.

7. The genetically modified plant of claim 1, wherein said at least two milk proteins are from a non-human mammal comprising a Bos taurus or Bubalus bubalis.

8. The genetically modified plant of claim 1, wherein
a) the amino acid sequence of the serum albumin is at least 90% c identical to the amino acid sequence set forth in SEQ ID NO: 36, or the polynucleotide sequence encoding the serum albumin is at least 90% identical to the polynucleotide sequence set forth in SEQ ID NO: 29;
b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
c) the amino acid sequence of the alpha-S2-casein is at least 90,% identical to the amino acid sequence set forth in SEQ ID NO: 38 or the polynucleotide sequence encoding the alpha-S2-casein is at least 90% identical to the polynucleotide sequence set forth in SEQ ID NO: 31;
d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

9. The genetically modified plant of claim 1, wherein said plant comprises
a) a Solanaceae family plant, a Fabaceae family plant, a Poaceae family plant, a Amaranthaceae family plant, a Lamiaceae family plant, a Pedaliaceae family plant, a Cucurbitaceae family plant, a Asteraceae family plant, a Linaceae family plant, a Cannabaceae family plant, a Juglandaceae family plant, a Rosaceae family plant, a Anacardiaceae family plant, a Betalaceae family plant, or a Aracaceae family plant;
b) an algal plant selected from the group consisting of a chlorophyte, a rhodophyte, and a phaeo-phyte; or
c) an algal plant wherein said alga is a C. reinhardtii.

10. The genetically modified plant of claim 9, wherein the plant is selected from
(a) the Cannabaceae family and is Cannabis sativa, Cannabis indica, or Cannabis ruderalis plant;
(b) the Solanaceae family and is a Nicotiana benthamiana plant;
(c) the Fabacea family and is a soybean plant (Glycine max);
(d) the Poaceae family and is an Asian rice (Oryza sativa) or an African rice (Oryza glaberrima) plant; or
(e) the Aracaceae family, Lemnoidea subfamily, and is duckweed.

11. The genetically modified plant of claim 1, wherein expression of each of said at least two milk proteins is independently under control of a seed promoter, wherein:
a) expression of beta-casein is under the control of Seed 1 promoter having a nucleotide sequence set forth in SEQ ID NO: 51;
b) expression of kappa-casein is under the control of Seed 2 promoter having a nucleotide sequence set forth in SEQ ID NO: 52;
c) expression of beta-lactoglobulin is under the control of Seed 2 promoter having a nucleotide sequence set forth in SEQ ID NO: 52;
d) expression of alpha-S2-casein is under the control of Seed 3 promoter having a nucleotide sequence set forth in SEQ ID NO: 53;
e) expression of alpha-S1-casein is under the control of Seed 4 promoter having a nucleotide sequence set forth in SEQ ID NO: 54;
f) expression of serum albumin is under the control of Seed 5 promoter having a nucleotide sequence set forth in SEQ ID NO: 55; and
g) expression of alpha-lactalbumin is under the control of Seed 6 promoter having a nucleotide sequence set forth in SEQ ID NO: 56).

12. A food, medicament, cosmetic or blocking composition comprising a genetically modified plant or a portion, product, isolate, exudate, secretion, or extract thereof, said genetically modified plant or portion, product, isolate, exudate, secretion, or extract thereof comprising at least one cell expressing at least two milk proteins from a mammal, the at least two milk proteins selected from the group consisting of serum albumin, alpha-S1-casein, alpha-S2-casein, beta-casein, kappa-casein, beta-lactoglobulin, and alpha-lactalbumin, wherein the amino acid sequence of each of said at least two proteins is at least 90% identical to the amino acid sequence of a corresponding mammalian milk protein from the same mammalian source.

13. The food, medicament, cosmetic or blocking composition of claim 12, wherein the at least one cell further comprises:
(a) reduced expression of at least one globulin protein as compared to the expression thereof in a corresponding unmodified plant and wherein said at least one cell comprises a reduced content of the at least one globulin protein as compared to the content thereof in a corresponding unmodified plant; or
(b) reduced expression of at least one desaturase protein as compared to the expression thereof in a corresponding unmodified plant, and wherein said at least one cell comprises an increased content of at least one oleic acid or derivative thereof or at least one stearic acid or derivative thereof, or a reduced content of at least one saturated fat, or any combination thereof, compared to the content thereof in a corresponding unmodified plant; or
(c) reduced expression of at least one seed storage protein as compared to the expression thereof in a corresponding unmodified plan and wherein said at least one cell comprises a reduced content of the at least one seed storage protein as compared to the content thereof in a corresponding unmodified plant; or
(d) a combination thereof.

14. The food, medicament, cosmetic or blocking composition of claim 13, wherein said at least one cell further comprises
(a) at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof;
(b) at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof; or
(c) at least one third series silencer targeted to a polynucleotide encoding at least one seed storage protein or a portion thereof; or
(d) a combination thereof.

15. The food, medicament, cosmetic or blocking composition of claim 12, wherein the relative protein content of each of said at least two milk proteins is at least 70% of the relative protein content of the corresponding mammalian milk protein in the mammal's milk.

16. The food, medicament, cosmetic or blocking composition of claim 12, wherein said at least one cell comprises a seed, or a bean, grain, fruit, nut, legume, leaf, stem, or root cell.

17. The food, medicament, cosmetic or blocking composition of claim 12, wherein
(a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
(d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
(e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

18. The food, medicament, cosmetic or blocking composition of claim 12, further comprising milk from a mammal for a final concentration of between 1%-60% milk from a mammal or further comprising an unmodified milk alternative from a plant.

19. A DNA binary vector or viral vector expressing at least two milk proteins from a mammal, the vector comprising:
(a) a selectable marker;
(b) polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under the control of a promoter, wherein the amino acid sequence of each of said at least two proteins is at least 90% identical to the amino acid sequence of a corresponding mammalian milk protein from the same mammalian source.

20. The DNA binary vector or viral vector of claim 19, wherein
(a) the amino acid sequence of the serum albumin is at least 90% identical to the amino acid sequence set forth in SEQ ID NO: 36, or the polynucleotide sequence encoding the serum albumin is at least 90% identical to the polynucleotide sequence set forth in SEQ ID NO: 29;
(b) the amino acid sequence of the alpha-S1-casein is at least 90% identical to the amino acid sequence set forth in SEQ ID NO: 37, or the polynucleotide sequence encoding the alpha-S1-casein is at least 90% identical to the polynucleotide sequence set forth in SEQ ID NO: 30;
(c) the amino acid sequence of the alpha-S2-casein is at least 90% identical to the amino acid sequence set forth in SEQ ID NO: 38 or the polynucleotide sequence encoding the alpha-S2-casein is at least 90% identical to the polynucleotide sequence set forth in SEQ ID NO: 31;
(d) the amino acid sequence of the beta-casein is at least 90% identical to the amino acid sequence set forth in SEQ ID NO: 39 or the polynucleotide sequence encoding the beta-casein is at least 90% identical to the polynucleotide sequence set forth in SEQ ID NO: 32;
(e) the amino acid sequence of the kappa-casein is at least 90% identical to the amino acid sequence set forth in SEQ ID NO: 40 or the polynucleotide sequence encoding the kappa-casein is at least 90% identical to the polynucleotide sequence set forth in SEQ ID NO: 33;
(f) the amino acid sequence of the beta-lactoglobulin is at least 90% identical to the amino acid sequence set forth in SEQ ID NO: 41 or the polynucleotide sequence encoding the beta-lactoglobulin is at least 90% identical to the polynucleotide sequence set forth in SEQ ID NO: 34; and
(g) the amino acid sequence of the alpha-lactalbumin is at least 90% identical to the amino acid sequence set forth in SEQ ID NO: 42 or the polynucleotide sequence encoding the alpha-lactalbumin is at least 90% identical to the polynucleotide sequence set forth in SEQ ID NO: 35.

21. The DNA binary vector or viral vector of claim 19, wherein expression of each of said at least two milk proteins is independently under control of a seed promoter, wherein
(a) expression of beta-casein is under the control of Seed 1 promoter having a nucleotide sequence set forth in SEQ ID NO: 51;
(b) expression of kappa-casein is under the control of Seed 2 promoter having a nucleotide sequence set forth in SEQ ID NO: 52;
(c) expression of beta-lactoglobulin is under the control of Seed 2 promoter having a nucleotide sequence set forth in SEQ ID NO: 52;
(d) expression of alpha-S2-casein is under the control of Seed 3 promoter having a nucleotide sequence set forth in SEQ ID NO: 53;
(e) expression of alpha-S1-casein is under the control of Seed 4 promoter having a nucleotide sequence set forth in SEQ ID NO: 54;
(f) expression of serum albumin is under the control of Seed 5 promoter having a nucleotide sequence set forth in SEQ ID NO: 55; and
(g) expression of alpha-lactalbumin is under the control of Seed 6 promoter having a nucleotide sequence set forth in SEQ ID NO: 56).

22. The DNA binary vector or viral vector of claim 19, further comprising a polynucleotide sequence comprising a silencing element under the control of a promotor targeted to at least one globulin gene; or at least one desaturase gene; or at least one seed storage protein; or a combination thereof.

23. The DNA binary vector or viral vector of claim 22, wherein said silencing element comprises
(a) at least one first series silencer targeted to a polynucleotide encoding at least one globulin protein or a portion thereof, selected from the group consisting of glycinin 1 (GY1) or a portion thereof, glycinin 2 (GY2) or a portion thereof, glycinin 3 (GY3) or a portion thereof, glycinin 4 (GLY4) or a portion thereof, glycinin 5 (GY5) or a portion thereof, alpha-conglycinin or a portion thereof, alpha-prime-conglycinin or a portion thereof, and beta-conglycinin or a portion thereof;
(b) at least one second series silencer targeted to a polynucleotide encoding at least one desaturase protein or a portion thereof selected from the group consisting of fatty acid desaturase 1A (FAD2-1A) or a portion thereof, fatty acid desaturase 1B (FAD2-1B) or a portion thereof, and a gene encoding delta-9-stearoyl-acyl-carrier protein desaturase (SACPD) or a portion thereof; or
(c) at least one third series silencer targeted to a polynucleotide encoding at least one seed storage protein or a portion thereof;
(d) or a combination thereof.

24. The DNA binary vector or viral vector of claim 19, wherein the selectable marker is a BASTA resistance marker.

25. The DNA binary vector or viral vector of claim 19, wherein said vector comprises a sequence at least 90% identical to the sequence set forth in SEQ ID NO: 50 or at least 90% identical to the sequence set forth in SEQ ID NO: 69.

26. A genetically modified plant cell comprising the vector of claim 19.

27. A method of producing a food, medicament, cosmetic or blocking composition comprising a genetically modified plant or portion, product, isolate, exudate, secretion, or extract thereof, the method comprising:
(a) providing a DNA binary vector or viral vector for differentially expressing in a plant, proteins from the milk of a mammal, the vector comprising:
(i) a selectable marker;
(ii) polynucleotide sequences encoding at least two milk proteins from a mammal, wherein said at least two milk proteins are selected from the group consisting of serum albumin, alpha-S1-casein, alpha-S2-casein, beta-casein, kappa-casein, beta-lactoglobulin, and alpha-lactalbumin, each independently under control of a promoter, wherein:
(1) wherein the amino acid sequence of each of said at least two proteins is at least 90% identical to the amino acid sequence of a corresponding mammalian milk protein from the same mammalian source; and
(2) wherein expression of each of said at least two milk proteins is independently under the control of a seed promoter for obtaining a relative protein content of each of said at least two milk proteins of at least 70% of the relative protein content of the corresponding mammalian milk protein in the mammal's milk;
(b) transfecting at least one cell of said plant with the DNA binary vector or viral vector;
(c) differentially expressing the at least two milk proteins in said at least one plant cell; and
(d) optionally adding milk of a mammal to the food, medicament, cosmetic or blocking composition of step (c).

28. The method of claim 27, wherein said polynucleotide sequences further comprises a polynucleotide sequence comprising a silencing element under the control of a promotor targeted to at least one globulin gene; at least one desaturase gene; or at least one seed storage protein; or a combination thereof.

29. The method of claim 28, wherein said vector comprises a sequence at least 90% identical to the sequence set forth in SEQ ID NO: 50 or at least 90% identical to the sequence set forth in SEQ ID NO: 69.
